(19) United States
(12) Patent Application Publication
(10) Pub. No.: US 2025/0213705 A1
(43) Pub. Date: Jul. 3, 2025

(54) PSMA TARGETING LIGANDS AND METHODS OF USE

(71) Applicant: Bright Peak Therapeutics AG, Allschwil (CH)

(72) Inventors: Vijaya Raghavan PATTABIRAMAN, Volketswil (CH); Bertolt KREFT, Kleinmachnow (DE); Grégory UPERT, Kembs (FR); Régis BOEHRINGER, Basel (CH); Valerio PIACENTI, Basel (CH)

(21) Appl. No.: 18/813,107

(22) Filed: Aug. 23, 2024

Related U.S. Application Data
(60) Provisional application No. 63/534,288, filed on Aug. 23, 2023.

(51) Int. Cl. A61K 47/64; A61K 47/54

(52) U.S. Cl. CPC A61K 47/64; A61K 47/542; A61K 47/545; A61K 47/547

(57) ABSTRACT

The present disclosure relates to ligands capable of binding to PSMA. Such ligands are useful for attaching to additional groups (e.g., payloads, such as proteins or radionuclides) in order to target the additional groups to cells expressing PSMA (e.g., prostate cancer cells).

### CROSS REFERENCE

This application claims the benefit of U.S. Provisional Application No. 63/534,288 filed Aug. 23, 2023, which application is incorporated herein by reference in their entirety.

### SEQUENCE LISTING

The instant application contains a Sequence Listing which has been submitted electronically in XML format and is hereby incorporated by reference in its entirety. Said XML copy, created on Aug. 21, 2024, is named 94917-0154_741201US_SL.xml and is 119,380 bytes in size.

### SUMMARY OF THE INVENTION

Provided herein ligands which target prostate-specific membrane antigen (PSMA). PSMA is a highly upregulated gene product in many prostate cancers. Given its upregulated status in prostate cancer and the generally low or non-detectable levels of it in other tissues, radiotherapies which utilize ligands which target PSMA have recently shown substantial promise as therapeutics. For example, Pluvicto® (Lutetium (¹⁷⁷Lu) vipivotide tetraxetan), which utilizes a PSMA targeting ligand to deliver a radioactive active ingredient to the cancer site, was approved for medical use in the United States and Europe in 2022. However, there exists a need for additional ligands which are capable of delivering other agents to the cancer site. For example, a PSMA targeting ligand with enhanced binding or affinity to PSMA could be particularly useful in the delivery of larger active molecules to the cancer site as lower quantities of these larger molecules (e.g., proteins or protein complexes) are able to be delivered as compared to the relatively small radionuclides of Pluvicto®. Moreover, a PSMA targeting ligand which could be readily and easily attached to other groups, such as via conjugation reactions, in order to allow for rapid generation of PSMA-targeted groups derived from other agents (e.g., drugs, therapeutic proteins, therapeutic nanoparticles, imaging agents, and the like).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Provided herein are PSMA targeting ligands with high affinity which are attached to additional groups or configured to be readily attached to additional groups (e.g., via a conjugation handle). In some embodiments, the PSMA targeting ligands comprise a urea-linked di-amino acid structure as a portion of the PSMA targeting ligand. Such structures are described in, for example, U.S. Pat. No. 10,039,845. Such structures have been incorporated into many PSMA-targeting compounds, including Pluvicto® (e.g., U.S. Pat. No. 10,398,791) and as described in, for example U.S. Pat. Nos. 10,046,054 and 10,975,089, U.S. Patent Application Publication Nos: US2021/0393809, US2020/0339625, US2021/0121585, US2022/0281852 and US2023/0201383, and Patent Cooperation Treaty (PCT) Publication Nos: WO2021080409 and WO2022253785. In some embodiments, the PSMA targeting ligands provided herein further comprise a peptide portion which, in some embodiments, allows for enhanced affinity of the PSMA targeting ligand compared to others known in the art. In some embodiments, this peptide portion forms the site of attachment to an additional group, optionally through a linker. In some embodiments, the peptide portion is capable of forming a helical structure (e.g., an alpha-helix).

In one aspect herein is a prostate-specific membrane antigen (PSMA) binding ligand having a structure of Formula (I):

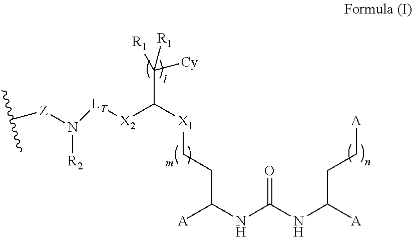

wherein:
each A is independently selected from carboxylic acid, sulphonic acid, phosphonic acid, tetrazole, or isoxazole;
m is an integer from 0 to 6;
n is an integer from 0 to 6;
l is an integer from 0 to 6;
Cy is optionally substituted cycloalkyl, optionally substituted heterocycloalkyl, optionally substituted aryl, or optionally substituted heteroaryl;
each of X₁ and X₂ is independently —N(R)C(═O)—, —C(═O)N(R)—, —C(═O)O—, —OC(═O)N(R)—, —N(R)C(═O)O—, or —N(R)C(═O)N(R)—;
L_{T} is optionally substituted C₁ to C₂₀ alkylene, optionally substituted C₁ to C₂₀ heteroalkylene,

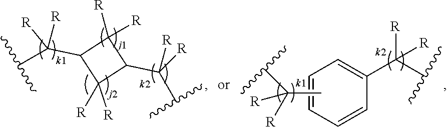

wherein
   k1 and k2 are each independently an integer from 0 to 6; and
   j1 and j2 are each independently an integer from 1 to 4;
Z is a peptide of at least two amino acids or a poly(ethylene glycol) group;
each R is independent selected from H and optionally substituted alkyl;
each R₁ and R₂ is independently selected from H and optionally substituted alkyl; and

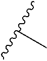

denotes a point of attachment to an additional group, optionally by a linker;
or a pharmaceutically acceptable salt, or solvate thereof.

In some embodiments, the ligand has a structure of Formula (Ia):

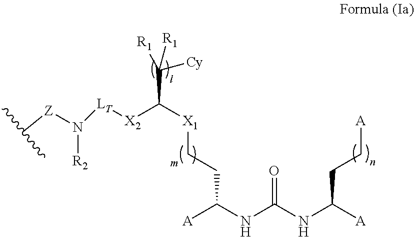

or a pharmaceutically acceptable salt, or solvate thereof.

In some embodiments (e.g., of ligands of Formula (I) or (Ia)), L_{T} is optionally substituted C₁ to C₂₀ alkylene, optionally substituted C₁ to C₂₀ heteroalkylene,

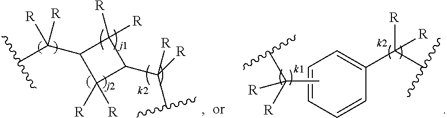

In some embodiments, L_{T} is C₁ to C₂₀ alkylene. In some embodiments, L_{T} is C₁ to C₁₀ alkylene. In some embodiments, L_{T} is C₁-C₆ alkylene. In some embodiments, L_{T} is C₁ to C₂₀ heteroalkylene. In some embodiments, L_{T} is C₁ to C₁₆ heteroalkylene, C₁ to C₁₂ heteroalkylene, C₁ to C₆ heteroalkylene, C₂ to C₂₀ heteroalkylene, C₂ to C₁₆ heteroalkylene, C₂ to C₁₂ heteroalkylene, or C₂ to C₆ heteroalkylene.

In some embodiments, L_{T} is

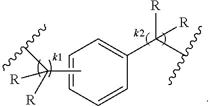

In some embodiments, L_{T} is

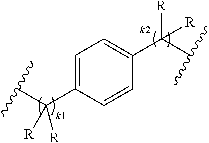

In some embodiments, k1 and k2 are each independently an integer from 0-6, 0-4, 0-2, or 0-1. In some embodiments, k1 and k2 are each independently 0, 1, or 2. In some embodiments, k1 and k2 are each independently 0 or 1. In some embodiments, k1 is 0, 1, or 2. In some embodiment, k2 is 0, 1, or 2. In some embodiments, k1 is 0 and k2 is 1. In some embodiments, k1 is 1 and k2 is 0. In some embodiments, k1 and k2 are both 1. In some embodiments, k1 and k2 are both 0.

In some embodiments, L_{T} is

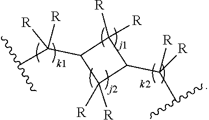

In some embodiments, L_{T} is

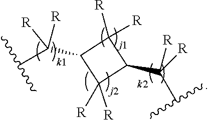

In some embodiments, j1 and j2 are each independently an integer from 1 to 4. In some embodiments, j1 and j2 are each independently 1 or 2. In some embodiments, j1 and j2 are each 2. In some embodiments, L_{T} is

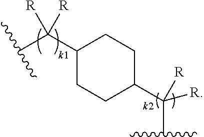

In some embodiments, L_{T} is

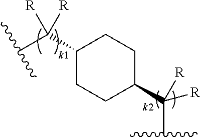

In some embodiments, L_{T} is

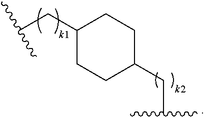

In some embodiments, L_{T} is

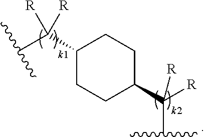

In some embodiments, k1 and k2 are each independently 0, 1, or 2. In some embodiments, k1 and k2 are each independently 0 or 1. In some embodiments, k1 is 0, 1, or 2. In some embodiment, k2 is 0, 1, or 2. In some embodiments, k1 is 0 and k2 is 1. In some embodiments, k1 is 1 and k2 is 0. In some embodiments, k1 and k2 are both 1. In some embodiments, k1 and k2 are both 0.

In some embodiments (e.g., of ligands of Formula (I) or (Ia)), L_{T} is C₁-C₃ alkylene. In some embodiments, L_{T} is methylene.

In some embodiments, the ligand has a structure of Formula (Ib):

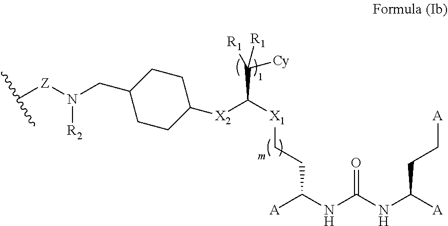

or a pharmaceutically acceptable salt, or solvate thereof.

In some embodiments, the ligand has a structure of Formula (If):

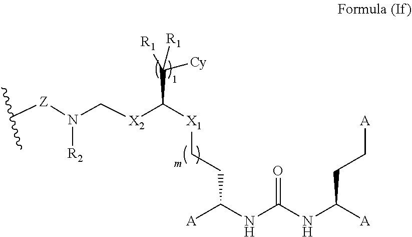

or a pharmaceutically acceptable salt, or solvate thereof.

In some embodiments, the ligand has a structure of Formula (Ic):

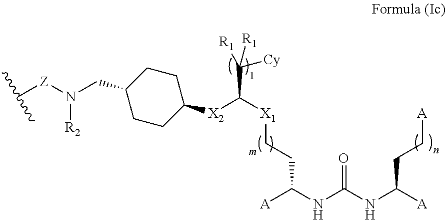

or a pharmaceutically acceptable salt, or solvate thereof.

In some embodiments (e.g., of a ligand of Formula (I), (Ia), (Ib), (If), or (Ic)), l is an integer from 0 to 6. In some embodiments l is an integer from 0 to 5, 0 to 4, 0 to 3, 0 to 2, 0 to 1, 1, to 6, 1 to 5, 1 to 4, 1 to 3, or 1 to 2. In some embodiments, l is 0, 1, or 2. In some embodiments, l is 0 or 1. In some embodiments, l is 1 or 2. In some embodiments, l is 1.

In some embodiments (e.g., of a ligand of Formula (I), (Ia), (Ib), (If), or (Ic)), each R₁ is independently H or optionally substituted alkyl. In some embodiments, each R₁ is independently H or methyl. In some embodiments, each R₁ is H.

In some embodiments, the ligand has a structure of Formula (Id):

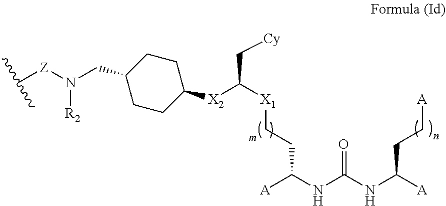

or a pharmaceutically acceptable salt, or solvate thereof.

In some embodiments, the ligand has a structure of Formula (Ig):

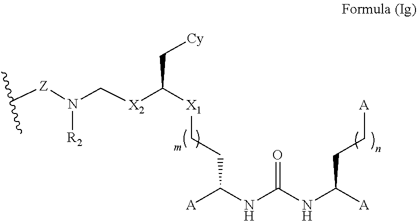

or a pharmaceutically acceptable salt, or solvate thereof.

In some embodiments (e.g., of ligands of Formula (I), (Ia), (Ib), (Ic), (If), (Id), or (Ig)), each A is independently selected from carboxylic acid, sulphonic acid, phosphonic acid, tetrazole, or isoxazole. In some embodiments, each A is carboxylic acid.

In some embodiments (e.g., of ligands of Formula (I), (Ia), (Ib), (Ic), (If), (Id), or (Ig)), m is an integer from 0 to 6. In some embodiments, m is an integer from 1 to 6, 1 to 5, 1 to 4, 1 to 3, 2 to 6, 2 to 5, 2 to 4, 2 to 3, 3 to 6, 3 to 5, or 3 to 4. In some embodiments, m is 1, 2, 3, 4, 5, or 6. In some embodiments, m is 2, 3, or 4. In some embodiments, m is 2 or 3. In some embodiments, m is 3 or 4. In some embodiments, m is 3.

In some embodiments (e.g., of ligands of Formula (I), (Ia), (Ib), (Ic), (If), (Id), or (Ig)), n is an integer from 0 to 6. In some embodiments n is an integer from 0 to 5, 0 to 4, 0 to 3, 0 to 2, 0 to 1, 1, to 6, 1 to 5, 1 to 4, 1 to 3, or 1 to 2. In some embodiments, n is 0, 1, or 2. In some embodiments, n is 0 or 1. In some embodiments, n is 1 or 2. In some embodiments, n is 1.

In some embodiments (e.g., of ligands of Formula (I), (Ia), (Ib), (Ic), (If), (Id), or (Ig)), each of X₁ and X₂ is independently —N(R)C(═O)—, —C(═O)N(R)—, —C(═O)O—, —OC(═O)N(R)—, —N(R)C(═O)O—, or —N(R)C(═O)N(R)—. In some embodiments, In some embodiments, each of X₁ and X₂ is independently —N(R)C(═O)— or —C(═O)N(R)—. In some embodiments, X₁ is —N(R)C(═O)— or —C(═O)N(R)—. In some embodiments, X₁ is —N(R)C(═O)—. In some embodiments, X₁ is —C(═O)N(R)—. In some embodiments, X₂ is —N(R)C(═O)— or —C(═O)N(R)—. In some embodiments, X₂ is —N(R)C(═O)—. In some embodiments, X₂ is —C(═O)N(R)—.

In some embodiments (e.g., of ligands of Formula (I), (Ia), (Ib), (Ic), (If), (Id), or (Ig)), each R is independently selected from H and optionally substituted alkyl. In some embodiments, each R is independently selected from H and optionally substituted C₁-C₆ alkyl. In some embodiments, each R is independently selected from H and C₁-C₆ alkyl. In some embodiments, each R is independently selected from H and optionally substituted C₁-C₃ alkyl. In some embodiments, each R is independently selected from H and C₁-C₃ alkyl. In some embodiments, each R is H or methyl. In some embodiments, each R is H.

In some embodiments (e.g., of ligands of Formula (I), (Ia), (Ib), (Ic), (If), (Id), or (Ig)), R₂ is selected from H and optionally substituted alkyl. In some embodiments, R₂ is H or methyl. In some embodiments, R₂ is H. In some embodiments, R₂ is C₁-C₆ alkyl substituted with an optionally substituted phenoxy. In some embodiments, the optionally substituted phenoxy is a 3,5-dicyanophenoxy group. In some embodiments, R₂ is C₃ alkyl substituted with an optionally substituted phenoxy. In some embodiments, R₂ is C₃ alkyl substituted with a 3,5-dicyanophenoxy group.

In some embodiments, the ligand has a structure of Formula (Ie):

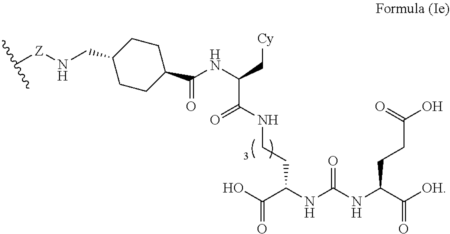

In some embodiments, the ligand has a structure of Formula (Ih):

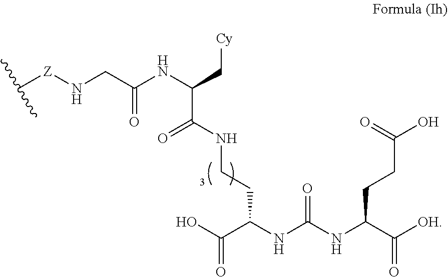

In some embodiments (e.g., of ligands of Formula (I), (Ia), (Ib), (Ic), (If), (Id), (Ig), (Ie), or (Ih)), Cy is optionally substituted cycloalkyl, optionally substituted heterocycloalkyl, optionally substituted aryl, or optionally substituted heteroaryl. In some embodiments, Cy is optionally substituted cycloalkyl or optionally substituted heterocycloalkyl. In some embodiments, Cy is optionally substituted C₃-C₁₂ cycloalkyl or optionally substituted C₂-C₁₂ heterocycloalkyl. In some embodiments, Cy is optionally substituted cyclopropyl, optionally substituted cyclobutyl, optionally substituted cyclopentyl, optionally substituted cyclohexyl, optionally substituted cycloheptyl, or optionally substituted cyclooctyl. In some embodiments, Cy is optionally substituted oxetane, optionally substituted tetrahydrofuran, optionally substituted pyrrolidone, optionally substituted piperidine, optionally substituted pyran (e.g., 2H-pyran), or optionally substituted morpholine.

In some embodiments (e.g., of ligands of Formula (I), (Ia), (Ib), (Ic), (If), (Id), (Ig), (Ie), or (Ih)), Cy is optionally substituted aryl or heteroaryl. In some embodiments, Cy is optionally substituted aryl. In some embodiments, Cy is optionally substituted C₆-C₂₀ aryl. In some embodiments, Cy is optionally substituted C₆-C₁₈, C₆-C₁₆, C₆-C₁₄, C₆-C₁₂, or C₆-C₁₀ aryl. In some embodiments, Cy is optionally substituted phenyl, optionally substituted napthyl (i.e., napthalene), optionally substituted anthracyl (i.e., anthracene), optionally substituted phenanthryl (i.e., phenanthrene), optionally substituted chrysyl (i.e., chrysene), or optionally substituted pyrryl (i.e., pyrene). In some embodiments, Cy is optionally substituted heteroaryl. In some embodiments, Cy is optionally substituted phenyl or optionally substituted napthyl. In some embodiments, Cy is optionally substituted phenyl. In some embodiments, Cy is optionally substituted napthyl.

In some embodiments (e.g., of ligands of Formula (I), (Ia), (Ib), (Ic), (If), (Id), (Ig), (Ie), or (Ih)), Cy is optionally substituted heteroaryl. In some embodiments, Cy is optionally substituted C₂-C₁₈ heteroaryl. In some embodiments, Cy is optionally substituted C₂-C₁₆, C₂-C₁₄, C₂-C₁₂, C₂-C₁₀, C₂-C₈, C₄-C₁₆, C₄-C₁₄, C₄-C₁₂, C₄-C₁₀, C₄-C₈, C₅-C₁₆, C₅-C₁₄, C₅-C₁₂, C₅-C₁₀, or C₅-C₈ heteroaryl. In some embodiments, Cy is optionally substituted furyl, optionally substituted pyrrolyl, optionally substituted thiophenyl, optionally substituted pyridinyl, optionally substituted indolyl, optionally substituted benzofuranyl, optionally substituted quinolinyl, optionally substituted isoquinolinyl, optionally substituted imidazolyl, optionally substituted oxazolyl, optionally substituted pyrazolyl, optionally substituted pyridazinyl, optionally pyrimidinyl, or optionally substituted purinyl. In some embodiments, Cy is optionally substituted indolyl. In some embodiments, Cy is optionally substituted indol-3-yl.

In some embodiments (e.g., of ligands of Formula (I), (Ia), (Ib), (Ic), (If), (Id), (Ig), (Ie), or (Ih)), the aryl or heteroaryl of Cy is optionally substituted with 1, 2, or 3 substituents R_{A}, wherein each R_{A} is independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —S C₁-C₄alkyl, —S(═O)C₁-C₄alkyl, and —S(═O)₂ C₁-C₄alkyl. In some embodiments, Cy has a structure

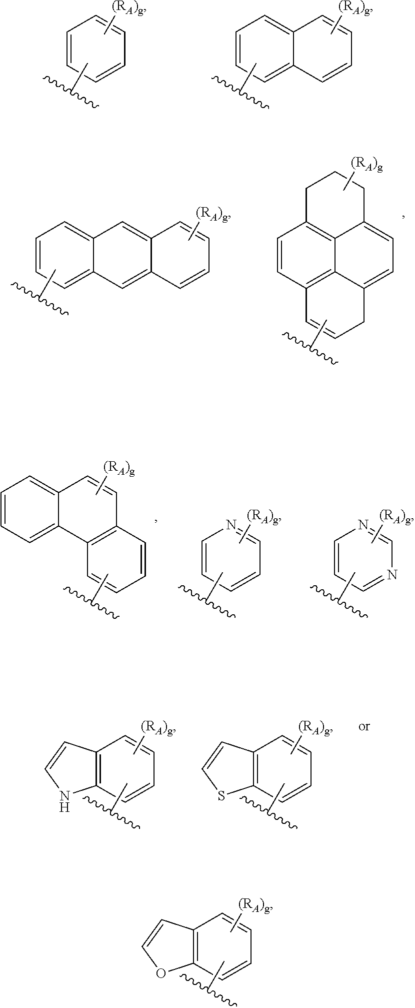

wherein each R_{A} is independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —S C₁-C₄alkyl, —S(═O)C₁-C₄alkyl, and —S(═O)₂ C₁-C₄alkyl, and g is an integer from 0 to 3. In some embodiments, Cy has a structure

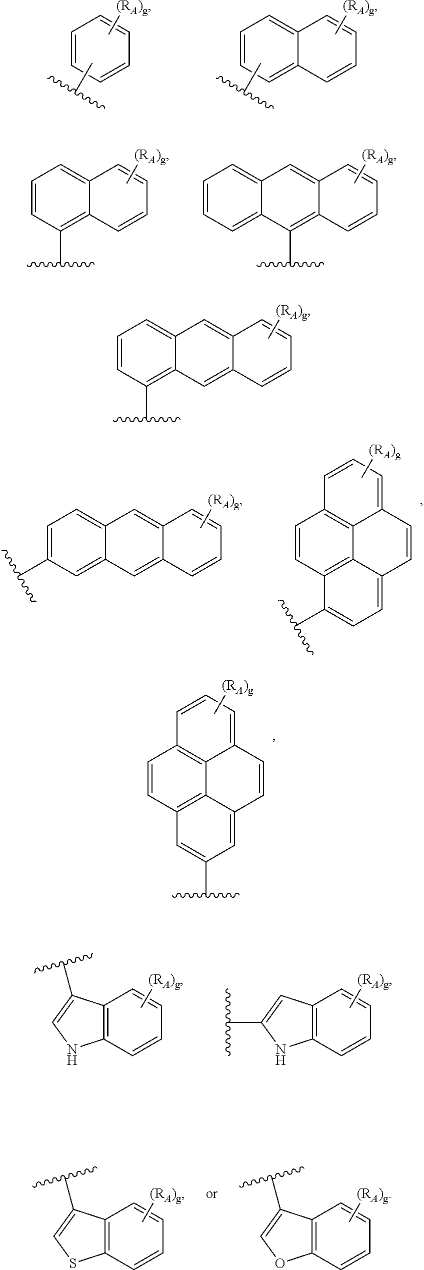

In some embodiments, Cy has a structure

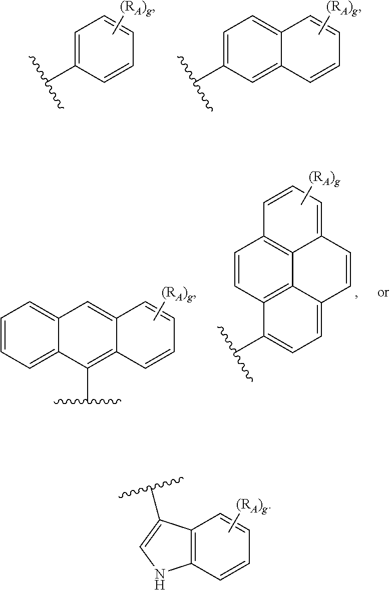

In some embodiments, Cy has a structure

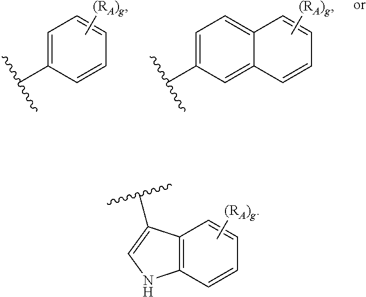

In some embodiments, Cy has a structure

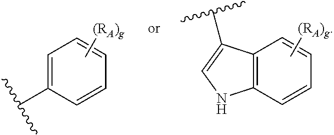

In some embodiments, Cy has a structure

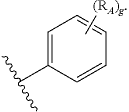

In some embodiments, Cy has a structure

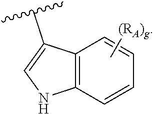

In some embodiments, each R_{A} is independently selected from halogen, —CN, —NH₂, —OH, —CO₂H, —S(═O)₂NH₂, C₁-C₄alkoxy, and —SC₁-C₄alkyl. In some embodiments, Cy has a structure In some embodiments, each R_{A} is independently selected from halogen, —CN, —CO₂H, and C₁-C₄alkoxy. In some embodiments, each R_{A} independently selected from F, Cl, Br, I, —CN, and —CO₂H. In some embodiments, each R_{A} is independently selected from F, Cl, Br, I, and —CO₂H. In some embodiments, each R_{A} is independently selected from F and —CO₂H. In some embodiments, g is 0, 1, or 2. In some embodiments, g is 0 or 1. In some embodiments, g is 0. In some embodiments, g is 1.

In some embodiments, Cy is

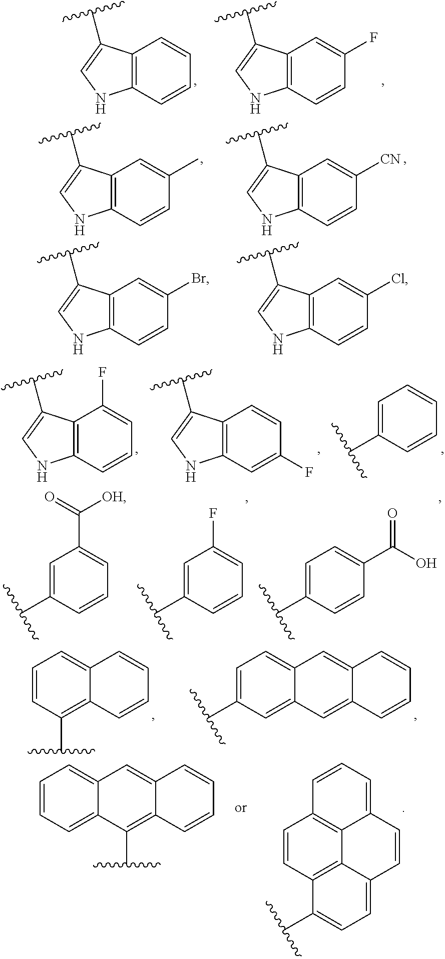

In some embodiments, Cy is

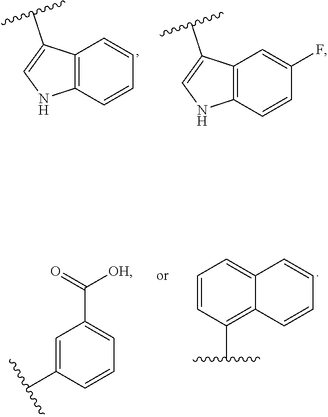 [SMILES: *c1c[nH]c2ccccc12 ; *c1c[nH]c2ccc(F)cc12 ; *c1cccc(C(=O)O)c1 ; *c1cccc2ccccc12]

In some embodiments, Cy is

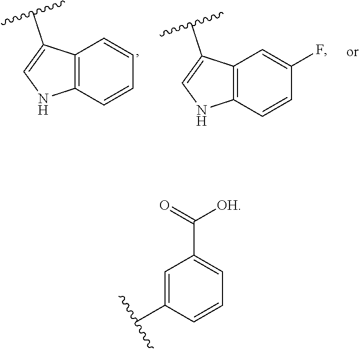 [SMILES: *c1c[nH]c2ccccc12 ; *c1c[nH]c2ccc(F)cc12 ; *c1cccc(C(=O)O)c1]

In some embodiments, Cy is

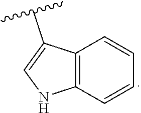 [SMILES: *c1c[nH]c2ccccc12]

In some embodiments, Cy is

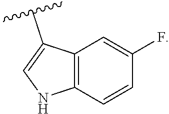 [SMILES: *c1c[nH]c2ccc(F)cc12]

In some embodiments, Cy is

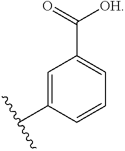 [SMILES: *c1cccc(C(=O)O)c1]

In some embodiments, Cy is

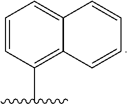 [SMILES: *c1cccc2ccccc12]

In some embodiments (e.g., of ligands of Formula (I), (Ia), (Ib), (Ic), (If), (Id), (Ig), (Ie), or (Ih)), Z is the peptide. In preferred embodiments, the C-terminus of the peptide is directly connected to the NR₂ amino of the ligand via an amide bond.

In some embodiments, the peptide has a structure: -AA₁-AA₂-AA₃-AA₄-AA₅-AA₆-AA₇-AA₈-AA₉-AA₁₀-AA₁₁-AA₁₂-AA₁₃-AA₁₄-AA₁₅-AA₁₆-, wherein each of AA₁-AA₉ and AA₁₂-AA₁₆ is independently any amino acid or absent; and AA₁₀ and AA₁₁ are each independently amino acids with polar side chains. In some embodiments, AA₁₀ and AA₁₁ are each amino acids with carboxylic acid or amide-containing side chains. In some embodiments, AA₁₀ and AA₁₁ are each glutamate. In some embodiments, AA₁₂ is glutamate or glycine. In some embodiments, AA₁₃ is optionally substituted phenylalanine. In some embodiments, AA₁₃ is carboxy substituted phenylalanine. In some embodiments, AA₁₃ is 3-carboxy-phenylalanine or 4-carboxy-phenylalanine. In some embodiments, each of AA₁₄-AA₁₆ are absent. In some embodiments, AA₁₄ is glycine and AA₁₅-AA₁₆ are absent. In some embodiments, each of AA₁-AA₉, if present, are independently selected from A, E, F, G, L, Q, S and Y. In some embodiments, each of AA₁-AA₉, if present, are independently selected from A, F, and S. In some embodiments, AA₁-AA₉ contain 1, 2, 3, 4, 5, or 6 amino acids which are present (e.g., only 1, 2, 3, 4, 5, or 6 of AA₁-AA₉ are present).

In some embodiments, the peptide has a structure: -AA₁-AA₂-AA₃-AA₄-AA₅-AA₆-AA₇-AA₈-AA₉-AA₁₀-AA₁₁-AA₁₂-AA₁₃-AA₁₄-AA₁₅-AA₁₆-, wherein each of AA₁-AA₅ and AA₁₂-AA₁₆ is independently any amino acid or absent; AA₆ and AA₇ are each independently amino acids with cyclic side chains; AA₈ and AA₉ are each independently amino acids with linear or branched alkyl side chains; and AA₁₀ and AA₁₁ are each independently amino acids with polar side chains. In some embodiments, the carboxy-terminus of the peptide portion of the ligand is connected to the rest of the molecule (i.e., at the N depicted as attached to the peptide in the above formulas)

In some embodiments (e.g., of ligands of Formula (I), (Ia), (Ib), (Ic), (If), (Id), (Ig), (Ie), or (Ih)), the peptide of the ligand has the structure -AA₁-AA₂-AA₃-AA₄-AA₅-AA₆-AA₇-AA₈-AA₉-AA₁₀-AA₁₁-AA₁₂-AA₁₃-AA₁₄-AA₁₅-AA₁₆-. In some embodiments, each of AA₁-AA₁₆ is independently a natural or unnatural alpha amino acid. In some embodiments, each of AA₁-AA₁₆ is independently absent or an L-amino acid. In some embodiments, each of AA₁-AA₅ and AA₁₂-AA₁₆ is independently any amino acid or absent. In some embodiments, each of AA₁-AA₅ and AA₁₂-AA₁₆ is independently any natural amino acid or absent. In some embodiments, the carboxy-terminus of the peptide portion of the ligand is connected to the rest of the molecule (i.e., at the N depicted as attached to Z in the above formulas).

In some embodiments (e.g., of ligands of Formula (I), (Ia), (Ib), (Ic), (If), (Id), (Ig), (Ie), or (Ih)), each of AA₁₂-AA₁₆ is independently glycine, serine, or absent. In some embodiments, each of AA₁₂-AA₁₆ is independently glycine or absent. In some embodiments, each of AA₁-AA₅ and AA₁₂-AA₁₆ is independently glycine, serine, or absent. In some embodiments, each of AA₁-AA₅ and AA₁₂-AA₁₆ is independently glycine or absent. In some embodiments, AA₁₂ is glycine. In some embodiments, each of AA₁-AA₅ and AA₁₃-AA₁₆ is absent.

In some embodiments (e.g., of ligands of Formula (I), (Ia), (Ib), (Ic), (If), (Id), (Ig), (Ie), or (Ih)), AA₆ and AA₇ are each independently amino acids with aromatic side chains. In some embodiments, AA₆ and AA₇ are each independently natural or unnatural amino acids with aromatic side chains. In some embodiments, AA₆ and AA₇ are each independently natural amino acids with aromatic side chains. In some embodiments, AA₆ and AA₇ are both the same amino acid. In some embodiments, the side chains of both AA₆ and AA₇ independently have the structure

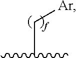

wherein f is an integer from 0 to 3 and Ar is optionally substituted aryl or heteroaryl. In some embodiments, f is 0, 1, or 2. In some embodiments, f is 1. In some embodiments, Ar is optionally substituted phenyl, optionally substituted indolyl (e.g., indol-3-yl), optionally substituted imidazolyl, optionally substituted napthyl, optionally substituted benzothiophenyl (e.g., benzothiophen-3-yl), optionally substituted benzofuranyl (e.g., benzofuran-3-yl), optionally substituted pyridinyl. In some embodiments, the optionally substituted aryl or heteroaryl is optionally substituted with halogen. In some embodiments, AA₆ and AA₇ are each independently phenylalanine, tyrosine, histidine, or tryptophan. In some embodiments, AA₆ and AA₇ are each phenylalanine.

In some embodiments (e.g., of ligands of Formula (I), (Ia), (Ib), (Ic), (If), (Id), (Ig), (Ie), or (Ih)), AA₈ and AA₉ are each independently amino acids with linear or branched alkyl side chains. In some embodiments, AA₈ and AA₉ are each independently amino acids with C₁-C₆ side chains. In some embodiments, AA₈ and AA₉ are each independently amino acids with C₁-C₃ side chains. In some embodiments, AA₈ and AA₉ are each independently valine, alanine, or homoalanine. In some embodiments, AA₈ and AA₉ are each alanine.

In some embodiments (e.g., of ligands of Formula (I), (Ia), (Ib), (Ic), (If), (Id), (Ig), (Ie), or (Ih)), AA₁₀ and AA₁₁ are each independently amino acids with polar side chains. In some embodiments, each polar side chain comprises a functional group selected from —OH, —COOH, —NH₂, and —CONH₂. In some embodiments, AA₁₀ and AA₁₁ are each independently serine, threonine, asparagine, aspartate, glutamine, or glutamate. In some embodiments, AA₁₀ and AA₁₁ are each independently asparagine, aspartate, glutamine, or glutamate. In some embodiments, AA₁₀ and AA₁₁ are each independently aspartate, glutamine, or glutamate. In some embodiments, AA₁₀ and AA₁₁ are each independently aspartate or glutamate. In some embodiments, AA₁₀ and AA₁₁ are each independently glutamine or glutamate. In some embodiments, AA₁₀ and AA₁₁ are each glutamate.

In some embodiments, any of the peptides described above (e.g., any of the peptides described as part of a ligand of Formula (I), (Ia), (Ib), (Ic), (If), (Id), (Ig), (Ie), or (Ih)) comprises at least two consecutive glutamate residues. In some embodiments, the peptide comprises at least three consecutive glutamate residues. In some embodiments, the peptide comprises two consecutive glutamate residues. In some embodiments, the peptide comprises three consecutive glutamate residues.

In some embodiments (e.g., of ligands of Formula (I), (Ia), (Ib), (Ic), (If), (Id), (Ig), (Ie), or (Ih)), the peptide has a sequence of FFAAEEG (SEQ ID NO: 9), FFAAEE (SEQ ID NO: 10), FFAADDG (SEQ ID NO: 11), FFAADD (SEQ ID NO: 12), FFAAQQG (SEQ ID NO: 13), FFAAQQ (SEQ ID NO: 14), FFAANNG (SEQ ID NO: 15), FFAANN (SEQ ID NO: 16), YYAAEEG (SEQ ID NO: 17), YYAAEE (SEQ ID NO: 18), YYAADDG (SEQ ID NO: 19), YYAADD (SEQ ID NO: 20), YYAAQQG (SEQ ID NO: 21), YYAAQQ (SEQ ID NO: 22), YYAANNG (SEQ ID NO: 23), YYAANN (SEQ ID NO: 24), WWAAEEG (SEQ ID NO: 25), WWAAEE (SEQ ID NO: 26), WWAADDG (SEQ ID NO: 27), WWAADD (SEQ ID NO: 28), WWAAQQG (SEQ ID NO: 29), WWAAQQ (SEQ ID NO: 30), WWAANNG (SEQ ID NO: 31), WWAANN (SEQ ID NO: 32). In some embodiments, the peptide has a sequence of FFAAEEG (SEQ ID NO: 9) or FFAAEE (SEQ ID NO: 10). In some embodiments, the peptide has a sequence of FFAAEEG (SEQ ID NO: 9).

In some embodiments, the peptide comprises any one of the following amino acid sequences: AAAAAA (SEQ ID NO: 33), (D-Ala)-(D-Ala)-(D-Ala)-(D-Ala)-(D-Ala)-(D-Ala) (SEQ ID NO: 34), AAAAEEG (SEQ ID NO: 35), AAAEE (SEQ ID NO: 36), AAAEEE (SEQ ID NO: 37), AAAEEEG (SEQ ID NO: 38), AAEE (SEQ ID NO: 39), AAEEG (SEQ ID NO: 40), AAFFAAEE (SEQ ID NO: 41), AAFFAAEEG (SEQ ID NO: 42), AAFFAEEE (SEQ ID NO: 43), AAFFAEEEG (SEQ ID NO: 44), AAQQEE (SEQ ID NO: 45), AAQQEEG (SEQ ID NO: 46), AEE, EE, EEE, EEEG (SEQ ID NO: 47), EEEG(3-carboxy-phenylalanine) (SEQ ID NO: 48), EEEG(4-carboxy-phenylalanine) (SEQ ID NO: 49), EEG, EEG(3-carboxy-phenylalanine) (SEQ ID NO: 103), FAAEE (SEQ ID NO: 50), FAAEEG (SEQ ID NO: 51), FAFAEE (SEQ ID NO: 52), FAFAEEG (SEQ ID NO: 53), FFAAEE (SEQ ID NO: 10), FFAAEE(3-carboxy-phenylalanine) (SEQ ID NO: 78), FFAAEEE (SEQ ID NO: 54), FFAAEEEG (SEQ ID NO: 55), FFAAEEG (SEQ ID NO: 9), FFAAEEG(3-carboxy-phenylalanine) (SEQ ID NO: 56), FFAEEE (SEQ ID NO: 57), FFAEEEG (SEQ ID NO: 58), FFFAAEE (SEQ ID NO: 59), FFFAAEEG (SEQ ID NO: 60), FFLLEE (SEQ ID NO: 61), FFLLEEG (SEQ ID NO: 62), FFQQEE (SEQ ID NO: 63), FFQQEEG (SEQ ID NO: 64), FFSSEE (SEQ ID NO: 65), FFSSEEG (SEQ ID NO: 66), FFSSEEG(3-carboxy-phenylalanine) (SEQ ID NO: 67), FFSSEEG(4-carboxy-phenylalanine) (SEQ ID NO: 68), FLFLEE (SEQ ID NO: 69), FLFLEEG (SEQ ID NO: 70), GGGGEE (SEQ ID NO: 71), GGGGEEG (SEQ ID NO: 72), PPPPPP (SEQ ID NO: 73), YYAAEE (SEQ ID NO: 18), YYAAEEG (SEQ ID NO: 17), YYSSEE (SEQ ID NO: 74), YYSSEEG (SEQ ID NO: 75), YYSSEEG(3-carboxy-phenylalanine) (SEQ ID NO: 76), or YYSSEEG(4-carboxy-phenylalanine) (SEQ ID NO: 77).

In some embodiments, Z comprises the poly(ethylene glycol). In some embodiments, the poly(ethylene glycol) is attached to the NR₂ group via an amide bond. In some embodiments, the poly(ethylene glycol) has a structure

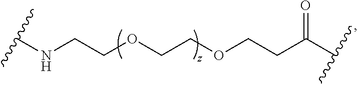

wherein the carboxyl group is attached to the NR₂ group of the ligand, z is an integer from 1 to 100, and the NH group forms the point of attachment to the additional group or the linker. In some embodiments, z is 1 to 100, 1 to 50, 1 to 25, 1 to 10, 2 to 100, 2 to 50, 2 to 25, 2 to 10, 5 to 100, 5 to 50, 5 to 25, or 5 to 10. In some embodiments, z is 2, 3, 4, 5, 6, 7, 8, 9, or 10.

In some embodiments (e.g., of ligands of Formula (I), (Ia), (Ib), (Ic), (If), (Id), (Ig), (Ie), or (Ih)), the linker is present and connects the rest of the ligand to the additional group. The linker can be any suitable structure which acts to connect the additional group with the rest of the molecule. In some embodiments, the linker is connected to the N-terminal amine of the peptide portion of the ligand.

In some embodiments, the linker comprises a chemical polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol. In some embodiments, the linker comprises from 2 to 100 ethylene glycol units. In some embodiments, the linker comprises from 2 to 100, 2 to 75, 2 to 50, 2 to 40, 2 to 35, 2 to 30, 2 to 25, 5 to 100, 5 to 75, 5 to 50, 5 to 40, 5 to 35, 5 to 25, 10 to 100, 10 to 75, 10 to 50, 10 to 40, 10 to 35, 10 to 30, or 10 to 25 ethylene glycol units.

In some embodiments, the linker is an alkylene chain or a heteroalkylene chain. In some embodiments, the linker is a C₁-C₁₀₀ alkylene chain, a C₁-C₅₀ alkylene chain, a C₁-C₄₀ alkylene chain, a C₁-C₃₀ alkylene chain, a C₁-C₂₀ alkylene chain, C₂-C₁₀₀ alkylene chain, a C₂-C₅₀ alkylene chain, a C₂-C₄₀ alkylene chain, a C₂-C₃₀ alkylene chain, or a C₂-C₂₀ alkylene chain. In some embodiments, the linker is a C₁-C₁₀₀ heteroalkylene chain, a C₁-C₅₀ heteroalkylene chain, a C₁-C₄₀ heteroalkylene chain, a C₁-C₃₀ heteroalkylene chain, a C₁-C₂₀ heteroalkylene chain, C₂-C₁₀₀ heteroalkylene chain, a C₂-C₅₀ heteroalkylene chain, a C₂-C₄₀ heteroalkylene chain, a C₂-C₃₀ heteroalkylene chain, or a C₂-C₂₀ heteroalkylene chain. In some embodiment, the linker can comprise cyclic structures (e.g., cyclohexyl, phenyl, or other groups which contain a cyclic structure as part of the linker).

In some embodiments, the linker comprises a linear chain of 1 to 500, 1 to 400, 1 to 300, 1 to 200, 1 to 100, 1 to 50, 1 to 40, or 1 to 30 atoms.

In some embodiments, the linker comprises a reaction product of one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole.

In some embodiments, the linker is a peptide linker. Non-limiting examples of peptide linkers include, but are not limited to (GS)ₙ (SEQ ID NO: 1), (GGS)ₙ (SEQ ID NO: 2), (GGGS)ₙ (SEQ ID NO: 3), (GGSG)ₙ (SEQ ID NO: 4), or (GGSGG)ₙ (SEQ ID NO: 5), (GGGGS)ₙ (SEQ ID NO: 6), wherein n is 1, 2, 3, 4, 5, 6, 7, 8, 9, or 10. For example, a linking peptide can be (GGGGS)₃ (SEQ ID NO: 7) or (GGGGS)₄ (SEQ ID NO: 8).

In some embodiments, it may be desirable that the additional group which is linked to the ligand is released from the ligand within or near the tumor environment. In such cases, a cleavable linker can be used (e.g., a protease cleavable linker).

In some embodiments, the ligand is attached to a conjugation handle. Any suitable reactive group capable of reacting with a complementary reactive group attached to the synthetic cytokine or derivative thereof can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, potassium acyl trifluoroborate (KAT) ligation, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne (e.g., dibenzocyclooctynes), tetrazine, halide (e.g., alpha-halo carbonyl groups), sulfhydryl, disulfide, maleimide, activated ester (e.g., N-hydroxysuccinimide ester), alkene (e.g., alpha,beta-unsaturated carbonyl), aldehyde, ketone, imine, hydrazine, and hydrazide. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety. In some embodiments, the conjugation handle is any of those described herein (see, e.g., the table of conjugation handles described below).

Non-limiting examples of the structures of conjugation handles with suitable linkers for attaching to the ligands described herein, for example,

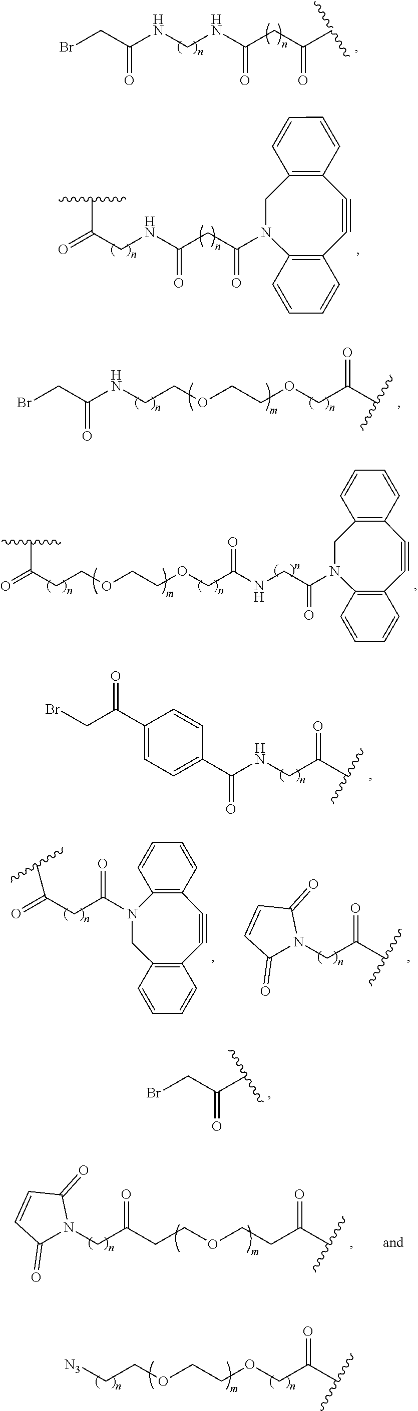

wherein each n is independently an integer from 1-6 and each m is independently an integer from 1-30, and related molecules (e.g., isomers).

The additional group to which the ligand is attached (for example, by using a conjugation handle as discussed above) can be any functionality or molecule which is desired to be targeted to PSMA (or to a tumor expressing PSMA). Non-limiting examples of such modalities include polypeptides (e.g., a therapeutic protein, such as a therapeutic enzyme, cytokine, antibody, etc.), a polysaccharide, a nucleic acid, a lipid, an organic biopolymer, a chemical polymer, a nanoparticle, a microparticle, a dye, a bio-organic molecule, or a drug (e.g., a small molecule drug, a toxic payload, a radionuclide (or a chelator for carrying the radionuclide)). In some embodiments, the additional group is not a chelator and does not comprise a radionuclide.

In some embodiments, the additional group is a therapeutic protein. In some embodiments, the therapeutic protein is a cytokine. Non-limiting examples of cytokines include interleukins (e.g., IL-2, IL-18, IL-7, IL-17), TNF family cytokines (e.g., TNFa, CD70, TNFSF14), interferons (e.g., IFNγ, IFNα. IFNβ), TGF-β family cytokines (e.g., TGFB1, TGFB2, TGFB3), chemokines (e.g., CCL2, CCL3, CXCL9, CXCL10) and others. In some embodiments, the cytokine is an interleukin. In some embodiments, the interleukin is an IL-1 family cytokine (e.g., IL-18, IL-1β, IL-33), an IL-2 family cytokine (e.g., IL-2, IL-4, IL-7, IL-15, IL-21), an IL-6 family interleukin (e.g., IL-6, IL-11, IL-31), an IL-10 family cytokine (e.g., IL-10, IL-19, IL-20, IL-22), an IL-12 family cytokine (e.g., IL-12, IL-23, IL-27, IL-35) and an IL-17 family cytokine (e.g., IL-17, IL-17F, IL-25). The cytokine can be the wild type version or a modified version of the cytokine (e.g., a version having at least 70%, 75%, 80%, 85%, 90%, or 95% sequence identity to the natural sequence or a portion thereof).

In some embodiments, the additional group is a combination of different components (e.g., a fusion protein comprising multiple payloads, or a conjugate comprising multiple payloads, such as a bifunctional cytokine conjugate (e.g., an IL-2/IL-18 conjugate).

In some embodiments, the additional group is a chelator, optionally containing a radionuclide. In some embodiments, the chelator is 1,4,7,10-tetraazacyclododecane-1,4,7,10-tetraacetic acid (DOTA), 1,4,7,10-tetraazacyclododecane-1,4,7,10-tetrapropionic acid (DOTPA), 1,4,7,10-tetraazacyclododecane-1,4,7,10-tetramethylene phosphoric acid (DOTMP), Hydroxypropyltetraazacyclododecane triacetic acid (HP-DO3A), (1R,4R,7R,10R)-α,α′,α″,α′″-tetramethyl-1,4,7,10-tetraazacyclododecane-1,4,7,10-tetraacetic acid (DOTAMA), 1,4,7,10-tetrakis(carbamoylmethyl)-1,4,7,10-tetraazacyclododecane (DOTAA), 1,4,7,10-tetraazacyclododecane-1,4,7,10-tetrakis(acetamidomethylene) phosphonic acid (DOTA-A-AMP), tetraazacyclododecane dimethane phosphonic acid (DO2P), α-(2-carboxyethyl)-1,4,7,10-tetraazacyclododecane-1,4,7,10-tetraacetic acid (DOTAGA), N,N′,N″,N′″-tetra(1,2-dihydro-1-hydroxy-2-oxopyridine-6-carbonyl)-1,5,10,14-tetraazatetradodecane (1,2-HOPO), 1,4,8,11-tetraazacyclotetradecane-1,4,8,11-tetraacetic acid (TETA), 1,4,8,11-tetraazacyclotetradecane-1,4,8,11-tetrapropionic acid (TETPA), 1,4,7,10,13-pentaazacyclopentadecane-N,N′,N″,N′″,N″″-pentaacetic acid (PEPA), or 2-[4,7-bis(carboxymethyl)-1,4,7-triazonan-1-yl] acetic acid (NOTA). In some embodiments, the chelator is a linear or open chain chelator, such as Ethylenediaminetetraacetic acid (EDTA), 6,6′-((ethane-1,2-diylbis((carboxymethyl)azanediyl)) bis(methylene)) dipicolinic acid (H₄octapa), 6,6′-({9-hydroxy-1,5-bis(methoxycarbonyl)-2,4-di(pyridine-2-yl)-3,7-diazabicyclo[3.3.1]nonane-3,7-diyl} bis(-methylene)) dipicolinic acid (H2bispa2), 1,2-[{6-(carboxy)-pyridine-2-yl}-methylamino]ethane (H₂dedpa), 6-(1,4,10,13-tetraoxa-7,16-diazacyclooctadecane-N,N′-dimethyl) picolinic acid (H₂macropa), N, N″-bis(6-carboxy-2-pyridylmethyl)-diethylenetriamine-N,N′,N″-triacetic acid (Hsdecapa), N,N′-(methylenephosphonate)-N,N′-[6-(methoxycarbonyl)pyridine-2-yl]-methyl-1,2-diaminoethane (H₆phospa), 6,6′-(((((4-isothiocyanate phenethyl) azanediyl) bis(ethane-2,1-diyl)) bis((carboxymethyl) azanediyl)) bis(methylene)) dipicolinic acid (p-SCN-Bn-H₄neunpa), 6,6′-(((((4-nitrophenethyl) azanediyl) bis(ethane-2,1-diyl)) bis((carboxymethyl) azanediyl)) bis(methylene)) dipicolinic acid (p-NO₂-Bn-H₄neunpa), or 6,6′-(((azanediylbis (ethane-2,1-diyl)) bis((carboxymethyl) azanediyl) bis(methylene)) dipicolinic acid (Hsneunpa). In some embodiments, the chelator is DOTA. In some embodiments, the chelator is directly attached to a terminal amine of the Z group via an amide bond with a carboxylic acid on the chelator.

In some embodiments, the chelator contains the radionuclide. In some embodiments, the radionuclide is a radioactive metal ion. In some embodiments, the radioactive metal ion is an ion of ⁴⁴Sc, ⁵¹Cr, ⁵⁷Co, ⁵⁸Co, ⁶⁰Co, ⁵⁹Fe, ⁶⁴Cu, ⁶⁷Cu, ⁶⁷Ga ⁶⁸Ga ⁸⁹Sr, ⁸⁹Zr, ⁹⁰Y, ^{99m}Tc, ¹⁰³Ru, ¹¹¹In, ¹⁵³Sm, ¹⁶⁵Dy, ¹⁶⁶Ho, ¹⁷⁷Lu, ¹⁸⁶Re, ¹⁸⁸Re, ¹⁹⁷Hg, ¹⁹⁸Au, ²⁰¹Tl, ²⁰³Hg, ²¹²Pb, ²¹²Bi, ²¹³Bi, ²²⁵Ac, or ²²⁷Th. In some embodiments, the radioactive metal ion is an ion of ²¹²Bi, ²¹³Bi, ²²⁵Ac, or ²²⁷Th. In some embodiments, the radioactive metal ion is an ion of ²²⁷Th or ²²⁵Ac. In some embodiments, the radioactive metal ion is an ion of ²²⁵Ac. In some embodiments, the radioactive metal ion is an ion of ¹⁷⁷Lu.

Non-limiting examples of PSMA targeting ligands are shown in Table 1. The targeting ligands shown in Table 1 can be further modified to incorporate a conjugation handle or otherwise form a point of attachment to an additional group. In some embodiments, the acetyl group at the left terminal end of each structure is removed and the remaining amine serves as the point of attachment to the additional group, optionally through a linker. Thus, in some embodiments, the instant disclosure provides any of the PSMA targeting ligands described in Table 1 below in which the N-terminal acetyl group is removed and to which an additional group as described herein is attached to the amine group (e.g., by an amide bond). Compounds 119 and 120 demonstrate how a chelator group (e.g., DOTA) can be attached to the ligands described herein.

TABLE 1
Examples of PSMA targeting ligands

| Compound | |
|---|---|
| No | Chemical structure |
| 1 | 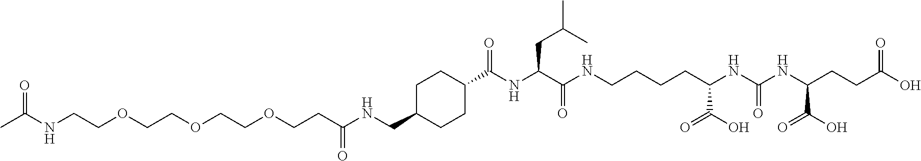 [SMILES: CC(=O)NCCOCCOCCOCCC(=O)NC[C@H]1CC[C@H](C(=O)N[C@@H](CC(C)C)C(=O)NCCCC[C@H](NC(=O)N[C@@H](CCC(=O)O)C(=O)O)C(=O)O)CC1] |
| 2 | 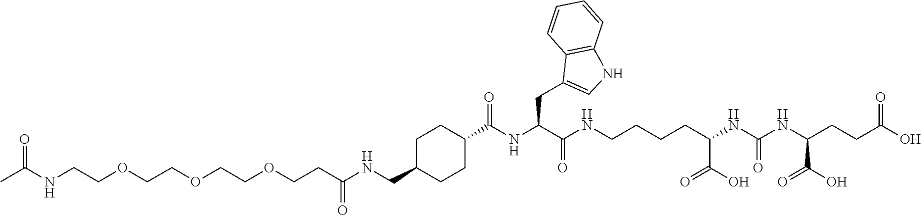 [SMILES: CC(=O)NCCOCCOCCOCCC(=O)NC[C@H]1CC[C@H](C(=O)N[C@@H](Cc2c[nH]c3ccccc23)C(=O)NCCCC[C@H](NC(=O)N[C@@H](CCC(=O)O)C(=O)O)C(=O)O)CC1] |
| 3 | 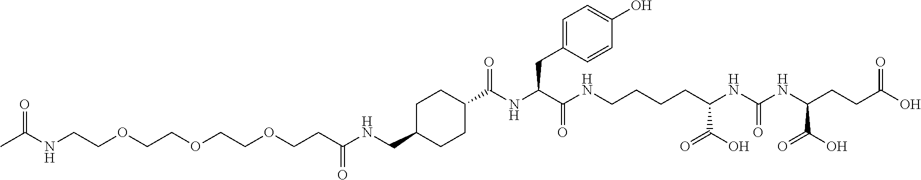 [SMILES: CC(=O)NCCOCCOCCOCCC(=O)NC[C@H]1CC[C@H](C(=O)N[C@@H](Cc2ccc(O)cc2)C(=O)NCCCC[C@H](NC(=O)N[C@@H](CCC(=O)O)C(=O)O)C(=O)O)CC1] |
| 4 | 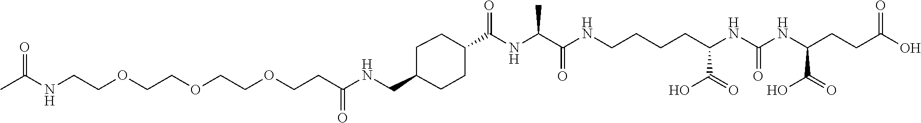 [SMILES: CC(=O)NCCOCCOCCOCCC(=O)NC[C@H]1CC[C@H](C(=O)N[C@@H](C)C(=O)NCCCC[C@H](NC(=O)N[C@@H](CCC(=O)O)C(=O)O)C(=O)O)CC1] |
| 5 | 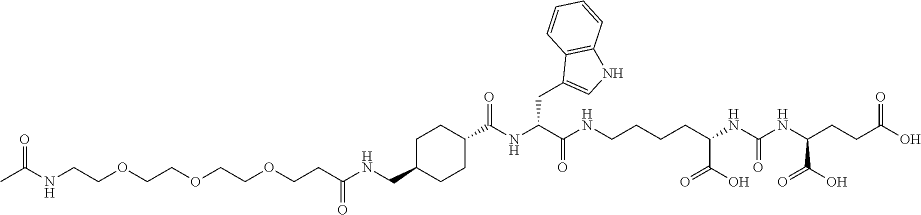 [SMILES: CC(=O)NCCOCCOCCOCCC(=O)NC[C@H]1CC[C@H](C(=O)N[C@H](Cc2c[nH]c3ccccc23)C(=O)NCCCC[C@H](NC(=O)N[C@@H](CCC(=O)O)C(=O)O)C(=O)O)CC1] |
| 6 | 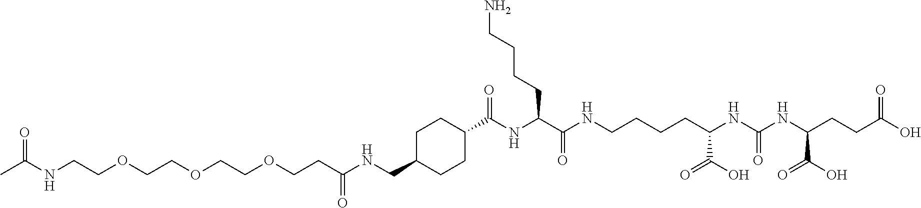 [SMILES: CC(=O)NCCOCCOCCOCCC(=O)NC[C@H]1CC[C@H](C(=O)N[C@@H](CCCCN)C(=O)NCCCC[C@H](NC(=O)N[C@@H](CCC(=O)O)C(=O)O)C(=O)O)CC1] |
| 7 | 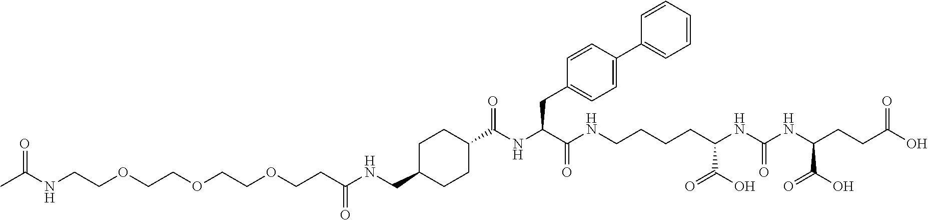 [SMILES: CC(=O)NCCOCCOCCOCCC(=O)NC[C@H]1CC[C@H](C(=O)N[C@@H](Cc2ccc(-c3ccccc3)cc2)C(=O)NCCCC[C@H](NC(=O)N[C@@H](CCC(=O)O)C(=O)O)C(=O)O)CC1] |
| 8 | 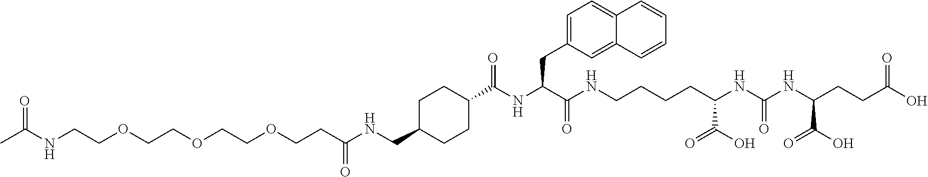 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 9 | 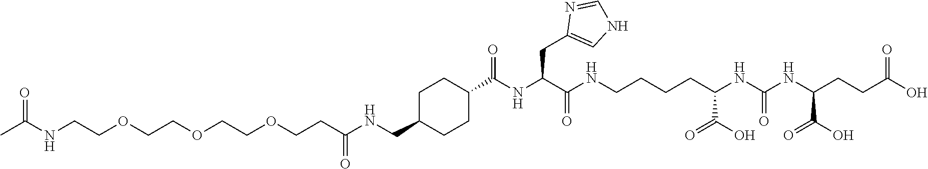 [SMILES: CC(=O)NCCOCCOCCOCCC(=O)NC[C@H]1CC[C@H](C(=O)N[C@@H](Cc2c[nH]cn2)C(=O)NCCCC[C@H](NC(=O)N[C@@H](CCC(=O)O)C(=O)O)C(=O)O)CC1] |
| 10 | 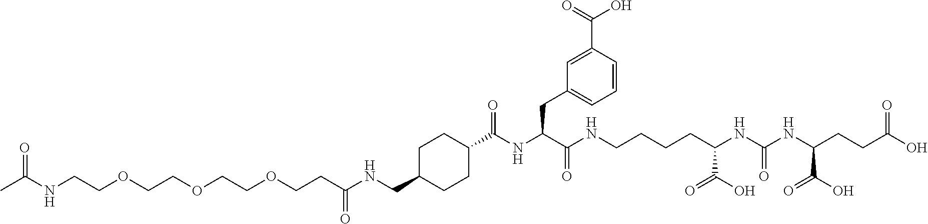 [SMILES: CC(=O)NCCOCCOCCOCCC(=O)NC[C@H]1CC[C@H](C(=O)N[C@@H](Cc2cccc(C(=O)O)c2)C(=O)NCCCC[C@H](NC(=O)N[C@@H](CCC(=O)O)C(=O)O)C(=O)O)CC1] |
| 11 | 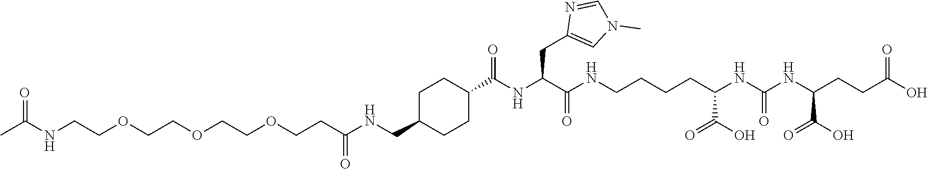 [SMILES: CC(=O)NCCOCCOCCOCCC(=O)NC[C@H]1CC[C@H](C(=O)N[C@@H](Cc2cn(C)cn2)C(=O)NCCCC[C@H](NC(=O)N[C@@H](CCC(=O)O)C(=O)O)C(=O)O)CC1] |
| 12 | 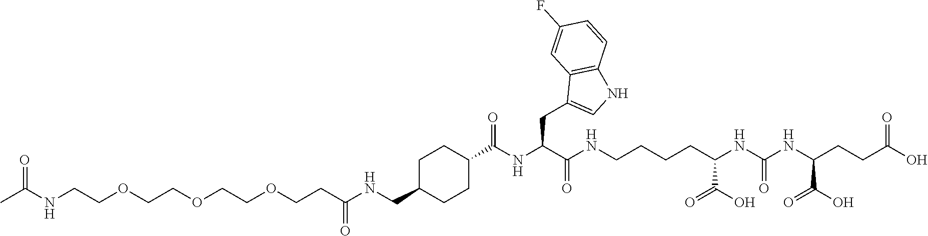 [SMILES: CC(=O)NCCOCCOCCOCCC(=O)NC[C@H]1CC[C@H](C(=O)N[C@@H](Cc2c[nH]c3ccc(F)cc23)C(=O)NCCCC[C@H](NC(=O)N[C@@H](CCC(=O)O)C(=O)O)C(=O)O)CC1] |
| 13 | 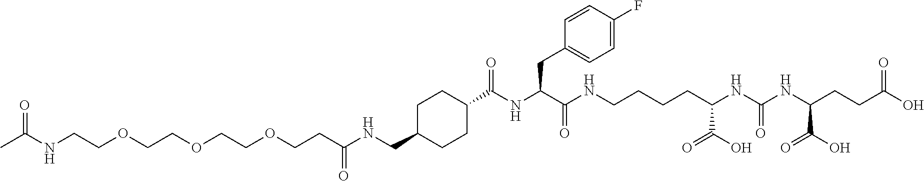 [SMILES: CC(=O)NCCOCCOCCOCCC(=O)NC[C@H]1CC[C@H](C(=O)N[C@@H](Cc2ccc(F)cc2)C(=O)NCCCC[C@H](NC(=O)N[C@@H](CCC(=O)O)C(=O)O)C(=O)O)CC1] |
| 14 | 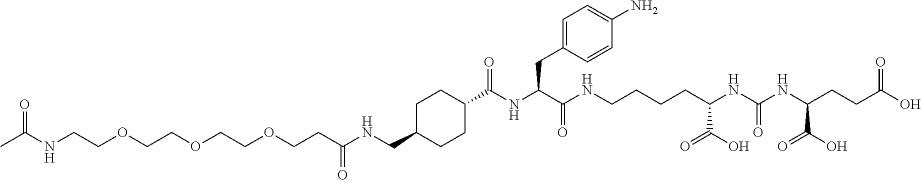 [SMILES: CC(=O)NCCOCCOCCOCCC(=O)NC[C@H]1CC[C@H](C(=O)N[C@@H](Cc2ccc(N)cc2)C(=O)NCCCC[C@H](NC(=O)N[C@@H](CCC(=O)O)C(=O)O)C(=O)O)CC1] |
| 15 | 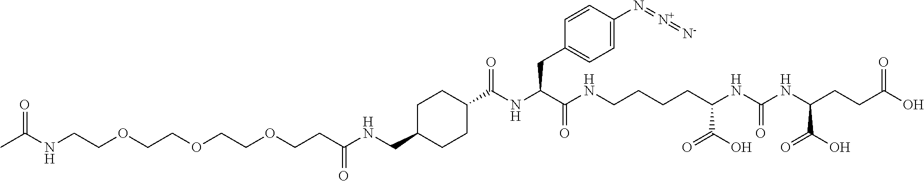 [SMILES: CC(=O)NCCOCCOCCOCCC(=O)NC[C@H]1CC[C@H](C(=O)N[C@@H](Cc2ccc(N=[N+]=[N-])cc2)C(=O)NCCCC[C@H](NC(=O)N[C@@H](CCC(=O)O)C(=O)O)C(=O)O)CC1] |
| 16 | 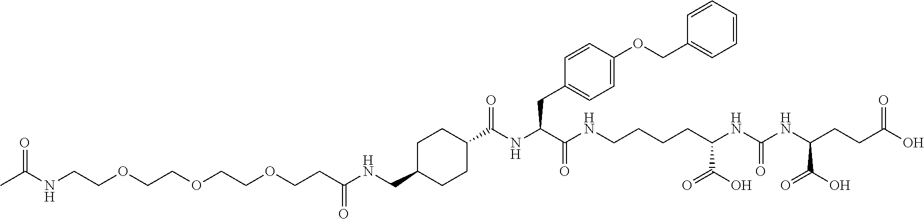 [SMILES: CC(=O)NCCOCCOCCOCCC(=O)NC[C@H]1CC[C@H](C(=O)N[C@@H](Cc2ccc(OCc3ccccc3)cc2)C(=O)NCCCC[C@H](NC(=O)N[C@@H](CCC(=O)O)C(=O)O)C(=O)O)CC1] |
| 17 | 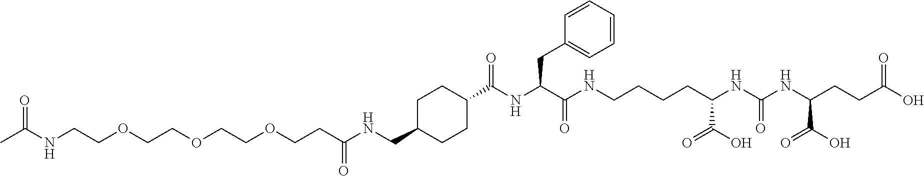 [SMILES: CC(=O)NCCOCCOCCOCCC(=O)NC[C@H]1CC[C@H](C(=O)N[C@@H](Cc2ccccc2)C(=O)NCCCC[C@H](NC(=O)N[C@@H](CCC(=O)O)C(=O)O)C(=O)O)CC1] |
| 18 | 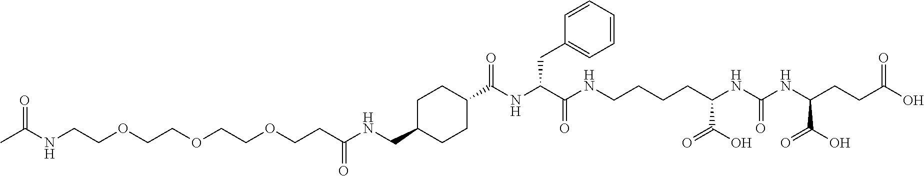 [SMILES: CC(=O)NCCOCCOCCOCCC(=O)NC[C@H]1CC[C@H](C(=O)N[C@H](Cc2ccccc2)C(=O)NCCCC[C@H](NC(=O)N[C@@H](CCC(=O)O)C(=O)O)C(=O)O)CC1] |
| 19 | 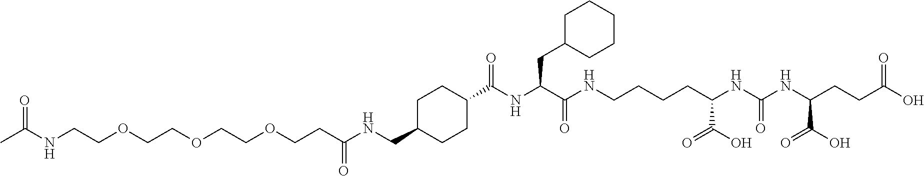 [SMILES: CC(=O)NCCOCCOCCOCCC(=O)NC[C@H]1CC[C@H](C(=O)N[C@@H](CC2CCCCC2)C(=O)NCCCC[C@H](NC(=O)N[C@@H](CCC(=O)O)C(=O)O)C(=O)O)CC1] |
| 20 | 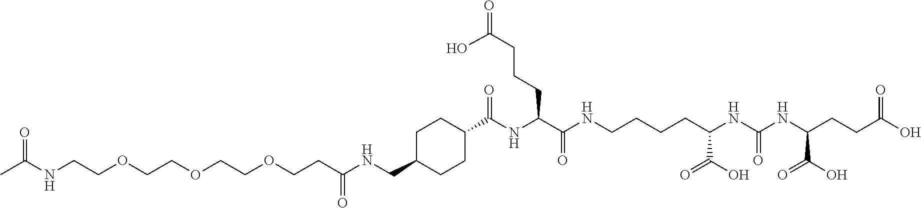 [SMILES: CC(=O)NCCOCCOCCOCCC(=O)NC[C@H]1CC[C@H](C(=O)N[C@@H](CCCC(=O)O)C(=O)NCCCC[C@H](NC(=O)N[C@@H](CCC(=O)O)C(=O)O)C(=O)O)CC1] |
| 21 | 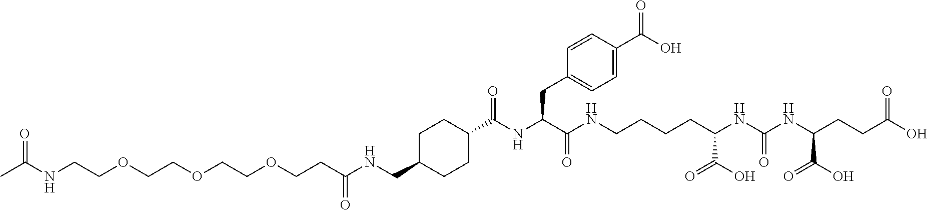 [SMILES: CC(=O)NCCOCCOCCOCCC(=O)NC[C@H]1CC[C@H](C(=O)N[C@@H](Cc2ccc(C(=O)O)cc2)C(=O)NCCCC[C@H](NC(=O)N[C@@H](CCC(=O)O)C(=O)O)C(=O)O)CC1] |
| 22 | 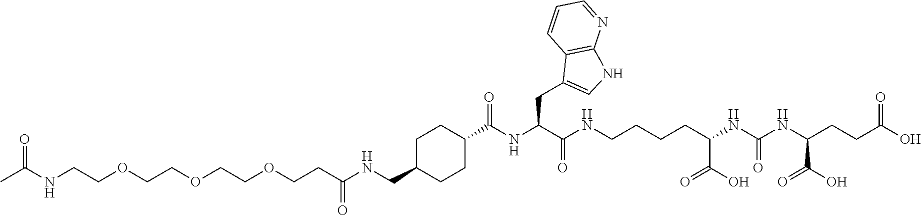 [SMILES: CC(=O)NCCOCCOCCOCCC(=O)NC[C@H]1CC[C@H](C(=O)N[C@@H](Cc2c[nH]c3ncccc23)C(=O)NCCCC[C@H](NC(=O)N[C@@H](CCC(=O)O)C(=O)O)C(=O)O)CC1] |
| 23 | 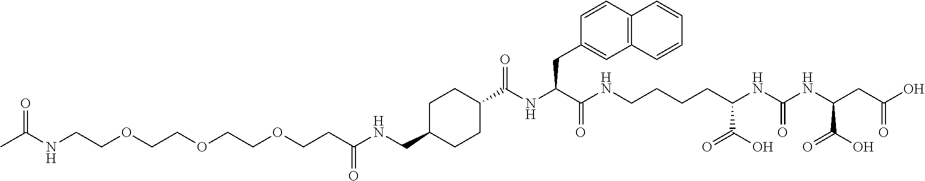 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CC(=O)O)C(=O)O)C(=O)O)CC1] |
| 24 | 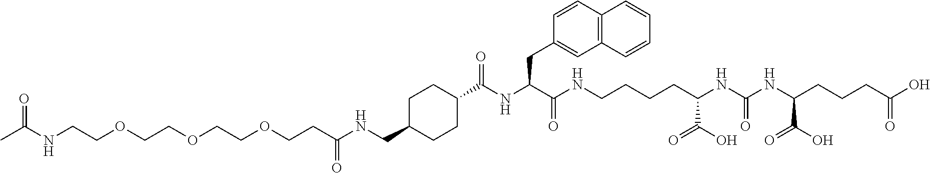 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCCC(=O)O)C(=O)O)C(=O)O)CC1] |
| 25 | 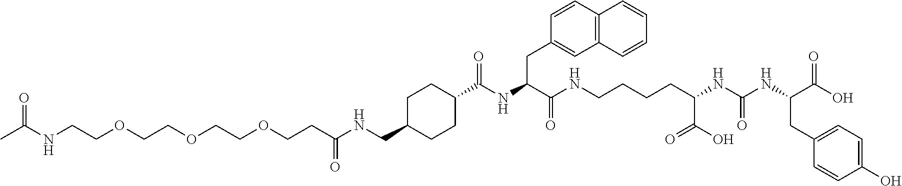 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2ccc(O)cc2)C(=O)O)C(=O)O)CC1] |
| 26 | 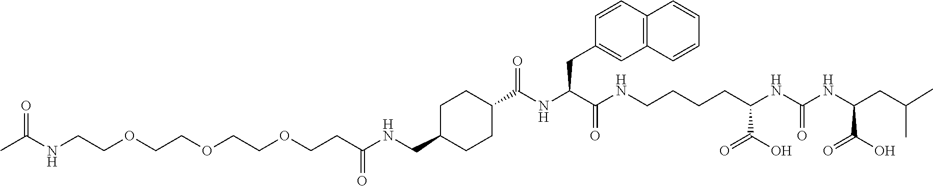 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CC(C)C)C(=O)O)C(=O)O)CC1] |
| 27 | 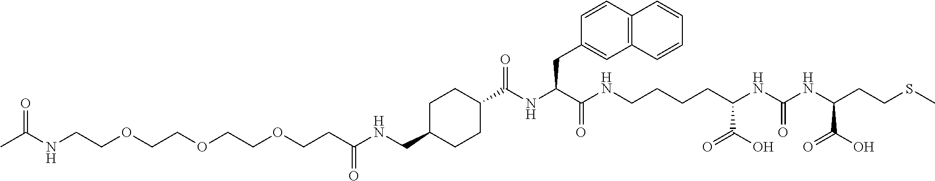 [SMILES: CSCC[C@H](NC(=O)N[C@@H](CCCCNC(=O)[C@H](Cc1ccc2ccccc2c1)NC(=O)[C@H]1CC[C@H](CNC(=O)CCOCCOCCOCCNC(C)=O)CC1)C(=O)O)C(=O)O] |
| 28 | 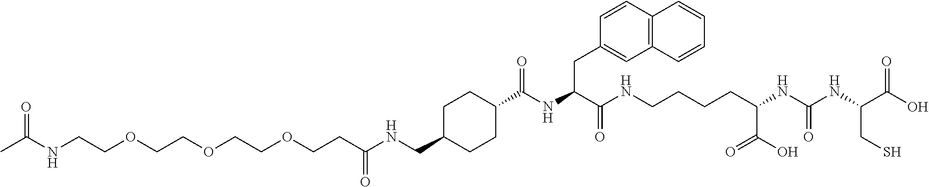 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CS)C(=O)O)C(=O)O)CC1] |
| 29 | 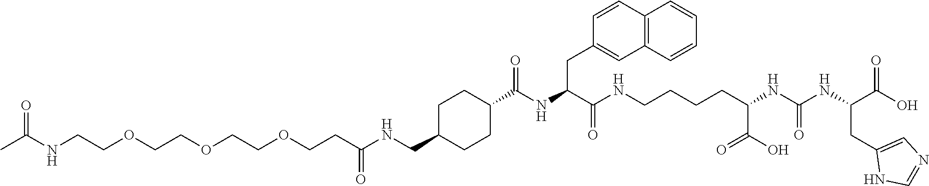 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2cnc[nH]2)C(=O)O)C(=O)O)CC1] |
| 30 | 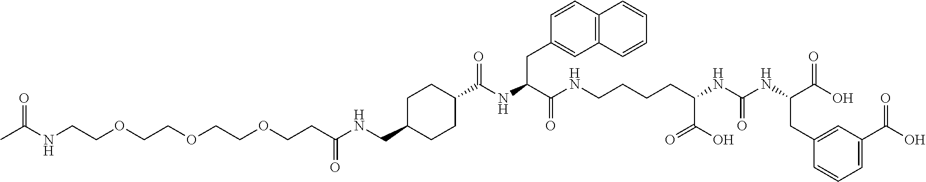 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2cccc(C(=O)O)c2)C(=O)O)C(=O)O)CC1] |
| 31 | 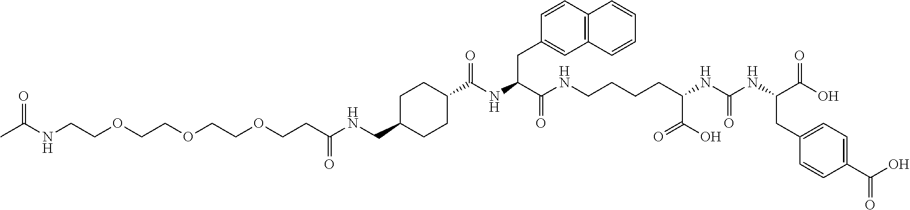 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2ccc(C(=O)O)cc2)C(=O)O)C(=O)O)CC1] |
| 32 | 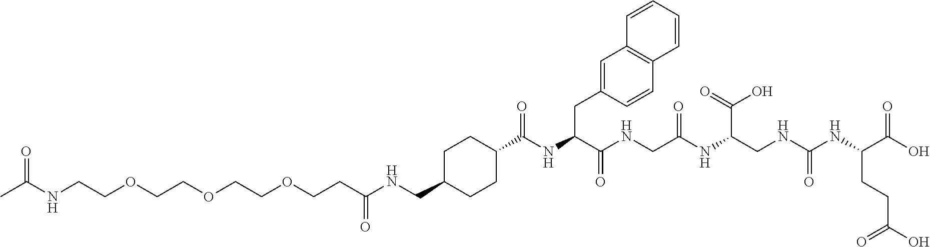 [SMILES: CC(=O)NCCOCCOCCOCCC(=O)NC[C@H]1CC[C@H](C(=O)N[C@@H](Cc2ccc3ccccc3c2)C(=O)NCC(=O)N[C@@H](CNC(=O)N[C@@H](CCC(=O)O)C(=O)O)C(=O)O)CC1] |
| 33 | 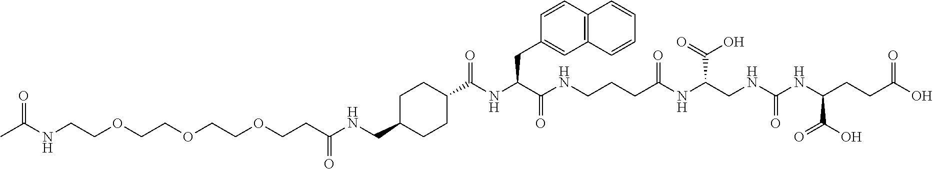 [SMILES: CC(=O)NCCOCCOCCOCCC(=O)NC[C@H]1CC[C@H](C(=O)N[C@@H](Cc2ccc3ccccc3c2)C(=O)NCCCC(=O)N[C@@H](CNC(=O)N[C@@H](CCC(=O)O)C(=O)O)C(=O)O)CC1] |
| 34 (SEQ ID NO: 79) | 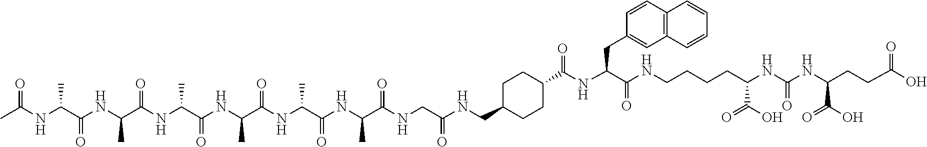 [SMILES: CC(=O)N[C@H](C)C(=O)N[C@H](C)C(=O)N[C@H](C)C(=O)N[C@H](C)C(=O)N[C@H](C)C(=O)N[C@H](C)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 35 (SEQ ID NO: 80) | 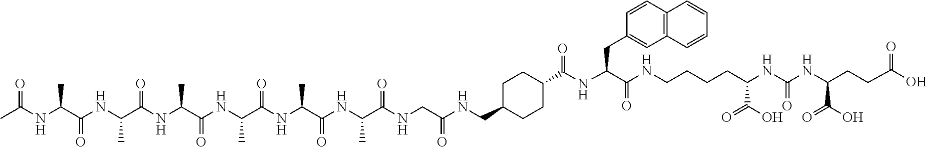 [SMILES: CC(=O)N[C@@H](C)C(=O)N[C@@H](C)C(=O)N[C@@H](C)C(=O)N[C@@H](C)C(=O)N[C@@H](C)C(=O)N[C@@H](C)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 36 (SEQ ID NO: 9) | 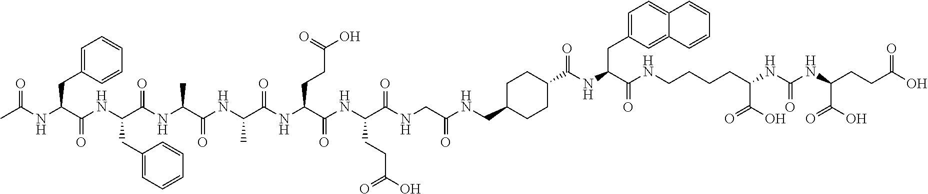 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 37 (SEQ ID NO: 107) | 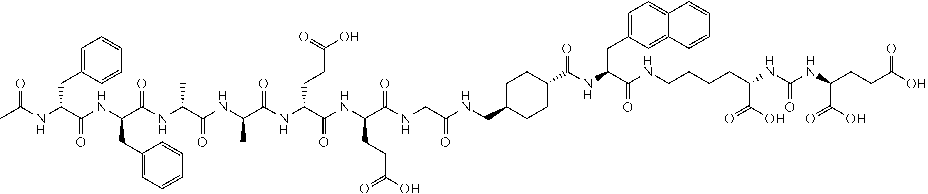 [SMILES: CC(=O)N[C@H](Cc1ccccc1)C(=O)N[C@H](Cc1ccccc1)C(=O)N[C@H](C)C(=O)N[C@H](C)C(=O)N[C@H](CCC(=O)O)C(=O)N[C@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 38 (SEQ ID NO: 81) | 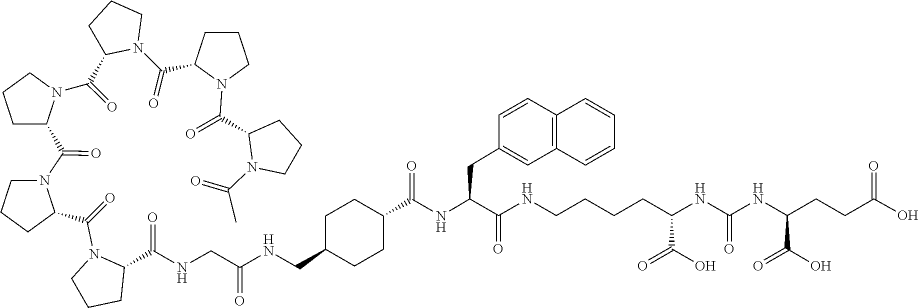 [SMILES: CC(=O)N1CCC[C@H]1C(=O)N1CCC[C@H]1C(=O)N1CCC[C@H]1C(=O)N1CCC[C@H]1C(=O)N1CCC[C@H]1C(=O)N1CCC[C@H]1C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 39 | 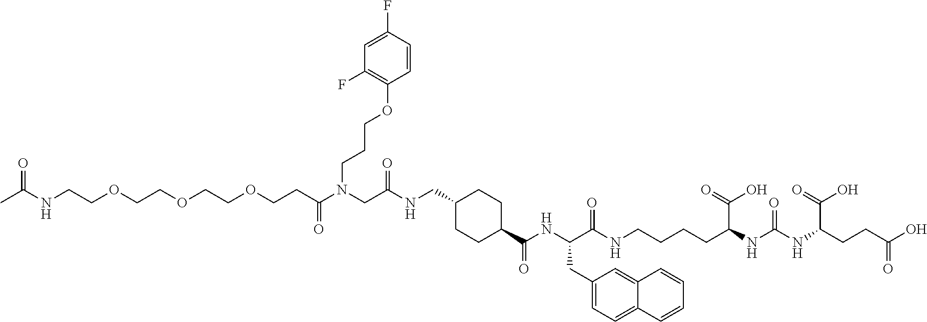 [SMILES: CC(=O)NCCOCCOCCOCCC(=O)N(CCCOc1ccc(F)cc1F)CC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 40 | 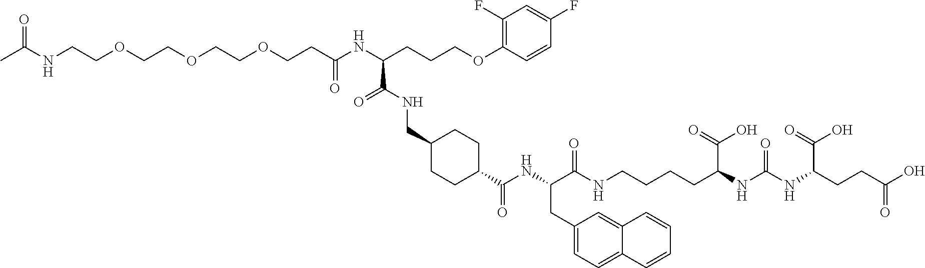 [SMILES: CC(=O)NCCOCCOCCOCCC(=O)N[C@@H](CCCOc1ccc(F)cc1F)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 41 | 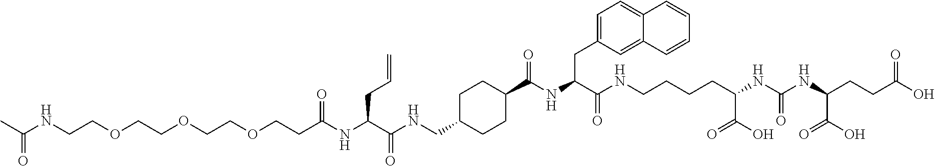 [SMILES: C=CC[C@H](NC(=O)CCOCCOCCOCCNC(C)=O)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 42 | 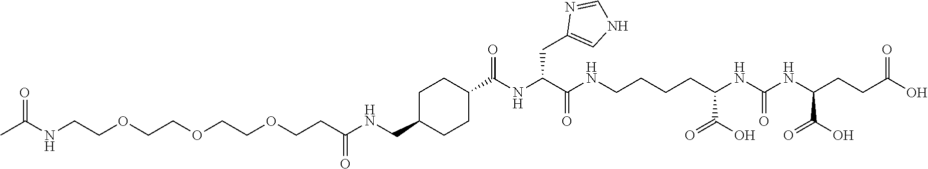 [SMILES: CC(=O)NCCOCCOCCOCCC(=O)NC[C@H]1CC[C@H](C(=O)N[C@H](Cc2c[nH]cn2)C(=O)NCCCC[C@H](NC(=O)N[C@@H](CCC(=O)O)C(=O)O)C(=O)O)CC1] |
| 43 | 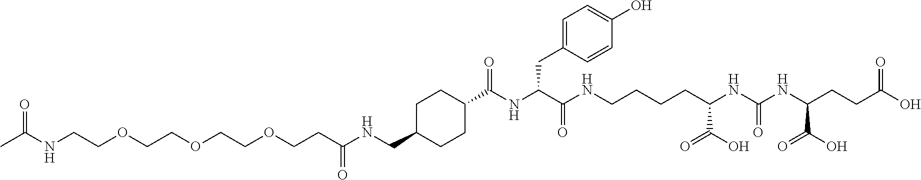 [SMILES: CC(=O)NCCOCCOCCOCCC(=O)NC[C@H]1CC[C@H](C(=O)N[C@H](Cc2ccc(O)cc2)C(=O)NCCCC[C@H](NC(=O)N[C@@H](CCC(=O)O)C(=O)O)C(=O)O)CC1] |
| 44 | 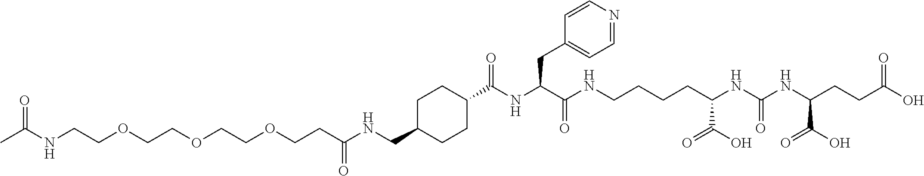 [SMILES: CC(=O)NCCOCCOCCOCCC(=O)NC[C@H]1CC[C@H](C(=O)N[C@@H](Cc2ccncc2)C(=O)NCCCC[C@H](NC(=O)N[C@@H](CCC(=O)O)C(=O)O)C(=O)O)CC1] |
| 45 | 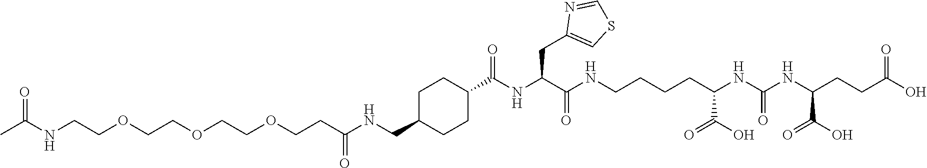 [SMILES: CC(=O)NCCOCCOCCOCCC(=O)NC[C@H]1CC[C@H](C(=O)N[C@@H](Cc2cscn2)C(=O)NCCCC[C@H](NC(=O)N[C@@H](CCC(=O)O)C(=O)O)C(=O)O)CC1] |
| 46 | 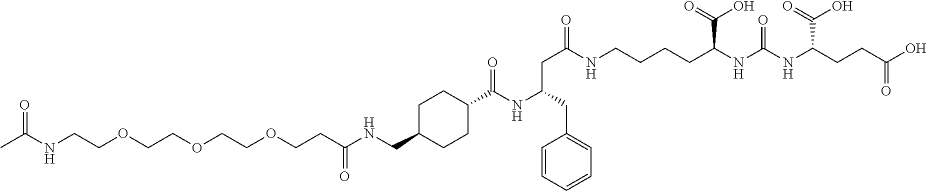 [SMILES: CC(=O)NCCOCCOCCOCCC(=O)NC[C@H]1CC[C@H](C(=O)N[C@H](CC(=O)NCCCC[C@H](NC(=O)N[C@@H](CCC(=O)O)C(=O)O)C(=O)O)Cc2ccccc2)CC1] |
| 47 | 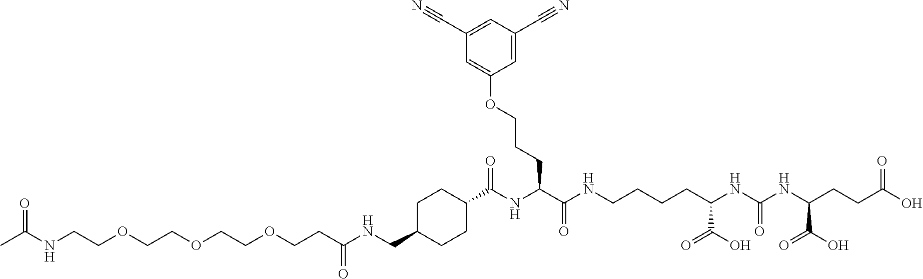 [SMILES: CC(=O)NCCOCCOCCOCCC(=O)NC[C@H]1CC[C@H](C(=O)N[C@@H](CCCOc2cc(C#N)cc(C#N)c2)C(=O)NCCCC[C@H](NC(=O)N[C@@H](CCC(=O)O)C(=O)O)C(=O)O)CC1] |
| 48 | 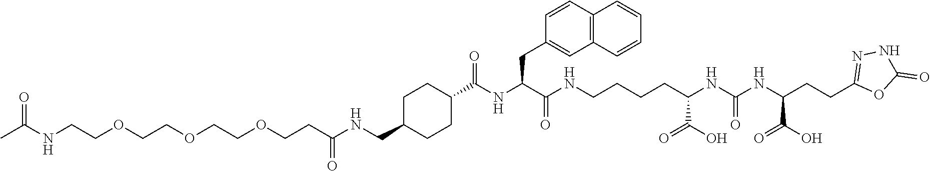 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCc2n[nH]c(=O)o2)C(=O)O)C(=O)O)CC1] |
| 49 | 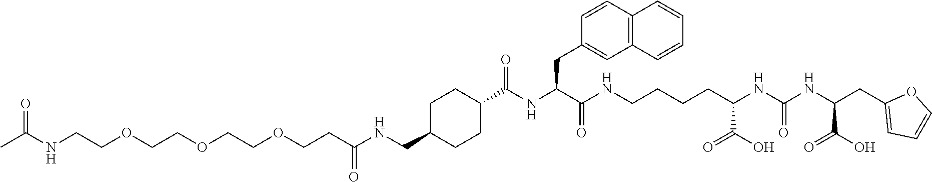 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2ccco2)C(=O)O)C(=O)O)CC1] |
| 50 | 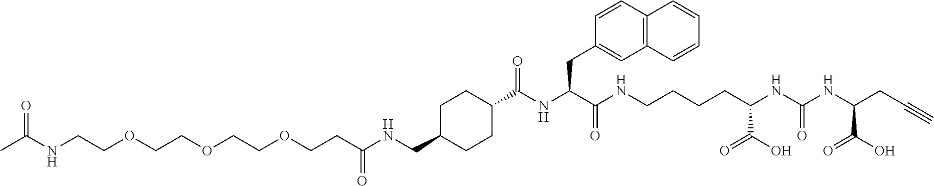 [SMILES: C#CC[C@H](NC(=O)N[C@@H](CCCCNC(=O)[C@H](Cc1ccc2ccccc2c1)NC(=O)[C@H]1CC[C@H](CNC(=O)CCOCCOCCOCCNC(C)=O)CC1)C(=O)O)C(=O)O] |
| 51 | 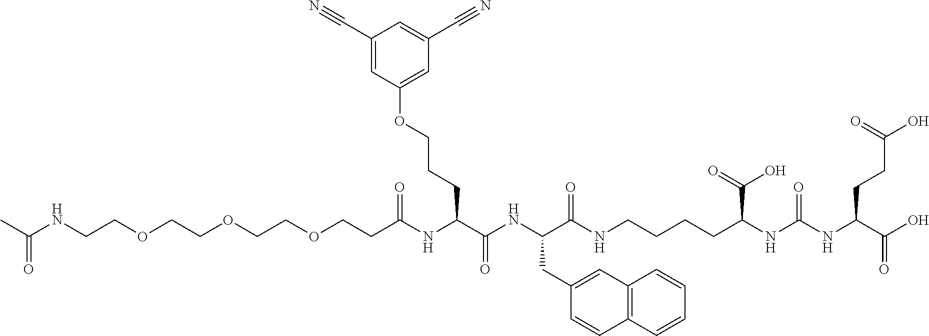 [SMILES: CC(=O)NCCOCCOCCOCCC(=O)N[C@@H](CCCOc1cc(C#N)cc(C#N)c1)C(=O)N[C@@H](Cc1ccc2ccccc2c1)C(=O)NCCCC[C@H](NC(=O)N[C@@H](CCC(=O)O)C(=O)O)C(=O)O] |
| 52 | 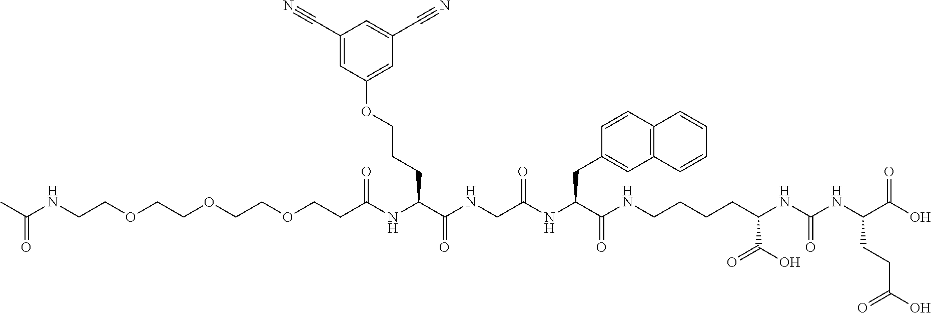 [SMILES: CC(=O)NCCOCCOCCOCCC(=O)N[C@@H](CCCOc1cc(C#N)cc(C#N)c1)C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 53 | 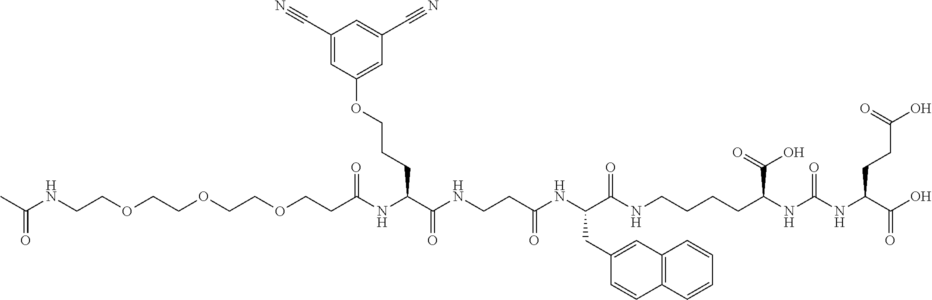 [SMILES: CC(=O)NCCOCCOCCOCCC(=O)N[C@@H](CCCOc1cc(C#N)cc(C#N)c1)C(=O)NCCC(=O)N[C@@H](Cc1ccc2ccccc2c1)C(=O)NCCCC[C@H](NC(=O)N[C@@H](CCC(=O)O)C(=O)O)C(=O)O] |
| 54 | 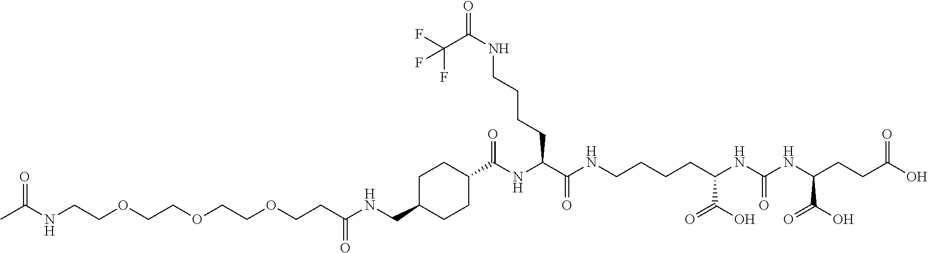 [SMILES: CC(=O)NCCOCCOCCOCCC(=O)NC[C@H]1CC[C@H](C(=O)N[C@@H](CCCCNC(=O)C(F)(F)F)C(=O)NCCCC[C@H](NC(=O)N[C@@H](CCC(=O)O)C(=O)O)C(=O)O)CC1] |
| 55 (SEQ ID NO: 110) | 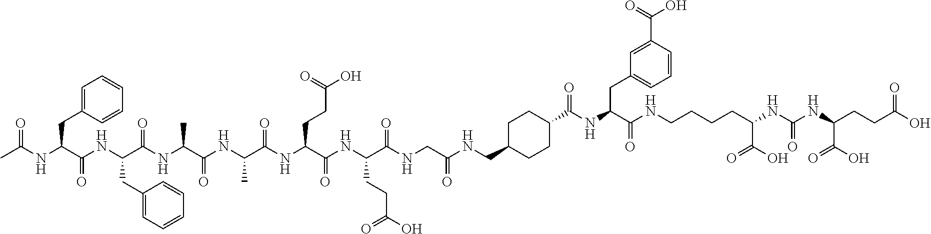 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2cccc(C(=O)O)c2)C(=O)NCCCC[C@H](NC(=O)N[C@@H](CCC(=O)O)C(=O)O)C(=O)O)CC1] |
| 56 (SEQ ID NO: 108) | 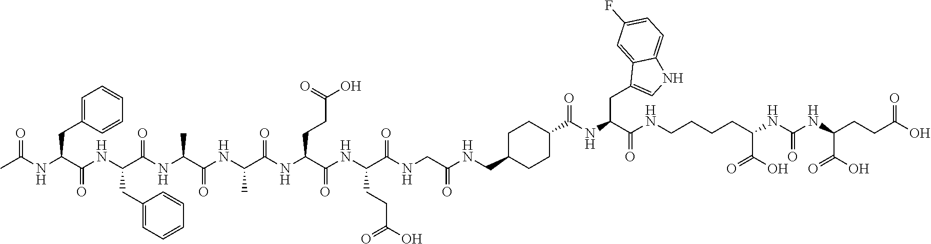 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2c[nH]c3ccc(F)cc23)C(=O)NCCCC[C@H](NC(=O)N[C@@H](CCC(=O)O)C(=O)O)C(=O)O)CC1] |
| 57 (SEQ ID NO: 109) | 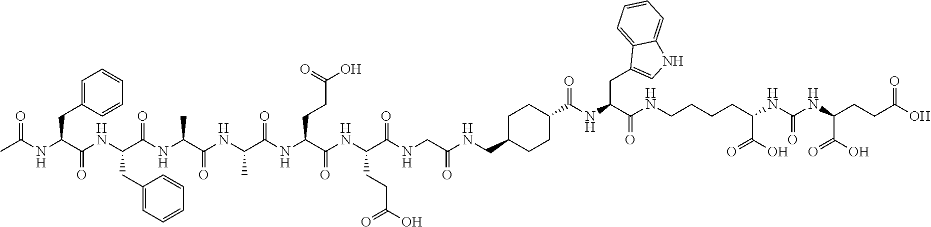 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2c[nH]c3ccccc23)C(=O)NCCCC[C@H](NC(=O)N[C@@H](CCC(=O)O)C(=O)O)C(=O)O)CC1] |
| 58 (SEQ ID NO: 51) | 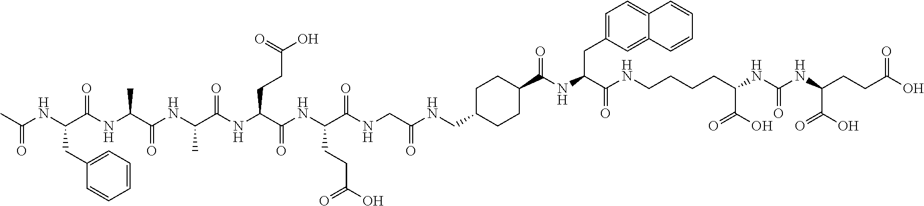 [SMILES: CC(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 59 (SEQ ID NO: 40) | 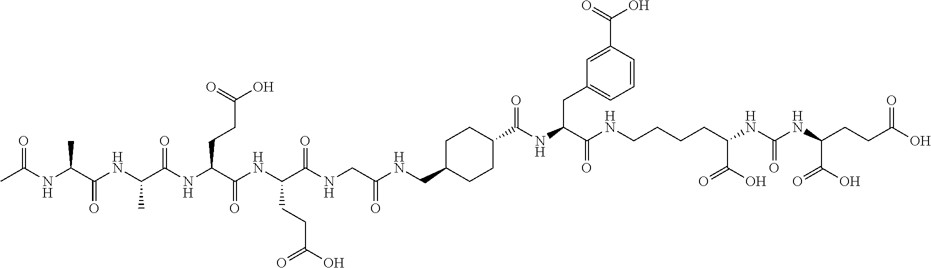 [SMILES: CC(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2cccc(C(=O)O)c2)C(=O)NCCCC[C@H](NC(=O)N[C@@H](CCC(=O)O)C(=O)O)C(=O)O)CC1] |
| 60 (SEQ ID NO: 82) | 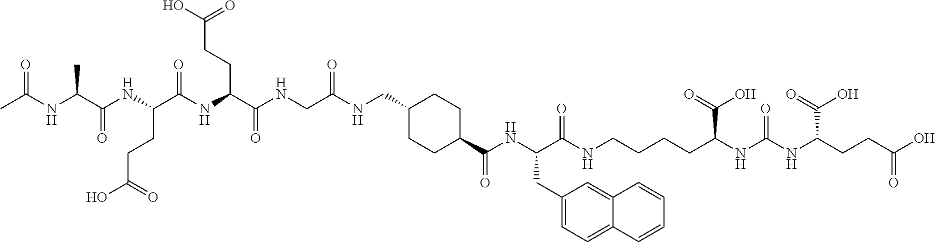 [SMILES: CC(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 61 (SEQ ID NO: 47) | 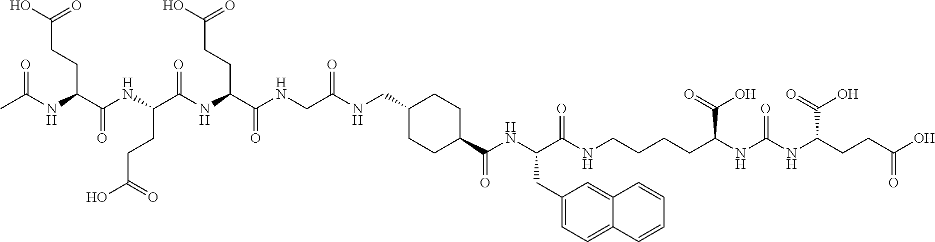 [SMILES: CC(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 62 (SEQ ID NO: 38) | 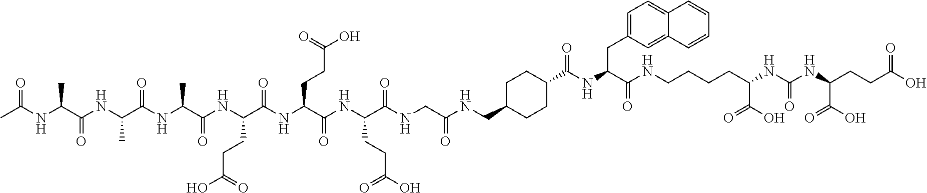 [SMILES: CC(=O)N[C@@H](C)C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 63 (SEQ ID NO: 35) | 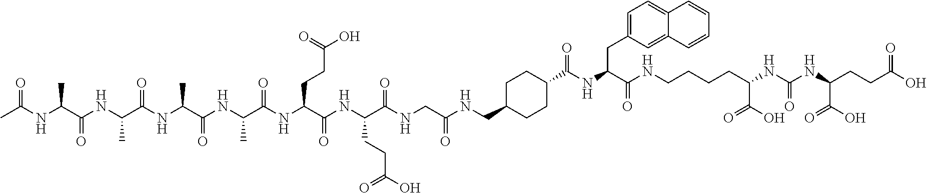 [SMILES: CC(=O)N[C@@H](C)C(=O)N[C@@H](C)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 64 (SEQ ID NO: 72) | 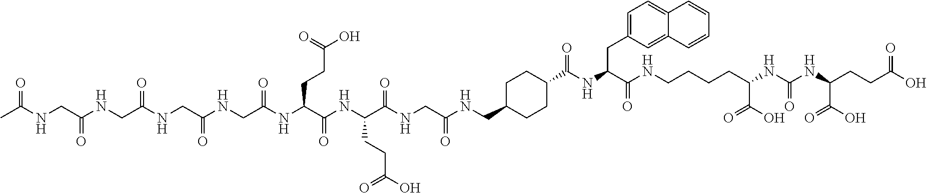 [SMILES: CC(=O)NCC(=O)NCC(=O)NCC(=O)NCC(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 65 (SEQ ID NO: 60) | 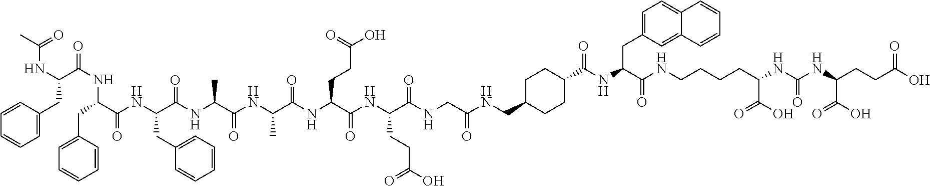 [SMILES: CC(=O)N[C@@H](Cc1ccccc1)C(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 66 (SEQ ID NO: 55) | 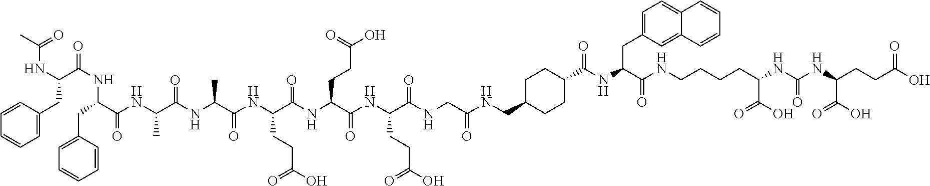 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 67 (SEQ ID NO: 42) | 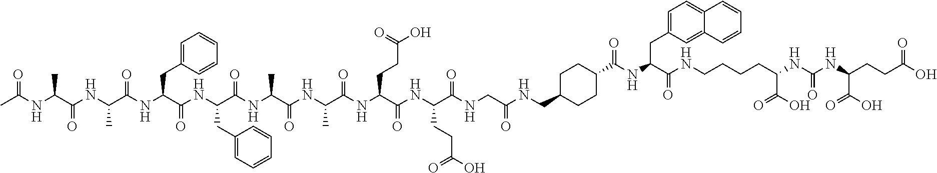 [SMILES: CC(=O)N[C@@H](C)C(=O)N[C@@H](C)C(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 68 (SEQ ID NO: 10) | 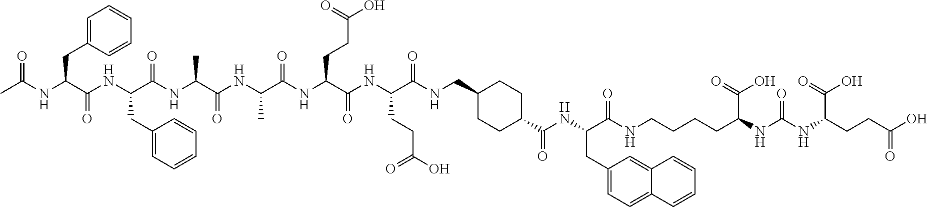 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 69 (SEQ ID NO: 17) | 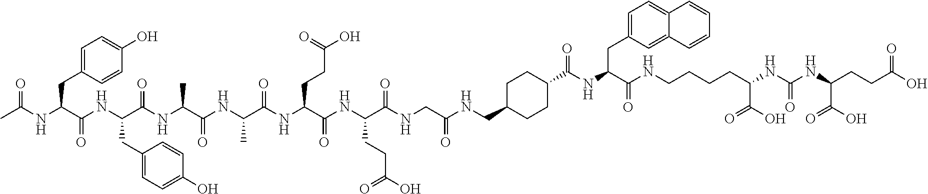 [SMILES: CC(=O)N[C@@H](Cc1ccc(O)cc1)C(=O)N[C@@H](Cc1ccc(O)cc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 70 (SEQ ID NO: 83) | 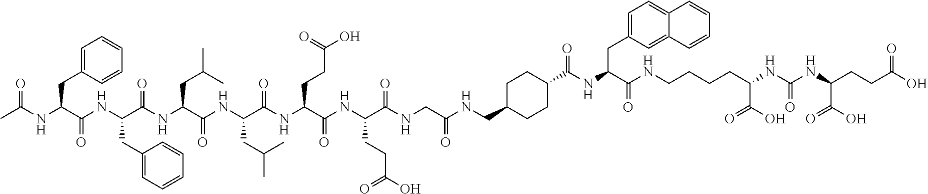 [SMILES: CC(=O)N[C@@H](Cc1ccccc1)C(=O)N[C@@H](Cc1ccccc1)C(=O)N[C@@H](CC(C)C)C(=O)N[C@@H](CC(C)C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 71 (SEQ ID NO: 66) | 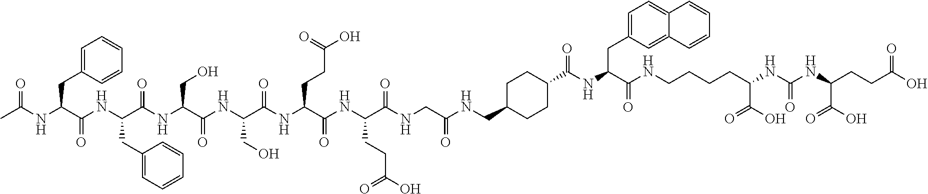 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 72 (SEQ ID NO: 46) | 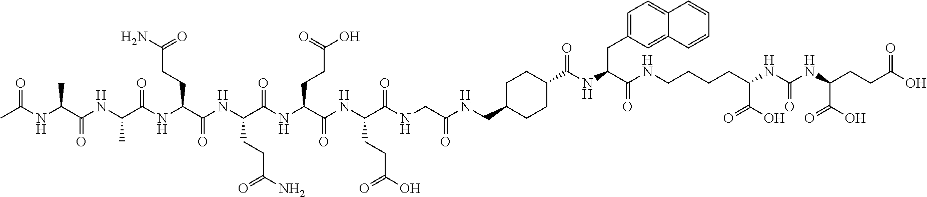 [SMILES: CC(=O)N[C@@H](C)C(=O)N[C@@H](C)C(=O)N[C@@H](CCC(N)=O)C(=O)N[C@@H](CCC(N)=O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 73 (SEQ ID NO: 64) | 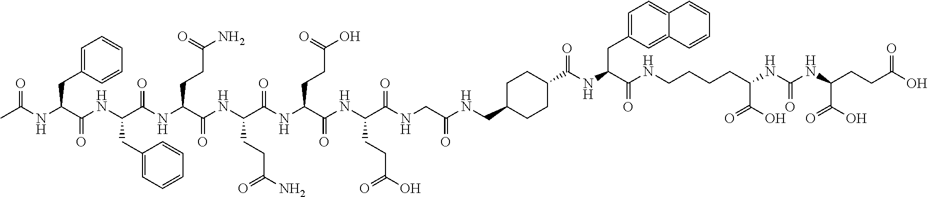 [SMILES: CC(=O)N[C@@H](Cc1ccccc1)C(=O)N[C@@H](Cc1ccccc1)C(=O)N[C@@H](CCC(N)=O)C(=O)N[C@@H](CCC(N)=O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 74 (SEQ ID NO: 58) | 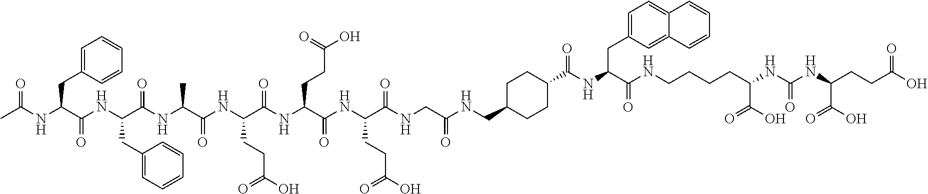 [SMILES: CC(=O)N[C@@H](Cc1ccccc1)C(=O)N[C@@H](Cc1ccccc1)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 75 (SEQ ID NO: 44) | 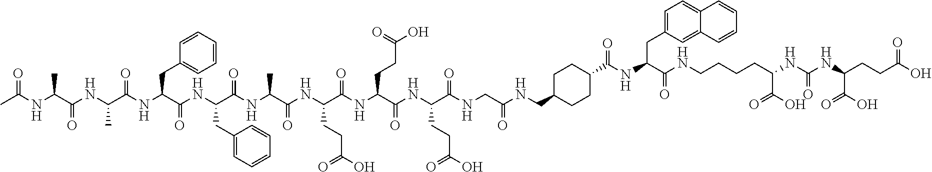 [SMILES: CC(=O)N[C@@H](C)C(=O)N[C@@H](C)C(=O)N[C@@H](Cc1ccccc1)C(=O)N[C@@H](Cc1ccccc1)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 76 (SEQ ID NO: 53) | 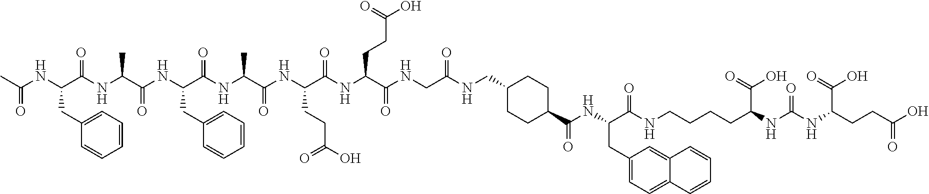 [SMILES: CC(=O)N[C@@H](Cc1ccccc1)C(=O)N[C@@H](C)C(=O)N[C@@H](Cc1ccccc1)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 77 (SEQ ID NO: 84) | 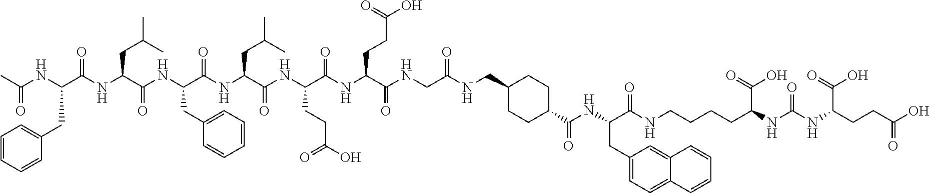 [SMILES: CC(=O)N[C@@H](Cc1ccccc1)C(=O)N[C@@H](CC(C)C)C(=O)N[C@@H](Cc1ccccc1)C(=O)N[C@@H](CC(C)C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1] |
| 78 | 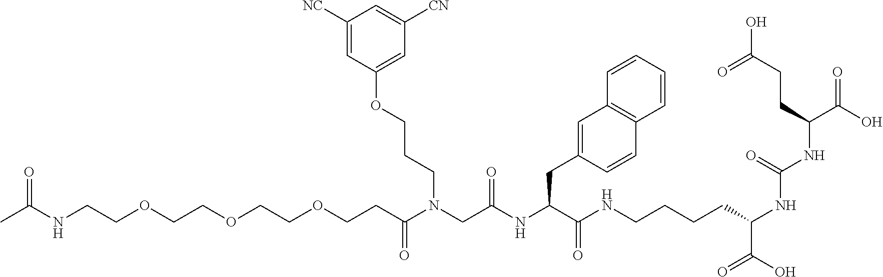 [SMILES: CC(=O)NCCOCCOCCOCCC(=O)N(CCCOc1cc(C#N)cc(C#N)c1)CC(=O)N[C@@H](Cc1ccc2ccccc2c1)C(=O)NCCCC[C@H](NC(=O)N[C@@H](CCC(=O)O)C(=O)O)C(=O)O] |
| 79 | 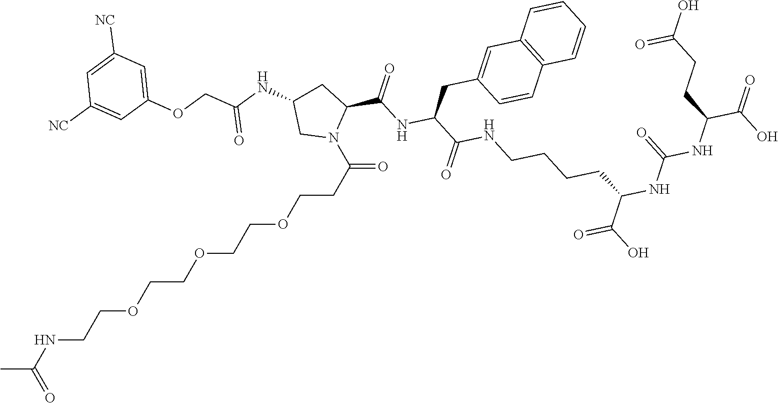 [SMILES: CC(=O)NCCOCCOCCOCCC(=O)N1C[C@H](NC(=O)COc2cc(C#N)cc(C#N)c2)C[C@H]1C(=O)N[C@@H](Cc1ccc2ccccc2c1)C(=O)NCCCC[C@H](NC(=O)N[C@@H](CCC(=O)O)C(=O)O)C(=O)O] |
| 80 | 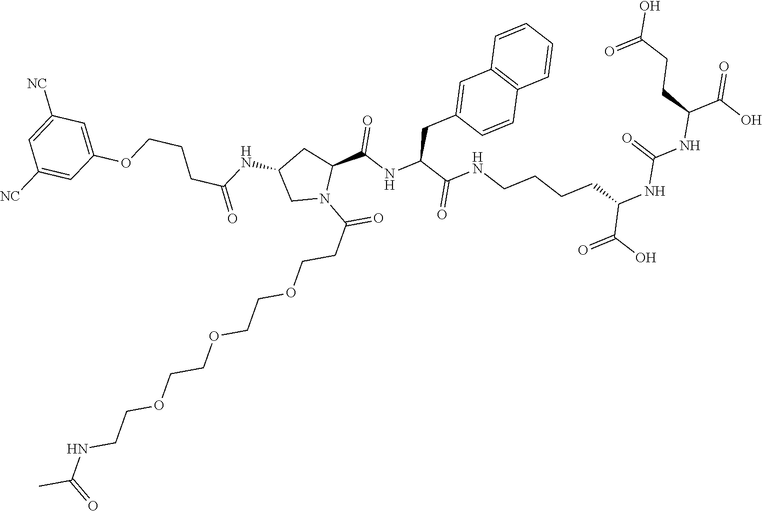 [SMILES: CC(=O)NCCOCCOCCOCCC(=O)N1C[C@H](NC(=O)CCCOc2cc(C#N)cc(C#N)c2)C[C@H]1C(=O)N[C@@H](Cc1ccc2ccccc2c1)C(=O)NCCCC[C@H](NC(=O)N[C@@H](CCC(=O)O)C(=O)O)C(=O)O] |
| 81 | 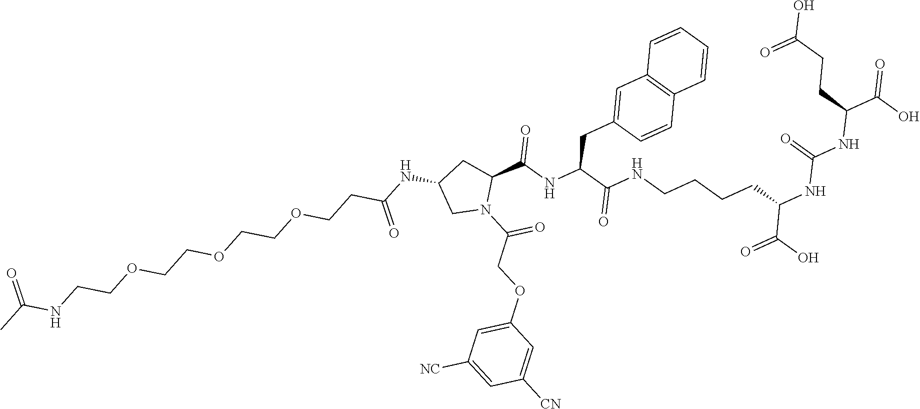 [SMILES: CC(=O)NCCOCCOCCOCCC(=O)N[C@@H]1C[C@@H](C(=O)N[C@@H](Cc2ccc3ccccc3c2)C(=O)NCCCC[C@H](NC(=O)N[C@@H](CCC(=O)O)C(=O)O)C(=O)O)N(C(=O)COc2cc(C#N)cc(C#N)c2)C1] |
| 82 | 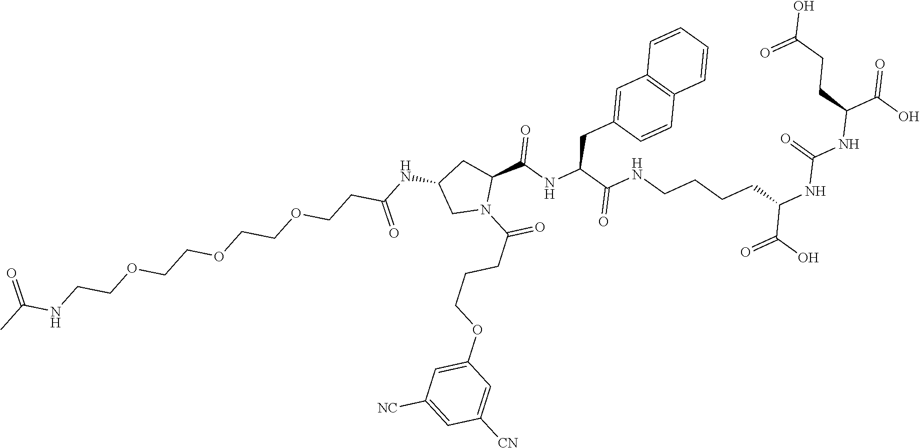 [SMILES: CC(=O)NCCOCCOCCOCCC(=O)N[C@@H]1C[C@@H](C(=O)N[C@@H](Cc2ccc3ccccc3c2)C(=O)NCCCC[C@H](NC(=O)N[C@@H](CCC(=O)O)C(=O)O)C(=O)O)N(C(=O)CCCOc2cc(C#N)cc(C#N)c2)C1] |
| 83 | 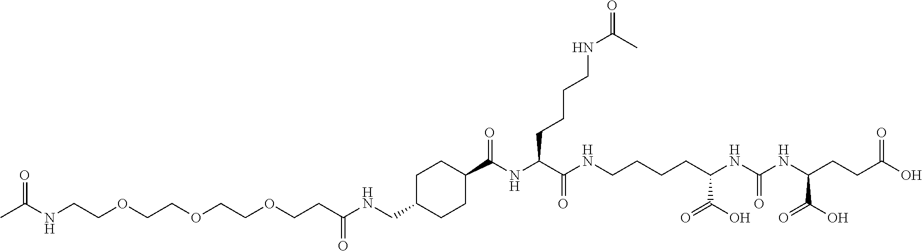 [SMILES: CC(=O)NCCCC[C@H](NC(=O)[C@H]1CC[C@H](CNC(=O)CCOCCOCCOCCNC(C)=O)CC1)C(=O)NCCCC[C@H](NC(=O)N[C@@H](CCC(=O)O)C(=O)O)C(=O)O] |
| 84 | 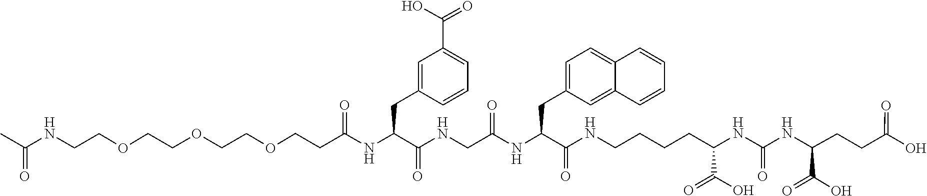 [SMILES: CC(=O)NCCOCCOCCOCCC(=O)N[C@@H](Cc1cccc(C(=O)O)c1)C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 85 | 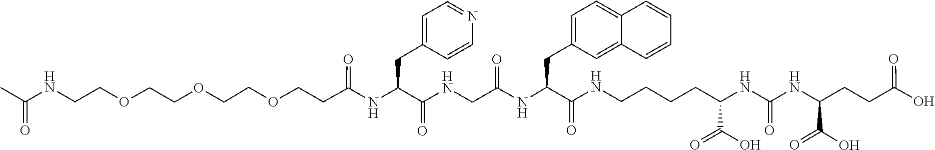 [SMILES: CC(=O)NCCOCCOCCOCCC(=O)N[C@@H](Cc1ccncc1)C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 86 | 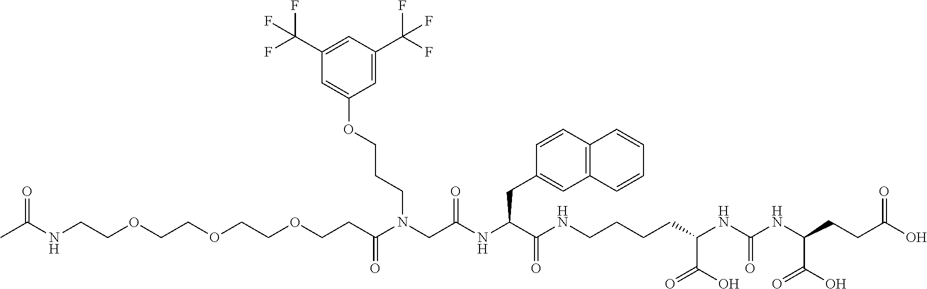 [SMILES: CC(=O)NCCOCCOCCOCCC(=O)N(CCCOc1cc(C(F)(F)F)cc(C(F)(F)F)c1)CC(=O)N[C@@H](Cc1ccc2ccccc2c1)C(=O)NCCCC[C@H](NC(=O)N[C@@H](CCC(=O)O)C(=O)O)C(=O)O] |
| 87 | 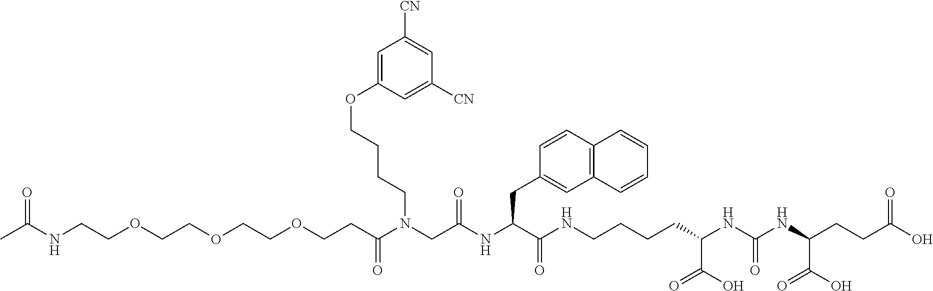 [SMILES: CC(=O)NCCOCCOCCOCCC(=O)N(CCCCOc1cc(C#N)cc(C#N)c1)CC(=O)N[C@@H](Cc1ccc2ccccc2c1)C(=O)NCCCC[C@H](NC(=O)N[C@@H](CCC(=O)O)C(=O)O)C(=O)O] |
| 88 | 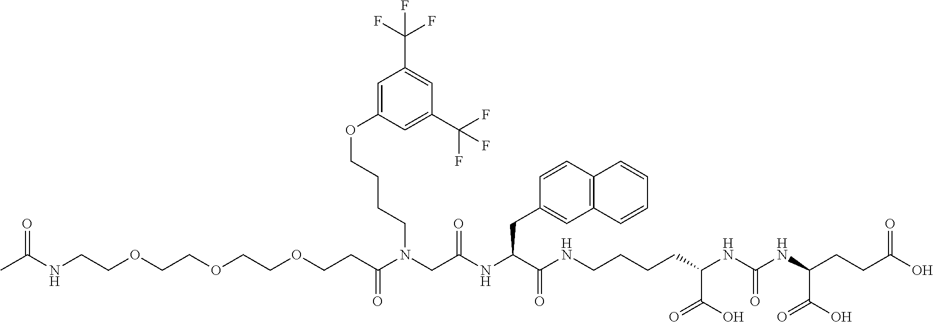 [SMILES: CC(=O)NCCOCCOCCOCCC(=O)N(CCCCOc1cc(C(F)(F)F)cc(C(F)(F)F)c1)CC(=O)N[C@@H](Cc1ccc2ccccc2c1)C(=O)NCCCC[C@H](NC(=O)N[C@@H](CCC(=O)O)C(=O)O)C(=O)O] |
| 89 | 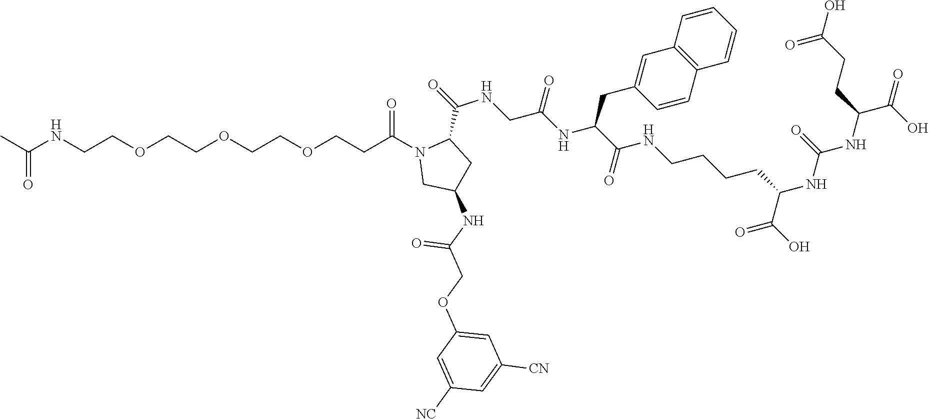 [SMILES: CC(=O)NCCOCCOCCOCCC(=O)N1C[C@H](NC(=O)COc2cc(C#N)cc(C#N)c2)C[C@H]1C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 90 | 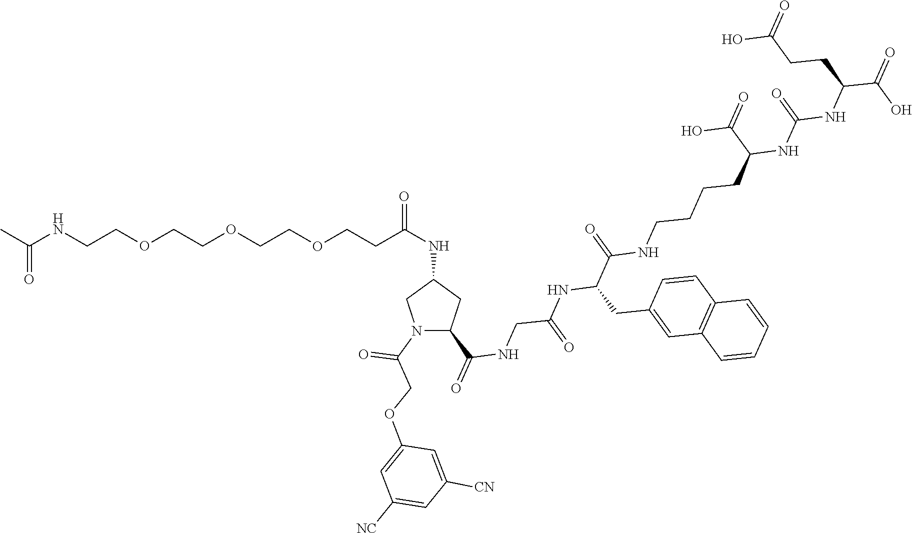 [SMILES: CC(=O)NCCOCCOCCOCCC(=O)N[C@@H]1C[C@@H](C(=O)NCC(=O)N[C@@H](Cc2ccc3ccccc3c2)C(=O)NCCCC[C@H](NC(=O)N[C@@H](CCC(=O)O)C(=O)O)C(=O)O)N(C(=O)COc2cc(C#N)cc(C#N)c2)C1] |
| 91 | 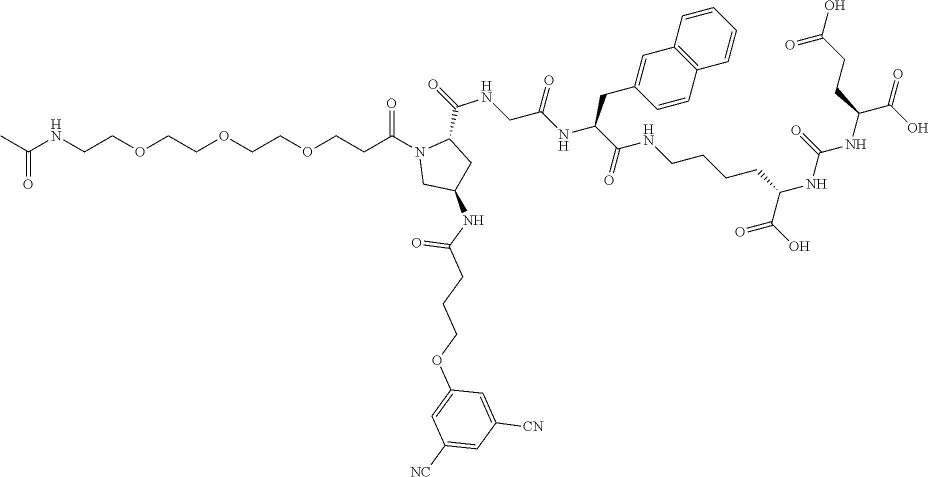 [SMILES: CC(=O)NCCOCCOCCOCCC(=O)N1C[C@H](NC(=O)CCCOc2cc(C#N)cc(C#N)c2)C[C@H]1C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 92 | 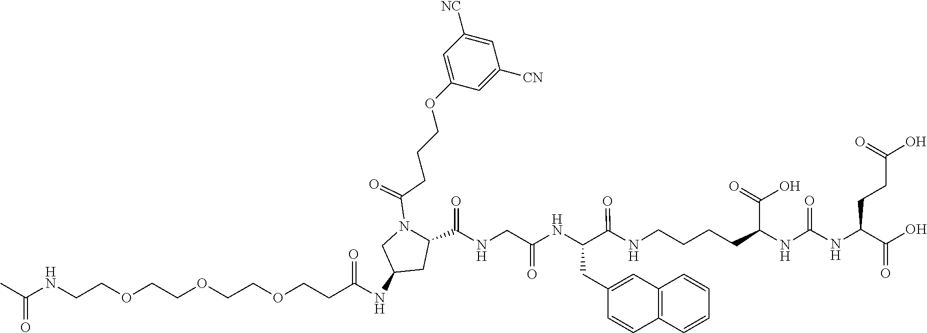 [SMILES: CC(=O)NCCOCCOCCOCCC(=O)N[C@@H]1C[C@@H](C(=O)NCC(=O)N[C@@H](Cc2ccc3ccccc3c2)C(=O)NCCCC[C@H](NC(=O)N[C@@H](CCC(=O)O)C(=O)O)C(=O)O)N(C(=O)CCCOc2cc(C#N)cc(C#N)c2)C1] |
| 93 | 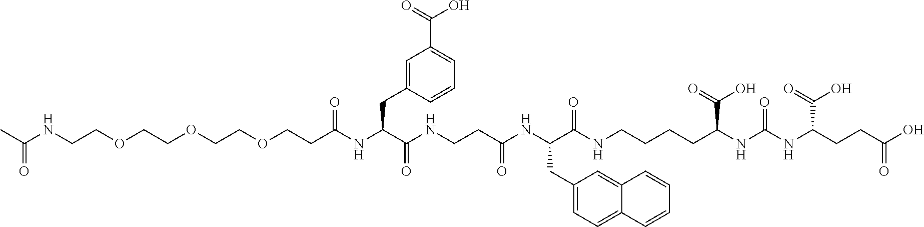 [SMILES: CC(=O)NCCOCCOCCOCCC(=O)N[C@@H](Cc1cccc(C(=O)O)c1)C(=O)NCCC(=O)N[C@@H](Cc1ccc2ccccc2c1)C(=O)NCCCC[C@H](NC(=O)N[C@@H](CCC(=O)O)C(=O)O)C(=O)O] |
| 94 | 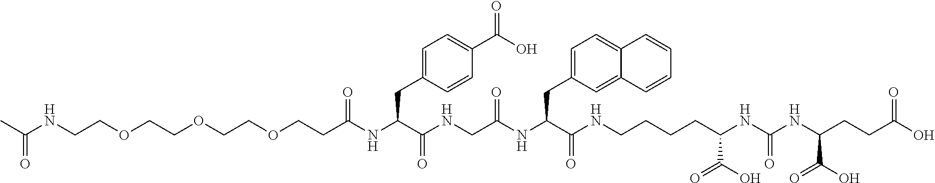 [SMILES: CC(=O)NCCOCCOCCOCCC(=O)N[C@@H](Cc1ccc(C(=O)O)cc1)C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 95 | 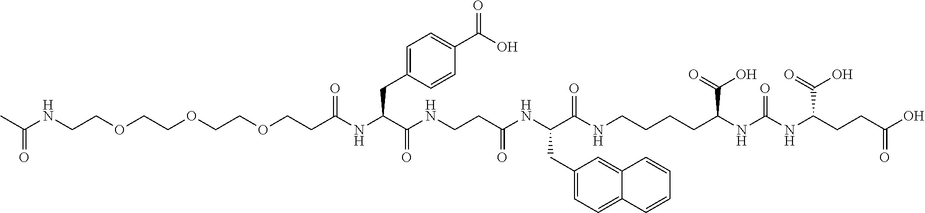 [SMILES: CC(=O)NCCOCCOCCOCCC(=O)N[C@@H](Cc1ccc(C(=O)O)cc1)C(=O)NCCC(=O)N[C@@H](Cc1ccc2ccccc2c1)C(=O)NCCCC[C@H](NC(=O)N[C@@H](CCC(=O)O)C(=O)O)C(=O)O] |
| 96 | 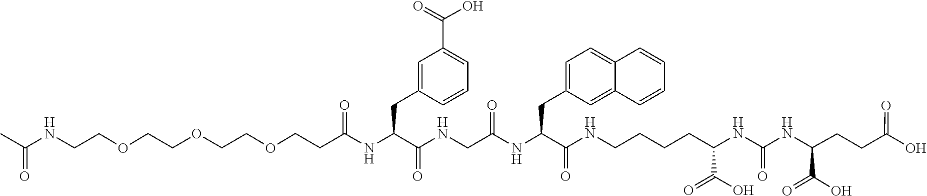 [SMILES: CC(=O)NCCOCCOCCOCCC(=O)N[C@@H](Cc1cccc(C(=O)O)c1)C(=O)NCC(=O)N[C@@H](Cc1ccc2ccccc2c1)C(=O)NCCCC[C@H](NC(=O)N[C@@H](CCC(=O)O)C(=O)O)C(=O)O] |
| 97 | 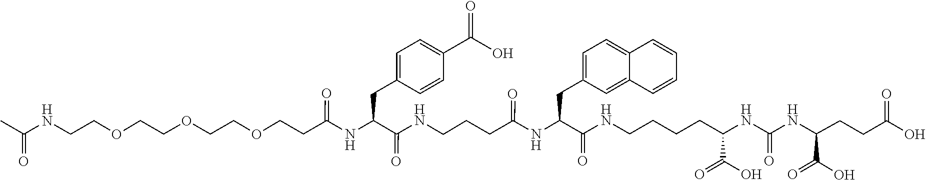 [SMILES: CC(=O)NCCOCCOCCOCCC(=O)N[C@@H](Cc1ccc(C(=O)O)cc1)C(=O)NCCCC(=O)N[C@@H](Cc1ccc2ccccc2c1)C(=O)NCCCC[C@H](NC(=O)N[C@@H](CCC(=O)O)C(=O)O)C(=O)O] |
| 98 (SEQ ID NO: 85) | 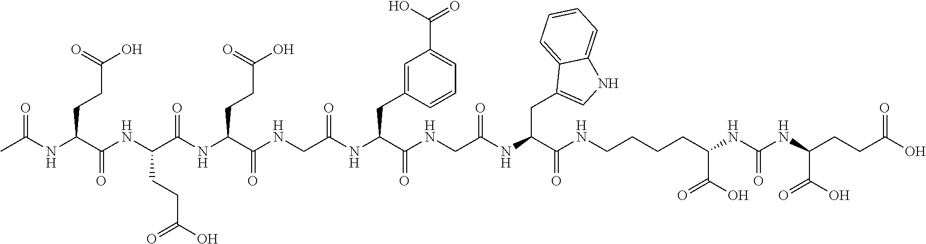 [SMILES: CC(=O)N[C@@H](CCC(=O)O)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 99 (SEQ ID NO: 86) | 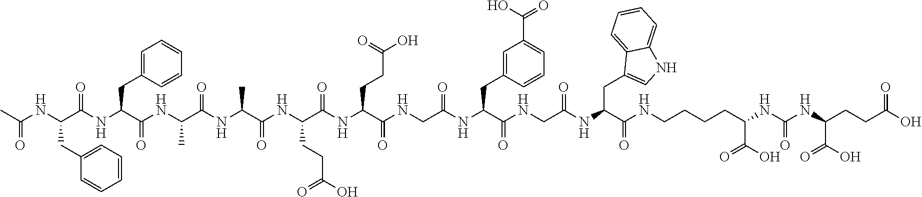 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 100 (SEQ ID NO: 87) | 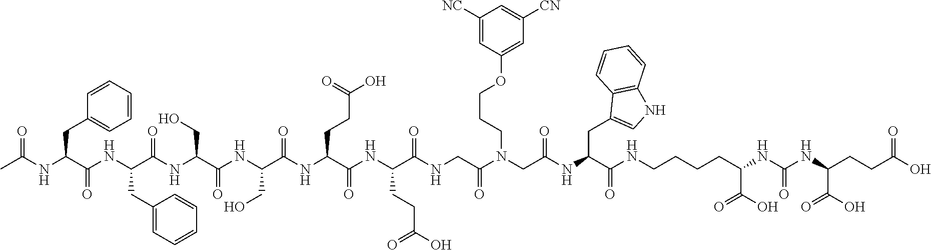 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O] |
| 101 (SEQ ID NO: 88) | 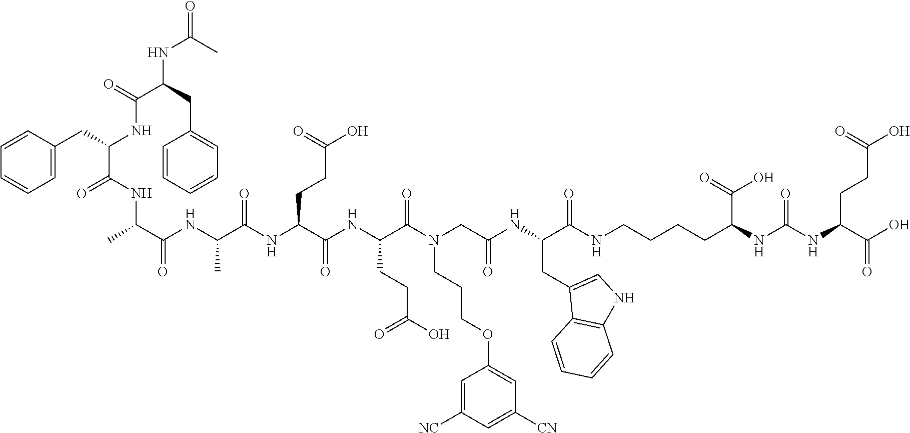 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O] |
| 102 (SEQ ID NO: 89) | 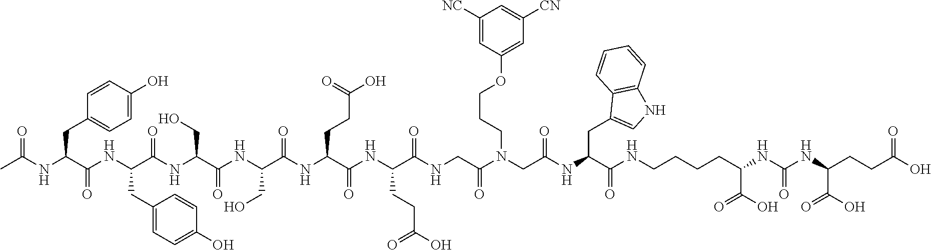 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O] |
| 103 (SEQ ID NO: 90) | 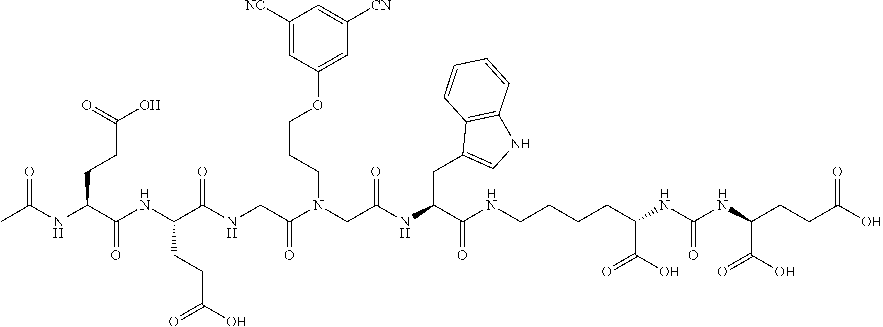 [SMILES: CC(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O] |
| 104 (SEQ ID NO: 91) | 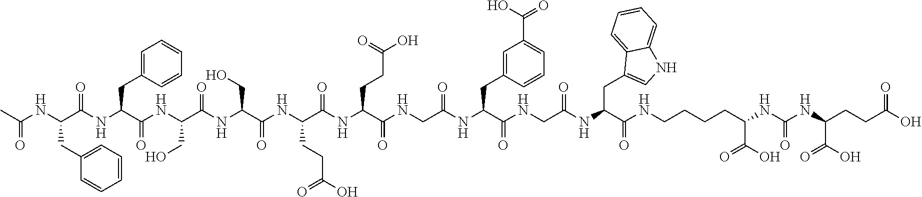 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 105 (SEQ ID NO: 92) | 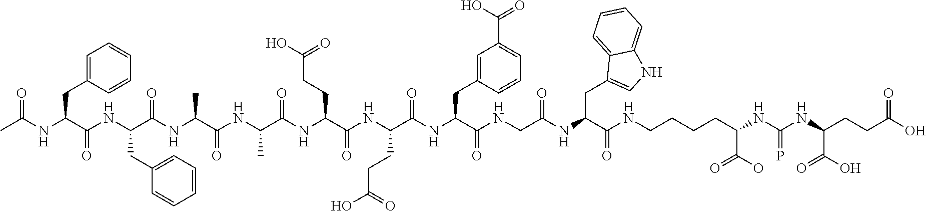 |
| 106 (SEQ ID NO: 93) | 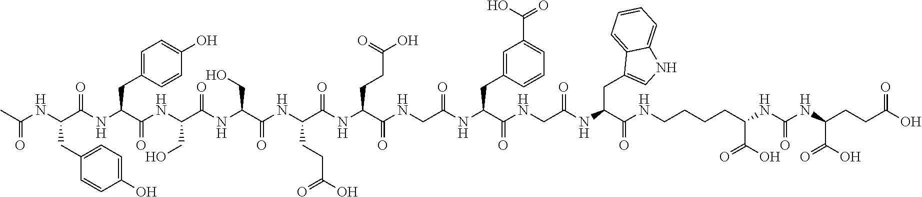 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 107 (SEQ ID NO: 94) | 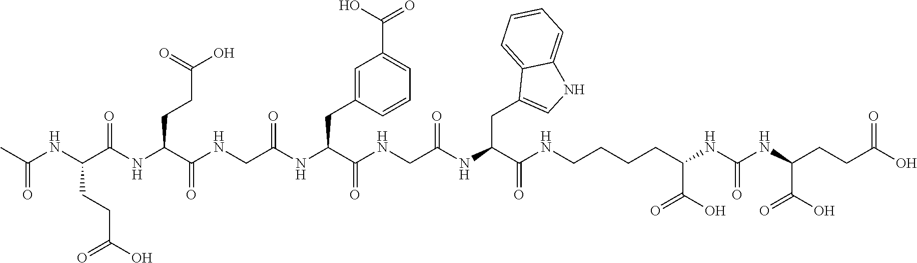 [SMILES: CC(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 108 (SEQ ID NO: 95) | 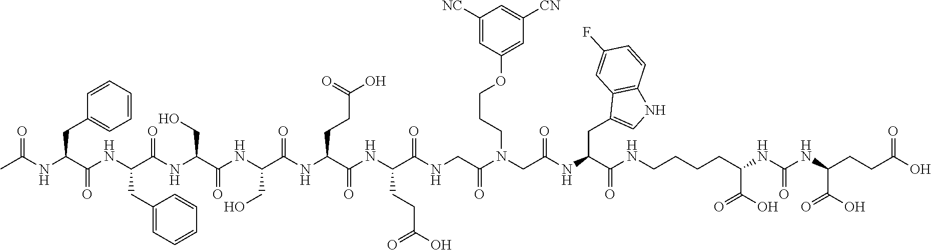 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O] |
| 109 (SEQ ID NO: 96) | 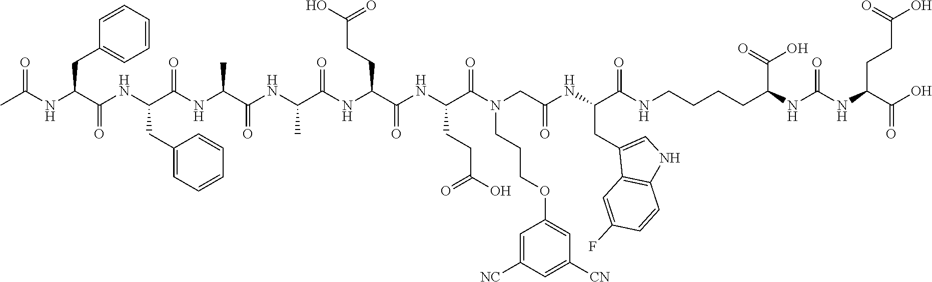 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O] |
| 110 (SEQ ID NO: 97) | 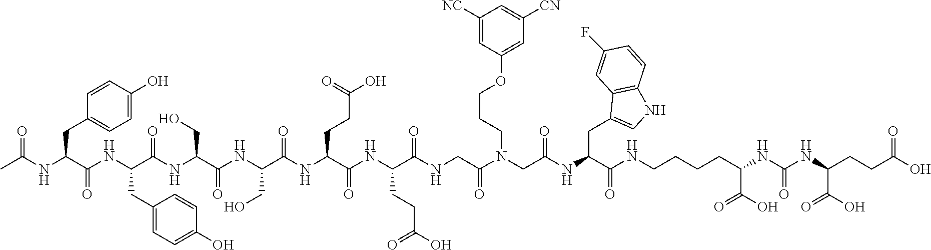 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O] |
| 111 (SEQ ID NO: 98) | 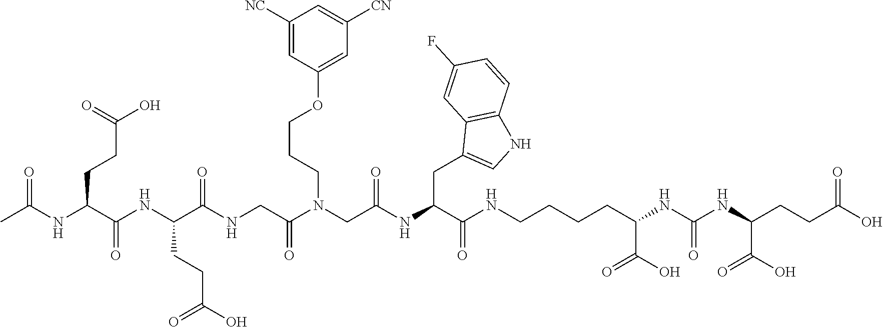 [SMILES: CC(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O] |
| 112 (SEQ ID NO: 99) | 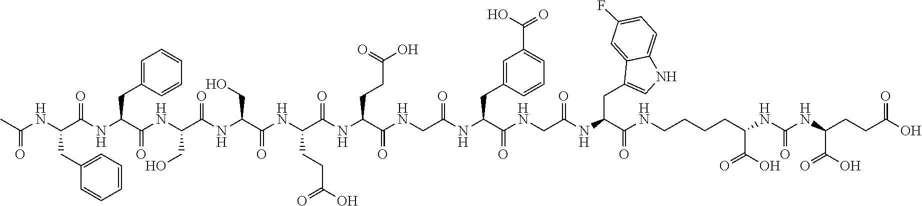 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O] |
| 113 (SEQ ID NO: 100) | 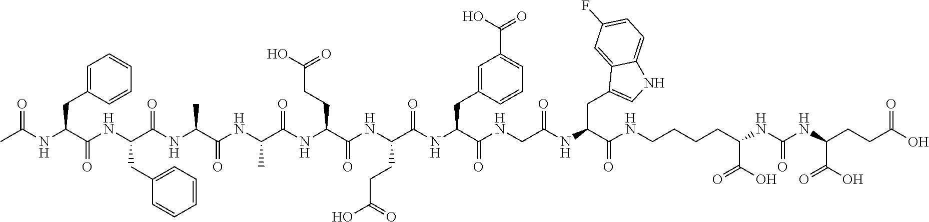 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O] |
| 114 (SEQ ID NO: 101) | 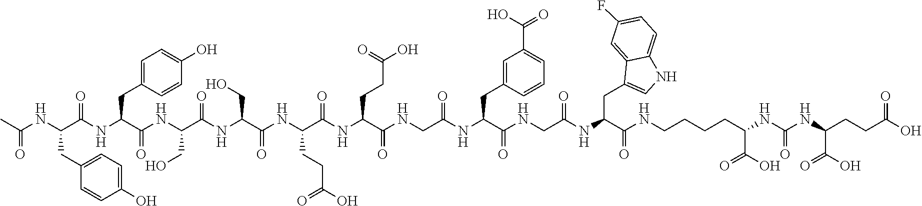 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O] |
| 115 (SEQ ID NO: 102) | 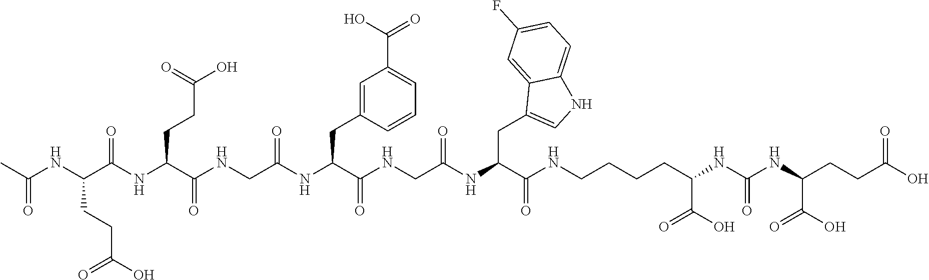 [SMILES: CC(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O] |
| 116 (SEQ ID NO: 105) | 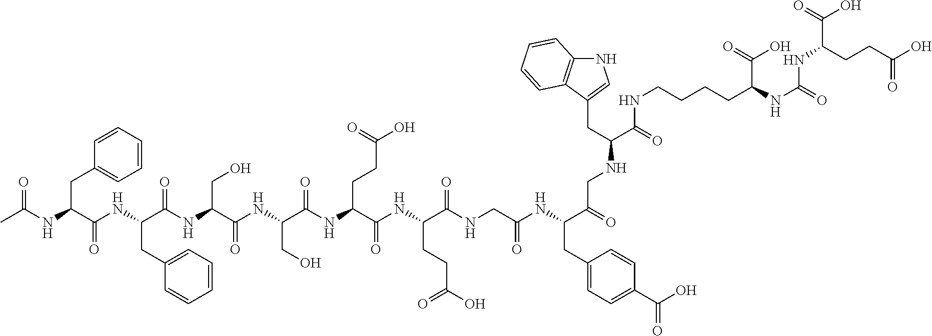 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1ccc(C(=O)O)cc1)C(=O)CN[C@@H](Cc1c[nH]c2ccccc12)C(=O)NCCCC[C@H](NC(=O)N[C@@H](CCC(=O)O)C(=O)O)C(=O)O] |
| 117 (SEQ ID NO: 106) | 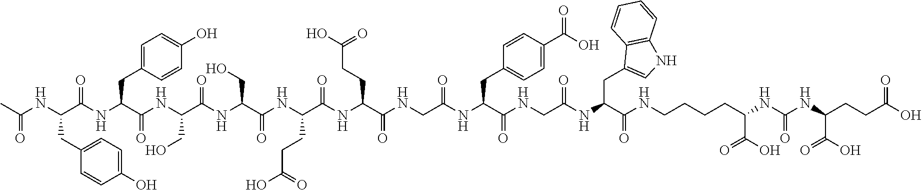 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1ccc(C(=O)O)cc1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 118 (SEQ ID NO: 104) | 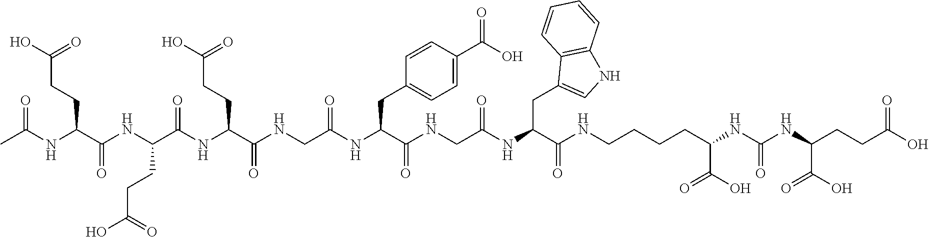 [SMILES: CC(=O)N[C@@H](CCC(=O)O)C(=O)N[C@@H](CCC(=O)O)C(=O)N[C@@H](CCC(=O)O)C(=O)NCC(=O)N[C@@H](Cc1ccc(C(=O)O)cc1)C(=O)NCC(=O)N[C@@H](Cc1c[nH]c2ccccc12)C(=O)NCCCC[C@H](NC(=O)N[C@@H](CCC(=O)O)C(=O)O)C(=O)O] |
| 119 (SEQ ID NO: 111) | 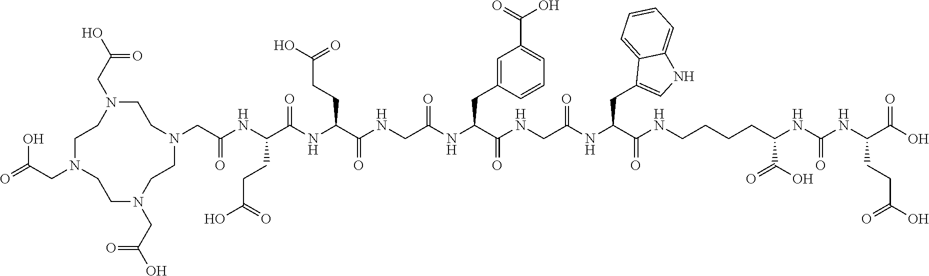 [SMILES: O=C(O)CC[C@H](NC(=O)N[C@@H](CCCCNC(=O)[C@H](Cc1c[nH]c2ccccc12)NC(=O)CNC(=O)[C@H](Cc1cccc(C(=O)O)c1)NC(=O)CNC(=O)[C@H](CCC(=O)O)NC(=O)[C@H](CCC(=O)O)NC(=O)CN1CCN(CC(=O)O)CCN(CC(=O)O)CCN(CC(=O)O)CC1)C(=O)O)C(=O)O] |
| 120 (SEQ ID NO: 112) | 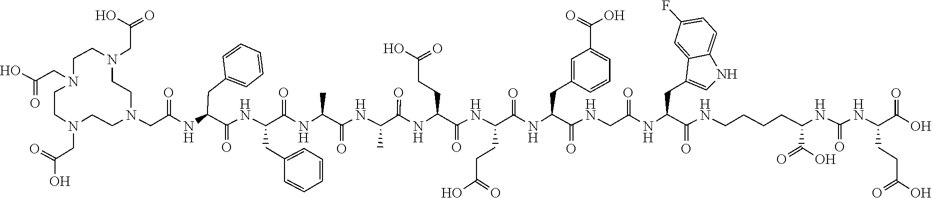 [SMILES: C[C@H](NC(=O)[C@H](C)NC(=O)[C@H](Cc1ccccc1)NC(=O)[C@H](Cc1ccccc1)NC(=O)CN1CCN(CC(=O)O)CCN(CC(=O)O)CCN(CC(=O)O)CC1)C(=O)N[C@@H](CCC(=O)O)C(=O)N[C@@H](CCC(=O)O)C(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O] |

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Throughout the instant description, certain numerical or other similar values may be described as, for example, “at least” or “at most” a set of values indicated in a list form (e.g., “at least 2, 3, 4, 5, or 6”). In such cases, unless context clearly indicates otherwise, it is intended that the phrase “at least,” “at most,” or other similar term is applied individually to each value in the list. For example, the phrase “at least 2, 3, 4, 5, or 6” is to be interpreted as “at least 2, at least 3, at least 4, at least 5, or at least 6.”

Referred to herein are additional groups which are “attached,” “covalently attached,” “linked,” or ligands described herein. As used herein, these terms means that the polymer is tethered to the indicated reside, and such tethering can include a linking group (i.e., a linker) unless otherwise specified. Thus, for an additional group (e.g., a protein) “attached,” “covalently attached,” or “linked” to a ligand, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the federal or a state government or listed in the U.S. Pharmacopeia (U.S.P.) or other generally recognized pharmacopeia for use in animals, including humans.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

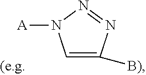

it is intended that the other regioisomer

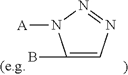

is is also encompassed.

The term “optional” or “optionally” denotes that a subsequently described event or circumstance can but need not occur, and that the description includes instances where the event or circumstance occurs and instances in which it does not.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted cyclooctyne | triazole |
| | (e.g., dibenzocycloocytne (DBCO), | |
| | difluorocyclooctyne, bicyclo[6.1.0]nonyne, etc.)) | |
| Phosphine | Azide/ester pair | amide |
| Tetrazine | trans-cyoclooctene | dihydropyridazine |
| Amine | Activated ester (e.g., N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methyl ethyl (i-propyl), n-butyl, i-butyl, s-butyl, n-pentyl, 1,1-dimethyl ethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CFF—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C^{o}C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like.

The term “alkynyl” refers to a type of alkyl group in which at least one carbon-carbon triple bond is present. In one embodiment, an alkenyl group has the formula —C^{o}C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like.

The term “aryl” refers to a radical comprising at least one aromatic ring wherein each of the atoms forming the ring is a carbon atom. Aryl groups can be optionally substituted. Examples of aryl groups include, but are not limited to phenyl and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[l.l.l]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —O CH₂CH₂ O CH₂CH₂ O CH₂CH₂ O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quatemized. The heterocycloalkyl radical is partially or fully saturated. Examples of heterocycloalkyl radicals include, but are not limited to, dioxolanyl, thienyl[l,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 0 atoms, 0-1 P atoms, and 0-1 S atoms in the ring. In some embodiments, a heteroaryl contains 1-4 N atoms, 0-1 0 atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C6-C9 heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is, unless otherwise specified, optionally substituted with one or more additional group(s) individually and independently selected from, for example, D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —S C₁-C₄alkyl, —S(═O) C₁-C₄alkyl, and —S(═O)₂ C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (=0).

In any instance in which a given entity is described herein as “optionally substituted,” it is expressly intended that the entity can also be “unsubstituted.”

The present disclosure is further illustrated in the following Examples which are given for illustration purposes only and are not intended to limit the disclosure in any way.

### EXAMPLES

### General Procedures

### Procedure 1: Manual Coupling of Fmoc-Amino Acid or SPPS Building Block (2-5 Equiv)

A solution of Fmoc-amino acid or SPPS building block (2-5 equiv), HATU (2-5 equiv) and DIPEA (4-10 equiv) in DMF (2-4 mL of DMF per 0.1 mmol of resin) was prepared (2 min of pre-activation at r.t.) and added to the resin. After 30-120 min under gentle agitation at r.t., the resin was filtered and washed with DMF, DCM and DMF (4 mL per 0.1 mmol of resin, for each washing step). In some cases, the resin was treated with acetic anhydride (6 equiv) in DMF (2-4 mL of DMF per 0.1 mmol of resin) in presence of DIPEA (6 equiv) for 6-10 min for capping any unreacted free amine.

### Procedure 2: Automated Solid-Phase (Peptide) Synthesis

Peptide-based compounds were synthesized on an automated peptide synthesizer using Fmoc-SPPS chemistry. Couplings were performed using Fmoc/^{t}Bu strategy including amino acids or building blocks (2-8 equiv. to resin substitution), HCTU or HATU (2-8 equiv.) as coupling reagents and DIPEA or NMM (4-16 equiv.) as bases in DMF or NMP (10V) at r.t. The solution containing the reagents was added to the resin and allowed to react for 30-120 min. In some cases, double couplings were required. In some cases, the resin was treated with 20% (v/v) acetic anhydride (10 equiv.) in DMF in presence of NMM (10 equiv.) for capping any unreacted free amine. Fmoc deprotections were performed twice (2-15 min) with 20% (v/v) 4-methylpiperidine in DMF. In some cases, N-term acetylation was performed with a solution of acetic anhydride (10 equiv.) and NMM (10 equiv.) in DMF at r.t. for 10-15 min. The peptidyl-resin was then washed with NMP, IPA.

### Procedure 3: On-Resin Urea Formation.

The resin was treated with a solution of CDI (3-5 equiv) and DIPEA (4-7 equiv) in DCM (2 mL for 0.1 mmol scale) at r.t. for 1.5 h. Reaction completion was monitored by TNBS test and by microcleavage analysis. A solution of H-Aa(PG)-OtBu hydrochloride (2-3 equiv) and DIPEA (5-8 equiv) in DCM/DMF (1:1, v/v, 1 mL for 0.1 mmol scale) was added onto the resin and the suspension was kept under gentle agitation at r.t. overnight (16-20 h). The resin was thoroughly washed with (2 mL for 0.1 mmol scale) DMF (2×), DCM (2×), IPA (1×), DMF (2×).

### Procedure 4: Fmoc Deprotection

A solution of 4-methylpiperidine (20%, v/v) in DMF (2-4 mL of DMF per 0.1 mmol of resin) was added to the resin. After 2-10 min under gentle agitation at r.t., the resin was filtered and washed with DMF (5×4 mL per 0.1 mmol of resin).

### Procedure 5: Preloading of Fmoc-Amino Acids on 2-Cl-Trt-Resin Chloride

2Cl-Trt-resin chloride were rinsed with DCM and allowed to swell in DCM (1-2 mL per 0.25 mmol of resin) for 10 min. Fmoc-amino acid (1 equiv.) and DIPEA (3 equiv.) were dissolved in 1.6 mL of DCM/DMF (4:1, v/v). The reaction mixture was added to the swollen resin. After 2-3 h under gentle agitation at r.t., the resin was washed once with (2 mL for 0.1 mmol scale) DMF (3×) and DCM (3×). Capping of unreacted sites on the resin was performed twice for 15 min by addition of a solution of DCM/DIPEA/MeOH (17:2:1, 1-2 mL per 0.25 mmol of resin). The loading was measured using conventional resin loading protocol.

### Procedure 6: TFA Cleavage

The resin was washed three times with DCM (5 mL per 0.1 mmol of resin, for each washing step) and dried under reduced pressure. The resin was then treated for 30 min with the cleavage mixture: TFA/water/TIS (9:0.5:0.25, v/v/v, 5 mL per 0.1 mmol of resin) or TFA/water (9:1, v/v, 5 mL per 0.1 mmol of resin). The reaction mixture was concentrated at reduced pressure. The crude was dissolved in 15 to 50 mL of CH₃CN/water (1:1, v/v), frozen and lyophilized.

### Procedure 7: Soft Cleavage of Protected Peptide

The resin was washed twice with DCM (10 mL per 0.1 mmol of resin, for each washing step). The resin was then treated twice with HFIP/DCM (1:4, v/v, 5 mL per 0.1 mmol of resin) for 20 min. The resin was washed twice with DCM (5 mL per 0.1 mmol of resin, for each washing step, 2 min/washing). The cleavage solutions and the washing fractions were gathered and concentrated under reduced pressure.

### Procedure 8: Purification of Precursors and Final Products:

Precursors and final products were purified by RP-HPLC. Different gradients were applied for the different molecules. The mobile phase was MilliQ-H₂O with 0.1% TFA (v/v) (Buffer A) and HPLC grade CH₃CN with 0.1% TFA (v/v) (Buffer B). Preparative HPLC was performed on a C4 (50×250 mm) or on a C18 column (50×250 mm) at a flow rate of 40 or 55 m/min at 40° C. or 50° C. A representative gradient used for the purification is given below:
Buffers: A: H₂O 0.1% TFA (v/v), B: CH₃CN 0.1% TFA (v/v)
Column: C18 5 μm; 50×250 mm
Temperature: 50° C.

| Time | Flow | | |
|---|---|---|---|
| (min) | (ml/min) | A % | B % |
| 0 | 55 | 95 | 5 |
| 2 | 55 | 90 | 10 |
| 10 | 55 | 90 | 10 |
| 15.1 | 55 | 68 | 32 |
| 51 | 55 | 58 | 42 |
| 52 | 55 | 5 | 95 |
| 60 | 55 | 5 | 95 |
| 61 | 55 | 95 | 5 |
| 65 | 55 | 95 | 5 |

### Procedure 9: Alloc Deprotection:

A solution of 1,3-dimethylbarbituric acid (24 equiv.) in DMSO/DCM (1:1, v/v, 4 mL per 0.1 mmol of resin) purged with nitrogen was added to the resin. Alloc removal was carried out upon the addition of Pd(PPh₃)₄ (0.5 equiv.) as a powder in the reaction mixture. After 20 min under gentle agitation at r.t. with nitrogen bubbling, the resin was filtered and washed twice with DCM and twice with DMF (5 mL per 0.1 mmol of resin, for each washing step). The resin was then treated with a solution of sodium diethyldithiocarbamate (5%, w/v) and DIPEA (5%, v/v) in DMF: 5 min wash (8×2.5 mL per 0.1 mmol of resin) with gentle stirring, 30 min wash (2×2.5 mL per 0.1 mmol of resin) with gentle stirring. The resin was then washed with DMF, IPA, DCM, IPA and DMF (5 mL per 0.1 mmol of resin, for each washing step).

### Procedure 10: Synthesis of Disubstituted Phenoxyalkyl Derivatives:

To a 0.3 M solution of disubstituted phenol derivative (1.05 equiv.) in DMF, cesium carbonate (2 equiv.) was added, and the mixture was stirred at 70° C. for 10 min. Then, a 0.75 M solution of tert-butyl (n-bromoalkyl)carbamate (1 equiv.) in DMF was added dropwise. The reactor was sealed, and the mixture was stirred at 70° C. for 2 h. The mixture was diluted 1 to 10 with EtOAc and debated with a saturated solution of NaHCO3(aq) (3×50 mL), 0.5 M aq. HCl(aq) (2×50 mL), brine (50 mL). The organic layer was dried over anhydrous Na2SO4, filtered, and concentrated to dryness under reduced pressure. The residual material was treated with 30% TFA in DCM (15 mL, v/v) at r.t. for 30 min, then the mixture was concentrated to dryness, diluted with ACN/H2O (1:1, v/v) and freeze-dried, to afford the desired precursor in the form of a TFA salt.

### Procedure 11: N-Term Acetylation:

The resin was treated with a solution of acetic anhydride (15 equiv.) and DIPEA (2 equiv.) in DMF at r.t. for 15 min. The resin was washed 3 times with the following solvents: DMF, DCM, MeOH, DEE, and dried under vacuum.

### Protocols

### Synthesis of Preloaded-Resin-I

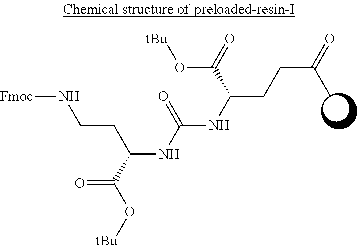

Loading of Fmoc-Glu-OtBu on 2-Cl-Trt resin (2.9 g, 1.1 mmol/g) was performed as described in Procedure 5. The loading was measured using conventional resin loading protocol. Fmoc deprotection was performed as described in Procedure 4. The resin was thoroughly washed with DMF, DCM, MeOH, DCM, three times 10 mL each solvent. The resin (2.3 g, 1.7 mmol) was treated with a solution of CDI (0.84 g, 5.2 mmol, 3.0 equiv) and DIPEA (0.67 g, 0.90 mL, 5.2 mmol, 3.0 equiv) in DCM (15 mL) at r.t. for 40 min. The resin was washed with 2×10 mL of DCM. A solution of H-Lys(Fmoc)-OtBu *HCl (1.6 g, 3.4 mmol, 2 equiv) and DIPEA (0.88 g, 1.2 mL, 6.8 mmol, 4 equiv) in 10 mL of 1:1 DCM/DMF was added onto the resin and the suspension was kept under gentle agitation at r.t. for 1 h. The resin was thoroughly washed three times with the following solvents: DMF, DCM, MeOH, DEE, and dried under vacuum and stored at 4° C.

### Synthesis of Building Block-I

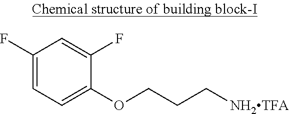

The synthesis of 3-(2,4-difluorophenoxy)propan-1-amine 2,2,2-trifluoroacetate (building block-I) was performed on a 1.5 mmol scale from tert-butyl (3-bromopropyl)carbamate (360 mg, 1.51 mmol) and 2,4-difluorophenol (207 mg, 1.59 mmol) following Procedure 10. building block-I was obtained as a as a brownish/purple solid (475 mg, 104% yield, 97% purity) in the form of TFA salt.

The purity of the crude material was estimated by analytical HPLC, using an Aeris C18 column (4.6×250 mm) at 50° C. and CH3CN/H2O containing 0.1% TFA as mobile phase, with a gradient of 10 to 85% CH3CN in 6 min. m/z calculated for C₉H₁₁F₂NO [M+H]⁺: 187.19; measured 188.08.

### Synthesis of Building Block-II

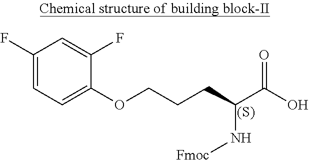

The synthesis of (S)-2-((((9H-fluoren-9-yl)methoxy)carbonyl)amino)-5-(2,4-difluorophenoxy)pentanoic acid (building block-II) was performed on a 1 mmol scale from tert-butyl (S)-5-bromo-2-((tert-butoxycarbonyl)amino)pentanoate (350 mg, 0.99 mmol) and 2,4-difluorophenol (136 mg, 1.04 mmol) following Procedure 10. The material was dissolved in a solution of 1,4-Dioxane (5 mL) and a solution of sodium carbonate (316 mg, 3 Eq, 2.98 mmol) in Water (5 mL) was added. Fmoc-OSu (235 mg, 0.7 Eq, 695 mol) was added as a solid, and the mixture was stirred at r.t. for 2 h. The mixture was diluted with EtOAc (100 mL) and debated with 0.5 M aq. HCl (2×30 mL), brine (50 mL). The organic layer was dried over Na2SO4, filtered, and concentrated to dryness under reduced pressure to afford a 50% mixture of desired building block-II and undesired (S)-2-((((9H-fluoren-9-yl)methoxy)carbonyl)amino)pent-4-enoic acid as a brownish solid (346 mg, 86% yield, 50% purity). The purity of the crude material was estimated by analytical HPLC, using an Aeris C18 column (4.6×250 mm) at 50° C. and CH3CN/H2O containing 0.1% TFA as mobile phase, with a gradient of 10 to 85% CH3CN in 6 min. m/z calculated for C₂₆H₂₃F₂NO₅ [M+H]⁺: 467.47; measured 468.15.

### Synthesis of Building Block-III

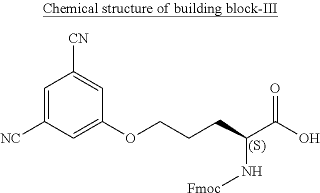

The synthesis of (S)-2-((((9H-fluoren-9-yl)methoxy)carbonyl)amino)-5-(3,5-dicyanophenoxy)pentanoic acid (building block-III) was performed on 2.8 mmol scale from tert-butyl (S)-5-bromo-2-((tert-butoxycarbonyl)amino)pentanoate (1.0 g, 2.84 mmol) and 5-hydroxyisophthalonitrile (429.6 mg, 2.98 mmol) following Procedure 10. The material was dissolved in a solution of 1,4-Dioxane (10 mL) and a solution of sodium carbonate (1.2 g, 3 Eq, 11.4 mmol) in Water (10 mL) was added. Fmoc-OSu (1.9 g, 1.48 Eq, 5.64 mmol) was added portionwise, as a solid, and the mixture was stirred at r.t. for 2 h. The mixture was diluted with EtOAc (150 mL) and debated with 0.5 M aq. HCl (2×70 mL), brine (70 mL). The organic layer was dried over Na2SO4, filtered, and concentrated to dryness under reduced pressure to afford a 50% mixture of desired building block-III and undesired (S)-2-((((9H-fluoren-9-yl)methoxy)carbonyl)amino)pent-4-enoic acid as a brownish solid (1.59 g, 116% yield, 23% purity). The resulting solid was dissolved in DMSO and purified using Procedure 8. Preparative HPLC fractions containing the purified product were combined and freeze-dried, yielding a white solid (590 mg, 32% yield, 95% purity).

The purity of the crude material was estimated by analytical HPLC, using an Aeris C18 column (4.6×250 mm) at 50° C. and CH₃CN/H₂O containing 0.1% TFA as mobile phase, with a gradient of 10 to 85% CH₃CN in 6 min. m/z calculated for C₂₈H₂₃N₃O₅ [M+H]⁺: 481.51; measured 482.37.

### Synthesis of Building Block-IV

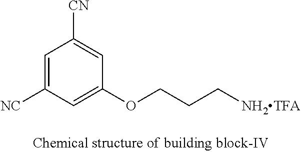

The synthesis of 5-(3-aminopropoxy)isophthalonitrile 2,2,2-trifluoroacetate (building block-IV) was performed on a 3 mmol scale from tert-butyl (3-bromopropyl)carbamate (714 mg, 3 mmol) and 5-hydroxyisophthalonitrile (454 mg, 3.15 mmol) following Procedure 10. building block-IV was obtained as a as a brownish/purple solid (865 mg, 85% yield, 97% purity) in the form of TFA salt. The purity of the crude material was estimated by analytical HPLC, using an Aeris C18 column (4.6×250 mm) at 50° C. and CH3CN/H2O containing 0.1% TFA as mobile phase, with a gradient of 10 to 85% CH₃CN in 6 min. m/z calculated for C₁₁H₁₁N₃O [M+H]+: 201.23; measured 202.33.

### Synthesis of Building Block-V

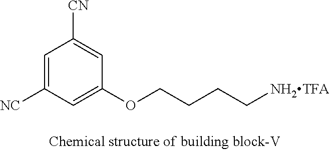

The synthesis of 5-(4-aminobutoxy)isophthalonitrile 2,2,2-trifluoroacetate (building block-V) was performed on a 3 mmol scale from tert-butyl (4-bromobutyl)carbamate (756 mg, 3 mmol) and 5-hydroxyisophthalonitrile (454 mg, 3.15 mmol) following Procedure 10. building block-V was obtained as a as a brownish/purple solid (848 mg, 89.7% yield, 93% purity) in the form of TFA salt. The purity of the crude material was estimated by analytical HPLC, using an Aeris C18 column (4.6×250 mm) at 50° C. and CH3CN/H2O containing 0.1% TFA as mobile phase, with a gradient of 10 to 85% CH₃CN in 6 min. m/z calculated for C₁₂H₁₃N₃O [M+H]+: 215.26; measured 216.34.

### Synthesis of Building Block-VI

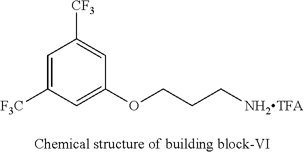

The synthesis of 3-(3,5-bis(trifluoromethyl)phenoxy)propan-1-amine 2,2,2-trifluoroacetate (building block-VI) was performed on a 3 mmol scale from tert-butyl (3-bromopropyl)carbamate (714 mg, 3 mmol) and 3,5-bis(trifluoromethyl)phenol (724 mg, 3.15 mmol) following Procedure 10. building block-VI was obtained as a as a white solid (1.12 g, 93% yield, 100% purity) in the form of TFA salt. The purity of the crude material was estimated by analytical HPLC, using an Aeris C18 column (4.6×250 mm) at 50° C. and CH3CN/H2O containing 0.1% TFA as mobile phase, with a gradient of 10 to 85% CH3CN in 6 min. m/z calculated for C₁₁H₁₁F₆NO [M+H]⁺: 287.21; measured 288.31.

### Synthesis of Building Block-VII

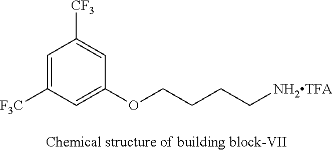

The synthesis of 4-(3,5-bis(trifluoromethyl)phenoxy)butan-1-amine 2,2,2-trifluoroacetate (building block-VII) was performed on a 3 mmol scale from tert-butyl (4-bromobutyl)carbamate (756 mg, 3 mmol) and 3,5-bis(trifluoromethyl)phenol (724 mg, 3.15 mmol) following Procedure 10. building block-VII was obtained as a as a white solid (1.17 g, 92% yield, 97.87% purity) in the form of TFA salt. The purity of the crude material was estimated by analytical HPLC, using an Aeris C18 column (4.6×250 mm) at 50° C. and CH₃CN/H₂O containing 0.1% TFA as mobile phase, with a gradient of 10 to 85% CH₃CN in 6 min. m/z calculated for C₁₂H₁₃F₆NO [M+H]⁺: 301.23 measured 302.36.

### Synthesis of Building Block-IV

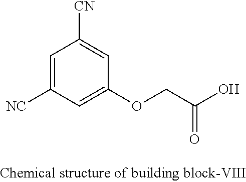 [SMILES: N#Cc1cc(C#N)cc(OCC(=O)O)c1]

The synthesis of 2-(3,5-dicyanophenoxy)acetic acid (building block-VIII) was performed on a 3 mmol scale from tert-butyl 2-bromoacetate (585 mg, 3 mmol) and 5-hydroxyisophthalonitrile (454 mg, 3.15 mmol) following Procedure 10. building block-VIII was obtained as a as a pale yellow solid (525 mg, 83.6% yield, 96.55% purity).

The purity of the crude material was estimated by analytical HPLC, using an Aeris C18 column (4.6×250 mm) at 50° C. and CH₃CN/H₂O containing 0.05% Formic acid as mobile phase, with a gradient of 10 to 85% CH₃CN in 6 min. m/z calculated for C₁₀H₆N₂O₃ [M−H]⁻: 201.16 measured 201.05.

### Synthesis of Building Block-IX

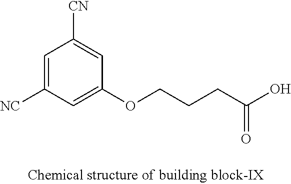 [SMILES: N#Cc1cc(C#N)cc(OCCCC(=O)O)c1]

The synthesis of 4-(3,5-dicyanophenoxy)butanoic acid (building block-IX) was performed on a 3 mmol scale from tert-butyl 4-bromobutanoate (670 mg, 3 mmol) and 5-hydroxyisophthalonitrile (454 mg, 3.15 mmol) following Procedure 10. building block-IX was obtained as a as a pale yellow solid (572 mg, 65.2% yield, 78.77% purity). The purity of the crude material was estimated by analytical HPLC, using an Aeris C18 column (4.6×250 mm) at 50° C. and CH₃CN/H₂O containing 0.05% Formic acid as mobile phase, with a gradient of 10 to 85% CH₃CN in 6 min. m/z calculated for C₁₂H₁₀N₂O₃ [M−H]⁻: 229.22 measured 229.20.

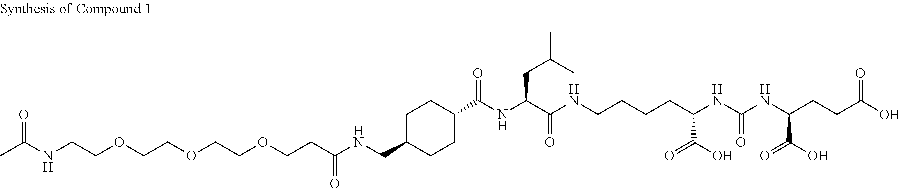 [SMILES: CC(=O)NCCOCCOCCOCCC(=O)NC[C@H]1CC[C@H](C(=O)N[C@@H](CC(C)C)C(=O)NCCCC[C@H](NC(=O)N[C@@H](CCC(=O)O)C(=O)O)C(=O)O)CC1]

The synthesis of Compound 1 was initiated on a 0.1 mmol scale. H-Leu-2-Cl-Trt resin (132 mg, 0.76 mmol/g) was allowed to swell in 2 mL DMF for 15 min. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using procedure 11. The resin was then treated with the cleavage mixture using Procedure 7 yielding Ac-PEG3-AMCHC-Leu-COOH precursor as a white solid. The precursor was used directly without purification. EuK(O^{t}Bu)₃ (24 mg; 48 μmol; 1 equiv), Ac-PEG3-AMCHC-Leu-COOH (25 mg; 48 μmol; 1 equiv) and HOOBt (16 mg; 97 μmol; 2.0 equiv) were dissolved in 2 mL of DMF. The coupling reaction was initiated upon addition of DIPEA (13 μL; 73 μmol; 1.5 equiv) and EDC*HCl (14 mg; 73 μmol; 1.5 equiv). After 3 h at r.t., the reaction mixture was then concentrated at reduced pressure. The resulting paste was treated with 5.0 mL of TFA/water/DCM (60:5:35, v/v/v). After 1 h, the reaction mixture was concentrated under reduced pressure. The resulting paste was dissolved in DMSO and purified using Procedure 8. Preparative HPLC fractions containing the purified product were combined, frozen and lyophilized yielding a white solid (9.1 mg, 23% yield, 97% purity).

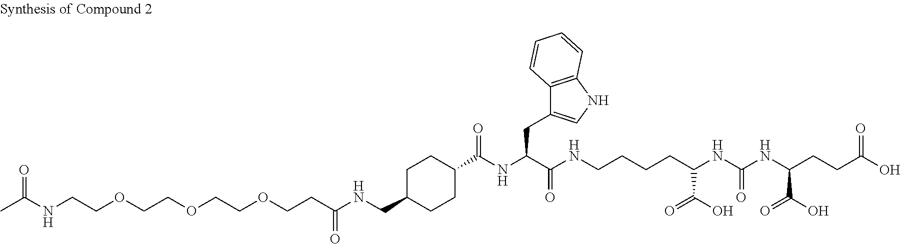

The synthesis of Compound 2 was initiated on a 0.1 mmol scale. H-Trp(Boc)-2-Cl-Trt resin (166 mg, 0.61 mmol/g) was allowed to swell in 2 mL DMF for 15 min. Fmoc-AMCHC-OH was coupled to the resin using Procedure 1. Fmoc deprotection was performed using Procedure 4. Fmoc-N-amido-PEG3-OH was coupled to the resin using Procedure 2. Fmoc deprotection was performed using Procedure 4. N-term acetylation was performed using Procedure 11. The resin was then treated with the cleavage mixture using Procedure 7 yielding Ac-PEG3-AMCHC-Trp(Boc)-COOH precursor as a white solid. The precursor was used directly without purification. EuK(O^{t}Bu)₃ (CAS: 1025796-31-9, 16 mg; 32 μmol; 1 equiv), Ac-PEG3-AMCHC-Trp(Boc)-COOH (22 mg; 32 μmol; 1 equiv) and HOOBt (10 mg; 64 μmol; 2.0 equiv) were dissolved in 1.3 mL of DMF. The coupling reaction was initiated upon addition of DIPEA (8 μL; 48 μmol; 1.5 equiv) and EDC*HCl (9 mg; 48 μmol; 1.5 equiv). After 3 h at r.t., the reaction mixture was concentrated at reduced pressure. The resulting paste was treated with 5.0 mL of TFA/water/DCM (60:5:35, v/v/v). After 1 h at r.t., the reaction mixture was concentrated under reduced pressure. The resulting paste was dissolved in DMSO and purified using Procedure 8. Preparative HPLC fractions containing the purified product were combined, frozen and lyophilized yielding a white solid (12 mg, 42% yield, 90% purity).

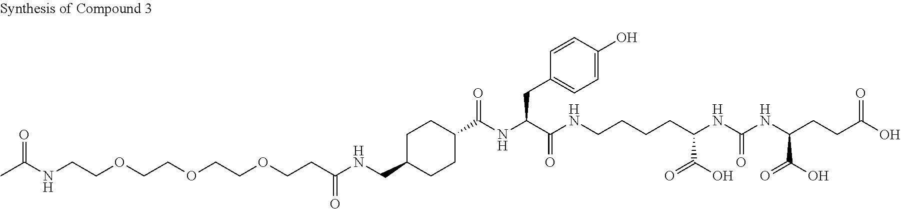 [SMILES: CC(=O)NCCOCCOCCOCCC(=O)NC[C@H]1CC[C@H](C(=O)N[C@@H](Cc2ccc(O)cc2)C(=O)NCCCC[C@H](NC(=O)N[C@@H](CCC(=O)O)C(=O)O)C(=O)O)CC1]

The synthesis of Compound 3 was performed on a 0.1 mmol scale. Precursor-I-resin was swelled in DCM for 15 min and washed with DMF. Fmoc deprotection was performed using Procedure 4. Fmoc-Tyr(^{t}Bu)-OH was coupled to the resin using Procedure 1. Fmoc deprotection was performed using Procedure 4. Fmoc-AMCHC-OH was coupled to the resin using Procedure 2. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO/water (2:1, v/v) and purified by preparative RP-HPLC using Procedure 8. Preparative HPLC fractions containing the purified product were combined, frozen and lyophilized yielding a white solid (46 mg, 53% yield, 97% purity).

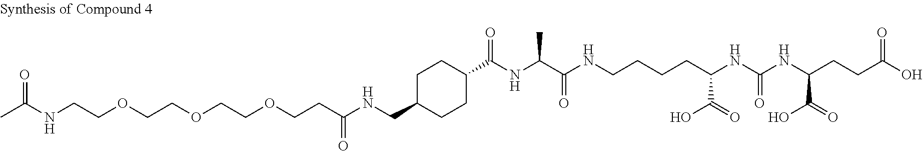 [SMILES: CC(=O)NCCOCCOCCOCCC(=O)NC[C@H]1CC[C@H](C(=O)N[C@@H](C)C(=O)NCCCC[C@H](NC(=O)N[C@@H](CCC(=O)O)C(=O)O)C(=O)O)CC1]

The synthesis of Compound 4 was performed on a 0.1 mmol scale. Precursor-I-resin was swelled in DCM for 15 min and washed with DMF. Fmoc deprotection was performed using Procedure 4. Fmoc-Ala-OH was coupled to the resin using Procedure 1. Fmoc deprotection was performed using Procedure 4. Fmoc-AMCHC-OH was coupled to the resin using Procedure 2. Fmoc deprotection was performed using Procedure 4. Fmoc-N-amido-PEG3-OH was coupled to the resin using Procedure 2. Fmoc deprotection was performed using Procedure 4. N-term acetylation was performed using Procedure 11. The resin was then treated with the cleavage mixture using Procedure 6. The crude paste was solubilized in DMSO/water (3:1, v/v) and purified by preparative RP-HPLC using Procedure 8. Preparative HPLC fractions containing the purified product were combined, frozen and lyophilized yielding a white solid (15 mg, 19% yield, 86% purity).

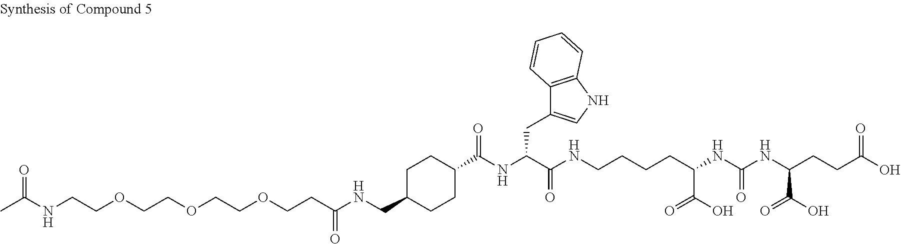 [SMILES: CC(=O)NCCOCCOCCOCCC(=O)NC[C@H]1CC[C@H](C(=O)N[C@H](Cc2c[nH]c3ccccc23)C(=O)NCCCC[C@H](NC(=O)N[C@@H](CCC(=O)O)C(=O)O)C(=O)O)CC1]

The synthesis of Compound 5 was performed on a 0.1 mmol scale. Precursor-I-resin was swelled in DCM for 15 min and washed with DMF. Fmoc deprotection was performed using Procedure 4. Fmoc-D-Trp(Boc)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The resin was then treated with the cleavage mixture using Procedure 6. The crude paste was solubilized in DMSO/water (2:1, v/v) and purified by preparative RP-HPLC using Procedure 8. Preparative HPLC fractions containing the purified product were combined, frozen and lyophilized yielding a white solid (64 mg, 72% yield, 94% purity).

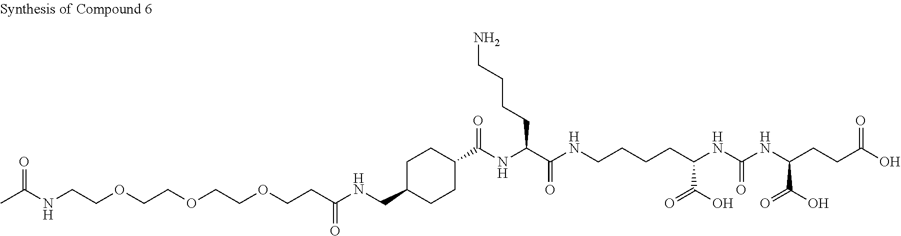 [SMILES: CC(=O)NCCOCCOCCOCCC(=O)NC[C@H]1CC[C@H](C(=O)N[C@@H](CCCCN)C(=O)NCCCC[C@H](NC(=O)N[C@@H](CCC(=O)O)C(=O)O)C(=O)O)CC1]

The synthesis of Compound 6 was performed on a 0.1 mmol scale. Precursor-I-resin was swelled in DCM for 15 min and washed with DMF. Fmoc deprotection was performed using Procedure 4. Fmoc-Lys(Boc)-OH was coupled to the resin using Procedure 2. Fmoc deprotection was performed using Procedure 4. Fmoc-AMCHC-OH was coupled to the resin using Procedure 2. Fmoc deprotection was performed using Procedure 4. Fmoc-N-PEG3-OH was coupled to the resin using Procedure 2. Fmoc deprotection was performed using Procedure 4. N-term acetylation was performed using Procedure 11. The resin was then treated with the cleavage mixture using Procedure 6. The crude paste was solubilized in 20 mL of DMSO/water (1:1, v/v) and purified by preparative RP-HPLC using Procedure 8. Preparative HPLC fractions containing the purified product were combined, frozen and lyophilized yielding a white solid (67 mg, 71% yield, 91% purity).

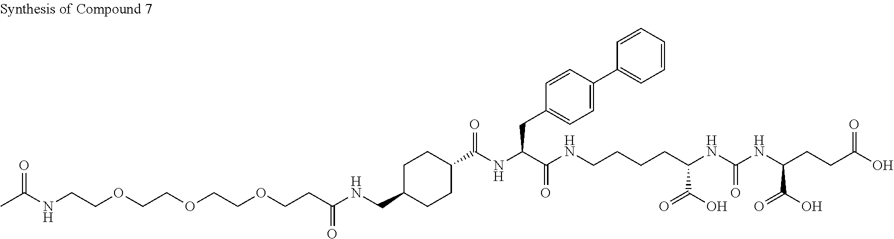 [SMILES: CC(=O)NCCOCCOCCOCCC(=O)NC[C@H]1CC[C@H](C(=O)N[C@@H](Cc2ccc(-c3ccccc3)cc2)C(=O)NCCCC[C@H](NC(=O)N[C@@H](CCC(=O)O)C(=O)O)C(=O)O)CC1]

The synthesis of Compound 7 was initiated on a 0.95 mmol scale. H-Bip-2-Cl-Trt resin was prepared using Procedure 5. The resin was allowed to swell in 3 mL DMF for 15 min. Fmoc-AMCHC-OH was coupled to the resin using Procedure 1. Fmoc deprotection was performed using Procedure 4. Fmoc-N-amido-PEG3-OH was coupled to the resin using Procedure 2. Fmoc deprotection was performed using Procedure 4. N-term acetylation was performed using Procedure 11. The resin was then treated with the cleavage mixture using Procedure 7 yielding Ac-PEG3-AMCHC-(2-Nal)-COOH precursor as a white solid. The precursor was used directly without purification. EuK(OtBu)₃ (CAS: 1025796-31-9, 48 mg; 0.1 mmol; 1 equiv), Ac-PEG3-AMCHC-(2-Nal)-COOH (59 mg; 0.1 mmol; 1 equiv) and HOOBt (19 mg; 0.12 mmol; 1.2 equiv) were dissolved in 3 mL of DMF. The coupling reaction was initiated upon addition of DIPEA (17 μL; 0.1 mmol; 1 equiv) and EDC*HCl (21 mg; 0.11 mmol; 1.1 equiv). After 30 min at r.t., the reaction mixture was concentrated at reduced pressure. The resulting paste was treated with 5 mL of TFA/water (90:10, v/v). After 30 min at r.t., the reaction mixture was concentrated under reduced pressure. The resulting paste was dissolved in DMSO and purified using Procedure 8. Preparative HPLC fractions containing the purified product were combined, frozen and lyophilized yielding a white solid (41 mg, 46% yield, 95% purity).

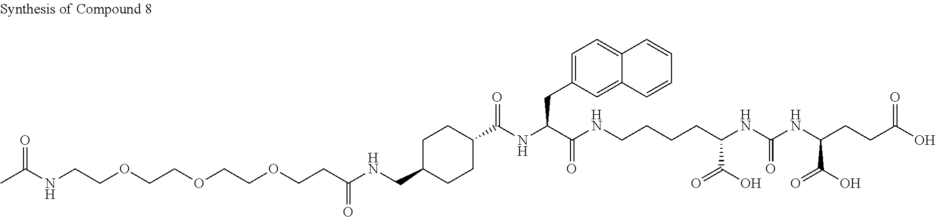 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 8 was initiated on a 0.38 mmol scale. H-2-Nal-2-Cl-Trt resin was prepared using Procedure 5. The resin was allowed to swell in 2 mL DMF for 15 min. Fmoc-AMCHC-OH was coupled to the resin using Procedure 1. Fmoc deprotection was performed using Procedure 4. Fmoc-N-amido-PEG3-OH was coupled to the resin using Procedure 2. Fmoc deprotection was performed using Procedure 4. N-term acetylation was performed using Procedure 11. The resin was then treated with the cleavage mixture using Procedure 7 yielding Ac-PEG3-AMCHC-Bip-COOH precursor as a white solid. The precursor was used directly without purification. EuK(OtBu)₃ (CAS: 1025796-31-9, 52 mg; 0.11 mmol; 1 equiv), Ac-PEG3-AMCHC-Bip-COOH (67 mg; 0.11 mmol; 1 equiv) and HOOBt (21 mg; 0.13 mmol; 1.2 equiv) were dissolved in 3 mL of DMF. The coupling reaction was initiated upon addition of DIPEA (19 μL; 0.11 mmol; 1 equiv) and EDC*HCl (23 mg; 0.12 mmol; 1.1 equiv). After 30 min at r.t., the reaction mixture was concentrated at reduced pressure. The resulting paste was treated with 5 mL of TFA/water (90:10, v/v). After 30 min at r.t., the reaction mixture was concentrated under reduced pressure. The resulting paste was dissolved in DMSO and purified using Procedure 8. Preparative HPLC fractions containing the purified product were combined, frozen and lyophilized yielding a white solid (41 mg, 41% yield, 98% purity).

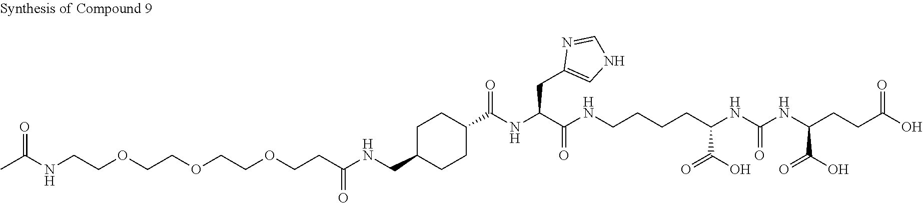 [SMILES: CC(=O)NCCOCCOCCOCCC(=O)NC[C@H]1CC[C@H](C(=O)N[C@@H](Cc2c[nH]cn2)C(=O)NCCCC[C@H](NC(=O)N[C@@H](CCC(=O)O)C(=O)O)C(=O)O)CC1]

The synthesis of Compound 9 was performed on a 0.15 mmol scale. Precursor-I-resin was swelled in DCM for 15 min and washed with DMF. Fmoc deprotection was performed using Procedure 4. Fmoc-His(Trt)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (64 mg, 51% yield, 96% purity).

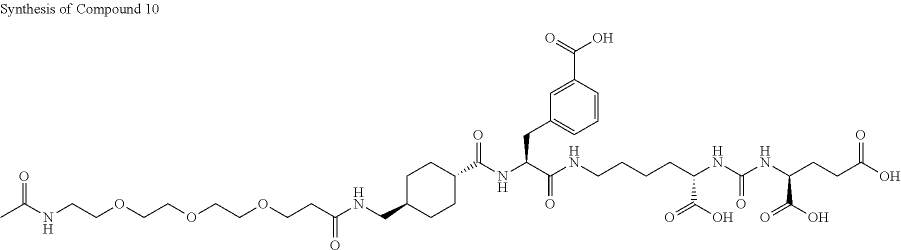 [SMILES: CC(=O)NCCOCCOCCOCCC(=O)NC[C@H]1CC[C@H](C(=O)N[C@@H](Cc2cccc(C(=O)O)c2)C(=O)NCCCC[C@H](NC(=O)N[C@@H](CCC(=O)O)C(=O)O)C(=O)O)CC1]

The synthesis of Compound 10 was performed on a 0.08 mmol scale. Precursor-I-resin was swelled in DCM for 15 min and washed with DMF. Fmoc deprotection was performed using Procedure 4. Fmoc-Phe(3-COO^{t}Bu)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed w using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (59 mg, 82% yield, 94% purity).

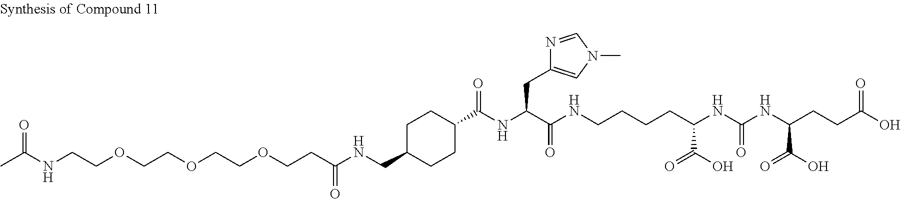 [SMILES: CC(=O)NCCOCCOCCOCCC(=O)NC[C@H]1CC[C@H](C(=O)N[C@@H](Cc2cn(C)cn2)C(=O)NCCCC[C@H](NC(=O)N[C@@H](CCC(=O)O)C(=O)O)C(=O)O)CC1]

The synthesis of Compound 11 was performed on a 0.08 mmol scale. Precursor-I-resin was swelled in DCM for 15 min and washed with DMF. Fmoc deprotection was performed using Procedure 4. Fmoc-His(3-Me)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (17 mg, 25% yield, 97% purity).

### Synthesis of Compound 12

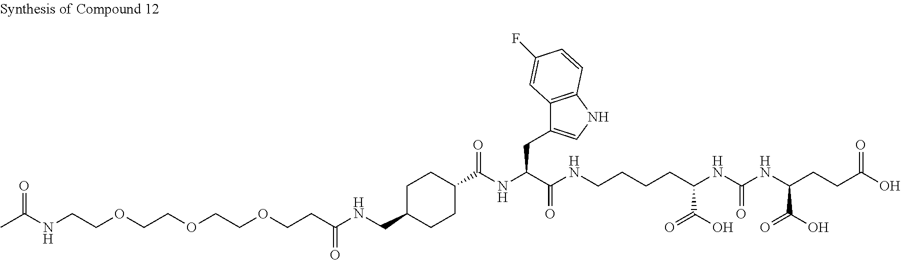 [SMILES: CC(=O)NCCOCCOCCOCCC(=O)NC[C@H]1CC[C@H](C(=O)N[C@@H](Cc2c[nH]c3ccc(F)cc23)C(=O)NCCCC[C@H](NC(=O)N[C@@H](CCC(=O)O)C(=O)O)C(=O)O)CC1]

The synthesis of Compound 12 was performed on a 0.1 mmol scale. Precursor-I-resin was swelled in DCM for 15 min and washed with DMF. Fmoc deprotection was performed using Procedure 4. Fmoc-Trp(5-F)—OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (53 mg, 58% yield, 94% purity).

### Synthesis of Compound 13

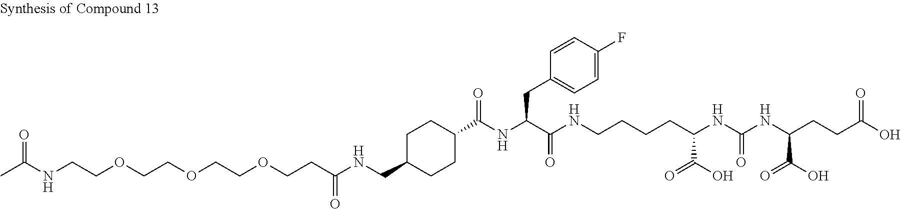 [SMILES: CC(=O)NCCOCCOCCOCCC(=O)NC[C@H]1CC[C@H](C(=O)N[C@@H](Cc2ccc(F)cc2)C(=O)NCCCC[C@H](NC(=O)N[C@@H](CCC(=O)O)C(=O)O)C(=O)O)CC1]

The synthesis of Compound 13 was performed on a 0.08 mmol scale. Precursor-I-resin was swelled in DCM for 15 min and washed with DMF. Fmoc deprotection was performed using Procedure 4. Fmoc-Phe(4-F)—OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (25 mg, 36% yield, 96% purity).

### Synthesis of Compound 14

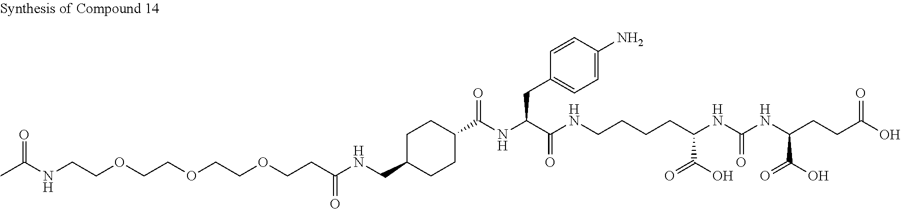 [SMILES: CC(=O)NCCOCCOCCOCCC(=O)NC[C@H]1CC[C@H](C(=O)N[C@@H](Cc2ccc(N)cc2)C(=O)NCCCC[C@H](NC(=O)N[C@@H](CCC(=O)O)C(=O)O)C(=O)O)CC1]

The synthesis of Compound 14 was performed on a 0.08 mmol scale. Precursor-I-resin was swelled in DCM for 15 min and washed with DMF. Fmoc deprotection was performed using Procedure 4. Fmoc-Phe(4-NHBoc)-OH was coupled to the resin using Procedure 1. Fmoc deprotection was performed using Procedure 4. was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (43 mg, 62% yield, 96% purity).

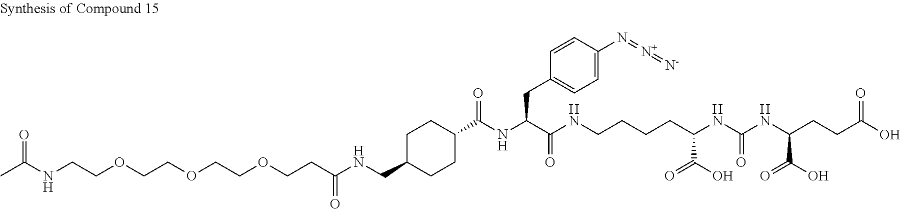 [SMILES: CC(=O)NCCOCCOCCOCCC(=O)NC[C@H]1CC[C@H](C(=O)N[C@@H](Cc2ccc(N=[N+]=[N-])cc2)C(=O)NCCCC[C@H](NC(=O)N[C@@H](CCC(=O)O)C(=O)O)C(=O)O)CC1]

The synthesis of Compound 15 was performed on a 0.08 mmol scale. Precursor-I-resin was swelled in DCM for 15 min and washed with DMF. Fmoc deprotection was performed using Procedure 4. Fmoc-Phe(4-N₃)—OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (33 mg, 46% yield, 97% purity).

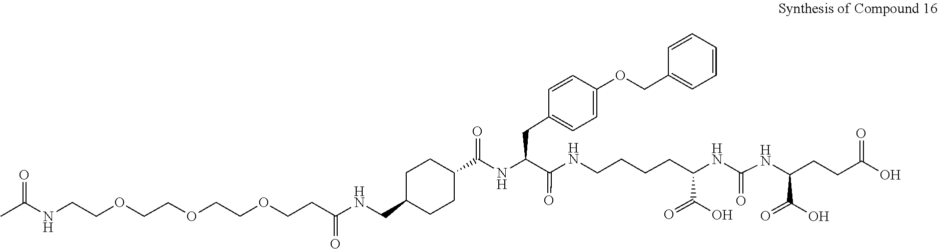 [SMILES: CC(=O)NCCOCCOCCOCCC(=O)NC[C@H]1CC[C@H](C(=O)N[C@@H](Cc2ccc(OCc3ccccc3)cc2)C(=O)NCCCC[C@H](NC(=O)N[C@@H](CCC(=O)O)C(=O)O)C(=O)O)CC1]

The synthesis of Compound 16 was performed on a 0.1 mmol scale. Precursor-I-resin was swelled in DCM for 15 min and washed with DMF. Fmoc deprotection was performed using Procedure 4. Fmoc-Tyr(OtBu)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (42 mg, 44% yield, 96% purity).

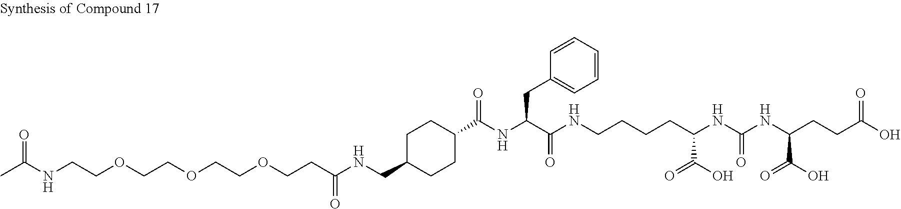 [SMILES: CC(=O)NCCOCCOCCOCCC(=O)NC[C@H]1CC[C@H](C(=O)N[C@@H](Cc2ccccc2)C(=O)NCCCC[C@H](NC(=O)N[C@@H](CCC(=O)O)C(=O)O)C(=O)O)CC1]

The synthesis of Compound 17 was performed on a 0.1 mmol scale. Precursor-I-resin was swelled in DCM for 15 min and washed with DMF. Fmoc deprotection was performed using Procedure 4. Fmoc-Phe-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (46 mg, 36% yield, 98% purity).

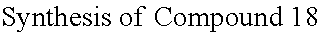

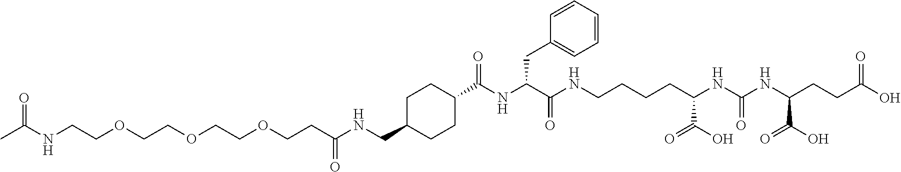 [SMILES: CC(=O)NCCOCCOCCOCCC(=O)NC[C@H]1CC[C@H](C(=O)N[C@H](Cc2ccccc2)C(=O)NCCCC[C@H](NC(=O)N[C@@H](CCC(=O)O)C(=O)O)C(=O)O)CC1]

The synthesis of Compound 18 was performed on a 0.1 mmol scale. Precursor-I-resin was swelled in DCM for 15 min and washed with DMF. Fmoc deprotection was performed using Procedure 4. Fmoc-D-Phe-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (48 mg, 56% yield, 95% purity).

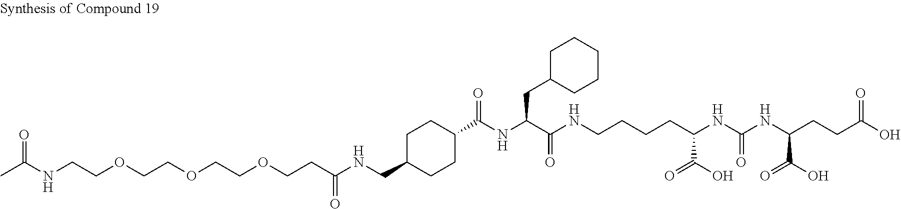 [SMILES: CC(=O)NCCOCCOCCOCCC(=O)NC[C@H]1CC[C@H](C(=O)N[C@@H](CC2CCCCC2)C(=O)NCCCC[C@H](NC(=O)N[C@@H](CCC(=O)O)C(=O)O)C(=O)O)CC1]

The synthesis of Compound 19 was performed on a 0.1 mmol scale. Precursor-I-resin was swelled in DCM for 15 min and washed with DMF. Fmoc deprotection was performed using Procedure 4. Fmoc-Cha-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (47 mg, 55% yield, 96% purity).

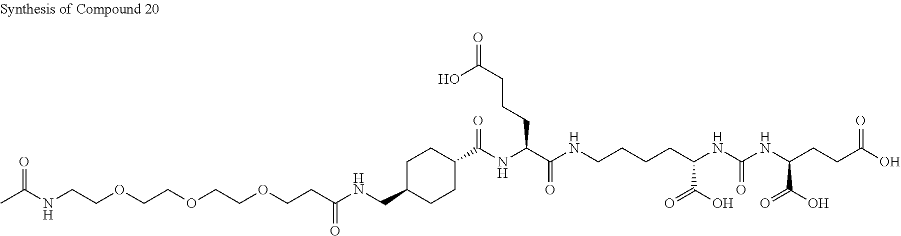 [SMILES: CC(=O)NCCOCCOCCOCCC(=O)NC[C@H]1CC[C@H](C(=O)N[C@@H](CCCC(=O)O)C(=O)NCCCC[C@H](NC(=O)N[C@@H](CCC(=O)O)C(=O)O)C(=O)O)CC1]

The synthesis of Compound 20 was performed on a 0.1 mmol scale. Precursor-I-resin was swelled in DCM for 15 min and washed with DMF. Fmoc deprotection was performed using Procedure 4. Fmoc-Aad(O^{t}Bu)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (54 mg, 43% yield, 92% purity).

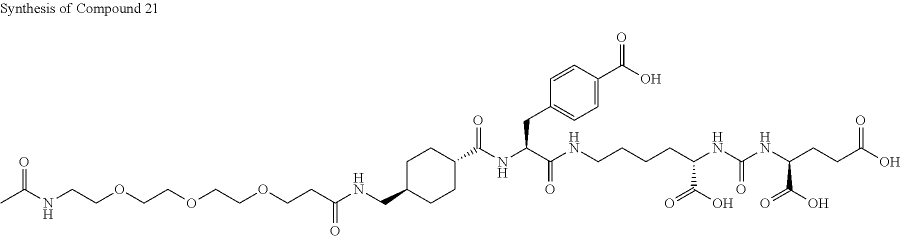 [SMILES: CC(=O)NCCOCCOCCOCCC(=O)NC[C@H]1CC[C@H](C(=O)N[C@@H](Cc2ccc(C(=O)O)cc2)C(=O)NCCCC[C@H](NC(=O)N[C@@H](CCC(=O)O)C(=O)O)C(=O)O)CC1]

The synthesis of Compound 21 was performed on a 0.08 mmol scale. Precursor-I-resin was swelled in DCM for 15 min and washed with DMF. Fmoc deprotection was performed using Procedure 4. Fmoc-homo-Phe(4-COO^{t}Bu)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (59 mg, 82% yield, 94% purity).

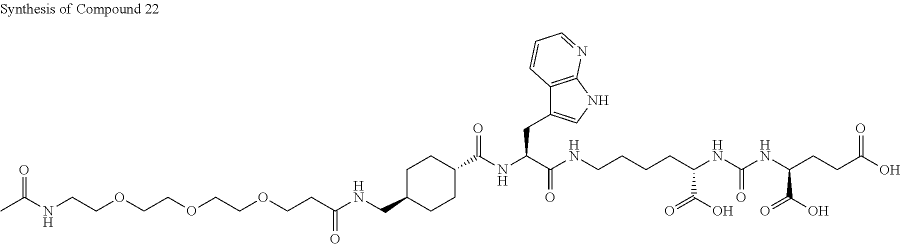 [SMILES: CC(=O)NCCOCCOCCOCCC(=O)NC[C@H]1CC[C@H](C(=O)N[C@@H](Cc2c[nH]c3ncccc23)C(=O)NCCCC[C@H](NC(=O)N[C@@H](CCC(=O)O)C(=O)O)C(=O)O)CC1]

The synthesis of Compound 22 was performed on a 0.08 mmol scale. Precursor-I-resin was swelled in DCM for 15 min and washed with DMF. Fmoc deprotection was performed using Procedure 4. Fmoc-Trp(7-aza)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (53 mg, 74% yield, 95% purity).

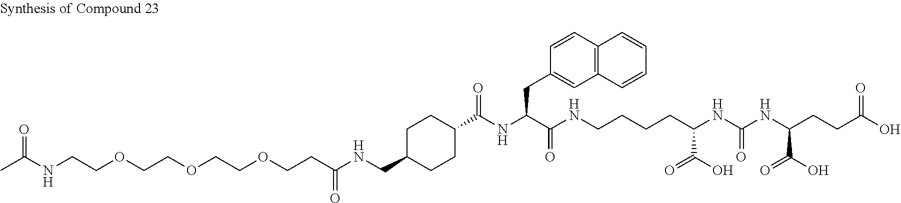 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 23 was performed on a 0.1 mmol scale on a 2-Cl-Trt resin. Fmoc-Asp(OtBu)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (44 mg, 23% yield, 97% purity).

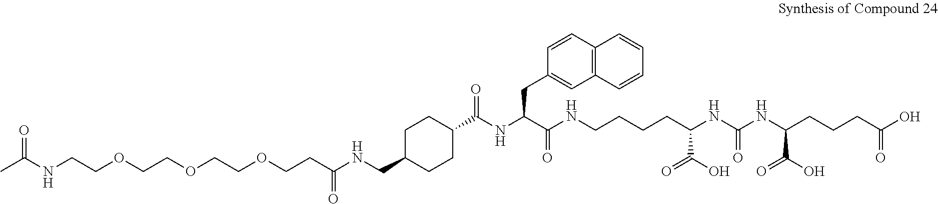 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCCC(=O)O)C(=O)O)C(=O)O)CC1]

The synthesis of Compound 23 was performed on a 0.1 mmol scale on a 2-Cl-Trt resin. Fmoc-Aad(O^{t}Bu)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (51 mg, 21% yield, 95% purity).

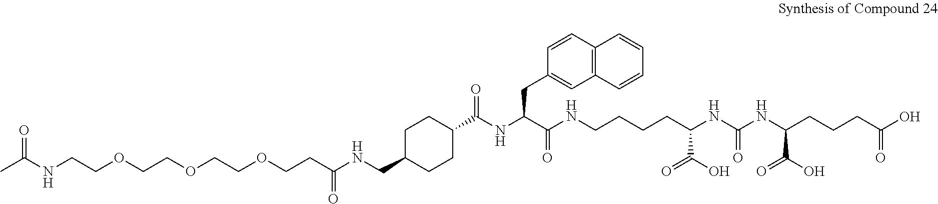 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCCC(=O)O)C(=O)O)C(=O)O)CC1]

The synthesis of Compound 24 was performed on a 0.1 mmol scale on a 2-Cl-Trt resin. Fmoc-Aad(O^{t}Bu)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (51 mg, 21% yield, 95% purity).

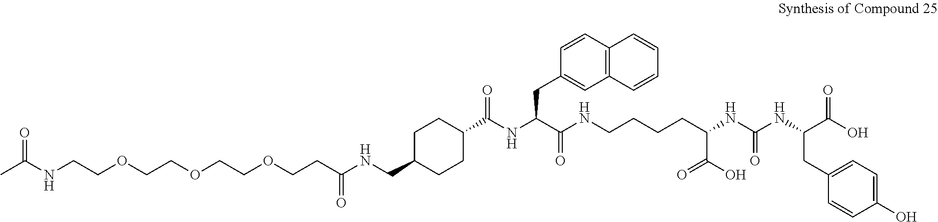 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2ccc(O)cc2)C(=O)O)C(=O)O)CC1]

The synthesis of Compound 25 was performed on a 0.1 mmol scale on a 2-Cl-Trt resin. Fmoc-Tyr(^{t}Bu)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (36 mg, 15% yield, 96% purity).

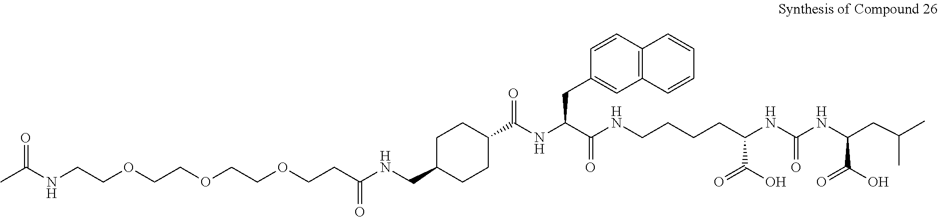 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CC(C)C)C(=O)O)C(=O)O)CC1]

The synthesis of Compound 26 was performed on a 0.21 mmol scale on a 2-Cl-Trt resin. Fmoc-Leu-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (92 mg, 23% yield, 97% purity).

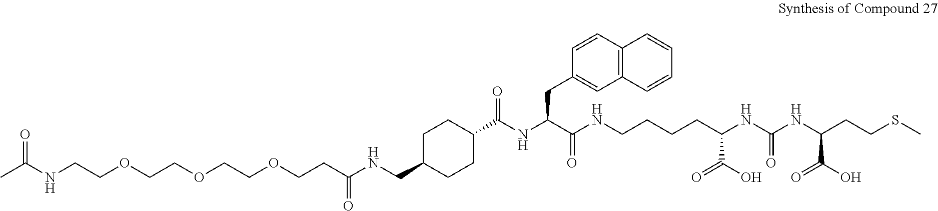 [SMILES: CSCC[C@H](NC(=O)N[C@@H](CCCCNC(=O)[C@H](Cc1ccc2ccccc2c1)NC(=O)[C@H]1CC[C@H](CNC(=O)CCOCCOCCOCCNC(C)=O)CC1)C(=O)O)C(=O)O]

The synthesis of Compound 27 was performed on a 0.22 mmol scale on a 2-Cl-Trt resin. Fmoc-Met-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (63 mg, 32% yield, 97% purity).

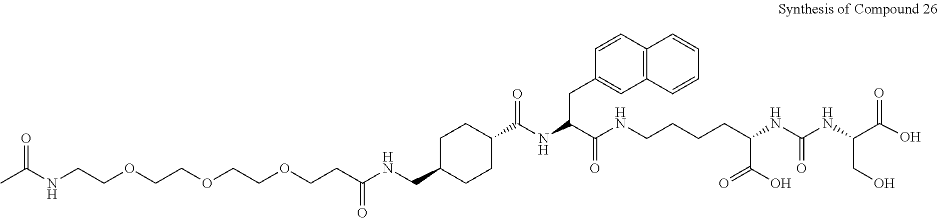

The synthesis of Compound 28 was performed on a 0.19 mmol scale on a 2-Cl-Trt resin. Fmoc-Cys(Trt)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. 4-AMCHC Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (68 mg, 42% yield, 97% purity).

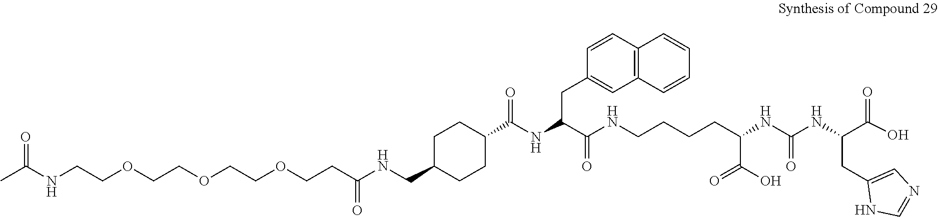

The synthesis of Compound 29 was performed on a 0.14 mmol scale on a 2-Cl-Trt resin. Fmoc-His(Trt)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (12 mg, 9% yield, 97% purity).

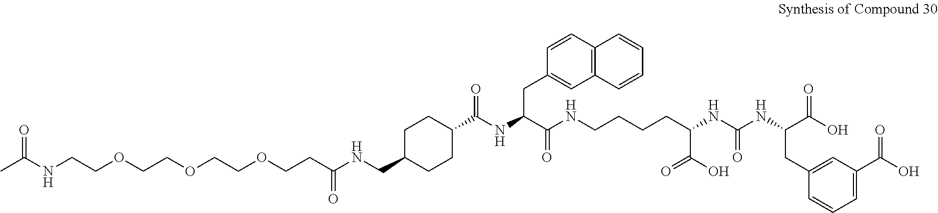 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2cccc(C(=O)O)c2)C(=O)O)C(=O)O)CC1]

The synthesis of Compound 30 was performed on a 0.18 mmol scale on a 2-Cl-Trt resin. Fmoc-Phe(3-COO^{t}Bu)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (56 mg, 32% yield, 97% purity).

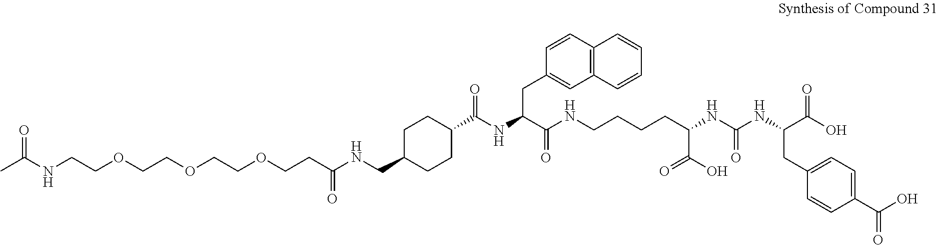 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2ccc(C(=O)O)cc2)C(=O)O)C(=O)O)CC1]

The synthesis of Compound 31 was performed on a 0.19 mmol scale on a 2-Cl-Trt resin. Fmoc-Phe(4-CO O^{t}Bu)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (36 mg, 19% yield, 96% purity).

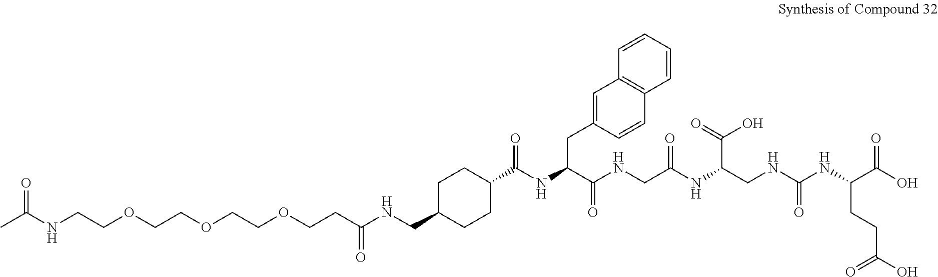 [SMILES: CC(=O)NCCOCCOCCOCCC(=O)NC[C@H]1CC[C@H](C(=O)N[C@@H](Cc2ccc3ccccc3c2)C(=O)NCC(=O)N[C@@H](CNC(=O)N[C@@H](CCC(=O)O)C(=O)O)C(=O)O)CC1]

The synthesis of Compound 32 was performed on a 0.21 mmol scale on a 2-Cl-Trt resin. Fmoc-Glu(O^{t}Bu)-OH was loaded on the resin using Procedure 5. Fmoc deprotection was performed using Procedure 4. The urea bond was formed with Fmoc-Dap-O^{t}Bu using Procedure 3. Fmoc deprotection was performed using Procedure 4. Fmoc-Gly-OH was coupled to the resin using Procedure 1. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (39 mg, 39% yield, 97% purity).

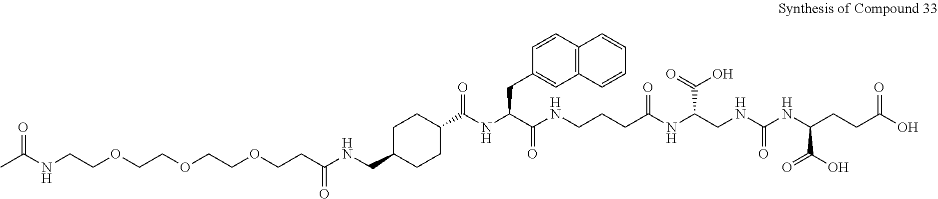 [SMILES: CC(=O)NCCOCCOCCOCCC(=O)NC[C@H]1CC[C@H](C(=O)N[C@@H](Cc2ccc3ccccc3c2)C(=O)NCCCC(=O)N[C@@H](CNC(=O)N[C@@H](CCC(=O)O)C(=O)O)C(=O)O)CC1]

The synthesis of Compound 33 was performed on a 0.11 mmol scale on a 2-Cl-Trt resin. Fmoc-Glu(O^{t}Bu)-OH was loaded on the resin using Procedure 5. Fmoc deprotection was performed using Procedure 4. The urea bond was formed with Fmoc-Dap-O^{t}Bu using Procedure 3. Fmoc deprotection was performed using Procedure 4. Fmoc-GABA-OH was coupled to the resin using Procedure 1. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (47 mg, 46% yield, 97% purity).

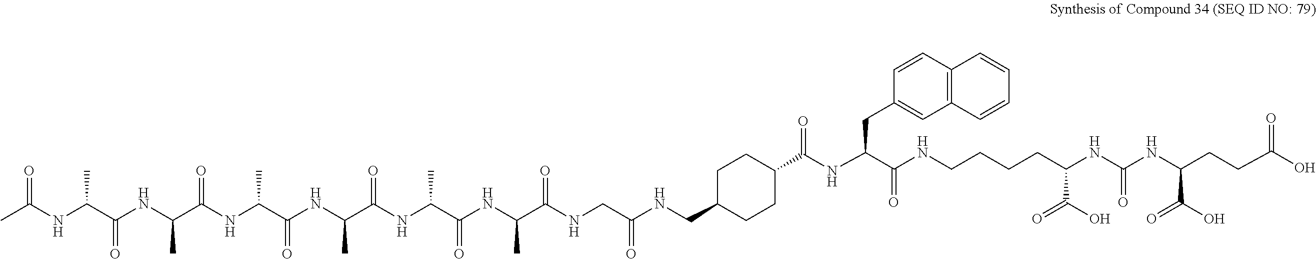 [SMILES: CC(=O)N[C@H](C)C(=O)N[C@H](C)C(=O)N[C@H](C)C(=O)N[C@H](C)C(=O)N[C@H](C)C(=O)N[C@H](C)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 34 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 34 was obtained as a white solid (4 mg, 3% yield, 87% purity).

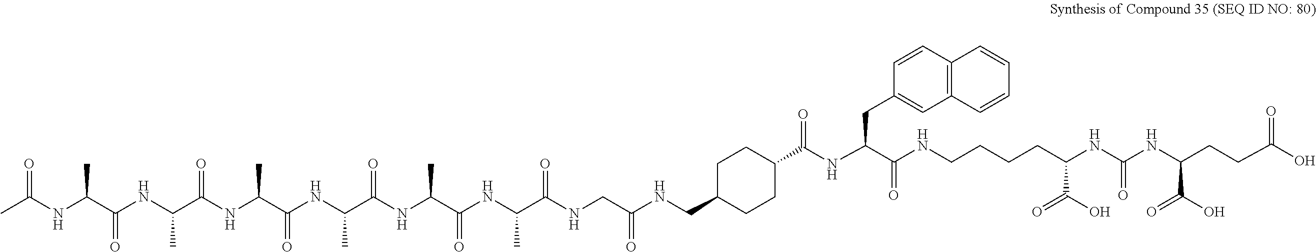 [SMILES: CC(=O)N[C@@H](C)C(=O)N[C@@H](C)C(=O)N[C@@H](C)C(=O)N[C@@H](C)C(=O)N[C@@H](C)C(=O)N[C@@H](C)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 35 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 35 was obtained as a white solid (2.5 mg, 2% yield, 86% purity).

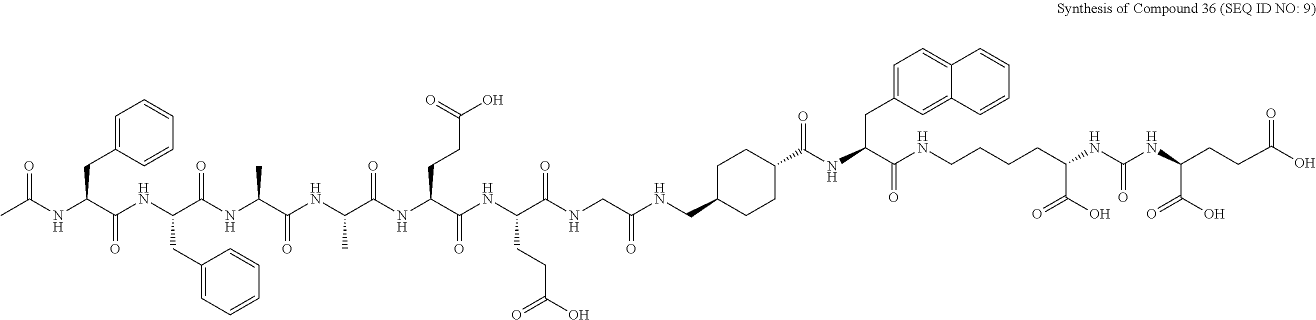 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 36 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 36 was obtained as a white solid (25 mg, 17% yield, 96% purity).

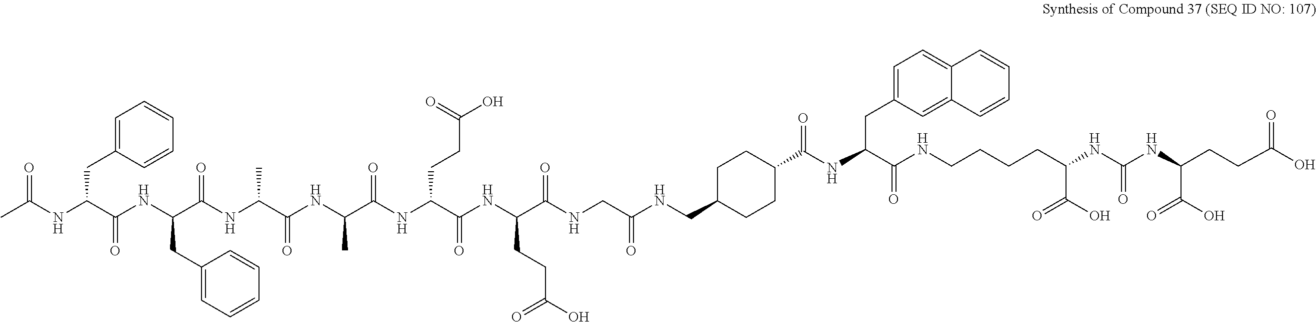 [SMILES: CC(=O)N[C@H](Cc1ccccc1)C(=O)N[C@H](Cc1ccccc1)C(=O)N[C@H](C)C(=O)N[C@H](C)C(=O)N[C@H](CCC(=O)O)C(=O)N[C@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 37 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 37 was obtained as a white solid (22 mg, 15% yield, 98% purity).

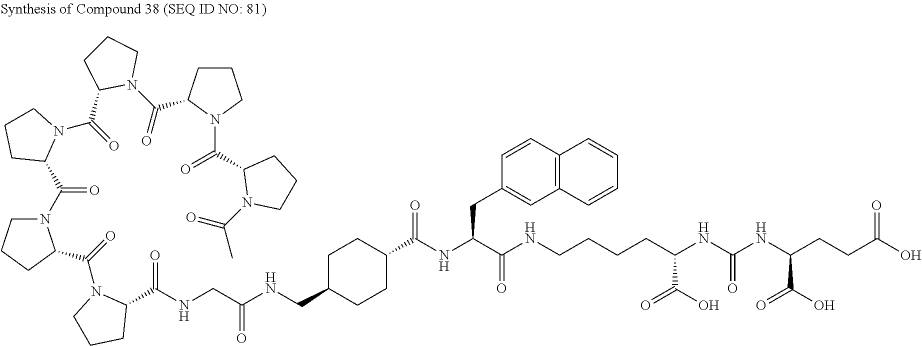 [SMILES: CC(=O)N1CCC[C@H]1C(=O)N1CCC[C@H]1C(=O)N1CCC[C@H]1C(=O)N1CCC[C@H]1C(=O)N1CCC[C@H]1C(=O)N1CCC[C@H]1C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 38 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 38 was obtained as a white solid (21 mg, 16% yield, 92% purity).

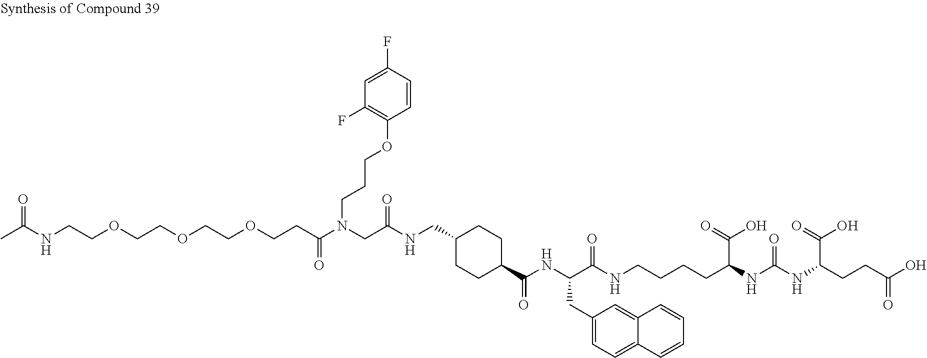 [SMILES: CC(=O)NCCOCCOCCOCCC(=O)N(CCCOc1ccc(F)cc1F)CC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 39 was performed on a 0.05 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. The resin was then treated with a solution of DIPEA (3 equiv.) and 2-bromoacetic anhydride (10 equiv.) in DMF at r.t. for 30 min. The resin was washed with DMF. The resin was treated with DIPEA (10 equiv.) and 3-(2,4-difluorophenoxy)propan-1-amine (building block-I, 5 equiv.) in DMF at r.t. for 2 h. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (7 mg, 10% yield, 93% purity).

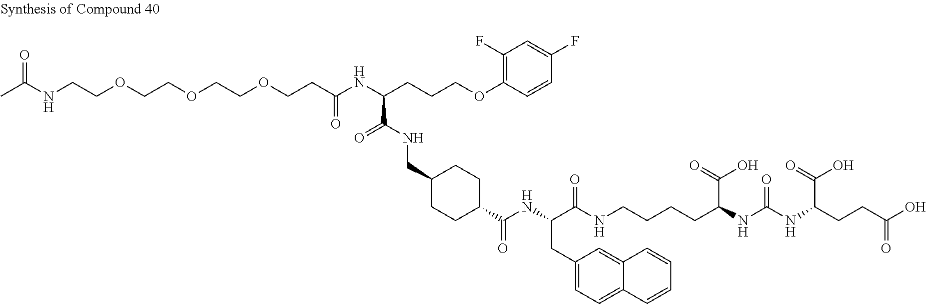 [SMILES: CC(=O)NCCOCCOCCOCCC(=O)N[C@@H](CCCOc1ccc(F)cc1F)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 40 was performed on a 0.18 mmol scale. was performed on a 0.18 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-(S)-2-amino-5-(2,4-difluorophenoxy)pentanoic acid (building block-II) was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (7 mg, 6% yield, 95% purity).

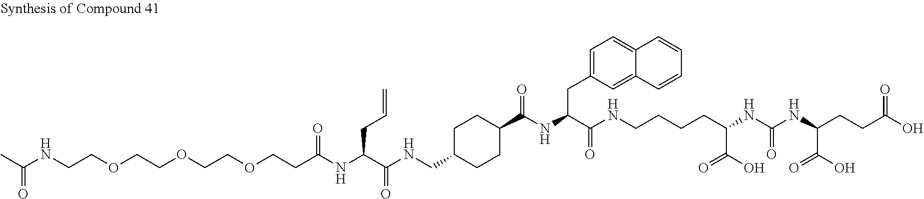 [SMILES: C=CC[C@H](NC(=O)CCOCCOCCOCCNC(C)=O)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

Compound 41 was obtained as a side-product from Compound 40 synthesis and was purified and isolated during Procedure 8. Preparative HPLC fractions containing the purified side-product were combined, frozen and lyophilized yielding a white solid (5 mg, 3% yield, 90% purity).

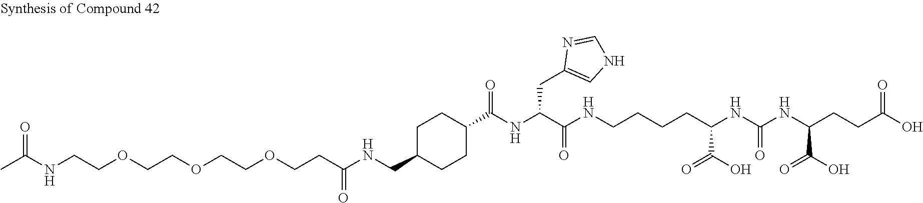 [SMILES: CC(=O)NCCOCCOCCOCCC(=O)NC[C@H]1CC[C@H](C(=O)N[C@H](Cc2c[nH]cn2)C(=O)NCCCC[C@H](NC(=O)N[C@@H](CCC(=O)O)C(=O)O)C(=O)O)CC1]

The synthesis of Compound 42 was performed on a 0.066 mmol scale. Precursor-I-resin was swollen in DCM for 15 min and washed with DMF. Fmoc deprotection was performed using Procedure 4. Fmoc-D-His(Trt)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (4.37 mg, 8% yield, 96% purity).

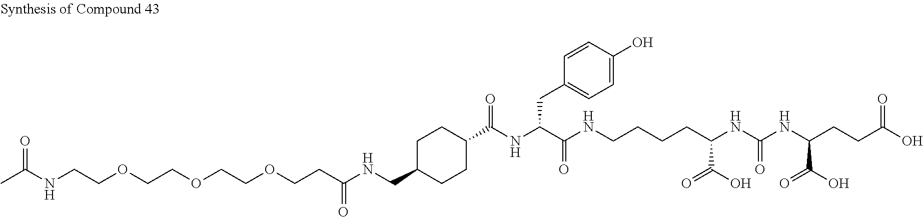 [SMILES: CC(=O)NCCOCCOCCOCCC(=O)NC[C@H]1CC[C@H](C(=O)N[C@H](Cc2ccc(O)cc2)C(=O)NCCCC[C@H](NC(=O)N[C@@H](CCC(=O)O)C(=O)O)C(=O)O)CC1]

The synthesis of Compound 43 was performed on a 0.066 mmol scale. Precursor-I-resin was swollen in DCM for 15 min and washed with DMF. Fmoc deprotection was performed using Procedure 4. Fmoc-D-Tyr(O^{t}Bu)-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in 4 mL of DMSO and purified by preparative RP-HPLC using Procedure 8. Preparative HPLC fractions containing the purified product were combined and freeze-dried, yielding a white solid (8.2 mg, 14% yield, 95% purity).

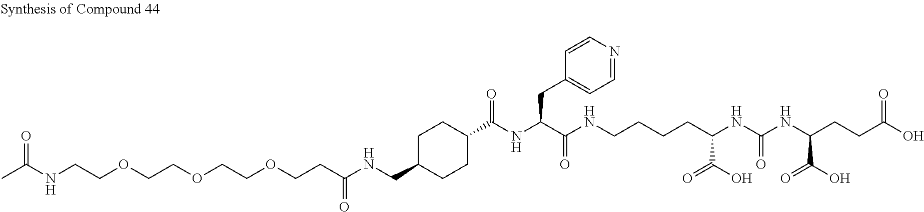 [SMILES: CC(=O)NCCOCCOCCOCCC(=O)NC[C@H]1CC[C@H](C(=O)N[C@@H](Cc2ccncc2)C(=O)NCCCC[C@H](NC(=O)N[C@@H](CCC(=O)O)C(=O)O)C(=O)O)CC1]

The synthesis of Compound 44 was performed on a 0.09 mmol scale. Precursor-I-resin was swelled in DCM for 15 min and washed with DMF. Fmoc deprotection was performed using Procedure 4. Fmoc-4-Pal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (14 mg, 19% yield, 95% purity).

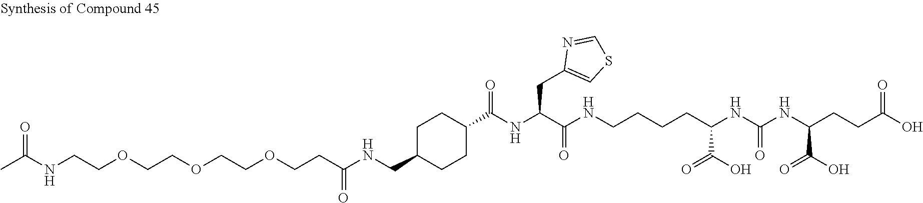 [SMILES: CC(=O)NCCOCCOCCOCCC(=O)NC[C@H]1CC[C@H](C(=O)N[C@@H](Cc2cscn2)C(=O)NCCCC[C@H](NC(=O)N[C@@H](CCC(=O)O)C(=O)O)C(=O)O)CC1]

The synthesis of Compound 45 was performed on a 0.09 mmol scale. Precursor-I-resin was swelled in DCM for 15 min and washed with DMF. Fmoc deprotection was performed using Procedure 4. Fmoc-(4-Thiazolyl)alanine was coupled to the resin using Procedure 3. Fmoc deprotection was performed using Procedure 4. Fmoc-AMCHC-OH was coupled to the resin using Procedure 3. Fmoc deprotection was performed using Procedure 4. Fmoc-N-PEG3-OH was coupled to the resin using Procedure 3. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (13 mg, 16% yield, 96% purity).

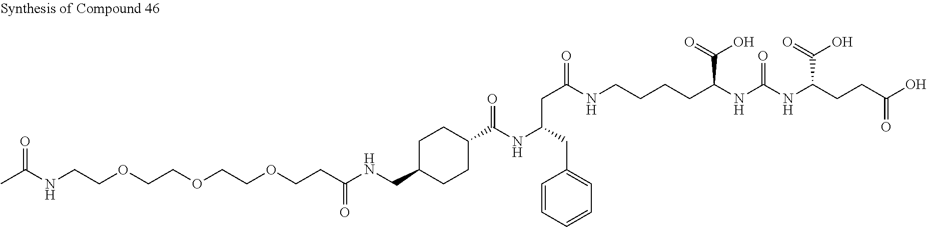 [SMILES: CC(=O)NCCOCCOCCOCCC(=O)NC[C@H]1CC[C@H](C(=O)N[C@H](CC(=O)NCCCC[C@H](NC(=O)N[C@@H](CCC(=O)O)C(=O)O)C(=O)O)Cc2ccccc2)CC1]

The synthesis of Compound 46 was performed on a 0.09 mmol scale. Precursor-I-resin was swelled in DCM for 15 min and washed with DMF. Fmoc deprotection was performed using Procedure 4. Fmoc-β-homo-Phe-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (17 mg, 22% yield, 98% purity).

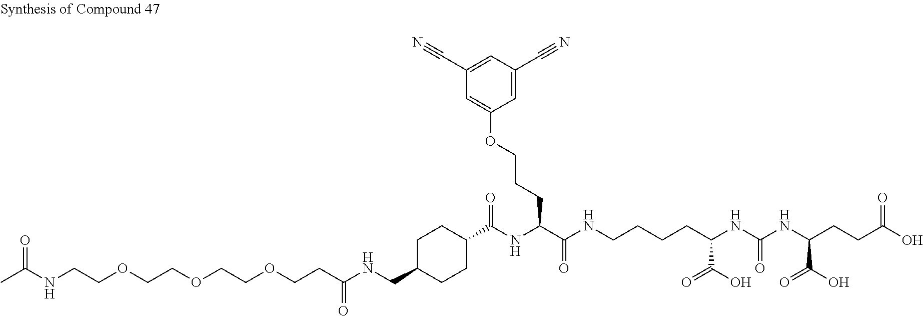 [SMILES: CC(=O)NCCOCCOCCOCCC(=O)NC[C@H]1CC[C@H](C(=O)N[C@@H](CCCOc2cc(C#N)cc(C#N)c2)C(=O)NCCCC[C@H](NC(=O)N[C@@H](CCC(=O)O)C(=O)O)C(=O)O)CC1]

The synthesis of Compound 47 was performed on a 66 μmol scale. Precursor-I-resin was swelled in DCM for 15 min and washed with DMF. Fmoc deprotection was performed using Procedure 4. Fmoc-(S)-2-amino-5-(3,5-dicyanophenoxy)pentanoic acid was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (18 mg, 29% yield, 96% purity).

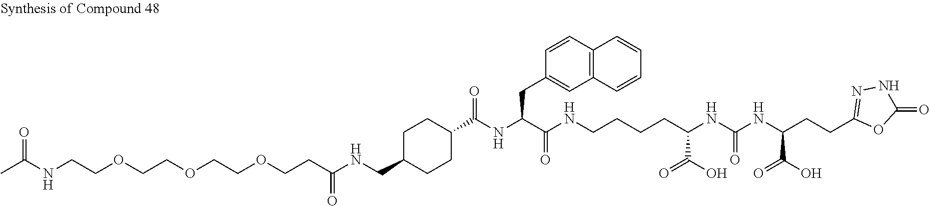 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](CCc2n[nH]c(=O)o2)C(=O)O)C(=O)O)CC1]

The synthesis of Compound 48 was performed on a 66 μmol scale. Precursor-I-resin was swelled in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 7. The cleaved product was treated with DIPEA (2.2 equiv), HATU (1.1 equiv) and hydrazine monohydrate (2 equiv) in DMF at r.t. for 2 h. It was then treated with CDI (20 equiv) in DMF at r.t. for 12 h. The crude paste was solubilized in DMSO and purified by preparative RP-HPLC using Procedure 8. Preparative HPLC fractions containing the purified product were combined, frozen and lyophilized yielding a white solid (52 mg, 78% yield, 95% purity).

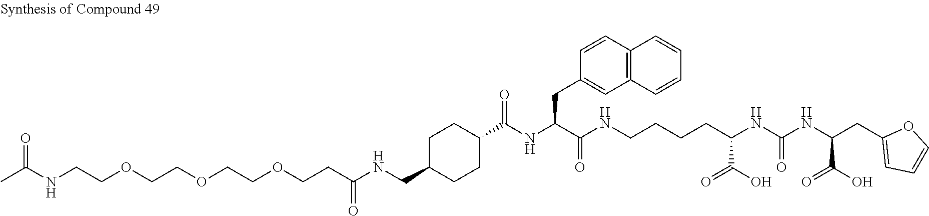 [SMILES: CC(=O)NCCOCCOCCOCCC(=O)NC[C@H]1CC[C@H](C(=O)N[C@@H](Cc2ccc3ccccc3c2)C(=O)NCCCC[C@H](NC(=O)N[C@@H](Cc2ccco2)C(=O)O)C(=O)O)CC1]

The synthesis of Compound 49 was performed on a 0.1 mmol scale on a 2-Cl-Trt resin. Fmoc-Ala(2-furyl)-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (4.5 mg, 5% yield, 90% purity).

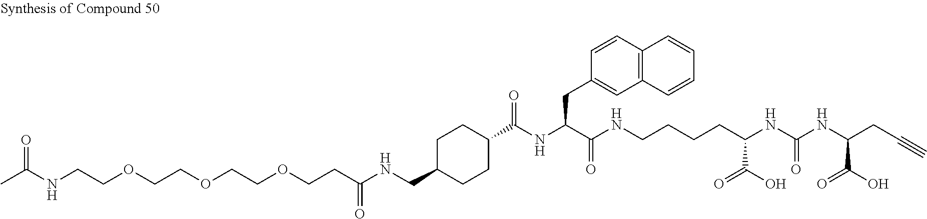 [SMILES: C#CC[C@H](NC(=O)N[C@@H](CCCCNC(=O)[C@H](Cc1ccc2ccccc2c1)NC(=O)[C@H]1CC[C@H](CNC(=O)CCOCCOCCOCCNC(C)=O)CC1)C(=O)O)C(=O)O]

The synthesis of Compound 50 was performed on a 0.1 mmol scale on a 2-Cl-Trt resin. Fmoc-Pra-OH was loaded on the resin using Procedure 5. Fmoc deprotection was performed using Procedure 4. The urea bond was formed with H-Lys(Fmoc)-O^{t}Bu using Procedure 3. Fmoc deprotection was performed using Procedure 4. Fmoc-2-Nal-OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (4.6 mg, 5% yield, 86% purity).

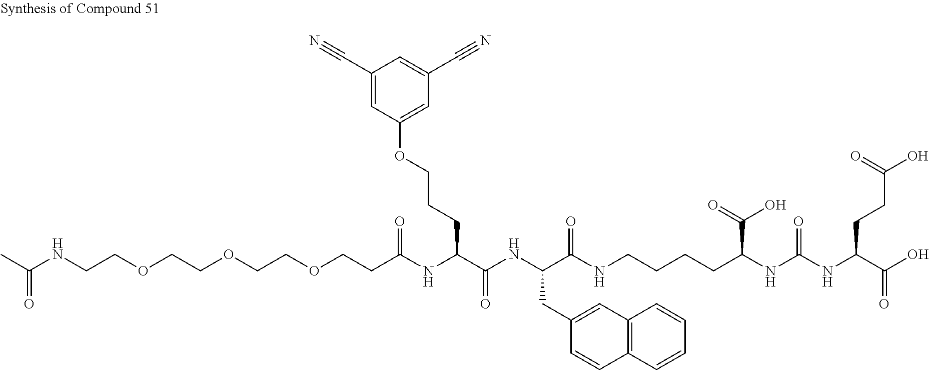 [SMILES: CC(=O)NCCOCCOCCOCCC(=O)N[C@@H](CCCOc1cc(C#N)cc(C#N)c1)C(=O)N[C@@H](Cc1ccc2ccccc2c1)C(=O)NCCCC[C@H](NC(=O)N[C@@H](CCC(=O)O)C(=O)O)C(=O)O]

The synthesis of Compound 51 was performed on a 0.2 mmol scale. Precursor-I-resin was swelled in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-(S)-2-amino-5-(3,5-dicyanophenoxy)pentanoic acid was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (38 mg, 43% yield, 98% purity).

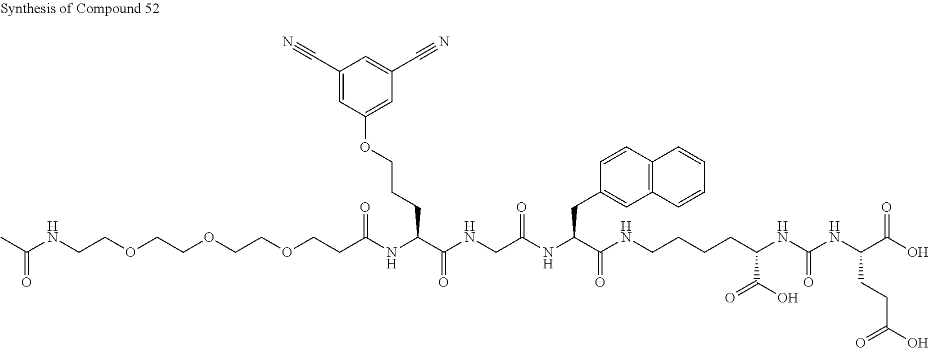 [SMILES: CC(=O)NCCOCCOCCOCCC(=O)N[C@@H](CCCOc1cc(C#N)cc(C#N)c1)C(=O)NCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 52 was performed on a 0.2 mmol scale. Precursor-I-resin was swelled in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-(S)-2-amino-5-(3,5-dicyanophenoxy)pentanoic acid was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (38 mg, 41% yield, 95% purity).

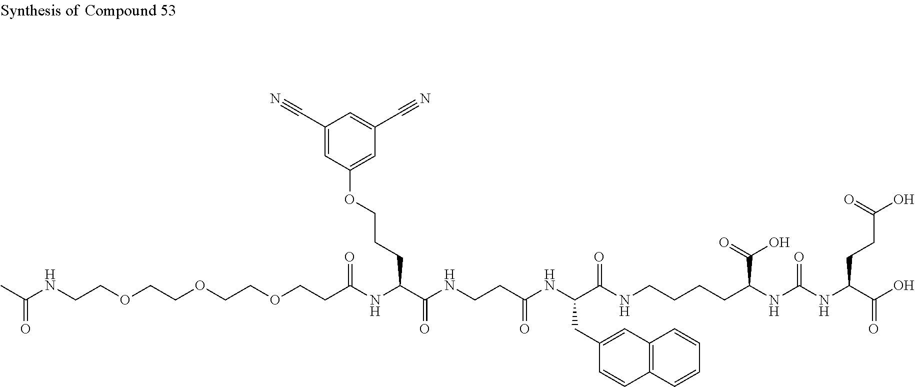 [SMILES: CC(=O)NCCOCCOCCOCCC(=O)N[C@@H](CCCOc1cc(C#N)cc(C#N)c1)C(=O)NCCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 53 was performed on a 0.2 mmol scale. Precursor-I-resin was swelled in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-β-Ala-OH was coupled to the resin using Procedure 1. Fmoc deprotection was performed using Procedure 4. Fmoc-(S)-2-amino-5-(3,5-dicyanophenoxy)pentanoic acid was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined, frozen and lyophilized yielding a white solid (66 mg, 70% yield, 92% purity).

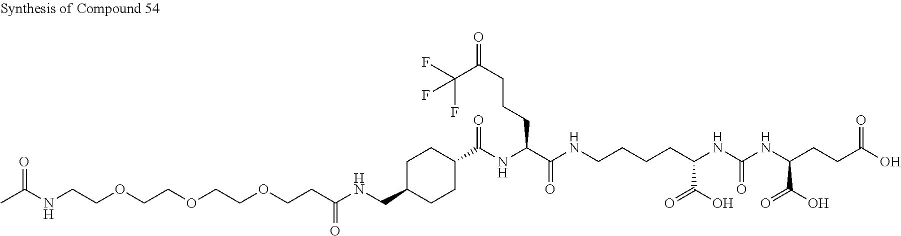 [SMILES: CC(=O)NCCOCCOCCOCCC(=O)NC[C@H]1CC[C@H](C(=O)N[C@@H](CCCC(=O)C(F)(F)F)C(=O)NCCCC[C@H](NC(=O)N[C@@H](CCC(=O)O)C(=O)O)C(=O)O)CC1]

The synthesis of Compound 54 was initiated on a 0.1 mmol scale. H-Lys(Boc)-2-Cl-Trt resin was swelled in DMF for 15 min. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed with a solution of acetic anhydride (10 equiv) in DMF for 15 min at r.t. The resin was washed with DMF, DCM, MeOH, DEE, and dried under vacuum. The resin was then treated with the cleavage mixture using Procedure 7 yielding Ac-PEG3-AMCHC-Lys(Boc)-COOH precursor as a white solid. m(precursor)=83 mg. The precursor was used directly without purification. EuK(O^{t}Bu)₃ (CAS: 1025796-31-9, 64 mg; 0.13 mmol; 1 equiv), Ac-PEG3-AMCHC-Lys(Boc)-COOH (83 mg; 0.13 mmol; 1 equiv) and HOOBt (43 mg; 0.26 mmol; 2 equiv) were dissolved in 5.3 mL of DMF. The coupling reaction was initiated upon addition of DIPEA (34 μL; 0.20 μmol; 1.5 equiv) and EDC*HCl (38 mg; 0.20 μmol; 1.5 equiv). It was let to react at r.t. for 3 h. The reaction mixture was then concentrated at reduced pressure. The resulting paste was treated with 13.1 mL of TFA/water/DCM (60:5:35, v/v/v). It was let to react at r.t. for 1 h. The reaction mixture was concentrated at reduced pressure. Trifluoroacetylation of lysine side-chain happened during the TFA cleavage step. The resulting paste was dissolved in DMSO and purified using Procedure 8. Preparative HPLC fractions containing the purified product were combined, frozen and lyophilized yielding a white solid (Compound 54, 1.5 mg, 1% yield, 94% purity).

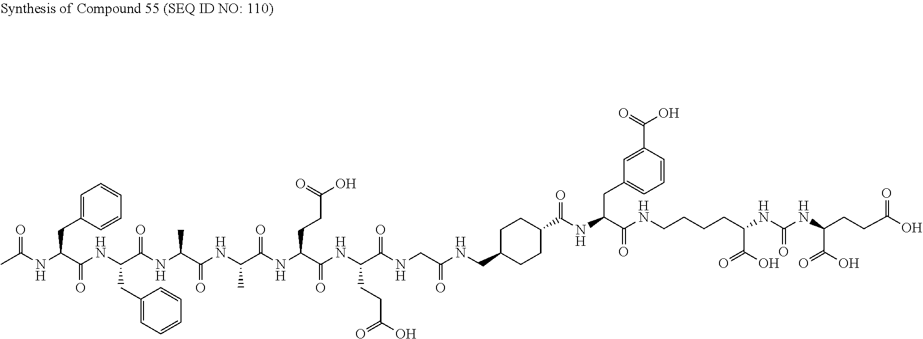 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2cccc(C(=O)O)c2)C(=O)NCCCC[C@H](NC(=O)N[C@@H](CCC(=O)O)C(=O)O)C(=O)O)CC1]

The synthesis of Compound 55 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 55 was obtained as a white solid (62 mg, 43% yield, 95% purity).

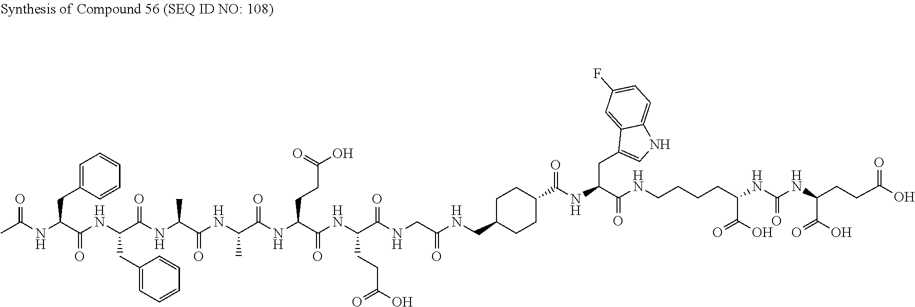 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2c[nH]c3ccc(F)cc23)C(=O)NCCCC[C@H](NC(=O)N[C@@H](CCC(=O)O)C(=O)O)C(=O)O)CC1]

The synthesis of Compound 56 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 56 was obtained as a white solid (39 mg, 27% yield, 90% purity).

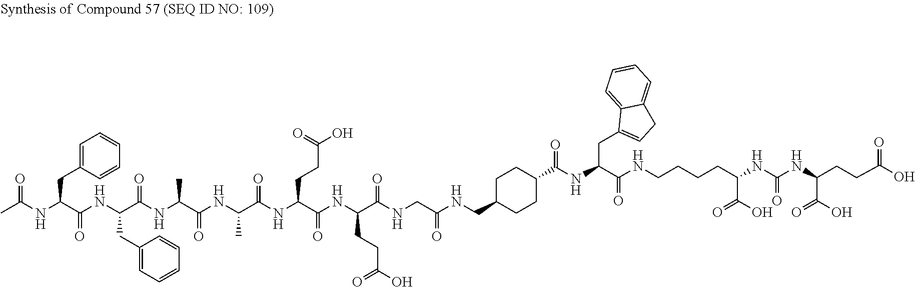 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@H](CCC(=O)O)C(=O)NCC(=O)NC[C@H]1CC[C@H](C(=O)N[C@@H](CC2=CCc3ccccc32)C(=O)NCCCC[C@H](NC(=O)N[C@@H](CCC(=O)O)C(=O)O)C(=O)O)CC1]

The synthesis of Compound 57 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 57 was obtained as a white solid (42 mg, 29% yield, 97% purity).

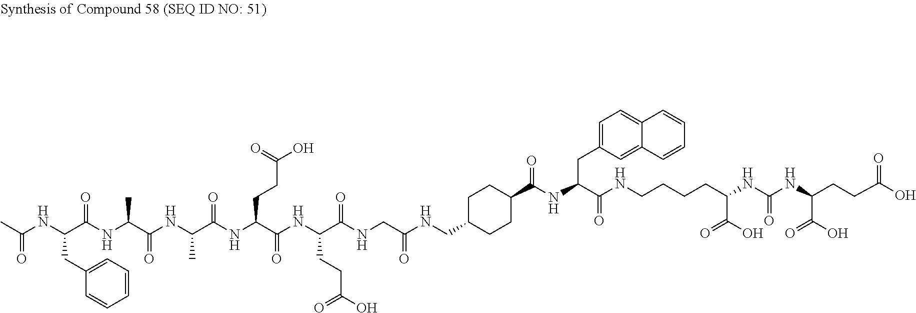 [SMILES: CC(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 58 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 58 was obtained as a white solid (48 mg, 37% yield, 92% purity).

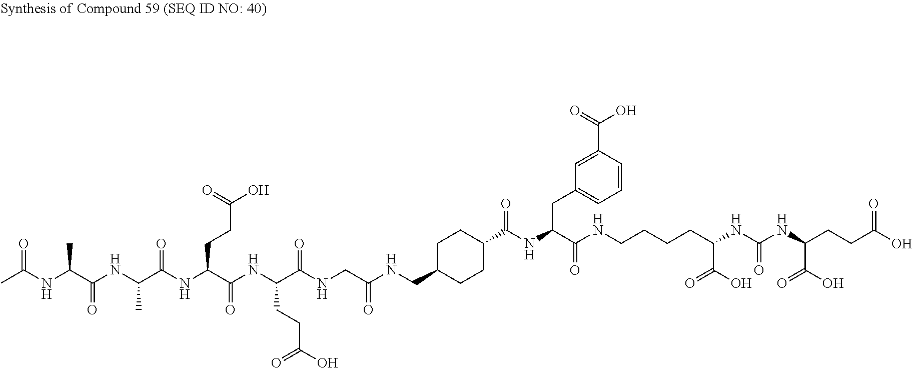 [SMILES: CC(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2cccc(C(=O)O)c2)C(=O)NCCCC[C@H](NC(=O)N[C@@H](CCC(=O)O)C(=O)O)C(=O)O)CC1]

The synthesis of Compound 59 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 59 was obtained as a white solid (60 mg, 52% yield, 95% purity).

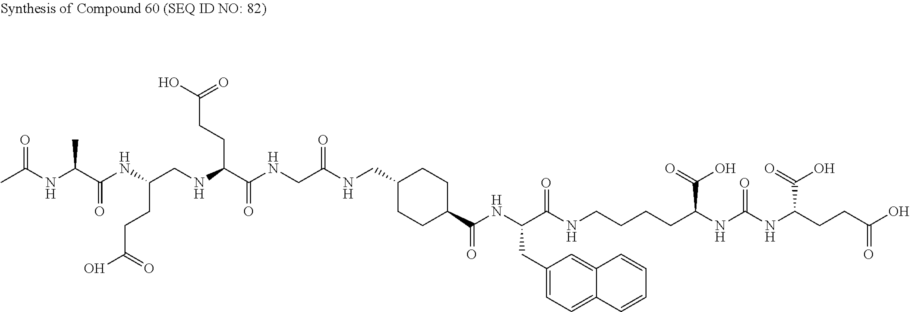 [SMILES: CC(=O)N[C@@H](C)C(=O)N[C@@H](CCC(=O)O)CN[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 60 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 60 was obtained as a white solid (49 mg, 45% yield, 91% purity).

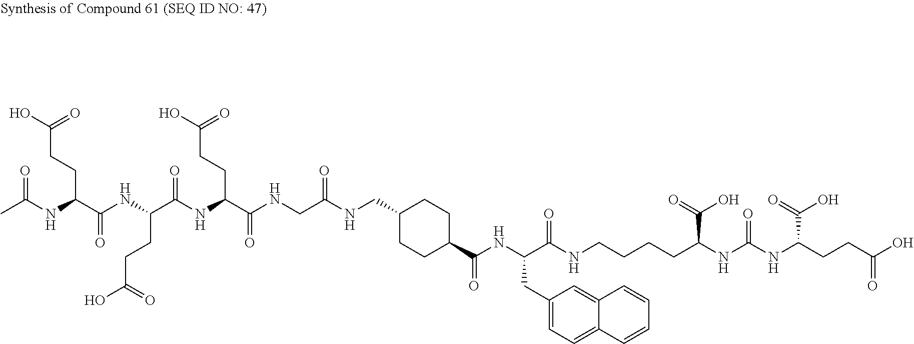 [SMILES: CC(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 61 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 61 was obtained as a white solid (51 mg, 45% yield, 89% purity).

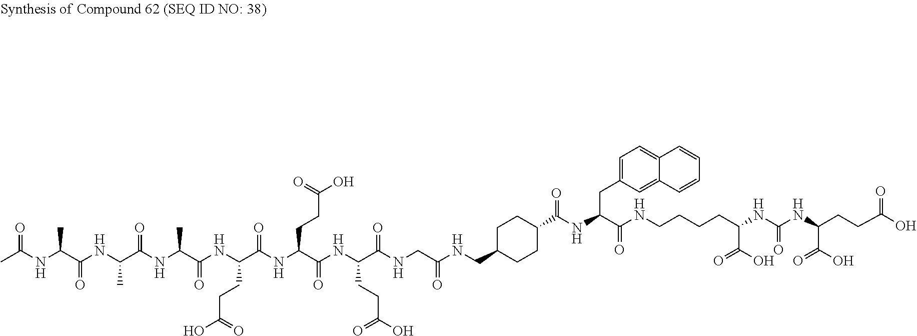 [SMILES: CC(=O)N[C@@H](C)C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 62 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 62 was obtained as a white solid (89 mg, 65% yield, 95% purity).

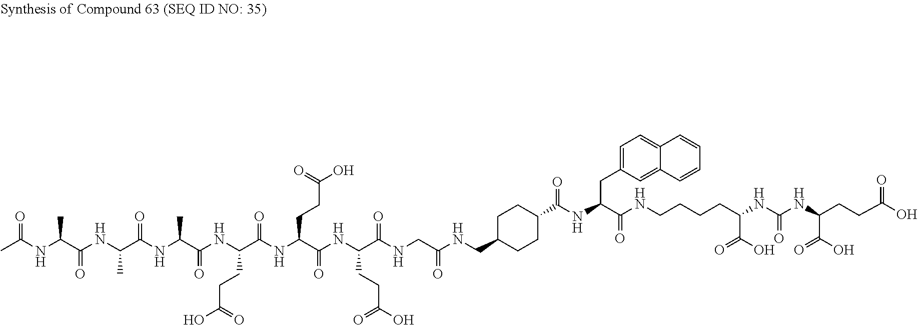 [SMILES: CC(=O)N[C@@H](C)C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 63 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 63 was obtained as a white solid (74 mg, 57% yield, 95% purity).

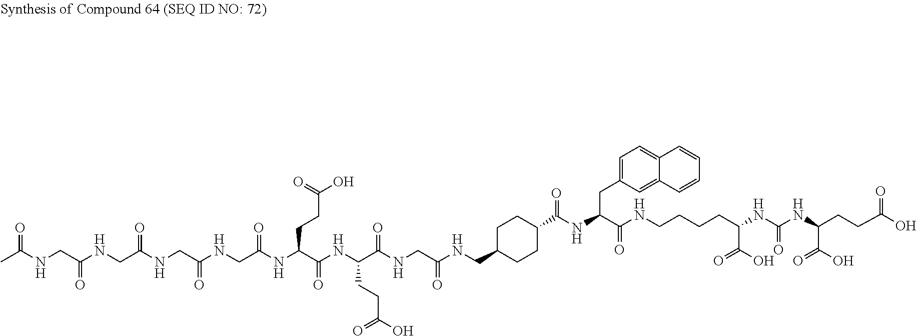 [SMILES: CC(=O)NCC(=O)NCC(=O)NCC(=O)NCC(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 64 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 64 was obtained as a white solid (68 mg, 55% yield, 95% purity).

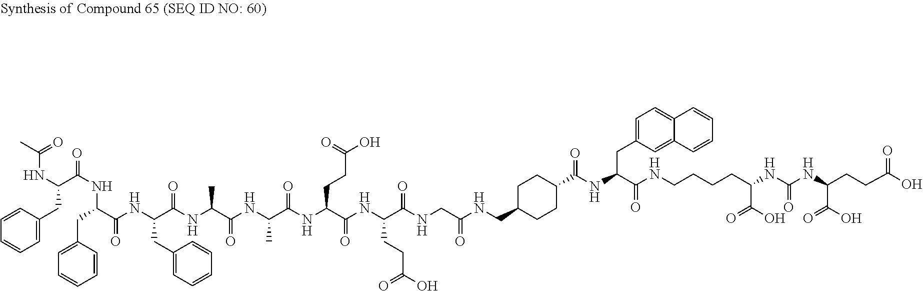 [SMILES: CC(=O)N[C@@H](Cc1ccccc1)C(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 65 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 65 was obtained as a white solid (54 mg, 34% yield, 93% purity).

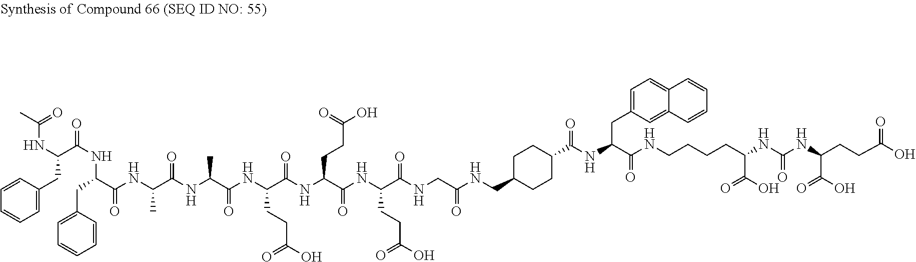 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 66 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 66 was obtained as a white solid (90 mg, 57% yield, 95% purity).

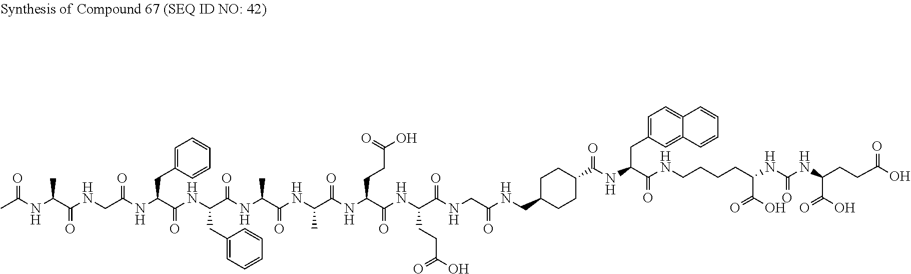 [SMILES: CC(=O)N[C@@H](C)C(=O)NCC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 67 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 67 was obtained as a white solid (60 mg, 38% yield, 92% purity).

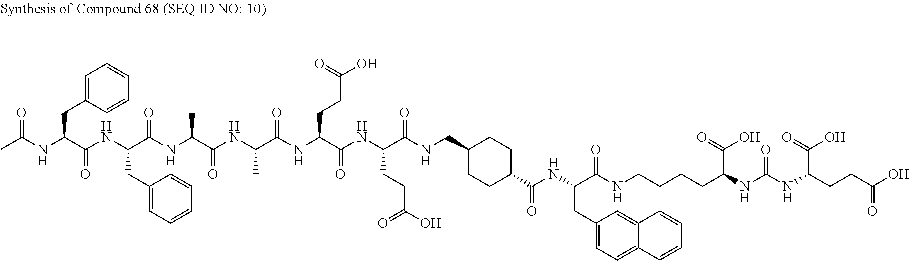 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 68 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 68 was obtained as a white solid (60 mg, 43% yield, 89% purity).

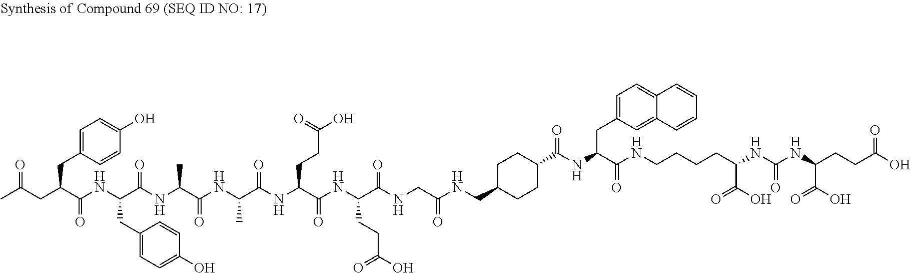 [SMILES: CC(=O)C[C@@H](Cc1ccc(O)cc1)C(=O)N[C@@H](Cc1ccc(O)cc1)C(=O)N[C@@H](C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 69 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 69 was obtained as a white solid (83 mg, 56% yield, 98% purity).

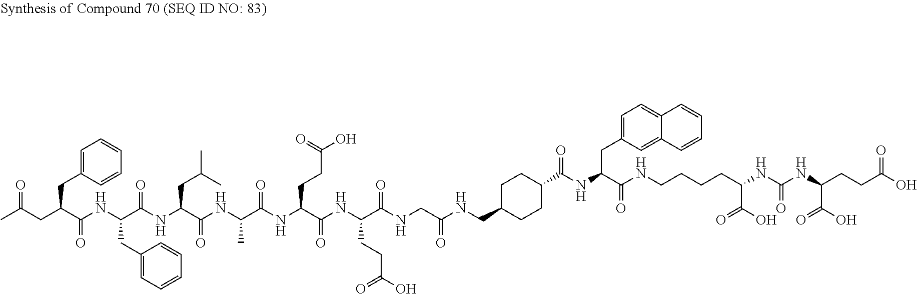 [SMILES: CC(=O)C[C@@H](Cc1ccccc1)C(=O)N[C@@H](Cc1ccccc1)C(=O)N[C@@H](CC(C)C)C(=O)N[C@@H](C)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 70 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 70 was obtained as a white solid (79 mg, 51% yield, 91% purity).

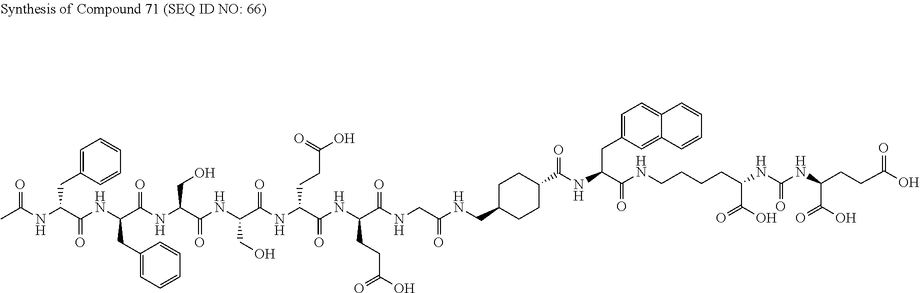 [SMILES: CC(=O)N[C@H](Cc1ccccc1)C(=O)N[C@H](Cc1ccccc1)C(=O)N[C@@H](CO)C(=O)N[C@@H](CO)C(=O)N[C@H](CCC(=O)O)C(=O)N[C@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 71 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 71 was obtained as a white solid (61 mg, 41% yield, 88% purity).

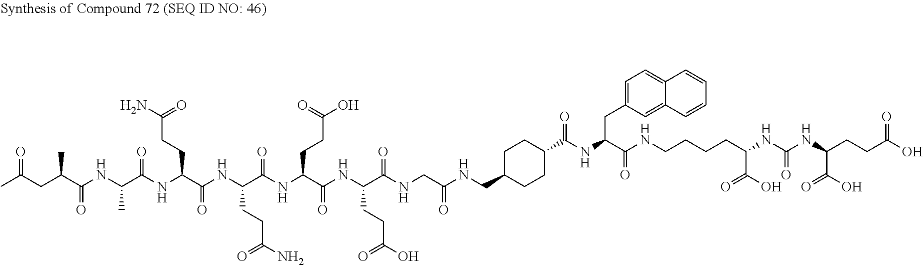 [SMILES: CC(=O)C[C@@H](C)C(=O)N[C@@H](C)C(=O)N[C@@H](CCC(N)=O)C(=O)N[C@@H](CCC(N)=O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 72 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 72 was obtained as a white solid (44 mg, 31% yield, 96% purity).

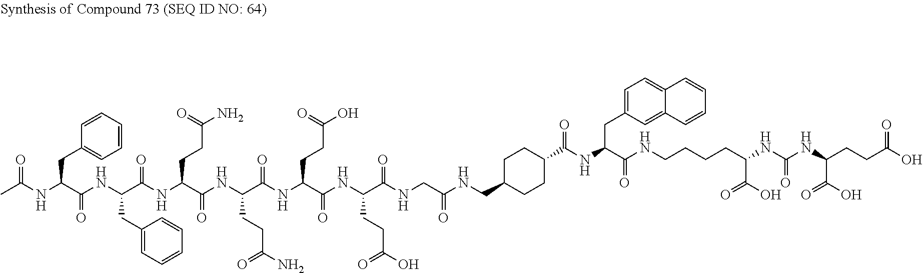 [SMILES: CC(=O)N[C@@H](Cc1ccccc1)C(=O)N[C@@H](Cc1ccccc1)C(=O)N[C@@H](CCC(N)=O)C(=O)N[C@@H](CCC(N)=O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 73 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 73 was obtained as a white solid (19 mg, 12% yield, 90% purity).

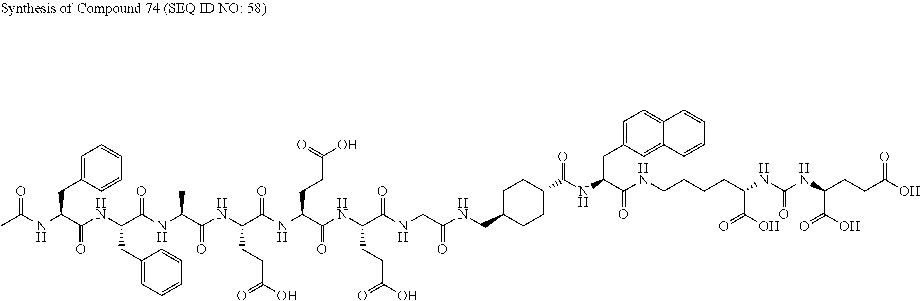 [SMILES: CC(=O)N[C@@H](Cc1ccccc1)C(=O)N[C@@H](Cc1ccccc1)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 74 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 74 was obtained as a white solid (81 mg, 54% yield, 88% purity).

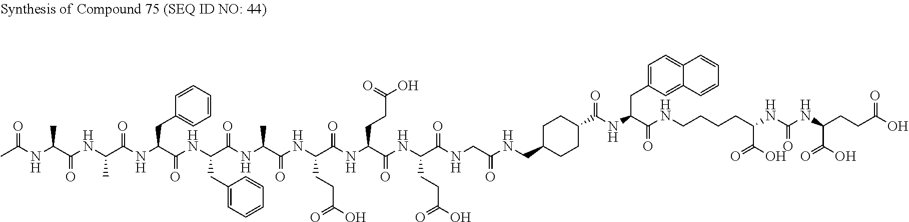 [SMILES: CC(=O)N[C@@H](C)C(=O)N[C@@H](C)C(=O)N[C@@H](Cc1ccccc1)C(=O)N[C@@H](Cc1ccccc1)C(=O)N[C@@H](C)C(=O)N[C@@H](CCC(=O)O)C(=O)N[C@@H](CCC(=O)O)C(=O)N[C@@H](CCC(=O)O)C(=O)NCC(=O)NC[C@H]1CC[C@H](C(=O)N[C@@H](Cc2ccc3ccccc3c2)C(=O)NCCCC[C@H](NC(=O)N[C@@H](CCC(=O)O)C(=O)O)C(=O)O)CC1]

The synthesis of Compound 75 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 75 was obtained as a white solid (39 mg, 23% yield, 89% purity).

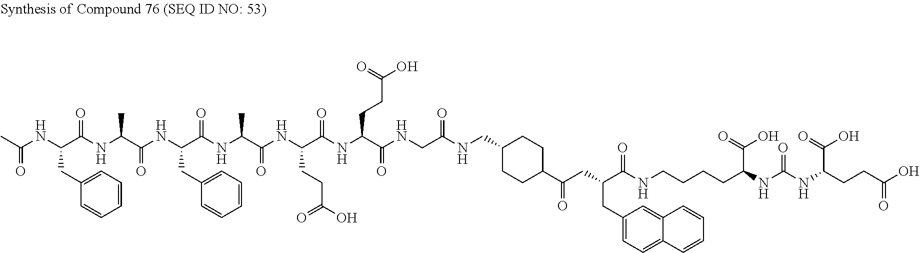

The synthesis of Compound 76 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 76 was obtained as a white solid (53 mg, 37% yield, 89% purity).

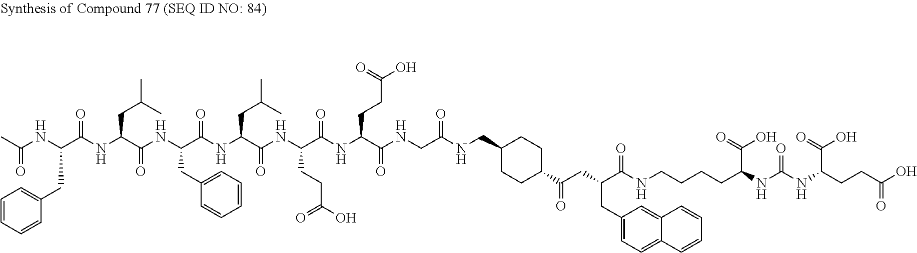 [SMILES: CC(=O)N[C@@H](Cc1ccccc1)C(=O)N[C@@H](CC(C)C)C(=O)N[C@@H](Cc1ccccc1)C(=O)N[C@@H](CC(C)C)C(=O)N[C@@H](CCC(=O)O)C(=O)N[C@@H](CCC(=O)O)C(=O)NCC(=O)NC[C@H]1CC[C@H](C(=O)C[C@@H](Cc2ccc3ccccc3c2)C(=O)NCCCC[C@H](NC(=O)N[C@@H](CCC(=O)O)C(=O)O)C(=O)O)CC1]

The synthesis of Compound 77 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 77 was obtained as a white solid (46 mg, 30% yield, 93% purity).

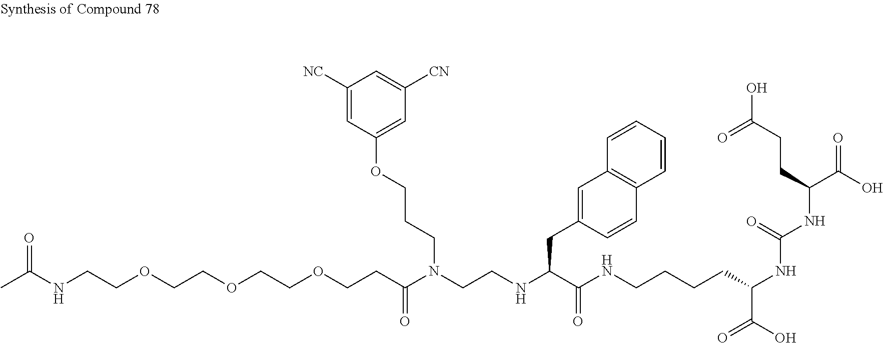 [SMILES: CC(=O)NCCOCCOCCOCCC(=O)N(CCCOc1cc(C#N)cc(C#N)c1)CCN[C@@H](Cc1ccc2ccccc2c1)C(=O)NCCCC[C@H](NC(=O)N[C@@H](CCC(=O)O)C(=O)O)C(=O)O]

The synthesis of Compound 78 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. The resin was then treated with a solution of DIPEA (3 equiv.) and 2-bromoacetic anhydride (3 equiv.) in DMF at r.t. for 30 min. The resin was washed with DMF. The resin was treated with DIPEA (5 equiv.) and 5-(3-aminopropoxy)isophthalonitrile (as a TFA salt, 3 equiv.) (building block-IV) in DMF at r.t. for 2 h. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (23 mg, 22% yield, 94.47% purity).

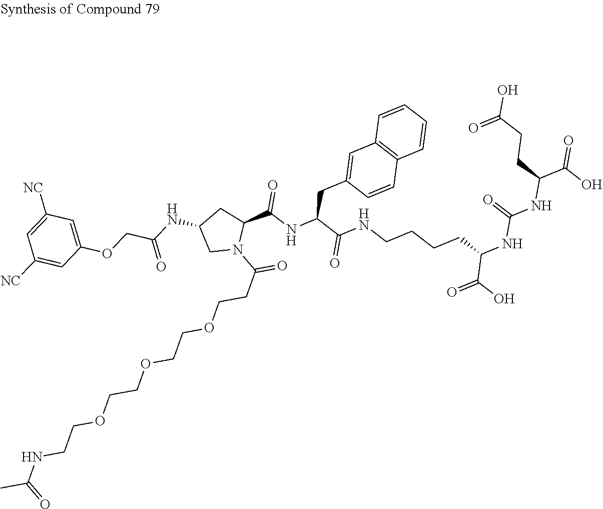 [SMILES: CC(=O)NCCOCCOCCOCCC(=O)N1C[C@H](NC(=O)COc2cc(C#N)cc(C#N)c2)C[C@H]1C(=O)N[C@@H](Cc1ccc2ccccc2c1)C(=O)NCCCC[C@H](NC(=O)N[C@@H](CCC(=O)O)C(=O)O)C(=O)O]

The synthesis of Compound 79 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed with a solution of acetic anhydride (15 equiv.) and DIPEA (2 equiv.) in DMF at r.t. for 15 min. Alloc deprotection was performed using Procedure 9. 2-(3,5-dicyanophenoxy)acetic acid (building block-VIII) was coupled to the resin using Procedure 1. The resin was washed 3 times with the following solvents: DMF, DCM, MeOH, DEE, and dried under vacuum. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (34.6 mg, 30.9% yield, 94.46% purity)

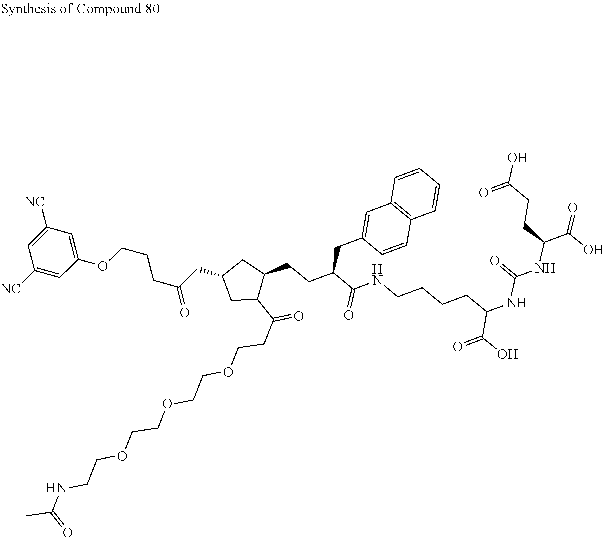 [SMILES: CC(=O)NCCOCCOCCOCCC(=O)C1C[C@H](CC(=O)CCCOc2cc(C#N)cc(C#N)c2)C[C@H]1CC[C@@H](Cc1ccc2ccccc2c1)C(=O)NCCCCC(NC(=O)N[C@@H](CCC(=O)O)C(=O)O)C(=O)O]

The synthesis of Compound 80 was performed on a 0.075 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed with a solution of acetic anhydride (15 equiv.) and DIPEA (2 equiv.) in DMF at r.t. for 15 min Alloc deprotection was performed using Procedure 9. 4-(3,5-dicyanophenoxy)butanoic acid (building block-IX) was coupled to the resin using Procedure 1. The resin was washed 3 times with the following solvents: DMF, DCM, MeOH, DEE and dried under vacuum. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid. (17.8 mg, 20% yield, 91.39% purity).

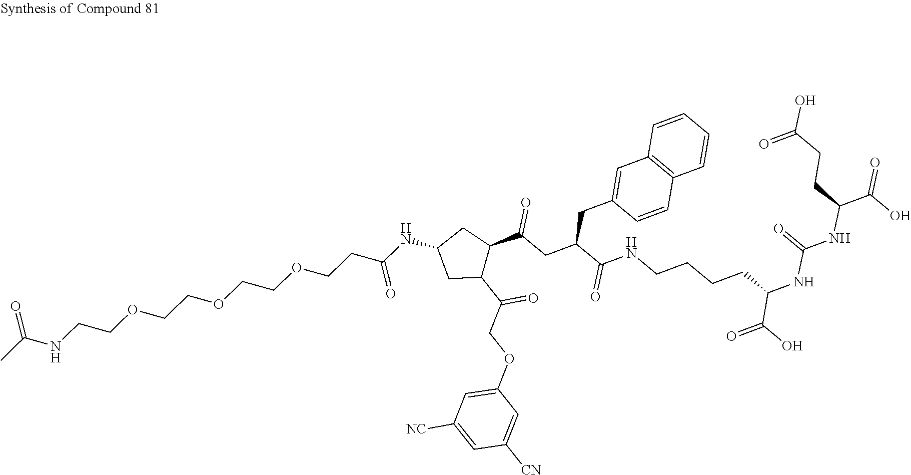 [SMILES: CC(=O)NCCOCCOCCOCCC(=O)N[C@H]1CC(C(=O)COc2cc(C#N)cc(C#N)c2)[C@H](C(=O)C[C@@H](Cc2ccc3ccccc3c2)C(=O)NCCCC[C@H](NC(=O)N[C@@H](CCC(=O)O)C(=O)O)C(=O)O)C1]

The synthesis of Compound 81 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. 2-(3,5-dicyanophenoxy)acetic acid (building block-VIII) was coupled to the resin using Procedure 1. Alloc deprotection was performed using Procedure 9. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (15.6 mg, 14.1% yield, 95.6% purity)

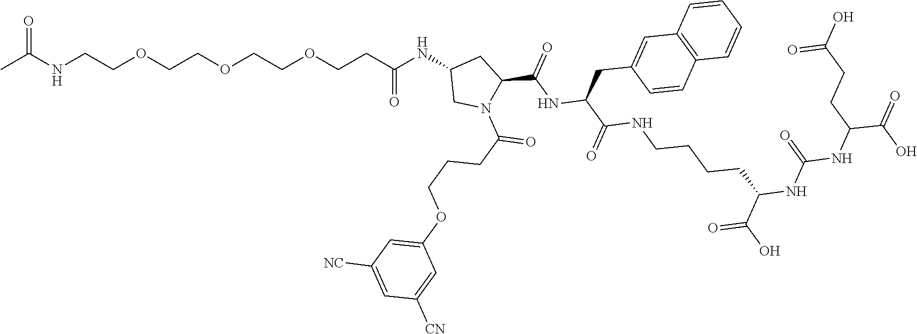 [SMILES: CC(=O)NCCOCCOCCOCCC(=O)N[C@@H]1C[C@@H](C(=O)N[C@@H](Cc2ccc3ccccc3c2)C(=O)NCCCC[C@H](NC(=O)NC(CCC(=O)O)C(=O)O)C(=O)O)N(C(=O)CCCOc2cc(C#N)cc(C#N)c2)C1]

The synthesis of Compound 82 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. 4-(3,5-dicyanophenoxy)butanoic acid (building block-IX) was coupled to the resin using Procedure 1. Alloc deprotection was performed using Procedure 9. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (31 mg, 27% yield, 96.28% purity).

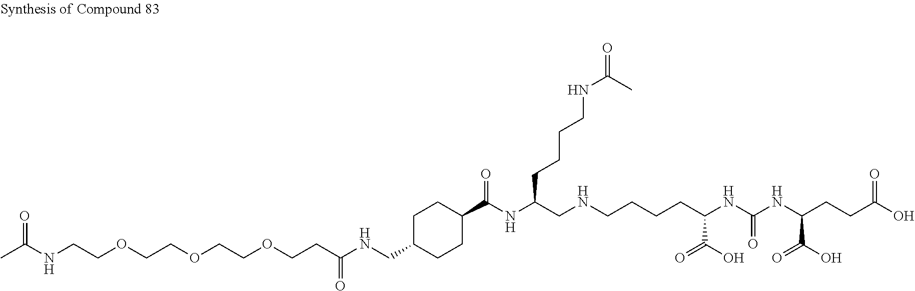 [SMILES: CC(=O)NCCCC[C@@H](CNCCCC[C@H](NC(=O)N[C@@H](CCC(=O)O)C(=O)O)C(=O)O)NC(=O)[C@H]1CC[C@H](CNC(=O)CCOCCOCCOCCNC(C)=O)CC1]

The synthesis of Compound 83 was performed on a 0.1 mmol scale on a Precursor-I-resin. Fmoc-Lys(Ac)—OH was coupled to the resin using Procedure 1. Fmoc deprotection was performed using Procedure 4. Fmoc-AMCHC-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (45 mg, 51% yield, 98% purity).

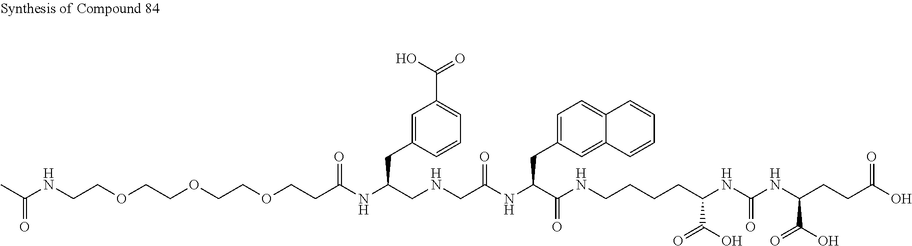 [SMILES: CC(=O)NCCOCCOCCOCCC(=O)N[C@H](CNCC(=O)N[C@@H](Cc1ccc2ccccc2c1)C(=O)NCCCC[C@H](NC(=O)N[C@@H](CCC(=O)O)C(=O)O)C(=O)O)Cc1cccc(C(=O)O)c1]

The synthesis of Compound 84 was performed on a 0.1 mmol scale on a Precursor-I-resin. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(3-COO^{t}Bu)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (79 mg, 76% yield, 97% purity).

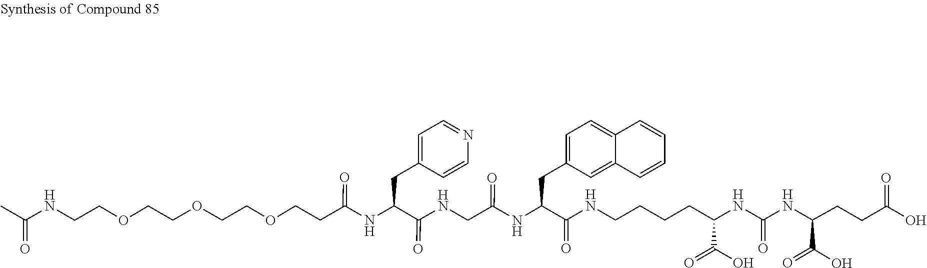 [SMILES: CC(=O)NCCOCCOCCOCCC(=O)N[C@@H](Cc1ccncc1)C(=O)NCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 85 was performed on a 0.1 mmol scale on a Precursor-I-resin. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-4-Pal-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (56 mg, 54% yield, 94% purity).

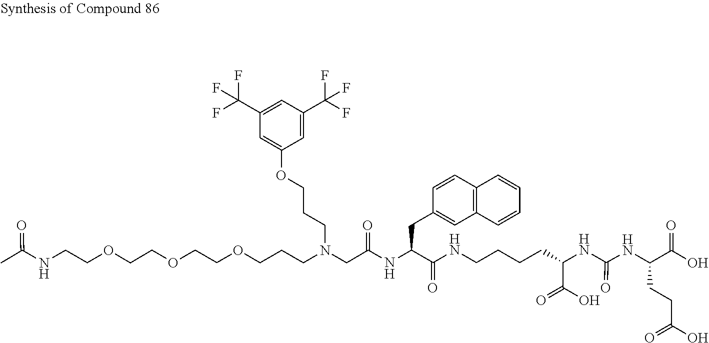 [SMILES: CC(=O)NCCOCCOCCOCCCN(CCCOc1cc(C(F)(F)F)cc(C(F)(F)F)c1)CC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 86 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. The resin was then treated with a solution of DIPEA (3 equiv.) and 2-bromoacetic anhydride (3 equiv.) in DMF at r.t. for 30 min. The resin was washed with DMF. The resin was treated with DIPEA (5 equiv.) and 3-(3,5-bis(trifluoromethyl)phenoxy)propan-1-amine (as a TFA salt, 3 equiv.) (building block-VI) in DMF at r.t. for 2 h. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (37.2 mg, 28.9% yield, 84.50% purity).

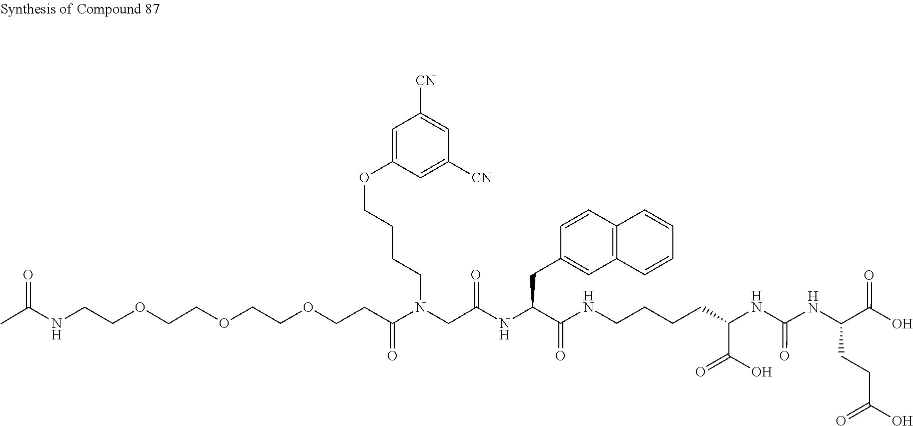 [SMILES: CC(=O)NCCOCCOCCOCCC(=O)N(CCCCOc1cc(C#N)cc(C#N)c1)CC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 87 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. The resin was then treated with a solution of DIPEA (3 equiv.) and 2-bromoacetic anhydride (3 equiv.) in DMF at r.t. for 30 min. The resin was washed with DMF. The resin was treated with DIPEA (5 equiv.) and 5-(4-aminobutoxy)isophthalonitrile (as a TFA salt, 3 equiv.) (building block-V) in DMF at r.t. for 2 h. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (9.5 mg, 8.9% yield, 95.15% purity).

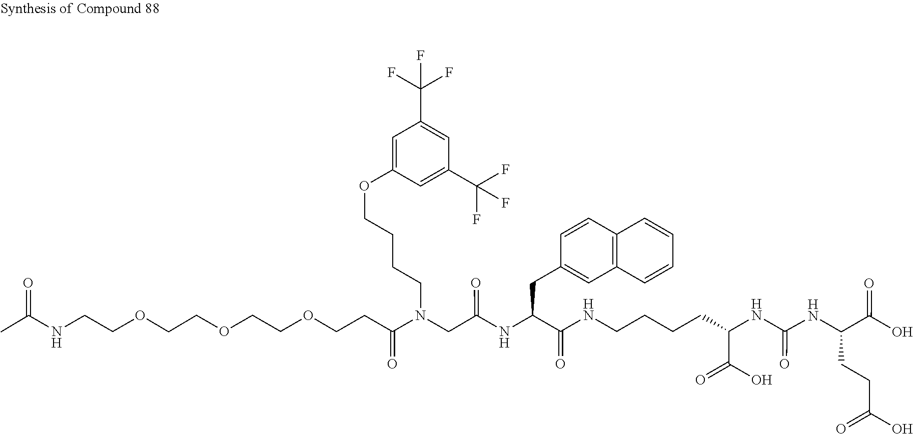 [SMILES: CC(=O)NCCOCCOCCOCCC(=O)N(CCCCOc1cc(C(F)(F)F)cc(C(F)(F)F)c1)CC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 88 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. The resin was then treated with a solution of DIPEA (3 equiv.) and 2-bromoacetic anhydride (3 equiv.) in DMF at r.t. for 30 min. The resin was washed with DMF. The resin was treated with DIPEA (5 equiv.) and 4-(3,5-bis(trifluoromethyl)phenoxy)butan-1-amine (as a TFA salt, 3 equiv.) (building block-VII) in DMF at r.t. for 2 h. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (27.0 mg, 23% yield, 92.84% purity).

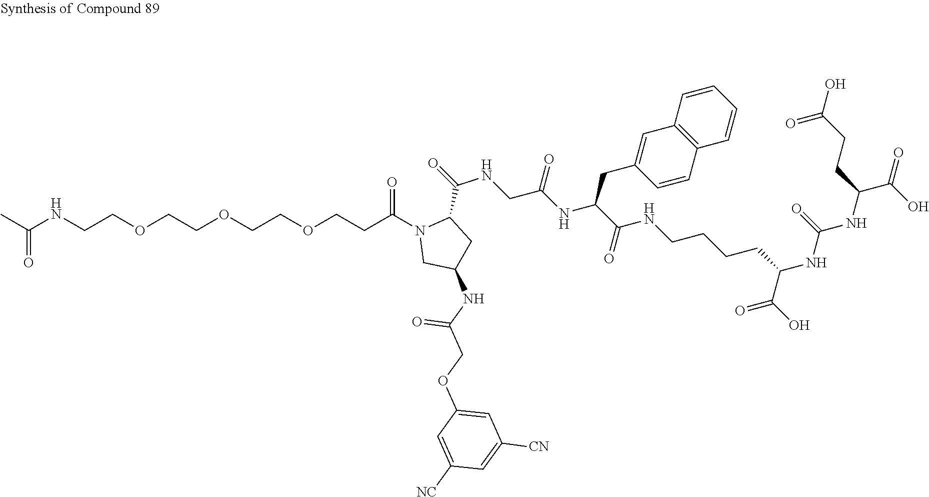 [SMILES: CC(=O)NCCOCCOCCOCCC(=O)N1C[C@H](NC(=O)COc2cc(C#N)cc(C#N)c2)C[C@H]1C(=O)NCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 89 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed with a solution of acetic anhydride (15 equiv.) and DIPEA (2 equiv.) in DMF at r.t. for 15 min. Alloc deprotection was performed using Procedure 9. 2-(3,5-dicyanophenoxy)acetic acid (building block-VIII) was coupled to the resin using Procedure 1. The resin was washed 3 times with the following solvents: DMF, DCM, MeOH, DEE, and dried under vacuum. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (36.2 mg, 28.9% yield, 89.17% purity).

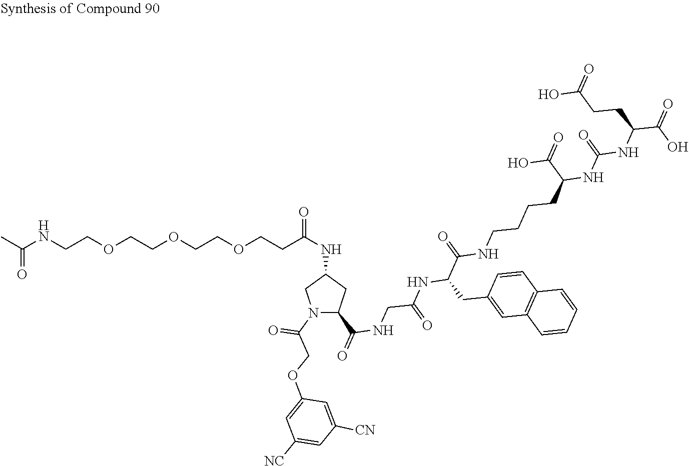 [SMILES: CC(=O)NCCOCCOCCOCCC(=O)N[C@@H]1C[C@@H](C(=O)NCC(=O)N[C@@H](Cc2ccc3ccccc3c2)C(=O)NCCCC[C@H](NC(=O)N[C@@H](CCC(=O)O)C(=O)O)C(=O)O)N(C(=O)COc2cc(C#N)cc(C#N)c2)C1]

The synthesis of Compound 90 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. 2-(3,5-dicyanophenoxy)acetic acid (building block-VIII) was coupled to the resin using Procedure 1. Alloc deprotection was performed using Procedure 9. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (32.7 mg, 27.6% yield, 94.06% purity).

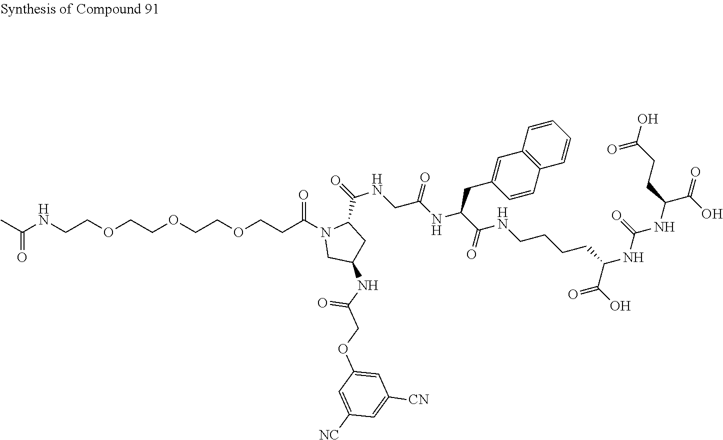 [SMILES: CC(=O)NCCOCCOCCOCCC(=O)N1C[C@H](NC(=O)COc2cc(C#N)cc(C#N)c2)C[C@H]1C(=O)NCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 91 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed with a solution of acetic anhydride (15 equiv.) and DIPEA (2 equiv.) in DMF at r.t. for 15 min. Alloc deprotection was performed using Procedure 9. 4-(3,5-dicyanophenoxy)butanoic acid (building block-IX) was coupled to the resin using Procedure 1. The resin was washed 3 times with the following solvents: DMF, DCM, MeOH, DEE, and dried under vacuum. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (52.6 mg, 42.3% yield, 92.03% purity).

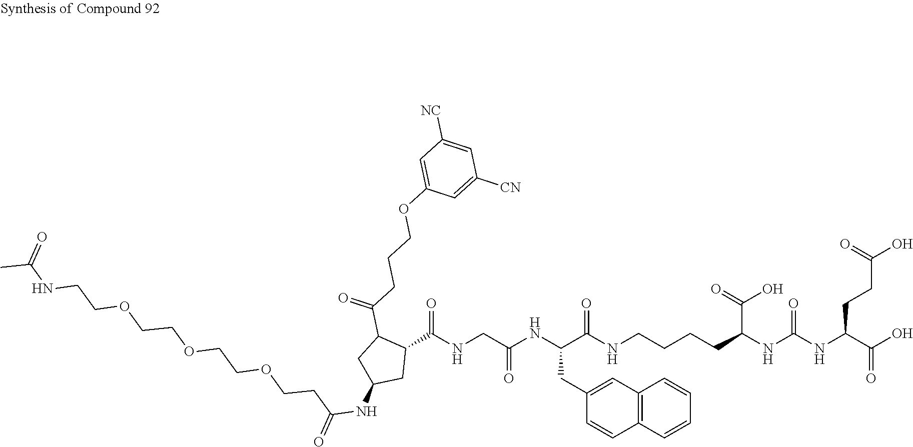 [SMILES: CC(=O)NCCOCCOCCOCCC(=O)N[C@H]1CC(C(=O)CCCOc2cc(C#N)cc(C#N)c2)[C@H](C(=O)NCC(=O)N[C@@H](Cc2ccc3ccccc3c2)C(=O)NCCCC[C@H](NC(=O)N[C@@H](CCC(=O)O)C(=O)O)C(=O)O)C1]

The synthesis of Compound 92 was performed on a 0.1 mmol scale. Precursor-I-resin was swollen in DCM for 15 min and washed with DMF. Fmoc deprotection was performed using Procedure 4. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-L-Pro(4-trans-NH-Alloc)-OH was coupled to the resin using Procedure 1. Fmoc deprotection was performed using Procedure 4. 4-(3,5-dicyanophenoxy)butanoic acid (building block-IX) was coupled to the resin using Procedure 1. Alloc deprotection was performed using Procedure 9. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (45.51 mg, 37.8% yield, 94.99% purity).

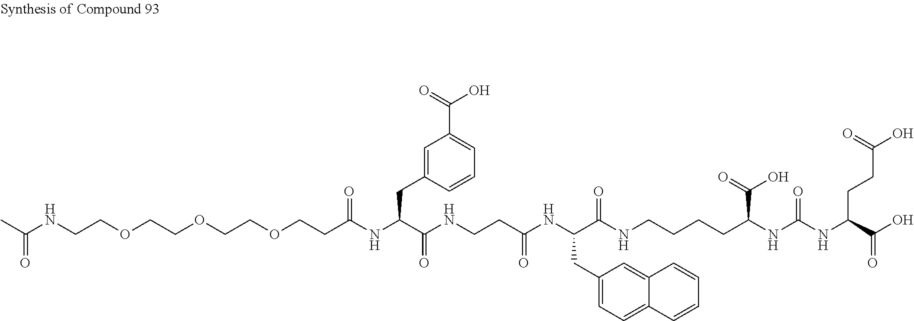 [SMILES: CC(=O)NCCOCCOCCOCCC(=O)N[C@@H](Cc1cccc(C(=O)O)c1)C(=O)NCCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 93 was performed on a 0.1 mmol scale on a Precursor-I-resin. Fmoc-2-Nal-OH was coupled to the resin using Procedure 1. Fmoc deprotection was performed using Procedure 4. Fmoc-β-Ala-OH was coupled to the resin using Procedure 1. Fmoc deprotection was performed using Procedure 4. Fmoc-Phe(3-COO^{t}Bu)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (75 mg, 70% yield, 95% purity).

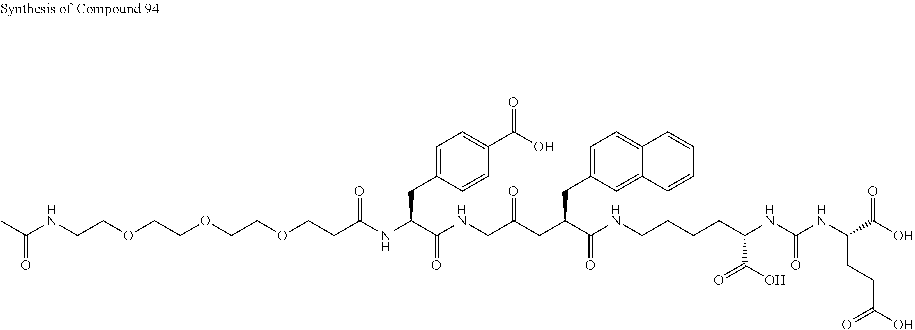 [SMILES: CC(=O)NCCOCCOCCOCCC(=O)N[C@@H](Cc1ccc(C(=O)O)cc1)C(=O)NCC(=O)C[C@@H](Cc1ccc2ccccc2c1)C(=O)NCCCC[C@H](NC(=O)N[C@@H](CCC(=O)O)C(=O)O)C(=O)O]

The synthesis of Compound 94 was performed on a 0.1 mmol scale on a Precursor-I-resin. Fmoc-2-Nal-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (81 mg, 80% yield, 95% purity).

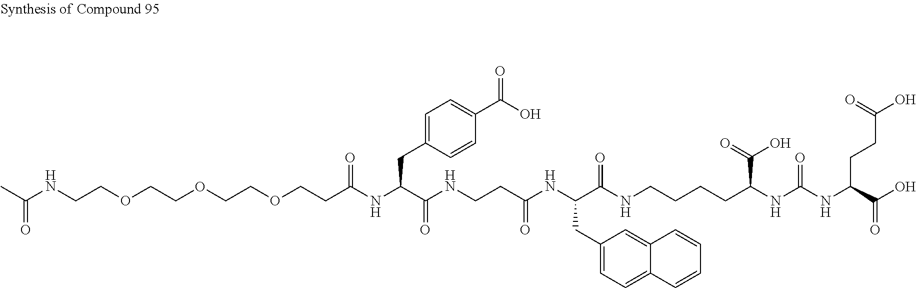 [SMILES: CC(=O)NCCOCCOCCOCCC(=O)N[C@@H](Cc1ccc(C(=O)O)cc1)C(=O)NCCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 95 was performed on a 0.1 mmol scale on a Precursor-I-resin. Fmoc-2-Nal-OH was coupled to the resin using Procedure 1. Fmoc deprotection was performed using Procedure 4. Fmoc-β-Ala-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (54 mg, 49% yield, 93% purity).

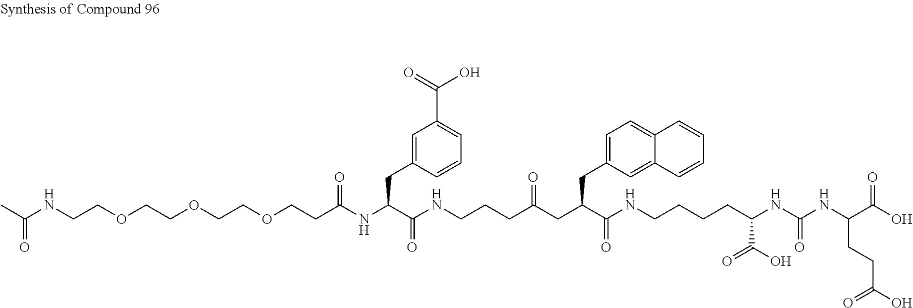 [SMILES: CC(=O)NCCOCCOCCOCCC(=O)N[C@@H](Cc1cccc(C(=O)O)c1)C(=O)NCCCC(=O)C[C@@H](Cc1ccc2ccccc2c1)C(=O)NCCCC[C@H](NC(=O)NC(CCC(=O)O)C(=O)O)C(=O)O]

The synthesis of Compound 96 was performed on a 0.1 mmol scale on a Precursor-I-resin. Fmoc-2-Nal-OH was coupled to the resin using Procedure 1. Fmoc deprotection was performed using Procedure 4. Fmoc-GABA-OH was coupled to the resin using Procedure 1. Fmoc deprotection was performed using Procedure 4. Fmoc-Phe(3-COO^{t}Bu)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (56 mg, 51% yield, 94% purity).

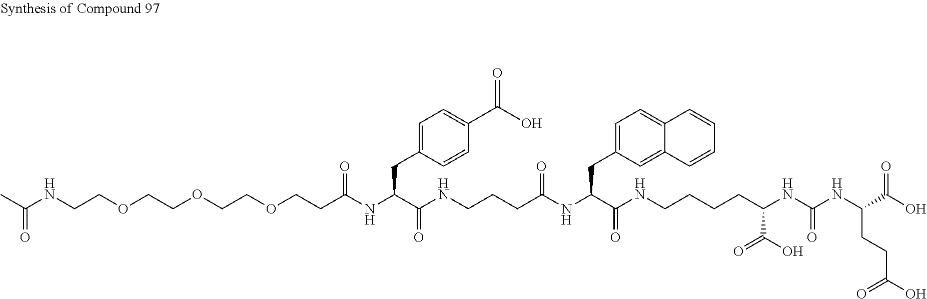 [SMILES: CC(=O)NCCOCCOCCOCCC(=O)N[C@@H](Cc1ccc(C(=O)O)cc1)C(=O)NCCCC(=O)N[C@@H](Cc1ccc2ccccc2c1)C(=O)NCCCC[C@H](NC(=O)N[C@@H](CCC(=O)O)C(=O)O)C(=O)O]

The synthesis of Compound 97 was performed on a 0.1 mmol scale on a Precursor-I-resin. Fmoc-2-Nal-OH was coupled to the resin using Procedure 1. Fmoc deprotection was performed using Procedure 4. Fmoc-GABA-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-N-amido-PEG3-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (65 mg, 63% yield, 95% purity).

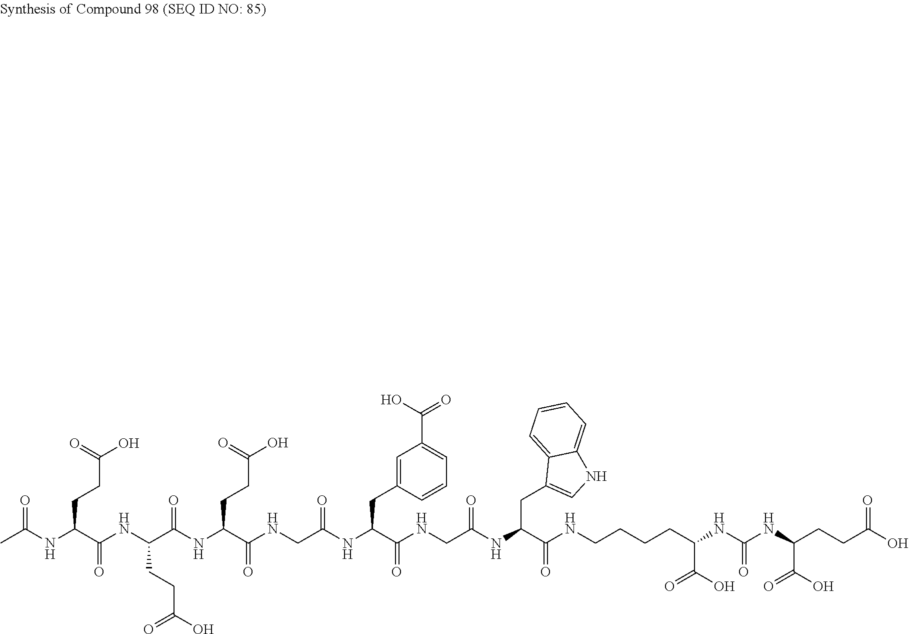 [SMILES: CC(=O)N[C@@H](CCC(=O)O)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 98 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 98 was obtained as a white solid (85.0 mg, 66% yield, 97.0% purity).

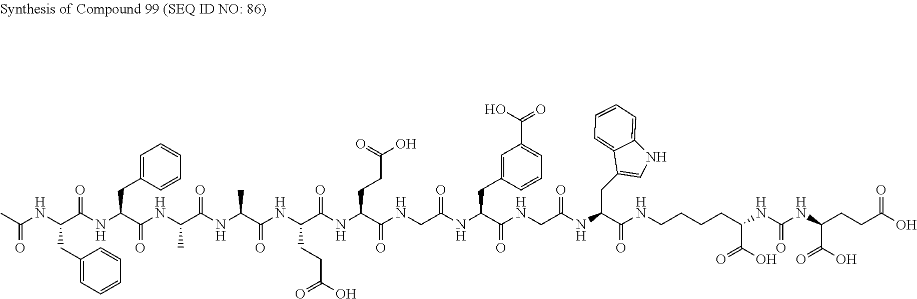 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 99 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 99 was obtained as a white solid (84.0 mg, 52% yield, 96.1% purity).

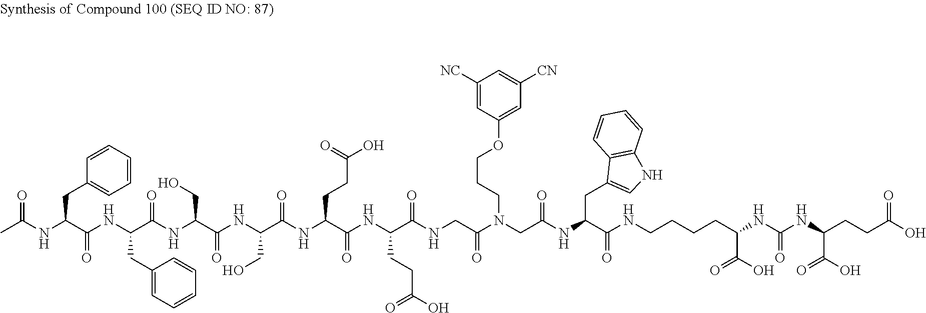 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 100 was performed on a 0.1 mmol scale on a Precursor-I-resin. Fmoc-Trp(Boc) was coupled using Procedure 1. Fmoc deprotection was performed using Procedure 4. The resin was treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. Fmoc-Gly-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Ser(^{t}Bu)-OH was coupled to the resin using Procedure 1. Fmoc deprotection was performed using Procedure 4. Fmoc-Ser(^{t}Bu)-OH was coupled to the resin using Procedure 1. Fmoc deprotection was performed using Procedure 4. Fmoc-Phe-OH was coupled to the resin using Procedure 1. Fmoc deprotection was performed using Procedure 4. Fmoc-Phe-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding The rest of the sequence was assembled using Procedure 1. Compound 100 was obtained as a white solid (27 mg, 15% yield, 96.46% purity).

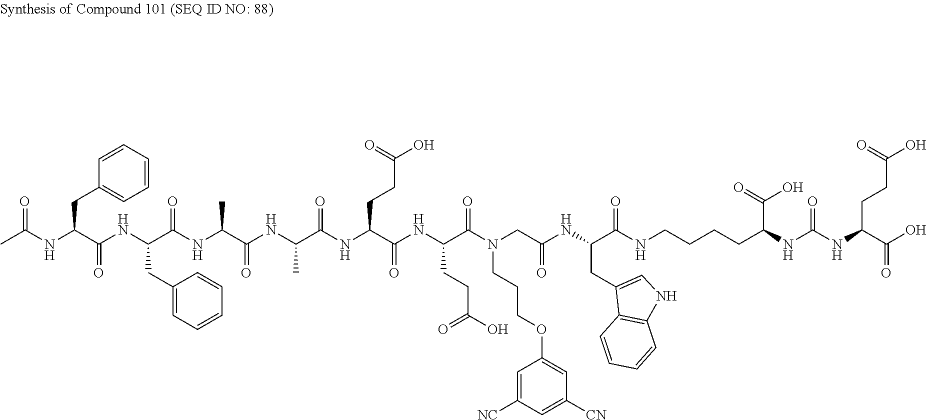 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 101 was performed on a 0.1 mmol scale on a Precursor-I-resin. The first tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 101 was obtained as a white solid (12.1 mg, 14.6% yield, 97.2% purity).

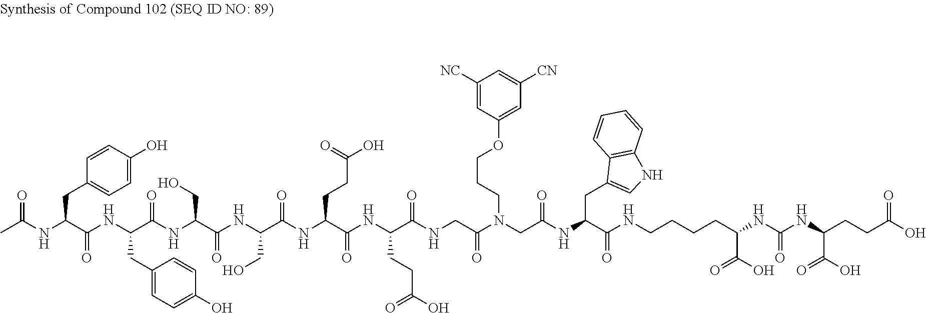 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 102 was performed on a 0.1 mmol scale on a Precursor-I-resin. The first tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 102 was obtained as a white solid (12.2 mg, 13.3% yield, 91.6% purity).

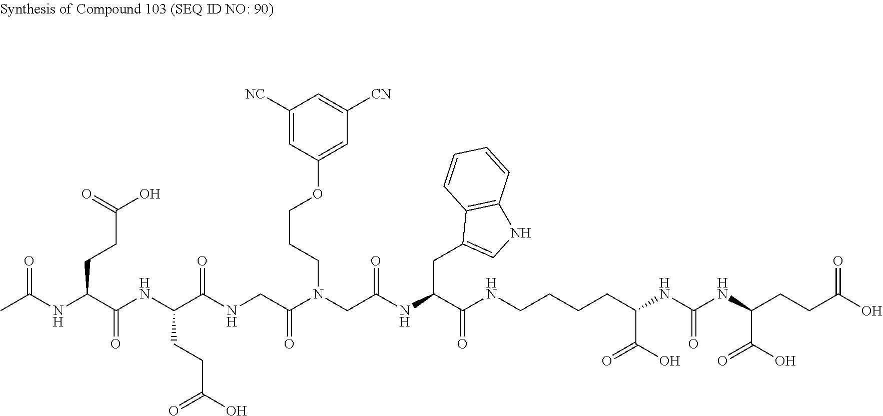 [SMILES: CC(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 103 was performed on a 0.1 mmol scale on a Precursor-I-resin. The first tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 103 was obtained as a white solid (8.8 mg, 12.2% yield, 97.4% purity).

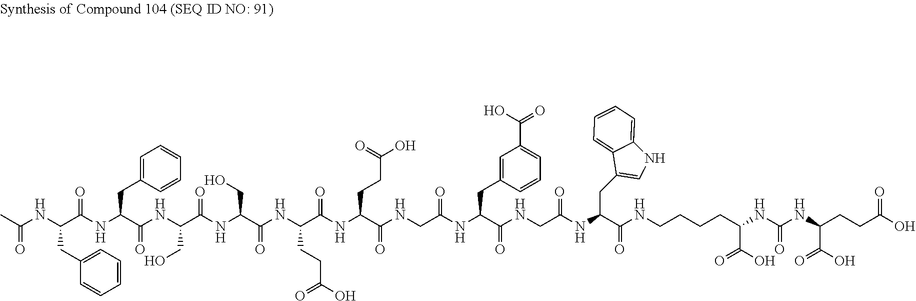 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 104 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 104 was obtained as a white solid (75.9 mg, 51.3% yield, 93.3% purity).

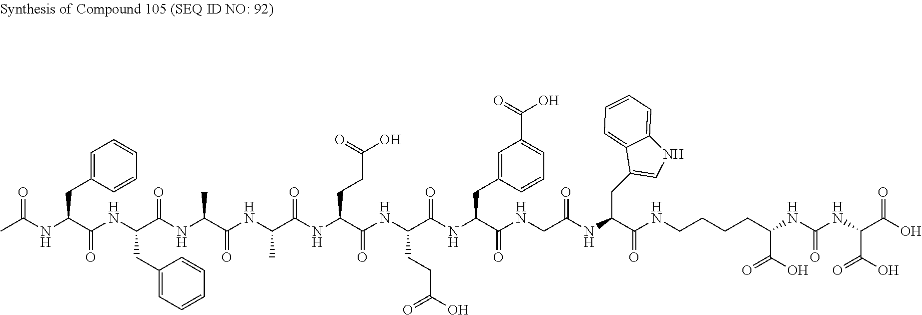

The synthesis of Compound 105 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 105 was obtained as a white solid (74.5 mg, 50.1% yield, 96.2% purity).

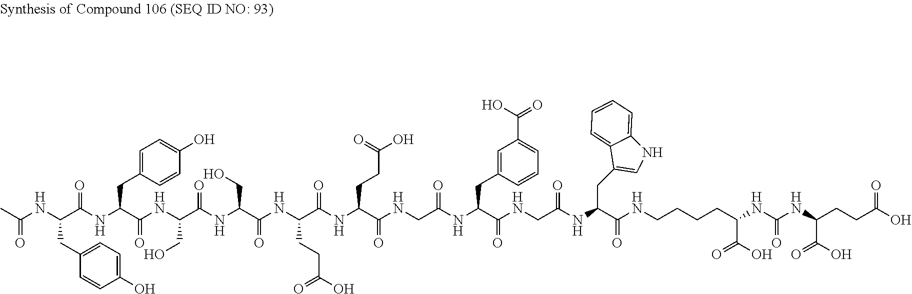 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 106 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 106 was obtained as a white solid (60.3 mg, 40.3% yield, 97.9% purity).

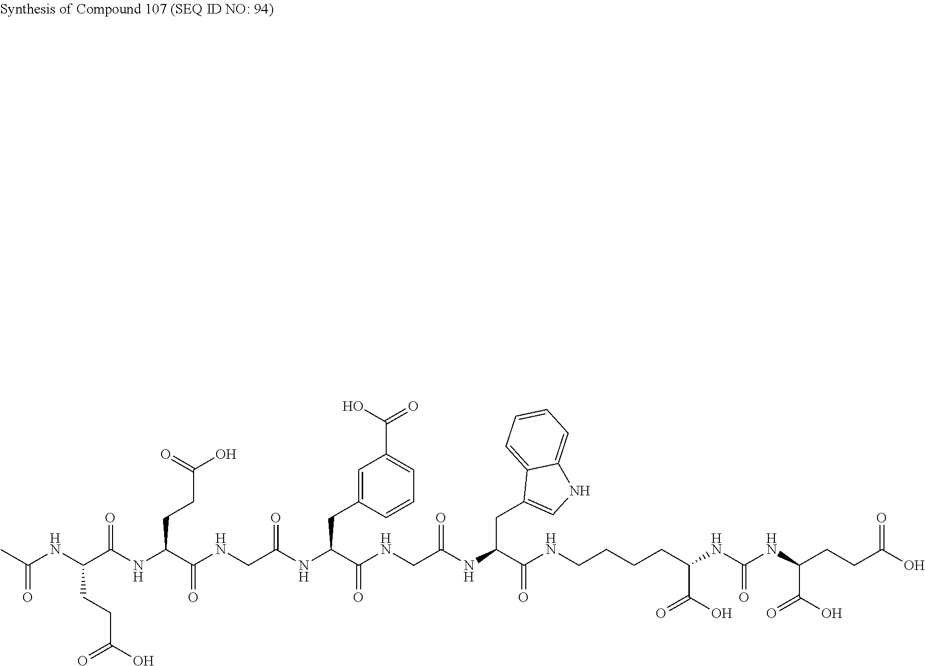 [SMILES: CC(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 107 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 107 was obtained as a white solid (71.4 mg, 63.5% yield, 91.9% purity).

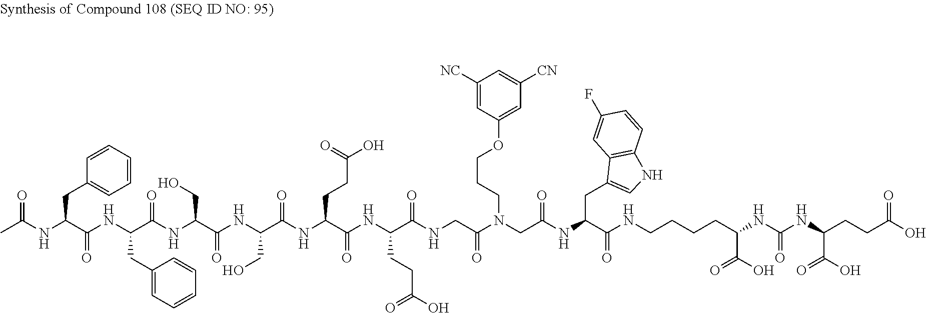 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 108 was performed on a 0.1 mmol scale on a Precursor-I-resin. The first 5F-tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 108 was obtained as a white solid (14.4 mg, 17.8% yield, 97.5% purity).

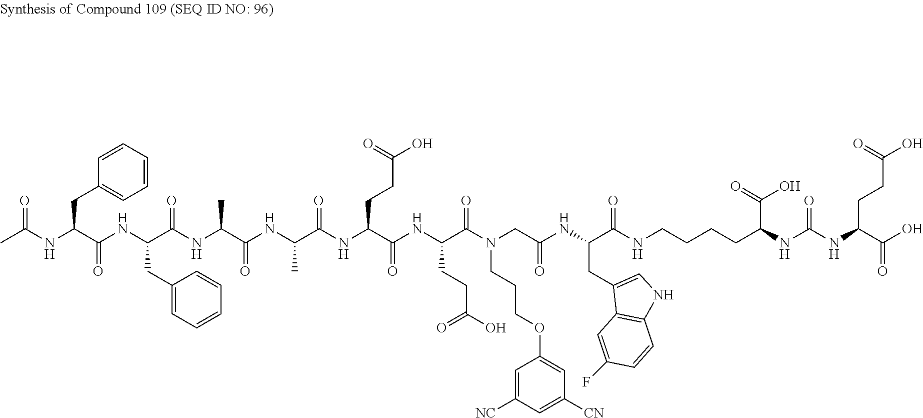 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 109 was performed on a 0.1 mmol scale on a Precursor-I-resin. The first 5F-tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 109 was obtained as a white solid (12.8 mg, 16.0% yield, 96.0% purity).

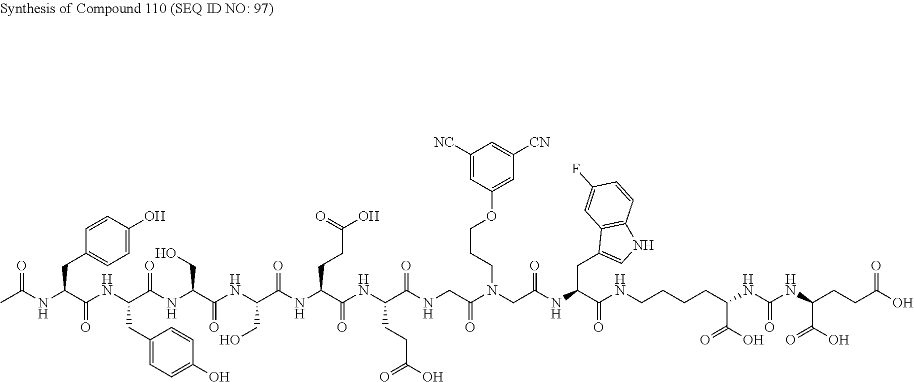 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 110 was performed on a 0.1 mmol scale on a Precursor-I-resin. The first 5F-tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 110 was obtained as a white solid (14.9 mg, 16.9% yield, 95.1% purity).

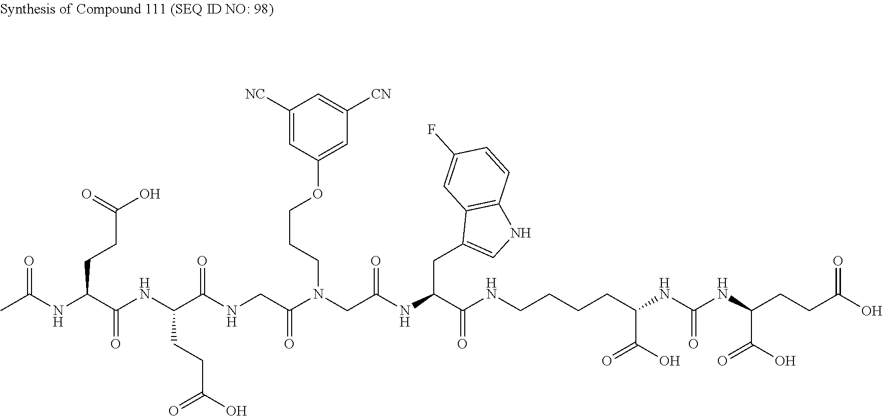 [SMILES: CC(=O)N[C@@H](CCC(=O)O)C(=O)N[C@@H](CCC(=O)O)C(=O)NCC(=O)N(CCCOc1cc(C#N)cc(C#N)c1)CC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 111 was performed on a 0.1 mmol scale on a Precursor-I-resin. The first 5F-tryptophan residue was added using Procedure 1. The resin was then treated with a solution of DIPEA (3 equiv.) and 2-bromoacetic anhydride (3 equiv.) in DMF at r.t. for 2 hours, then washed with DMF. The resin was treated with DIPEA (5 equiv.) and 5-(3-aminopropoxy)isophthalonitrile (as a TFA salt, 2.9 equiv.) (building block-IV) in DMF at r.t. for 2 h, then washed with DMF. The rest of the sequence was assembled using Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 111 was obtained as a white solid (11.1 mg, 15.2% yield, 90.7% purity).

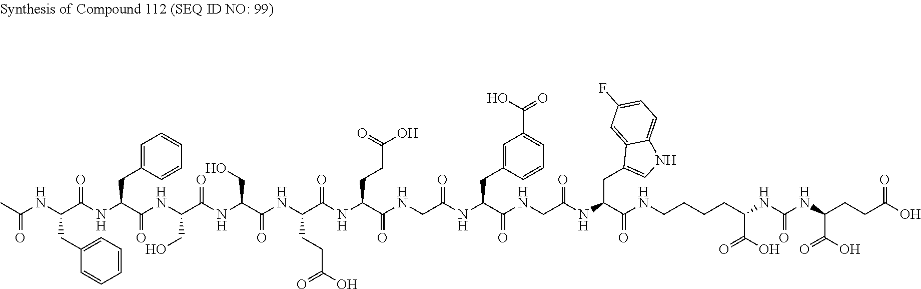 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 112 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 112 was obtained as a white solid (79.8 mg, 53.6% yield, 95.8% purity).

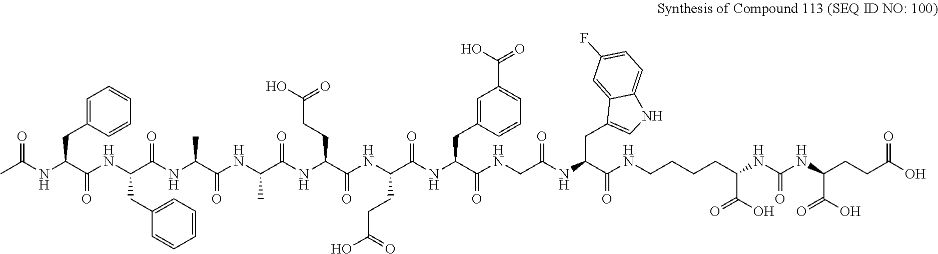 [SMILES: CC(=O)N[C@@H](Cc1ccccc1)C(=O)N[C@@H](Cc1ccccc1)C(=O)N[C@@H](C)C(=O)N[C@@H](C)C(=O)N[C@@H](CCC(=O)O)C(=O)N[C@@H](CCC(=O)O)C(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 113 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 113 was obtained as a white solid (65.8 mg, 46.3% yield, 94.0% purity).

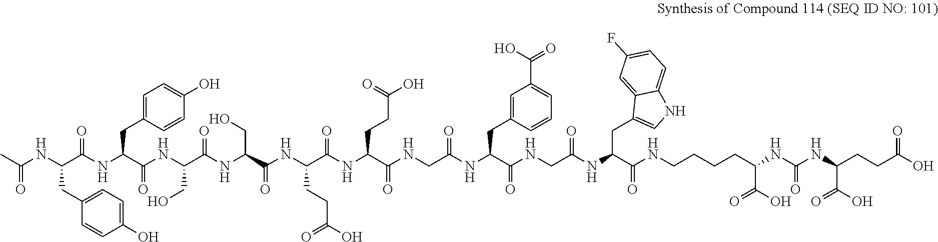 [SMILES: CC(=O)N[C@@H](Cc1ccc(O)cc1)C(=O)N[C@@H](Cc1ccc(O)cc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 114 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 114 was obtained as a white solid (58 mg, 37.4% yield, 97.1% purity).

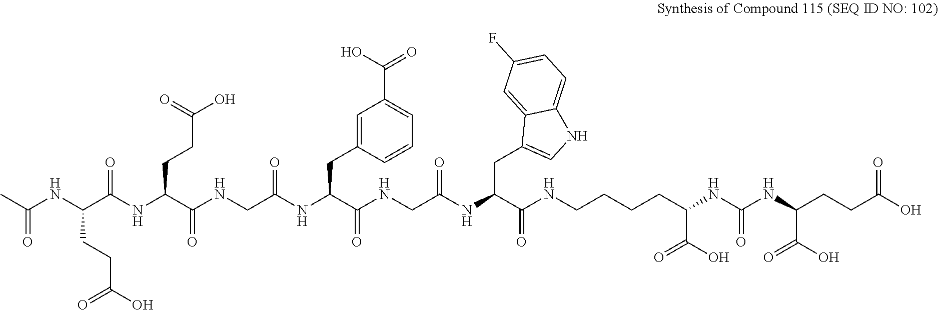 [SMILES: CC(=O)N[C@@H](CCC(=O)O)C(=O)N[C@@H](CCC(=O)O)C(=O)NCC(=O)N[C@@H](Cc1cccc(C(=O)O)c1)C(=O)NCC(=O)N[C@@H](Cc1c[nH]c2ccc(F)cc12)C(=O)NCCCC[C@H](NC(=O)N[C@@H](CCC(=O)O)C(=O)O)C(=O)O]

The synthesis of Compound 115 was performed on a 0.1 mmol scale on a Precursor-I-resin following Procedure 2. The peptidyl-resin was then cleaved and fully deprotected using Procedure 6. The resulting paste was dissolved in DMSO and purified using Procedure 8. Preparative HPLC fractions containing the corresponding purified products were combined and freeze-dried. Compound 115 was obtained as a white solid (60.3 mg, 56.4% yield, 97.2% purity).

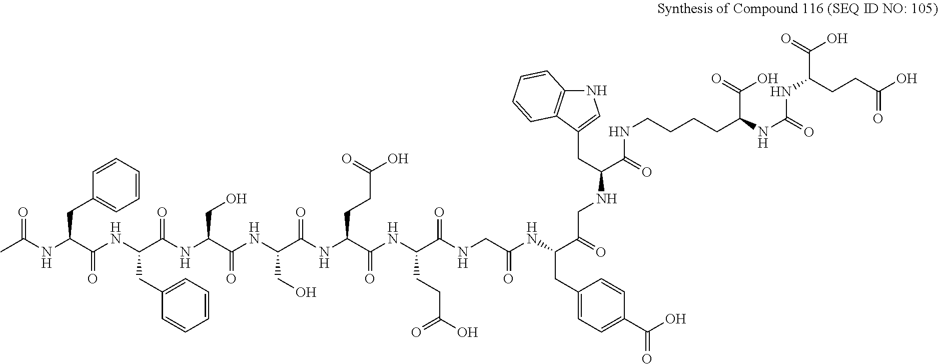 [SMILES: CC(=O)N[C@@H](Cc1ccccc1)C(=O)N[C@@H](Cc1ccccc1)C(=O)N[C@@H](CO)C(=O)N[C@@H](CO)C(=O)N[C@@H](CCC(=O)O)C(=O)N[C@@H](CCC(=O)O)C(=O)NCC(=O)N[C@@H](Cc1ccc(C(=O)O)cc1)C(=O)CN[C@@H](Cc1c[nH]c2ccccc12)C(=O)NCCCC[C@H](NC(=O)N[C@@H](CCC(=O)O)C(=O)O)C(=O)O]

The synthesis of Compound 116 was performed on a 0.1 mmol scale on a Precursor-I-resin. Fmoc-Trp(Boc)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Ser(^{t}Bu)-OH was coupled to the resin using Procedure 1. Fmoc deprotection was performed using Procedure 4. Fmoc-Ser(^{t}Bu)-OH was coupled to the resin using Procedure 1. Fmoc deprotection was performed using Procedure 4. Fmoc-Phe-OH was coupled to the resin using Procedure 1. Fmoc deprotection was performed using Procedure 4. Fmoc-Phe-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding Compound 116 as a white solid (9.3 mg, 11% yield, 98.74% purity).

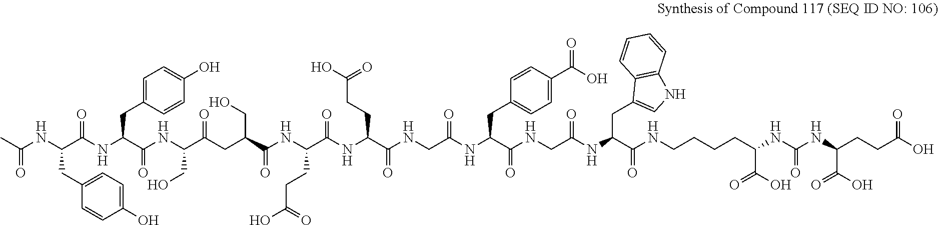 [SMILES: CC(=O)N[C@@H](Cc1ccc(O)cc1)C(=O)N[C@@H](Cc1ccc(O)cc1)C(=O)N[C@@H](CO)C(=O)C[C@@H](CO)C(=O)N[C@@H](CCC(=O)O)C(=O)N[C@@H](CCC(=O)O)C(=O)NCC(=O)N[C@@H](Cc1ccc(C(=O)O)cc1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 117 was performed on a 0.1 mmol scale on a Precursor-I-resin. Fmoc-Trp(Boc)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Ser(^{t}Bu)-OH was coupled to the resin using Procedure 1. Fmoc deprotection was performed using Procedure 4. Fmoc-Ser(^{t}Bu)-OH was coupled to the resin using Procedure 1. Fmoc deprotection was performed using Procedure 4. Fmoc-Tyr(^{t}Bu)-OH was coupled to the resin using Procedure 1. Fmoc deprotection was performed using Procedure 4. Fmoc-Tyr(^{t}Bu)-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (9.9 mg, 13% yield, 98.24% purity).

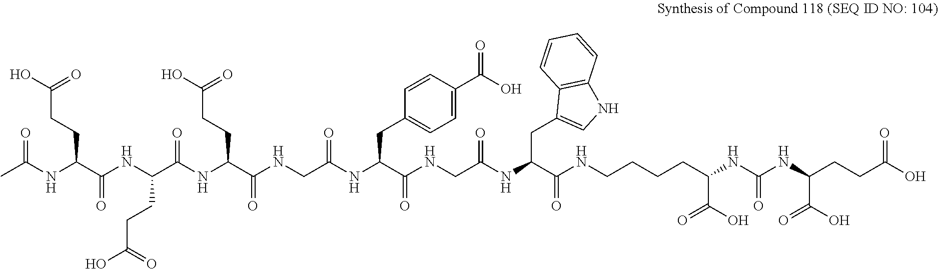 [SMILES: CC(=O)N[C@@H](CCC(=O)O)C(=O)N[C@@H](CCC(=O)O)C(=O)N[C@@H](CCC(=O)O)C(=O)NCC(=O)N[C@@H](Cc1ccc(C(=O)O)cc1)C(=O)NCC(=O)N[C@@H](Cc1c[nH]c2ccccc12)C(=O)NCCCC[C@H](NC(=O)N[C@@H](CCC(=O)O)C(=O)O)C(=O)O]

The synthesis of Compound 118 was performed on a 0.1 mmol scale on a Precursor-I-resin. Fmoc-Trp(Boc)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. N-term acetylation was performed using Procedure 11. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (28.3 mg, 22.4% yield, 98.02% purity).

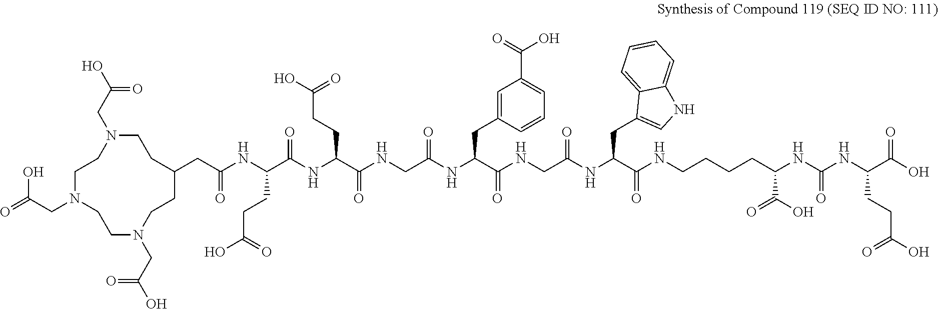 [SMILES: O=C(O)CC[C@H](NC(=O)N[C@@H](CCCCNC(=O)[C@H](Cc1c[nH]c2ccccc12)NC(=O)CNC(=O)[C@H](Cc1cccc(C(=O)O)c1)NC(=O)CNC(=O)[C@H](CCC(=O)O)NC(=O)[C@H](CCC(=O)O)NC(=O)CC1CCN(CC(=O)O)CCN(CC(=O)O)CCN(CC(=O)O)CC1)C(=O)O)C(=O)O]

The synthesis of Compound 119 was performed on a 0.1 mmol scale on a Precursor-I-resin. Fmoc-Trp(Boc)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. DOTA(OtBu)₃ was coupled to the resin using Procedure 1. Acetylation of unreacted amines was performed with a solution of acetic anhydride (10 equiv.) in DMF for 15 min at r.t. The resin was washed with DMF, DCM, MeOH, DEE, and dried under vacuum. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (69 mg, 34% yield, 94.11% purity).

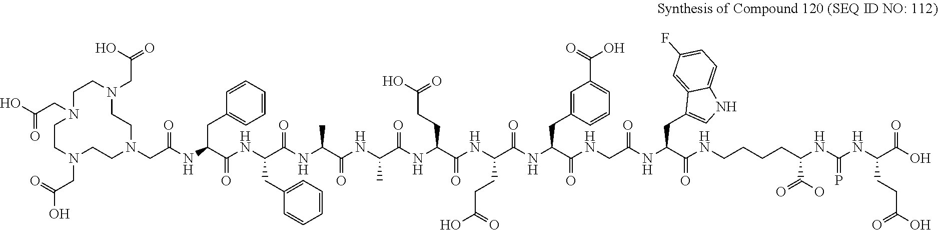

The synthesis of Compound 120 was performed on a 0.1 mmol scale on a Precursor-I-resin. Fmoc-Trp(5-fluoro)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Phe(4-COO^{t}Bu)-OH was coupled to the resin using Procedure 1. Fmoc deprotection was performed using Procedure 4. Fmoc-Gly-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Glu(O^{t}Bu)-OH was coupled to the resin using Procedure 1. Fmoc deprotection was performed using Procedure 4. Fmoc-Ala-OH was coupled to the resin using Procedure 1. Fmoc deprotection was performed using Procedure 4. Fmoc-Ala-OH was coupled to the resin using Procedure 1. Fmoc deprotection was performed using Procedure 4. Fmoc-Phe-OH was coupled to the resin using Procedure 1. Fmoc deprotection was performed using Procedure 4. Fmoc-Phe-OH was coupled to the resin using Procedure 1. Fmoc deprotection was performed using Procedure 4. DOTA(OtBu)₃ was coupled to the resin using Procedure 1. Acetylation of unreacted amines was performed with a solution of acetic anhydride (10 equiv.) in DMF for 15 min at r.t. The resin was washed with DMF, DCM, MeOH, DEE, and dried under vacuum. The resin was then treated with the cleavage mixture using Procedure 6. The crude paste was solubilized in DMSO and purified by preparative RP-HPLC using Procedure 8. Preparative HPLC fractions containing the purified product were combined and freeze-dried, yielding a white solid (22 mg, 8.8% yield, 92.17% purity).

### Analytical Methods

### HPLC Methods:

### General Conditions:

Temperature: 50° C. for methods A, B, C, D, F and G; 70° C. for method E.
Solvent A: H₂O milliQ+0.1% TFA (v/v)
Solvent B: Acetonitrile HPLC grad+0.1% TFA (v/v)

Method A:
Column: Aeris WIDEPORE XB-C18 3.6 μm; 4.6 × 150 mm

| Time | Flow | | |
|---|---|---|---|
| (min) | (mL/min) | % A | % B |
| 0 | 1 | 98 | 2 |
| 2 | 1 | 98 | 2 |
| 7 | 1 | 15 | 85 |
| 7.2 | 1 | 5 | 95 |
| 10 | 1 | 5 | 95 |
| 10.2 | 1 | 98 | 2 |
| 12 | 1 | 98 | 2 |

Method B:
Column: Waters XBridge C18 3.5 μm; 3 × 150 mm

| Time | Flow | | |
|---|---|---|---|
| (min) | (mL/min) | % A | % B |
| 0 | 0.5 | 98 | 2 |
| 4 | 0.5 | 78 | 22 |
| 22 | 0.5 | 38 | 62 |
| 25 | 0.5 | 5 | 95 |
| 30 | 0.5 | 5 | 95 |
| 30.1 | 0.5 | 90 | 10 |
| 35 | 0.5 | 90 | 10 |

Method C:
Column: Waters XBridge C18 3.5 μm; 3 × 150 mm

| Time | Flow | | |
|---|---|---|---|
| (min) | (mL/min) | % A | % B |
| 0 | 0.5 | 98 | 2 |
| 22 | 0.5 | 48 | 52 |
| 25 | 0.5 | 5 | 95 |
| 30 | 0.5 | 5 | 95 |
| 30.1 | 0.5 | 98 | 2 |
| 35 | 0.5 | 98 | 2 |

Method D:
Column: Waters XBridge C18 3.5 μm; 3 × 150 mm

| Time | Flow | | |
|---|---|---|---|
| (min) | (mL/min) | % A | % B |
| 0 | 0.5 | 90 | 10 |
| 4 | 0.5 | 70 | 30 |
| 22 | 0.5 | 30 | 70 |
| 25 | 0.5 | 5 | 95 |
| 30 | 0.5 | 5 | 95 |
| 30.1 | 0.5 | 90 | 10 |
| 35 | 0.5 | 90 | 10 |

Method E:
Column: Waters XBridge C18 3.5 μm; 3 × 150 mm

| Time | Flow | | |
|---|---|---|---|
| (min) | (mL/min) | % A | % B |
| 0 | 0.5 | 98 | 2 |
| 22 | 0.5 | 48 | 52 |
| 25 | 0.5 | 5 | 95 |
| 30 | 0.5 | 5 | 95 |
| 30.1 | 0.5 | 98 | 2 |
| 35 | 0.5 | 98 | 2 |

Method F:
Column: Waters XBridge C18 3.5 μm; 3 × 150 mm

| Time | Flow | | |
|---|---|---|---|
| (min) | (mL/min) | % A | % B |
| 0 | 0.5 | 90 | 10 |
| 4 | 0.5 | 75 | 25 |
| 22 | 0.5 | 35 | 65 |
| 25 | 0.5 | 5 | 95 |
| 30 | 0.5 | 5 | 95 |
| 30.1 | 0.5 | 90 | 10 |
| 35 | 0.5 | 90 | 10 |

Method G:
Column: Waters XBridge C4 3.5 μm; 3 × 150 mm

| Time | Flow | | |
|---|---|---|---|
| (min) | (mL/min) | % A | % B |
| 0 | 0.5 | 98 | 2 |
| 22 | 0.5 | 48 | 52 |
| 25 | 0.5 | 5 | 95 |
| 30 | 0.5 | 5 | 95 |
| 30.1 | 0.5 | 98 | 2 |
| 35 | 0.5 | 98 | 2 |

### Mass Spectrometry Analysis

Molecular weight of the molecules was determined using ESI mass spectrometer (ISQ EM single quadrupole mass spectrometer for liquid chromatography with extended mass range, HESI source, Thermo Fischer Scientific).

### Analytical Results

| Compound | Chemical | MW | [M + nH]ⁿ⁺/n | | HPLC | RT |
|---|---|---|---|---|---|---|
| No. | formula | (calculated) | (found) | n | methods | (min) |
| 1 | C₃₇H₆₄N₆O₁₄ | 816.95 | 817.38 | 1 | F | 7.125 |
| 2 | C₄₂H₆₃N₇O₁₄ | 890.00 | 890.44 | 1 | F | 7.342 |
| 3 | C₄₀H₆₂N₆O₁₅ | 866.96 | 867.44 | 1 | F | 5.725 |
| 4 | C₃₄H₅₈N₆O₁₄ | 774.87 | 775.46 | 1 | F | 4.825 |
| 5 | C₄₂H₆₃N₇O₁₄ | 890.00 | 890.46 | 1 | H | 7.325 |
| 6 | C₃₇H₆₅N₇O₁₄ | 831.96 | 832.43 | 1 | C | 8.375 |
| 7 | C₄₆H₆₆N₆O₁₄ | 927.06 | 927.52 | 1 | B | 12.275 |
| 8 | C₄₄H₆₄N₆O₁₄ | 901.02 | 901.57 | 1 | C | 15.275 |
| 9 | C₃₇H₆₀N₈O₁₄ | 840.93 | 841.42 | 1 | C | 8.242 |
| 10 | C₄₁H₆₂N₆O₁₆ | 894.97 | 895.58 | 1 | C | 11.208 |
| 11 | C₃₈H₆₂N₈O₁₄ | 854.96 | 855.57 | 1 | C | 8.425 |
| 12 | C₄₂H₆₂FN₇O₁₄ | 907.99 | 908.55 | 1 | C | 13.408 |
| 13 | C₄₀H₆₁FN₆O₁₄ | 868.95 | 869.39 | 1 | B | 8.892 |
| 14 | C₄₀H₆₃N₇O₁₄ | 865.98 | 866.53 | 1 | E | 8.392 |
| 15 | C₄₀H₆₁N₉O₁₄ | 891.98 | 892.68 | 1 | C | 14.358 |
| 16 | C₄₇H₆₈N₆O₁₅ | 957.09 | 957.65 | 1 | C | 9.325 |
| 17 | C₄₀H₆₂N₆O₁₄ | 850.96 | 851.54 | 1 | C | 12.825 |
| 18 | C₄₀H₆₂N₆O₁₄ | 850.96 | 851.48 | 1 | C | 8.458 |
| 19 | C₄₀H₆₈N₆O₁₄ | 857.01 | 857.66 | 1 | D | 7.808 |
| 20 | C₃₇H₆₂N₆O₁₆ | 846.93 | 847.43 | 1 | C | 9.192 |
| 21 | C₄₁H₆₂N₆O₁₆ | 894.97 | 895.38 | 1 | C | 10.742 |
| 22 | C₄₁H₆₂N₈O₁₄ | 890.99 | 446.46 | 1 | C | 9.225 |
| 23 | C₄₃H₆₂N₆O₁₄ | 887.00 | 887.56 | 1 | C | 15.175 |
| 24 | C₄₅H₆₆N₆O₁₄ | 915.05 | 915.66 | 1 | C | 15.475 |
| 25 | C₄₈H₆₆N₆O₁₃ | 935.09 | 936.67 | 1 | C | 16.458 |
| 26 | C₄₅H₆₈N₆O₁₂ | 885.07 | 885.63 | 1 | C | 17.958 |
| 27 | C₄₄H₆₆N₆O₁₂S | 903.10 | 903.57 | 1 | C | 16.925 |
| 28 | C₄₂H₆₂N₆O₁₂S | 875.05 | 875.54 | 1 | C | 16.158 |
| 29 | C₄₅H₆₄N₈O₁₂ | 909.05 | 909.45 | 1 | C | 14.758 |
| 30 | C₄₉H₆₆N₆O₁₄ | 963.10 | 963.52 | 1 | C | 16.708 |
| 31 | C₄₉H₆₆N₆O₁₄ | 963.10 | 963.64 | 1 | C | 16.392 |
| 32 | C₄₃H₆₁N₇O₁₅ | 916.00 | 916.56 | 1 | C | 14.575 |
| 33 | C₄₅H₆₅N₇O₁₅ | 944.05 | 944.56 | 1 | C | 14.658 |
| 34 | C₅₅H₈₀N₁₂O₁₇ | 1181.31 | 1181.82 | 1 | C | 14.708 |
| 35 | C₅₅H₈₀N₁₂O₁₇ | 1181.31 | 1181.75 | 1 | C | 14.708 |
| 36 | C₇₁H₉₂N₁₂O₂₁ | 1449.58 | 1449.91 | 1 | C | 18.625 |
| 37 | C₇₁H₉₂N₁₂O₂₁ | 1449.58 | 1449.85 | 1 | C | 18.625 |
| 38 | C₆₇H₉₂N₁₂O₁₇ | 1337.54 | 1337.95 | 1 | C | 14.842 |
| 39 | C₅₅H₇₅F₂N₇O₁₆ | 1128.23 | 1128.56 | 1 | C | 19.808 |
| 40 | C₅₅H₇₅F₂N₇O₁₆ | 1128.23 | 1128.58 | 1 | C | 19.825 |
| 41 | C₄₉H₇₁N₇O₁₅ | 998.14 | 998.61 | 1 | G | 14.492 |
| 42 | C₃₇H₆₀N₈O₁₄ | 840.93 | 841.15 | 1 | E | 8.025 |
| 43 | C₄₀H₆₂N₆O₁₅ | 866.96 | 867.7 | 1 | C | 10.492 |
| 44 | C₃₉H₆₁N₇O₁₄ | 851.95 | 852.55 | 1 | C | 8.375 |
| 45 | C₃₇H₅₉N₇O₁₄S | 857.97 | 858.25 | 1 | C | 10.025 |
| 46 | C₄₁H₆₄N₆O₁₄ | 864.99 | 865.58 | 1 | C | 12.908 |
| 47 | C₅₂H₈₀N₈O₁₅ | 945.04 | 945.29 | 1 | C | 14.442 |
| 48 | C₄₅H₆₄N₈O₁₄ | 941.05 | 942.3 | 1 | C | 15.425 |
| 49 | C₅₀H₇₂N₆O₁₃ | 909.05 | 909.26 | 1 | C | 17.292 |
| 50 | C₄₄H₆₂N₆O₁₂ | 867.01 | 867.71 | 1 | C | 16.224 |
| 51 | C₄₉H₆₂N₈O₁₅ | 1003.08 | 1003.7 | 1 | C | 18.325 |
| 52 | C₅₁H₆₅N₉O₁₆ | 1060.13 | 1060.57 | 1 | C | 17.708 |
| 53 | C₅₂H₆₇N₉O₁₆ | 1074.16 | 1074.63 | 1 | C | 17.408 |
| 54 | C₃₉H₆₄F₃N₇O₁₅ | 927.97 | 464.99 | 2 | A | 5.55 |
| 55 | C₆₈H₉₀N₁₂O₂₃ | 1443.53 | 1443.82 | 1 | C | 15.792 |
| 56 | C₆₉H₉₀FN₁₃O₂₁ | 1456.55 | 1456.99 | 1 | C | 17.408 |
| 57 | C₆₉H₉₁N₁₃O₂₁ | 1438.56 | 1438.82 | 1 | C | 17.042 |
| 58 | C₆₂H₈₃N₁₁O₂₀ | 1302.40 | 1302.91 | 1 | C | 16.408 |
| 59 | C₅₀H₇₂N₁₀O₂₁ | 1149.18 | 1149.47 | 1 | C | 10.308 |
| 60 | C₅₀H₆₉N₉O₁₈ | 1084.15 | 1084.75 | 1 | C | 14.175 |
| 61 | C₅₂H₇₁N₉O₂₀ | 1142.18 | 1142.75 | 1 | C | 13.958 |
| 62 | C₆₁H₈₆N₁₂O₂₃ | 1355.42 | 1355.97 | 1 | C | 14.108 |
| 63 | C₅₉H₈₄N₁₂O₂₁ | 1297.38 | 1298.48 | 1 | C | 14.375 |
| 64 | C₅₅H₇₆N₁₂O₂₁ | 1241.28 | 1241.73 | 1 | C | 13.442 |
| 65 | C₈₀H₁₀₁N₁₃O₂₂ | 1596.76 | 1597.11 | 1 | G | 18.992 |
| 66 | C₇₆H₉₉N₁₃O₂₄ | 1578.70 | 1578.93 | 1 | C | 18.342 |
| 67 | C₇₇H₁₀₂N₁₄O₂₃ | 1591.74 | 1592.54 | 1 | C | 18.775 |
| 68 | C₆₉H₈₉N₁₁O₂₀ | 1392.53 | 1392.93 | 1 | C | 18.858 |
| 69 | C₇₁H₉₂N₁₂O₂₃ | 1481.58 | 1481.62 | 1 | C | 15.558 |
| 70 | C₇₇H₁₀₄N₁₂O₂₁ | 1533.74 | 1533.98 | 1 | F | 16.658 |
| 71 | C₇₁H₉₂N₁₂O₂₃ | 1481.58 | 1481.91 | 1 | C | 17.858 |
| 72 | C₆₃H₉₀N₁₄O₂₃ | 1411.49 | 1411.8 | 1 | C | 13.508 |
| 73 | C₇₅H₉₈N₁₄O₂₃ | 1563.68 | 1564.01 | 1 | C | 17.392 |
| 74 | C₇₃H₉₄N₁₂O₂₃ | 1507.62 | 1507.86 | 1 | G | 16.792 |
| 75 | C₇₉H₁₀₄N₁₄O₂₅ | 1649.77 | 1650.75 | 1 | C | 18.258 |
| 76 | C₇₁H₉₂N₁₂O₂₁ | 1449.58 | 1649.86 | 1 | C | 18.625 |
| 77 | C₇₇H₁₀₄N₁₂O₂₁ | 1533.74 | 1534.23 | 1 | G | 20.375 |
| 78 | C₄₉H₆₂N₈O₁₅ | 1003.08 | 1003.36 | 1 | C | 18.525 |
| 79 | C₅₁H₆₃N₉O₁₆ | 1058.11 | 1058.36 | 1 | C | 17.013 |
| 80 | C₅₃H₆₇N₉O₁₆ | 1086.17 | 1086.40 | 1 | E | 19.004 |
| 81 | C₅₁H₆₃N₉O₁₆ | 1058.11 | 1058.28 | 1 | C | 16.704 |
| 82 | C₅₃H₆₇N₉O₁₆ | 1086.17 | 1086.53 | 1 | C | 17.842 |
| 83 | C₃₉H₆₇N₇O₁₅ | 874.00 | 874.24 | 1 | C | 9.325 |
| 84 | C₄₈H₆₃N₇O₁₇ | 1010.06 | 1010.78 | 1 | E | 13.958 |
| 85 | C₄₆H₆₂N₈O₁₅ | 967.04 | 967.79 | 1 | E | 11.558 |
| 86 | C₄₉H₆₂F₆N₆O₁₅ | 1089.05 | 1089.36 | 1 | C | 24.325 |
| 87 | C₅₀H₆₄N₈O₁₅ | 1017.10 | 1018.46 | 1 | C | 19.225 |
| 88 | C₅₀H₆₄F₆N₆O₁₅ | 1103.08 | 1103.35 | 1 | E | 21.458 |
| 89 | C₅₃H₆₆N₁₀O₁₇ | 1115.16 | 1116.45 | 1 | E | 15.208 |
| 90 | C₅₃H₆₆N₁₀O₁₇ | 1115.16 | 1115.59 | 1 | E | 17.313 |
| 91 | C₅₅H₇₀N₁₀O₁₇ | 1143.22 | 1143.60 | 1 | E | 15.992 |
| 92 | C₅₅H₇₀N₁₀O₁₇ | 1143.22 | 1143.73 | 1 | C | 17.421 |
| 93 | C₄₉H₆₅N₇O₁₇ | 1024.09 | 1024.25 | 1 | E | 14.104 |
| 94 | C₄₈H₆₃N₇O₁₇ | 1010.06 | 1010.35 | 1 | E | 13.888 |
| 95 | C₄₉H₆₅N₇O₁₇ | 1024.09 | 1024.35 | 1 | E | 13.763 |
| 96 | C₅₀H₆₇N₇O₁₇ | 1038.12 | 1038.36 | 1 | E | 14.329 |
| 97 | C₅₀H₆₇N₇O₁₇ | 1038.12 | 1038.35 | 1 | E | 14.021 |
| 98 | C₅₄H₆₉N₁₁O₂₃ | 1240.20 | 1240.53 | 1 | C | 11.692 |
| 99 | C₇₃H₉₀N₁₄O₂₄ | 1547.60 | 775.01 | 2 | C | 17.025 |
| 100 | C₇₄H₈₉N₁₅O₂₄ | 1572.61 | 1572.89 | 1 | C | 18.758 |
| 101 | C₇₂H₈₆N₁₄O₂₁ | 1483.56 | 1483.70 | 1 | C | 19.725 |
| 102 | C₇₄H₈₉N₁₅O₂₆ | 1604.60 | 802.70 | 2 | E | 14.558 |
| 103 | C₅₀H₆₁N₁₁O₁₈ | 1104.10 | 1104.40 | 1 | E | 13.558 |
| 104 | C₇₃H₉₀N₁₄O₂₆ | 1579.60 | 790.54 | 2 | C | 16.208 |
| 105 | C₇₁H₈₇N₁₃O₂₃ | 1490.54 | 1491.70 | 1 | C | 17.442 |
| 106 | C₇₃H₉₀N₁₄O₂₈ | 1611.59 | 1612.80 | 1 | C | 12.875 |
| 107 | C₄₉H₆₂N₁₀O₂₀ | 1111.08 | 1112.04 | 1 | C | 11.775 |
| 108 | C₇₄H₈₈FN₁₅O₂₄ | 1590.60 | 796.1 | 2 | C | 19.025 |
| 109 | C₇₂H₈₅FN₁₄O₂₁ | 1501.55 | 1501.7 | 1 | C | 19.992 |
| 110 | C₇₄H₈₈FN₁₅O₂₆ | 1622.60 | 811.96 | 2 | E | 14.492 |
| 111 | C₅₀H₆₀FN₁₁O₁₈ | 1122.09 | 1122.38 | 1 | E | 14.042 |
| 112 | C₇₃H₈₉FN₁₄O₂₆ | 1597.59 | 799.26 | 2 | C | 16.558 |
| 113 | C₇₁H₈₆FN₁₃O₂₃ | 1508.54 | 754.84 | 2 | C | 17.792 |
| 114 | C₇₃H₈₉FN₁₄O₂₈ | 1629.58 | 815.4 | 2 | E | 12.675 |
| 115 | C₄₉H₆₁FN₁₀O₂₀ | 1129.08 | 1129.29 | 1 | E | 11.325 |
| 116 | C₇₃H₉₀N₁₄O₂₆ | 1579.60 | 791.17 | 2 | E | 14.592 |
| 117 | C₇₃H₉₀N₁₄O₂₈ | 1611.59 | 807.08 | 2 | E | 12.042 |
| 118 | C₅₄H₆₉N₁₁O₂₃ | 1240.20 | 1240.56 | 1 | C | 11.325 |
| 119 | C₆₃H₈₆N₁₄O₂₆ | 1455.45 | 728.36 | 2 | C | 11.008 |
| 120 | C₈₅H₁₁₀FN₁₇O₂₉ | 1852.90 | 926.87 | 2 | C | 14.575 |

The structure of the PSMA targeting ligands are shown in Table 1

### General Procedure for SPR Analysis.

The interaction of PSMA-binding variants with human FOLH1 was measured with Surface Plasmon Resonance (SPR) technology. Biotinylated FOLH1 was captured at a concentration of 30 μg/mL onto a Streptavidin chip.

Binding kinetics of the analytes were measured with a Biacore 8K instrument in two-fold serial dilutions starting at 500 nM down to 1.95 nM in single cycle kinetic measurements. After each cycle, needles were washed with 50% DMSO. To measure the association to the FOLH1 protein, the samples were injected with a flow rate of 30 μL/min for 90 s, followed by 1200 s buffer only to detect the dissociation. The used running buffer was 1×PBS with 0.05% Tween20 and 2% DMSO. The relative response units (RU, Y-axis) are plotted against time (s, X-axis) and analyzed in a kinetic 1:1 binding model.

In a different setup, biotinylated FOLH1 was captured at a concentration of 0.8 μg/mL and the running buffer was 1×PBS with 0.05% Tween20.

Table Below Shows Kd Data for the PSMA Targeting Ligands from SPR Analysis

| Compound | | | | | |
|---|---|---|---|---|---|
| No. | KD (nM) | Kon (1.ms) | Koff (1/s) | Ligand | Specie |
| 2 | 0.208 | 158000 | 0.000033 | PSMA | Human |
| 5 | 2.78 | 195000 | 0.000543 | PSMA | Human |
| 7 | 5.72 | 293000 | 0.00167 | PSMA | Human |
| 8 | 0.654 | 159000 | 0.000104 | PSMA | Human |
| 10 | 2.02 | 130000 | 0.000262 | PSMA | Human |
| 12 | 0.568 | 250000 | 0.000142 | PSMA | Human |
| 14 | 4.71 | 131000 | 0.000618 | PSMA | Human |
| 16 | 7.73 | 461000 | 0.00356 | PSMA | Human |
| 17 | 1.65 | 125000 | 0.000207 | PSMA | Human |
| 21 | 10.6 | 183000 | 0.00194 | PSMA | Human |
| 23 | 120 | 291000 | 0.0349 | PSMA | Human |
| 24 | 35.2 | 305000 | 0.0107 | PSMA | Human |
| 34 | 0.85 | 224000 | 0.000191 | PSMA | Human |
| 35 | 1.49 | 105000 | 0.000157 | PSMA | Human |
| 36 | 0.996 | 143000 | 0.000143 | PSMA | Human |
| 37 | 1.07 | 136000 | 0.000145 | PSMA | Human |
| 38 | 1.81 | 107000 | 0.000193 | PSMA | Human |
| 39 | 1.08 | 223000 | 0.00024 | PSMA | Human |
| 40 | 1.62 | 153000 | 0.000249 | PSMA | Human |
| 41 | 1.95 | 102000 | 0.000199 | PSMA | Human |
| 48 | 1650 | n.d | n.d | PSMA | Human |
| 49 | 3080 | n.d | n.d | PSMA | Human |
| 52 | 1.12 | 291000 | 0.000326 | PSMA | Human |
| 78 | 0.43 | 487000 | 0.000208 | PSMA | Human |
| 79 | 3.76 | 137000 | 0.000515 | PSMA | Human |
| 80 | 2.46 | 243000 | 0.000599 | PSMA | Human |
| 81 | 4.12 | 90000 | 0.000371 | PSMA | Human |
| 82 | 5.27 | 72200 | 0.000381 | PSMA | Human |
| 55 | 1.15 | 191000 | 0.00022 | PSMA | Human |
| 56 | 1.15 | 192000 | 0.000221 | PSMA | Human |
| 57 | 1.59 | 165000 | 0.000262 | PSMA | Human |
| 84 | 1.05 | 269000 | 0.000283 | PSMA | Human |
| 58 | 1.33 | 178000 | 0.000237 | PSMA | Human |
| 59 | 0.98 | 237000 | 0.000231 | PSMA | Human |
| 60 | 1.28 | 166000 | 0.000212 | PSMA | Human |
| 61 | 1.1 | 147000 | 0.000162 | PSMA | Human |
| 62 | 1.11 | 170000 | 0.000188 | PSMA | Human |
| 63 | 1.2 | 143000 | 0.000172 | PSMA | Human |
| 64 | 0.92 | 249000 | 0.00023 | PSMA | Human |
| 67 | 0.6 | 205000 | 0.000122 | PSMA | Human |
| 68 | 0.46 | 225000 | 0.000104 | PSMA | Human |
| 69 | 0.98 | 210000 | 0.000205 | PSMA | Human |
| 71 | 0.56 | 238000 | 0.000134 | PSMA | Human |
| 74 | 0.92 | 165000 | 0.000152 | PSMA | Human |
| 86 | 1.17 | 339000 | 0.000397 | PSMA | Human |
| 87 | 0.5 | 272000 | 0.000136 | PSMA | Human |
| 88 | 1.53 | 215000 | 0.000329 | PSMA | Human |
| 89 | 2.58 | 121000 | 0.000311 | PSMA | Human |
| 90 | 1.27 | 183000 | 0.000232 | PSMA | Human |
| 91 | 3.6 | 71500 | 0.000258 | PSMA | Human |
| 92 | 1.01 | 297000 | 0.000301 | PSMA | Human |
| 93 | 1.73 | 149000 | 0.000258 | PSMA | Human |
| 94 | 2.33 | 112000 | 0.000261 | PSMA | Human |
| 95 | 1.95 | 124000 | 0.000242 | PSMA | Human |
| 96 | 2.75 | 103000 | 0.000282 | PSMA | Human |
| 97 | 3.64 | 90100 | 0.000328 | PSMA | Human |
| 98 | 1.56 | 86800 | 0.000135 | PSMA | Human |
| 99 | 0.73 | 191000 | 0.00014 | PSMA | Human |
| 100 | 0.477 | 290000 | 0.000138 | PSMA | Human |
| 101 | 0.249 | 151000 | 3.75E−05 | PSMA | Human |
| 102 | 0.157 | 342000 | 5.36E−05 | PSMA | Human |
| 103 | 0.169 | 339000 | 5.73E−05 | PSMA | Human |
| 104 | 0.346 | 184000 | 6.35E−05 | PSMA | Human |
| 106 | 0.288 | 189000 | 5.44E−05 | PSMA | Human |
| 107 | 0.379 | 273000 | 0.000103 | PSMA | Human |
| 108 | 0.294 | 331000 | 9.74E−05 | PSMA | Human |
| 109 | 0.164 | 277000 | 4.56E−05 | PSMA | Human |
| 110 | 0.252 | 330000 | 8.32E−05 | PSMA | Human |
| 111 | 0.512 | 332000 | 0.00017 | PSMA | Human |
| 112 | 0.417 | 219000 | 9.13E−05 | PSMA | Human |
| 113 | 0.142 | 258000 | 3.67E−05 | PSMA | Human |
| 114 | 0.294 | 257000 | 7.55E−05 | PSMA | Human |
| 115 | 0.536 | 236000 | 0.000126 | PSMA | Human |
| 116 | 2.17 | 96700 | 0.00021 | PSMA | Human |
| 117 | 1.19 | 101000 | 0.00012 | PSMA | Human |
| 118 | 1.78 | 82300 | 0.000146 | PSMA | Human |
| 119 | 0.161 | 186000 | 0.00003 | PSMA | Human |
| 120 | 0.141 | 247000 | 0.0000346 | PSMA | Human |

### General Procedure for Inhibition Assay.

### Materials:

Amplex™ Red Glutamic Acid/Glutamate Oxidase Assay Kit Catalog number: A12221 (Thermofisher); Recombinant Human PSMA/FOLH1 Protein, CF (rnd systems); N-Acetyl-Asp-Glu (NAAG) (Sigma Aldrich; 96 well half area plate; Enspire plate reader.

### Procedure

Prepare a working solution of 100 μM Amplex® Red reagent containing 0.25 U/mL HRP, 0.08 U/mL L-glutamate oxidase, 0.5 U/mL 1-glutamate-pyruvate transaminase, 200 μM L-alanine and keep protected from light. Add 10 μl/well of the PSMA solution at 25 nM. Final concentration is 5 nM. Transfer 10 μl/well of the inhibitor solution previously prepared in a separate 96 wells/plate. Incubate PSMA and inhibitors for 20′ at 37° C. in the dark After, add 10 μl of substrate solution NAAG to have a final concentration of 40 μM.

Pipette 20 μl/well of the working solution prepared above. Incubate the reactions for 30 min at 37° C., protected from light. Because the assay is continuous (not terminated), fluorescence may be measured at multiple time points to follow the kinetics of the reactions. Measure the fluorescence in a fluorescence microplate reader using excitation in the range of 530-560 nm and emission detection at ˜590 nm. Ec50s were determined with GraphPad Prism. Kᵢ is calculated based on Cheng-Prusoff equation.

| Com- | | | | |
|---|---|---|---|---|
| pound | IC50 | Ki | | |
| No. | (nM) | (nM) | Target | Average |
| 1 | 2394.333 | 17.11596 | PSMA | Avg (N = 3) |
| 2 | 25.124 | 0.1796 | PSMA | Avg (N = 4) |
| 3 | 420.9333 | 3.009055 | PSMA | Avg (N = 3) |
| 4 | 536.7667 | 3.837093 | PSMA | Avg (N = 3) |
| 5 | 81.7375 | 0.584303 | PSMA | Avg (N = 4) |
| 6 | 58240.5 | 416.334 | PSMA | Avg (N = 2) |
| 7 | 171.4 | 1.225258 | PSMA | Avg (N = 3) |
| 8 | 29.33115 | 0.209675 | PSMA | Avg (N = 13) |
| 9 | 1082.967 | 7.74162 | PSMA | Avg (N = 3) |
| 10 | 21.256 | 0.151949 | PSMA | Avg (N = 5) |
| 11 | 3239.4 | 23.15695 | PSMA | Avg (N = 2) |
| 12 | 41.52125 | 0.296816 | PSMA | Avg (N = 4) |
| 13 | 951.1 | 6.798967 | PSMA | Avg (N = 2) |
| 14 | 286.5 | 2.048054 | PSMA | Avg (N = 2) |
| 15 | 934.6 | 6.681017 | PSMA | Avg (N = 2) |
| 16 | 331.295 | 2.368273 | PSMA | Avg (N = 2) |
| 17 | 92.59075 | 0.661888 | PSMA | Avg (N = 4) |
| 18 | 539.605 | 3.857383 | PSMA | Avg (N = 2) |
| 19 | 2159 | 15.43368 | PSMA | Avg (N = 2) |
| 20 | 1494.6 | 10.6842 | PSMA | Avg (N = 2) |
| 21 | 200.965 | 1.436605 | PSMA | Avg (N = 2) |
| 22 | 487.85 | 3.487411 | PSMA | Avg (N = 2) |
| 23 | 7005.5 | 50.07903 | PSMA | Avg (N = 2) |
| 24 | 3353 | 23.96902 | PSMA | Avg (N = 2) |
| 25 | 44559.5 | 318.535 | PSMA | Avg (N = 2) |
| 26 | 1545431 | 11047.56 | PSMA | Avg (N = 2) |
| 27 | 191123.5 | 1366.252 | PSMA | Avg (N = 2) |
| 28 | 21272.5 | 152.0671 | PSMA | Avg (N = 2) |
| 29 | 576064.5 | 4118.014 | PSMA | Avg (N = 2) |
| 30 | 602067 | 4303.894 | PSMA | Avg (N = 2) |
| 31 | 260351 | 1861.127 | PSMA | Avg (N = 2) |
| 32 | 433756.5 | 3100.722 | PSMA | Avg (N = 2) |
| 33 | 583118 | 4168.437 | PSMA | Avg (N = 2) |
| 34 | 243.05 | 1.73745 | PSMA | Avg (N = 2) |
| 35 | 158.62 | 1.1339 | PSMA | Avg (N = 2) |
| 36 | 7.014667 | 0.050145 | PSMA | Avg (N = 3) |
| 37 | 11.788 | 0.084267 | PSMA | Avg (N = 2) |
| 38 | 49.66 | 0.354996 | PSMA | Avg (N = 2) |
| 39 | 50.48 | 0.360858 | PSMA | Avg (N = 2) |
| 40 | 154.6 | 1.105163 | PSMA | Avg (N = 2) |
| 41 | 88.07 | 0.629571 | PSMA | Avg (N = 2) |
| 42 | 2240.85 | 16.01879 | PSMA | Avg (N = 2) |
| 43 | 679.3 | 4.855997 | PSMA | Avg (N = 2) |
| 44 | 177.2 | 1.26672 | PSMA | Avg (N = 3) |
| 45 | 554.4667 | 3.963622 | PSMA | Avg (N = 3) |
| 46 | 445.9667 | 3.188006 | PSMA | Avg (N = 3) |
| 47 | 2057.4 | 14.70739 | PSMA | Avg (N = 2) |
| 48 | 10286.5 | 73.53336 | PSMA | Avg (N = 2) |
| 49 | 16539 | 118.2295 | PSMA | Avg (N = 2) |
| 50 | 17644.5 | 126.1322 | PSMA | Avg (N = 2) |
| 51 | 1706.168 | 12.19659 | PSMA | Avg (N = 4) |
| 52 | 26.141 | 0.18687 | PSMA | Avg (N = 4) |
| 53 | 11.21033 | 0.080131 | PSMA | Avg (N = 6) |
| 54 | 88.9525 | 0.63588 | PSMA | Avg (N = 4) |
| 55 | 3032.948 | 21.68112 | PSMA | Avg (N = 5) |
| 56 | 150.2367 | 1.073971 | PSMA | Avg (N = 3) |
| 57 | 130.9767 | 0.936291 | PSMA | Avg (N = 3) |
| 58 | 120.8123 | 0.863631 | PSMA | Avg (N = 3) |
| 59 | 15.3282 | 0.109574 | PSMA | Avg (N = 2) |
| 60 | 3.484714 | 0.020724 | PSMA | Avg (N = 7) |
| 61 | 3.739886 | 0.026735 | PSMA | Avg (N = 7) |
| 62 | 3.6697 | 0.03042 | PSMA | Avg (N = 7) |
| 63 | 70265.33 | 502.2939 | PSMA | Avg (N = 3) |
| 64 | 10.407 | 0.074395 | PSMA | Avg (N = 3) |
| 65 | 108.7987 | 0.777751 | PSMA | Avg (N = 3) |
| 66 | 1.829 | 0.013075 | PSMA | Avg (N = 2) |
| 67 | 2.7764 | 0.019847 | PSMA | Avg (N = 3) |
| 68 | 2.29205 | 0.016385 | PSMA | Avg (N = 4) |
| 69 | 1.6292 | 0.011646 | PSMA | Avg (N = 4) |
| 70 | 1.362675 | 0.009741 | PSMA | Avg (N = 4) |
| 71 | 5.177164 | 0.037009 | PSMA | Avg (N = 5) |
| 72 | 1.4069 | 0.010057 | PSMA | Avg (N = 4) |
| 73 | 8.0784 | 0.057749 | PSMA | Avg (N = 3) |
| 74 | 5.725867 | 0.040932 | PSMA | Avg (N = 3) |
| 75 | 10.3775 | 0.074184 | PSMA | Avg (N = 2) |
| 76 | 5.483 | 0.039195 | PSMA | Avg (N = 2) |
| 77 | 1.555133 | 0.011117 | PSMA | Avg (N = 3) |
| 78 | 6.948833 | 0.049674 | PSMA | Avg (N = 3) |
| 79 | 7.791 | 0.055694 | PSMA | Avg (N = 2) |
| 80 | 2.346 | 0.01677 | PSMA | Avg (N = 2) |
| 81 | 10.67933 | 0.076342 | PSMA | Avg (N = 3) |
| 82 | 1.800233 | 0.012869 | PSMA | Avg (N = 3) |
| 83 | 2.8685 | 0.020506 | PSMA | Avg (N = 2) |
| 84 | 5.106667 | 0.036505 | PSMA | Avg (N = 3) |
| 85 | 10.61633 | 0.075891 | PSMA | Avg (N = 3) |
| 86 | 59.235 | 0.423441 | PSMA | Avg (N = 6) |
| 87 | 1479.294 | 10.57477 | PSMA | Avg (N = 6) |
| 88 | 63.27167 | 0.452287 | PSMA | Avg (N = 6) |
| 89 | 93.55 | 0.668745 | PSMA | Avg (N = 3) |
| 90 | 87.57667 | 0.626044 | PSMA | Avg (N = 3) |
| 91 | 26.14667 | 0.18691 | PSMA | Avg (N = 3) |
| 92 | 79.68 | 0.569595 | PSMA | Avg (N = 3) |
| 93 | 9.598 | 0.068612 | PSMA | Avg (N = 3) |
| 94 | 1.865767 | 0.013337 | PSMA | Avg (N = 3) |
| 95 | 11.517 | 0.08233 | PSMA | Avg (N = 3) |
| 96 | 7.943667 | 0.056786 | PSMA | Avg (N = 3) |
| 97 | 10.76867 | 0.07698 | PSMA | Avg (N = 3) |
| 98 | 1.3089 | 0.009357 | PSMA | Avg (N = 3) |
| 99 | 2.995333 | 0.021412 | PSMA | Avg (N = 3) |
| 100 | 0.385033 | 0.002752 | PSMA | Avg (N = 3) |
| 101 | 0.6677 | 0.004773 | PSMA | Avg (N = 2) |
| 102 | 1.5374 | 0.01099 | PSMA | Avg (N = 2) |
| 103 | 0.4351 | 0.00311 | PSMA | Avg (N = 3) |
| 104 | 0.288413 | 0.002062 | PSMA | Avg (N = 3) |
| 105 | 0.55134 | 0.003941 | PSMA | Avg (N = 3) |
| 106 | 0.29546 | 0.002112 | PSMA | Avg (N = 3) |
| 107 | 0.4762 | 0.003404 | PSMA | Avg (N = 5) |
| 108 | 1.1056 | 0.007903 | PSMA | Avg (N = 3) |
| 109 | 0.6394 | 0.004571 | PSMA | Avg (N = 3) |
| 110 | 1.360797 | 0.009728 | PSMA | Avg (N = 3) |
| 111 | 0.625033 | 0.004468 | PSMA | Avg (N = 3) |
| 112 | 0.706467 | 0.00505 | PSMA | Avg (N = 3) |
| 113 | 0.79335 | 0.005671 | PSMA | Avg (N = 4) |
| 114 | 0.5839 | 0.004174 | PSMA | Avg (N = 3) |
| 115 | 0.389967 | 0.002788 | PSMA | Avg (N = 3) |
| 116 | 0.279617 | 0.001999 | PSMA | Avg (N = 3) |
| 117 | 0.29257 | 0.002091 | PSMA | Avg (N = 3) |
| 118 | 0.3119 | 0.00223 | PSMA | Avg (N = 3) |
| 119 | 0.59495 | 0.004253 | PSMA | Avg (N = 2) |
| 120 | 0.3337 | 0.002385 | PSMA | Avg (N = 2) |

### Cellular Competition Assay on PSMA+ Cells

### Material

| | |
|---|---|
| FACS plates: | 96 well v bottom polypropylene |
| FACS Buffer | PBS−/− 2% FBS |
| Primary: | anti-IL2 MAB202 mIgG1 RnD #5334; |
| | Anti-IL-18 MBL #D044-3 |
| Secondary antibody: | anti-mIgG1 AF388 Biolegend #406626 |
| Live/Dead staining: | Zombie NIR 1:2000 in PBS |

### Procedure:

The 22rv1 (ATCC CRL-2505) human prostate carcinoma epithelial cell line carcinoma was cultured in RPMI medium supplemented with 10% FBS The PC3 cell line (ATCC CRL-1435) was cultured in F12K medium supplemented with 10% FBS. On the day of experiment, cells were harvested with Trypsin and washed with assay buffer consisting of 1×PBS supplemented with 2% FBS and stained with viability dye (Biolegend Zombie NIR) diluted 1:1000 in PBS for 20 minutes. After washing and counting, cells were seeded at 50,000 cells/well in a 96 well V bottom polypropylene plate and kept on ice. Twelve 4-fold serial dilutions of analytes were prepared in cold assay buffer and added to cell suspension. After incubating for 60 minutes on ice, saturation concentration of BPT1288 was added on cells and incubated for 60′. Then, fixed and washed cells were stained with 1 ug/mL solution of anti-IL18 and anti-IL2 antibodies (MBL #D044-3; RnD biosystems #MAB202) and detection was performed with anti-mIgG1 AF488-labelled secondary antibody (Biolegend #406626) at a concentration of 2.5 ug/mL in assay buffer.

Single-cell AlexaFluor488 fluorescence emission was measured with a flow cytometer and plotted against analyte concentration in X-axis. Half maximal inhibition concentrations (IC50) were calculated based on a variable slope and four parameter analysis using GraphPad PRISM software

| Compound | IC50 (nM) |
|---|---|
| 8 | 2.645 |
| BPT883 | 1.065 |
| 55 | 1.496 |
| 56 | 0.829 |
| 57 | 1.990 |
| 98 | 2.123 |
| 99 | 1.747 |
| 100 | 0.992 |
| 101 | 0.575 |
| 102 | 1.026 |
| 103 | 0.734 |
| 104 | 0.639 |
| 105 | 0.215 |
| 106 | 0.139 |
| 107 | 0.187 |
| 108 | 0.368 |
| 109 | 0.128 |
| 110 | 0.149 |
| 111 | 0.111 |
| 112 | 0.181 |
| 113 | 0.099 |
| 114 | 0.125 |
| 115 | 1.003 |
| 116 | 1.256 |
| 117 | 1.259 |
| 118 | 1.000 |
| 119 | 1.418 |
| 120 | 0.804. |

### Abbreviations Used in Examples

Aa amino acid
Aad aminoadipic acid
Alloc allyloxycarbonyl
AMCHC (1r,4r)-4-(aminomethyl)cyclohexane-1-carboxylic acid
Bip (4-biphenylyl)-alanine
Boc tert-butyloxycarbonyl
° C. degree Celsius
CDI carbonyldiimidazole
Cha cyclohexylalanine
DCM dichloromethane
DEE diethyl ether
DIPEA N,N-diisopropylethylamine
DMF N,N-dimethylformamide
DMSO dimethyl sulfoxide
EDC 1-ethyl-3-(3-dimethylaminopropyl)carbodiimide
EuK glutamate-urea-lysine
equiv equivalent
FOLH1 folate hydrolase 1
Fmoc 9-fluorenylmethyloxycarbonyl
h hour
GABA gamma-aminobutyric acid
HATU O-(7-azabenzotriazole-1-yl)-N,N,N′,N′-tetramethyluronium hexafluorophosphate
HFIP 1,1,1,3,3,3-hexafluoroisopropanol
HPLC high-performance liquid chromatography
IPA isopropyl alcohol
Kᵢ inhibitor constant
LC-MS liquid chromatography-mass spectrometry
M molecular mass
M mole per litter
m/z mass-to-charge ratio
Me methyl
MeOH methanol
min minute
MW molecular weight
N^{o} number
NAAG N-acetylaspartylglutamate
Nal naphthylalanine
NMM 4-methylmorpholine
Pal pyridylalanine
PBS phosphate-buffered saline
PEG polyethylene glycol
PG protecting group
Pra propargylglycine
PSMA prostate-specific membrane antigen
r.t. room temperature
RT retention time
RP reverse-phase
SPPS solid-phase peptide synthesis
SPR surface plasmon resonance
^{t}Bu tert-butyl
TFA trifluoroacetic acid
TIS triisopropylsilane
Trt trityl
v/v volume per volume percentage concentration

Although the present disclosure and its advantages have been described in detail, it should be understood that various changes, substitutions and alterations can be made herein without departing from the spirit and scope of the disclosure as defined in the appended claims.

1. A prostate-specific membrane antigen (PSMA) binding ligand having a structure of Formula (I): 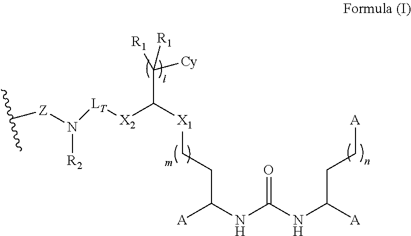
wherein:
each A is independently selected from carboxylic acid, sulphonic acid, phosphonic acid, tetrazole, or isoxazole;
m is an integer from 0 to 6;
n is an integer from 0 to 6;
l is an integer from 0 to 6;
Cy is optionally substituted cycloalkyl, optionally substituted heterocycloalkyl, optionally substituted aryl, or optionally substituted heteroaryl;
each of X₁ and X₂ is independently —N(R)C(═O)—, —C(═O)N(R)—, —C(═O)O—, —OC(═O)N(R)—, —N(R)C(═O)O—, or —N(R)C(═O)N(R)—;
L_{T} is optionally substituted C₁ to C₂₀ alkylene, optionally substituted C₁ to C₂₀ heteroalkylene, 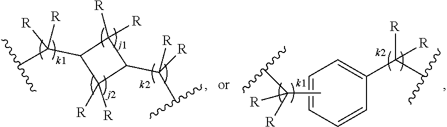
wherein
k1 and k2 are each independently an integer from 0 to 6; and
j1 and j2 are each independently an integer from 1 to 4
Z is a peptide of at least two amino acids or a poly(ethylene glycol) group;
each R is independently selected from H and optionally substituted alkyl;
each R₁ and R₂ is independently selected from H and optionally substituted alkyl; and 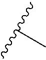
denotes a point of attachment to an additional group, optionally by a linker;
or a pharmaceutically acceptable salt, or solvate thereof.

2. The ligand of claim 1, wherein each A is independently carboxylic acid.

3. (canceled)

4. The ligand of claim 1, wherein m is 3 and n is 1.

5-7. (canceled)

8. The ligand of claim 1, wherein l is 1.

9-12. (canceled)

13. The ligand of claim 1, wherein Cy is

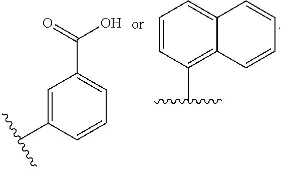 [SMILES: *c1cccc(C(=O)O)c1 ; *c1cccc2ccccc12]

14-16. (canceled)

17. The ligand of claim 1, wherein Cy is

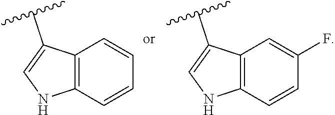 [SMILES: *c1c[nH]c2ccccc12 ; *c1c[nH]c2ccc(F)cc12]

18. The ligand of claim 1, wherein X₁ is —N(R)C(═O)— or —C(═O)N(R)—.

19. The ligand of claim 1, wherein X₂ is —N(R)C(═O)— or —C(═O)N(R)—.

20. The ligand of claim 1, wherein L_{T} is 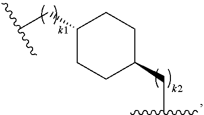
and wherein k1 is 1 and k2 is 0.

21-24. (canceled)

25. The ligand of claim 1, wherein L_{T} is —CH₂—.

26. The ligand of claim 1, wherein reach R and R₁ is independently H or methyl.

27. The ligand of claim 1, wherein each R and R₁ is H.

28. The ligand of claim 1, wherein R₂ is H.

29. The ligand of claim 1, wherein R₂ is C₁-C₆ alkyl substituted with an optionally substituted phenoxy.

30-53. (canceled)

54. The ligand of claim 1, wherein Z is the peptide and comprises any one of the following amino acid sequences:
(SEQ ID NO: 33)
AAAAAA,
(SEQ ID NO: 34)
(D-Ala)-(D-Ala)-(D-Ala)-(D-Ala)-(D-Ala)-(D-Ala),
(SEQ ID NO: 35)
AAAAEEG,
(SEQ ID NO: 36)
AAAEE,
(SEQ ID NO: 37)
AAAEEE,
(SEQ ID NO: 38)
AAAEEEG,
(SEQ ID NO: 39)
AAEE,
(SEQ ID NO: 40)
AAEEG,
(SEQ ID NO: 41)
AAFFAAEE,
(SEQ ID NO: 42)
AAFFAAEEG,
(SEQ ID NO: 43)
AAFFAEEE,
(SEQ ID NO: 44)
AAFFAEEEG,
(SEQ ID NO: 45)
AAQQEE,
(SEQ ID NO: 46)
AAQQEEG,
(SEQ ID NO: 47)
AEE, EE, EEE, EEEG,
(SEQ ID NO: 48)
EEEG(3-caroboxy-phenylalanine),
(SEQ ID NO: 49)
EEEG(4-carboxy-phenylalanine),
(SEQ ID NO: 103)
EEG, EEG(3-carboxy-phenylalanine),
(SEQ ID NO: 50)
FAAEE,
(SEQ ID NO: 51)
FAAEEG,
(SEQ ID NO: 52)
FAFAEE,
(SEQ ID NO: 53)
FAFAEEG,
(SEQ ID NO: 10)
FFAAEE,
(SEQ ID NO: 78)
FFAAEE(3-carboxy-phenylalanine),
(SEQ ID NO: 54)
FFAAEEE,
(SEQ ID NO: 55)
FFAAEEEG,
(SEQ ID NO: 9)
FFAAEEG,
(SEQ ID NO: 56)
FFAAEEG(3-carboxy-phenylalanine),
(SEQ ID NO: 57)
FFAEEE,
(SEQ ID NO: 58)
FFAEEEG,
(SEQ ID NO: 59)
FFFAAEE,
(SEQ ID NO: 60)
FFFAAEEG,
(SEQ ID NO: 61)
FFLLEE,
(SEQ ID NO: 62)
FFLLEEG,
(SEQ ID NO: 63)
FFQQEE,
(SEQ ID NO: 64)
FFQQEEG,
(SEQ ID NO: 65)
FFSSEE,
(SEQ ID NO: 66)
FFSSEEG,
(SEQ ID NO: 67)
FFSSEEG(3-carboxy-phenylalanine),
(SEQ ID NO: 68)
FFSSEEG(4-carboxy-phenylalanine),
(SEQ ID NO: 69)
FLFLEE,
(SEQ ID NO: 70)
FLFLEEG,
(SEQ ID NO: 71)
GGGGEE,
(SEQ ID NO: 72)
GGGGEEG,
(SEQ ID NO: 73)
PPPPPP,
(SEQ ID NO: 18)
YYAAEE,
(SEQ ID NO: 17)
YYAAEEG,
(SEQ ID NO: 74)
YYSSEE,
(SEQ ID NO: 75)
YYSSEEG,
(SEQ ID NO: 76)
YYSSEEG(3-carboxy-phenylalanine)
or
(SEQ ID NO: 77)
YYSSEEG(4-carboxy-phenylalanine).

55. The ligand of claim 1, wherein the ligand comprises any of the following structures (SEQ ID NOS 110, 108-109, 100, and 94 disclosed below, respectively, in order of appearance):

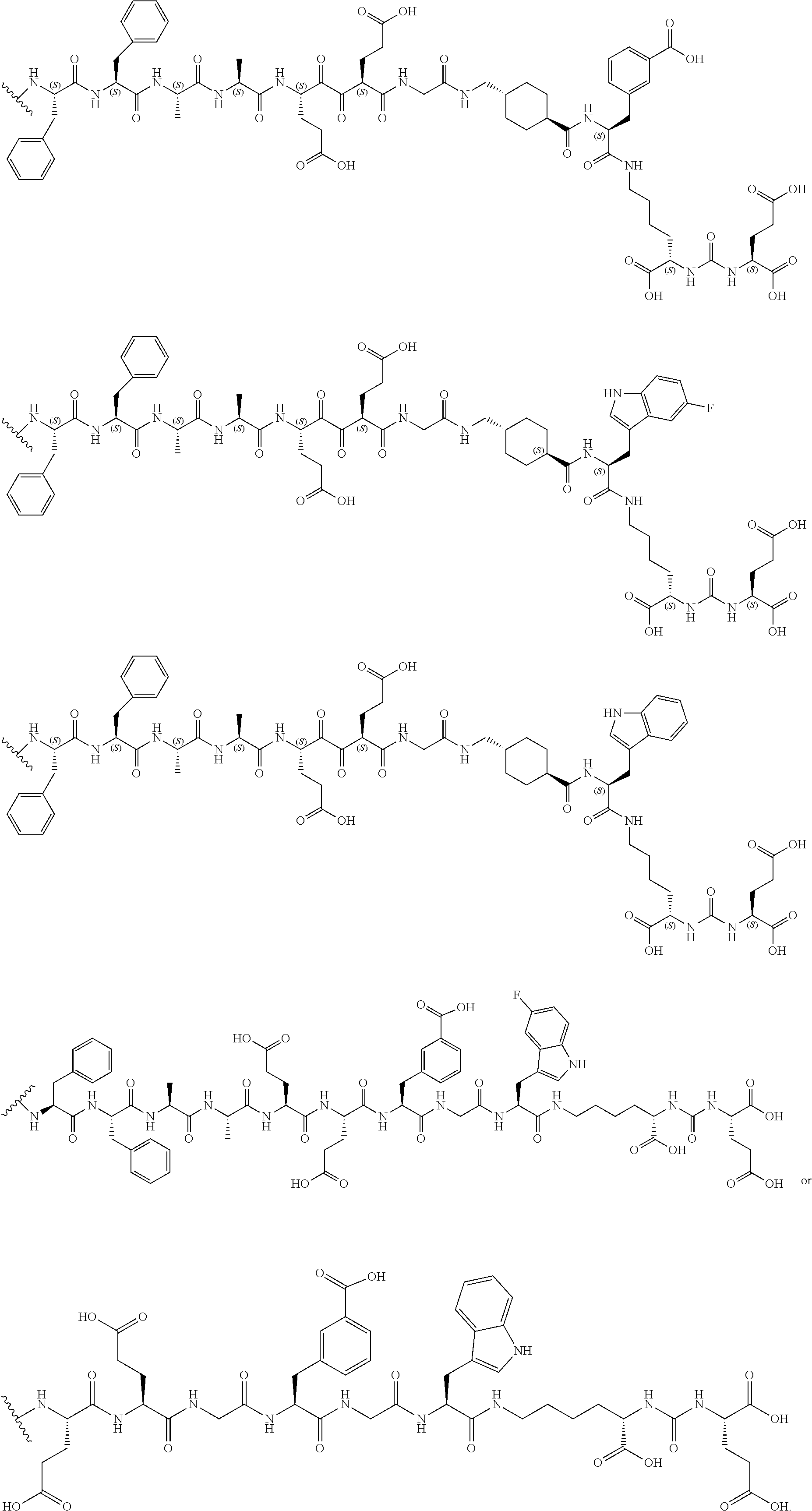

56. The ligand of claim 1, wherein Z comprises the poly(ethylene glycol).

57. The ligand of claim 56, wherein the poly(ethylene glycol) has a structure 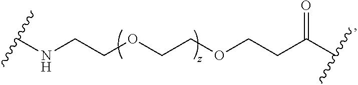
wherein the carboxyl group is attached to the NR₂ group of the ligand, z is an integer from 1 to 100, and the NH group forms the point of attachment to the additional group or the linker.

58-61. (canceled)

62. The ligand of claim 1, wherein the additional group comprises a chelator optionally comprising a radionuclide.

63-65. (canceled)

66. The ligand of claim 62, wherein the chelator is 1,4,7,10-tetraazacyclododecane-1,4,7,10-tetraacetic acid (DOTA), 1,4,7,10-tetraazacyclododecane-1,4,7,10-tetrapropionic acid (DOTPA), 1,4,7,10-tetraazacyclododecane-1,4,7,10-tetramethylene phosphoric acid (DOTMP), Hydroxypropyltetraazacyclododecane triacetic acid (HP-DO3A), (1R,4R,7R,10R)-α,α′,α″,α′″-tetramethyl-1,4,7,10-tetraazacyclododecane-1,4,7,10-tetraacetic acid (DOTAMA), 1,4,7,10-tetrakis(carbamoylmethyl)-1,4,7,10-tetraazacyclododecane (DOTAA), 1,4,7,10-tetraazacyclododecane-1,4,7,10-tetrakis(acetamidomethylene) phosphonic acid (DOTA-A-AMP), tetraazacyclododecane dimethane phosphonic acid (DO2P), α-(2-carboxyethyl)-1,4,7,10-tetraazacyclododecane-1,4,7,10-tetraacetic acid (DOTAGA), N,N′,N″,N′″-tetra(1,2-dihydro-1-hydroxy-2-oxopyridine-6-carbonyl)-1,5,10,14-tetraazatetradodecane (1,2-HOPO), 1,4,8,11-tetraazacyclotetradecane-1,4,8,11-tetraacetic acid (TETA), 1,4,8,11-tetraazacyclotetradecane-1,4,8,11-tetrapropionic acid (TETPA), 1,4,7,10,13-pentaazacyclopentadecane-N,N′,N″,N′″,N″″-pentaacetic acid (PEPA), or 2-[4,7-bis(carboxymethyl)-1,4,7-triazonan-1-yl] acetic acid (NOTA).


SEQUENCE LISTING (112 sequences)

SEQ ID NO: 1 - protein, 20 aa, synthetic construct
  VARIANT 1..20: note = This sequence may encompass 1-10 GS repeating units
GSGSGSGSGSGSGSGSGSGS

SEQ ID NO: 2 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 3 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 4 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 5 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 6 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 7 - protein, 15 aa, synthetic construct
GGGGSGGGGSGGGGS

SEQ ID NO: 8 - protein, 20 aa, synthetic construct
GGGGSGGGGSGGGGSGGGGS

SEQ ID NO: 9 - protein, 7 aa, synthetic construct
FFAAEEG

SEQ ID NO: 10 - protein, 6 aa, synthetic construct
FFAAEE

SEQ ID NO: 11 - protein, 7 aa, synthetic construct
FFAADDG

SEQ ID NO: 12 - protein, 6 aa, synthetic construct
FFAADD

SEQ ID NO: 13 - protein, 7 aa, synthetic construct
FFAAQQG

SEQ ID NO: 14 - protein, 6 aa, synthetic construct
FFAAQQ

SEQ ID NO: 15 - protein, 7 aa, synthetic construct
FFAANNG

SEQ ID NO: 16 - protein, 6 aa, synthetic construct
FFAANN

SEQ ID NO: 17 - protein, 7 aa, synthetic construct
YYAAEEG

SEQ ID NO: 18 - protein, 6 aa, synthetic construct
YYAAEE

SEQ ID NO: 19 - protein, 7 aa, synthetic construct
YYAADDG

SEQ ID NO: 20 - protein, 6 aa, synthetic construct
YYAADD

SEQ ID NO: 21 - protein, 7 aa, synthetic construct
YYAAQQG

SEQ ID NO: 22 - protein, 6 aa, synthetic construct
YYAAQQ

SEQ ID NO: 23 - protein, 7 aa, synthetic construct
YYAANNG

SEQ ID NO: 24 - protein, 6 aa, synthetic construct
YYAANN

SEQ ID NO: 25 - protein, 7 aa, synthetic construct
WWAAEEG

SEQ ID NO: 26 - protein, 6 aa, synthetic construct
WWAAEE

SEQ ID NO: 27 - protein, 7 aa, synthetic construct
WWAADDG

SEQ ID NO: 28 - protein, 6 aa, synthetic construct
WWAADD

SEQ ID NO: 29 - protein, 7 aa, synthetic construct
WWAAQQG

SEQ ID NO: 30 - protein, 6 aa, synthetic construct
WWAAQQ

SEQ ID NO: 31 - protein, 7 aa, synthetic construct
WWAANNG

SEQ ID NO: 32 - protein, 6 aa, synthetic construct
WWAANN

SEQ ID NO: 33 - protein, 6 aa, synthetic construct
AAAAAA

SEQ ID NO: 34 - protein, 6 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
AAAAAA

SEQ ID NO: 35 - protein, 7 aa, synthetic construct
AAAAEEG

SEQ ID NO: 36 - protein, 5 aa, synthetic construct
AAAEE

SEQ ID NO: 37 - protein, 6 aa, synthetic construct
AAAEEE

SEQ ID NO: 38 - protein, 7 aa, synthetic construct
AAAEEEG

SEQ ID NO: 39 - protein, 4 aa, synthetic construct
AAEE

SEQ ID NO: 40 - protein, 5 aa, synthetic construct
AAEEG

SEQ ID NO: 41 - protein, 8 aa, synthetic construct
AAFFAAEE

SEQ ID NO: 42 - protein, 9 aa, synthetic construct
AAFFAAEEG

SEQ ID NO: 43 - protein, 8 aa, synthetic construct
AAFFAEEE

SEQ ID NO: 44 - protein, 9 aa, synthetic construct
AAFFAEEEG

SEQ ID NO: 45 - protein, 6 aa, synthetic construct
AAQQEE

SEQ ID NO: 46 - protein, 7 aa, synthetic construct
AAQQEEG

SEQ ID NO: 47 - protein, 4 aa, synthetic construct
EEEG

SEQ ID NO: 48 - protein, 5 aa, synthetic construct
  SITE 5: note = 3-carboxy-phenylalanine
EEEGF

SEQ ID NO: 49 - protein, 5 aa, synthetic construct
  SITE 5: note = 4-carboxy-phenylalanine
EEEGF

SEQ ID NO: 50 - protein, 5 aa, synthetic construct
FAAEE

SEQ ID NO: 51 - protein, 6 aa, synthetic construct
FAAEEG

SEQ ID NO: 52 - protein, 6 aa, synthetic construct
FAFAEE

SEQ ID NO: 53 - protein, 7 aa, synthetic construct
FAFAEEG

SEQ ID NO: 54 - protein, 7 aa, synthetic construct
FFAAEEE

SEQ ID NO: 55 - protein, 8 aa, synthetic construct
FFAAEEEG

SEQ ID NO: 56 - protein, 8 aa, synthetic construct
  SITE 8: note = 3-carboxy-phenylalanine
FFAAEEGF

SEQ ID NO: 57 - protein, 6 aa, synthetic construct
FFAEEE

SEQ ID NO: 58 - protein, 7 aa, synthetic construct
FFAEEEG

SEQ ID NO: 59 - protein, 7 aa, synthetic construct
FFFAAEE

SEQ ID NO: 60 - protein, 8 aa, synthetic construct
FFFAAEEG

SEQ ID NO: 61 - protein, 6 aa, synthetic construct
FFLLEE

SEQ ID NO: 62 - protein, 7 aa, synthetic construct
FFLLEEG

SEQ ID NO: 63 - protein, 6 aa, synthetic construct
FFQQEE

SEQ ID NO: 64 - protein, 7 aa, synthetic construct
FFQQEEG

SEQ ID NO: 65 - protein, 6 aa, synthetic construct
FFSSEE

SEQ ID NO: 66 - protein, 7 aa, synthetic construct
FFSSEEG

SEQ ID NO: 67 - protein, 8 aa, synthetic construct
  SITE 8: note = 3-carboxy-phenylalanine
FFSSEEGF

SEQ ID NO: 68 - protein, 8 aa, synthetic construct
  SITE 8: note = 4-carboxy-phenylalanine
FFSSEEGF

SEQ ID NO: 69 - protein, 6 aa, synthetic construct
FLFLEE

SEQ ID NO: 70 - protein, 7 aa, synthetic construct
FLFLEEG

SEQ ID NO: 71 - protein, 6 aa, synthetic construct
GGGGEE

SEQ ID NO: 72 - protein, 7 aa, synthetic construct
GGGGEEG

SEQ ID NO: 73 - protein, 6 aa, synthetic construct
PPPPPP

SEQ ID NO: 74 - protein, 6 aa, synthetic construct
YYSSEE

SEQ ID NO: 75 - protein, 7 aa, synthetic construct
YYSSEEG

SEQ ID NO: 76 - protein, 8 aa, synthetic construct
  SITE 8: note = 3-carboxy-phenylalanine
YYSSEEGF

SEQ ID NO: 77 - protein, 8 aa, synthetic construct
  SITE 8: note = 4-carboxy-phenylalanine
YYSSEEGF

SEQ ID NO: 78 - protein, 7 aa, synthetic construct
  SITE 7: note = 3-carboxy-phenylalanine
FFAAEEF

SEQ ID NO: 79 - protein, 7 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
AAAAAAG

SEQ ID NO: 80 - protein, 7 aa, synthetic construct
AAAAAAG

SEQ ID NO: 81 - protein, 7 aa, synthetic construct
PPPPPPG

SEQ ID NO: 82 - protein, 4 aa, synthetic construct
AEEG

SEQ ID NO: 83 - protein, 7 aa, synthetic construct
FFIIEEG

SEQ ID NO: 84 - protein, 7 aa, synthetic construct
FIFIEEG

SEQ ID NO: 85 - protein, 7 aa, synthetic construct
  SITE 5: note = 3-carboxy-phenylalanine
EEEGFGW

SEQ ID NO: 86 - protein, 10 aa, synthetic construct
  SITE 8: note = 3-carboxy-phenylalanine
FFAAEEGFGW

SEQ ID NO: 87 - protein, 9 aa, synthetic construct
FFSSEEGGW

SEQ ID NO: 88 - protein, 8 aa, synthetic construct
FFAAEEGW

SEQ ID NO: 89 - protein, 9 aa, synthetic construct
YYSSEEGGW

SEQ ID NO: 90 - protein, 5 aa, synthetic construct
EEGGW

SEQ ID NO: 91 - protein, 10 aa, synthetic construct
  SITE 8: note = 3-carboxy-phenylalanine
FFSSEEGFGW

SEQ ID NO: 92 - protein, 9 aa, synthetic construct
  SITE 7: note = 3-carboxy-phenylalanine
FFAAEEFGW

SEQ ID NO: 93 - protein, 10 aa, synthetic construct
  SITE 8: note = 3-carboxy-phenylalanine
YYSSEEGFGW

SEQ ID NO: 94 - protein, 6 aa, synthetic construct
  SITE 4: note = 3-carboxy-phenylalanine
EEGFGW

SEQ ID NO: 95 - protein, 9 aa, synthetic construct
  SITE 9: note = 5-fluoro-tryptophan
FFSSEEGGX

SEQ ID NO: 96 - protein, 8 aa, synthetic construct
  SITE 8: note = 5-fluoro-tryptophan
FFAAEEGX

SEQ ID NO: 97 - protein, 9 aa, synthetic construct
  SITE 9: note = 5-fluoro-tryptophan
YYSSEEGGX

SEQ ID NO: 98 - protein, 5 aa, synthetic construct
  SITE 5: note = 5-fluoro-tryptophan
EEGGX

SEQ ID NO: 99 - protein, 10 aa, synthetic construct
  SITE 8: note = 3-carboxy-phenylalanine
  SITE 10: note = 5-fluoro-tryptophan
FFSSEEGFGX

SEQ ID NO: 100 - protein, 9 aa, synthetic construct
  SITE 7: note = 3-carboxy-phenylalanine
  SITE 9: note = 5-fluoro-tryptophan
FFAAEEFGX

SEQ ID NO: 101 - protein, 10 aa, synthetic construct
  SITE 8: note = 3-carboxy-phenylalanine
  SITE 10: note = 5-fluoro-tryptophan
YYSSEEGFGX

SEQ ID NO: 102 - protein, 6 aa, synthetic construct
  SITE 4: note = 3-carboxy-phenylalanine
  SITE 6: note = 5-fluoro-tryptophan
EEGFGX

SEQ ID NO: 103 - protein, 4 aa, synthetic construct
  SITE 4: note = 3-carboxy-phenylalanine
EEGF

SEQ ID NO: 104 - protein, 7 aa, synthetic construct
  SITE 5: note = 4-carboxy-phenylalanine
EEEGFGW

SEQ ID NO: 105 - protein, 10 aa, synthetic construct
  SITE 8: note = 4-carboxy-phenylalanine
FFSSEEGFGW

SEQ ID NO: 106 - protein, 10 aa, synthetic construct
  SITE 8: note = 4-carboxy-phenylalanine
YYSSEEGFGW

SEQ ID NO: 107 - protein, 7 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
  SITE 7: note = D-amino acid
FFAAEEG

SEQ ID NO: 108 - protein, 7 aa, synthetic construct
FFAAEEG

SEQ ID NO: 109 - protein, 7 aa, synthetic construct
FFAAEEG

SEQ ID NO: 110 - protein, 7 aa, synthetic construct
FFAAEEG

SEQ ID NO: 111 - protein, 6 aa, synthetic construct
  SITE 4: note = 3-carboxy-phenylalanine
EEGFGW

SEQ ID NO: 112 - protein, 9 aa, synthetic construct
  SITE 7: note = 3-carboxy-phenylalanine
  SITE 9: note = 5-fluoro-tryptophan
FFAAEEFGX